(19) United States
(12) Patent Application Publication
(10) Pub. No.: US 2026/0183393 A1
(43) Pub. Date: Jul. 2, 2026

(54) IMMUNOSUPPRESSANT-RESISTANT MODIFIED ALLOGENEIC MODIFIED IMMUNE CELLS AND METHODS FOR USE THEREOF

(71) Applicant: Beam Therapeutics Inc., Cambridge, MA (US)

(72) Inventors: Angelica Messana, Cambridge, MA (US); Yinmeng Yang, Cambridge, MA (US); Colby Maldini, Cambridge, MA (US)

(73) Assignee: Beam Therapeutics Inc., Cambridge, MA (US)

(21) Appl. No.: 19/331,836

(22) Filed: Sep. 17, 2025

Related U.S. Application Data
(63) Continuation of application No. PCT/US2024/020699, filed on Mar. 20, 2024.
(60) Provisional application No. 63/491,364, filed on Mar. 21, 2023.
(60) Provisional application No. 63/621,953, filed on Jan. 17, 2024.
(60) Provisional application No. 63/563,135, filed on Mar. 8, 2024.

(51) Int. Cl. A61K 40/11; A61K 31/56; A61K 35/17; A61K 40/31; A61K 40/42; A61K 40/50; A61K 45/06; A61P 35/00; C12N 5/0783; C12N 5/10; C12N 15/11; C12N 15/113

(52) U.S. Cl. CPC A61K 40/11; A61K 31/56; A61K 35/17; A61K 40/31; A61K 40/42; A61K 40/50; A61K 45/06; A61P 35/00; C12N 5/0636; C12N 5/10; C12N 15/111; C12N 15/113; C12N 2510/00

(57) ABSTRACT

As described below, the present disclosure features modified immune effector cells (e.g., T or NK cells) having increased resistance to inhibition by immunosuppressant agents relative to unmodified immune effector cells, compositions containing the same, and methods for use thereof.

### CROSS REFERENCE TO RELATED APPLICATIONS

This application is a continuation under 35 U.S.C. § 111(a) of PCT International Patent Application No. PCT/US2024/020699, filed Mar. 20, 2024, designating the United States and published in English, which claims priority to and the benefit of U.S. Provisional Application No. 63/563,135, filed Mar. 8, 2024, U.S. Provisional Application No. 63/621,953, filed Jan. 17, 2024, and U.S. Provisional Application No. 63/491,364, filed Mar. 21, 2023, the entire contents of each of which are incorporated by reference herein.

### SEQUENCE LISTING

This application contains a Sequence Listing that has been submitted electronically in XML format and is hereby incorporated by reference in its entirety. The Sequence Listing XML file, created on Mar. 11, 2026, is named 180802-049505_US_SL.xml and is 1,762,890 bytes in size.

### BACKGROUND OF THE DISCLOSURE

Allogeneic immunotherapy provides for the treatment of diseases or disorders (e.g., a neoplasia) in which immune effector cells (e.g., T cells, NK cells) expressing chimeric antigen receptors that bind an antigen present on a target cell (e.g., neoplastic cell) are administered to a subject. To generate an immune effector cell that expresses a chimeric antigen receptor (CAR), the immune effector cell is first collected from a donor separate from the subject receiving treatment (allogeneic) and genetically modified to express the chimeric antigen receptor. The resulting cell expresses the chimeric antigen receptor on its cell surface (e.g., CAR-T cell), and upon administration to the subject, the chimeric antigen receptor binds to the antigen expressed by a target cell associated with the disease or disorder. This interaction with the antigen activates the CAR-expressing immune effector cell (e.g., a CAR-T cell), which then kills, inactivates, or neutralizes target cells or molecules associated with the disease or disorder. Because allogenic CAR-expressing immune cells are obtained from a donor, the recipient's immune system may attack the foreign cells (e.g., host rejection of CAR-T cells). Thus, there is a significant need for techniques for improving immune effector cell resistance to rejection by a host.

### SUMMARY OF THE DISCLOSURE

As described below, the present disclosure features modified immune effector cells (e.g., T or NK cells) having increased resistance to inhibition by immunosuppressant agents relative to unmodified immune effector cells, pharmaceutical compositions containing such cells, and methods of using the modified immune effector cells to treat a disease, for example, by killing a target cell associated with the disease.

In one aspect, the disclosure features a method for treating a neoplasia in a subject in need thereof, the method involves administering to the subject (i) an immunosuppressant agent and (ii) a modified allogeneic immune effector cell. The modified allogeneic immune effector cell contains a chimeric antigen receptor capable of specifically binding a marker expressed by a neoplastic cell present in the subject. The allogeneic immune effector cell further contains a base edit in its genome that confers resistance to the immunosuppressant agent relative to an unmodified allogeneic immune effector cell.

In another aspect, the disclosure features a method for producing a modified allogeneic immune effector cell having increased resistance to an immunosuppressant agent. The method involves contacting the cell with (i) a base editor, or a polynucleotide encoding the base editor, and (ii) a guide polynucleotide, or a polynucleotide encoding the guide polynucleotide. The base editor contains a programmable DNA binding domain and a deaminase domain. The guide polynucleotide directs the base editor to effect a nucleobase alteration in a polynucleotide encoding a polypeptide selected from one or more of FK506-binding protein 1A (FKBP1A), nuclear receptor subfamily 3, group C, member 1 (NR3C1), and peptidyl-prolyl isomerase A (PPIA). Each nucleobase alteration effects a reduction in expression or activity of the encoded polypeptide, thereby increasing resistance of the modified immune effector cell to immunosuppression by an immunosuppressant agent selected from one or more of an mTOR inhibitor, calcineurin inhibitor, and glucocorticoid relative to an unmodified allogeneic immune effector cell.

In another aspect, the disclosure features a cell prepared according to the method of any aspect of the disclosure delineated herein, or embodiments thereof.

In another aspect, the disclosure features a pharmaceutical composition containing the cell of any aspect of the disclosure delineated herein, or embodiments thereof, and a pharmaceutically acceptable excipient.

In another aspect, the disclosure features a base editor system containing (i) a base editor, or a polynucleotide encoding the base editor, and (ii) a guide polynucleotide, or a polynucleotide encoding the guide polynucleotide. The base editor contains a programmable DNA binding domain and a deaminase domain. The guide polynucleotide directs the base editor to effect a nucleobase alteration in a polynucleotide encoding a polypeptide selected from one or more of FK506-binding protein 1A (FKBP1A), nuclear receptor subfamily 3, group C, member 1 (NR3C1), and peptidyl-prolyl isomerase A (PPIA).

In another aspect, the disclosure features a guide polynucleotide containing a spacer with a sequence containing at least 10 contiguous nucleotides selected from those sequences listed in Table 2A.

In another aspect, the disclosure features a polynucleotide encoding the base editor system, or a component thereof, or the guide polynucleotide of any aspect of the disclosure delineated herein, or embodiments thereof.

In another aspect, the disclosure features a vector containing the polynucleotide of any aspect of the disclosure delineated herein, or embodiments thereof.

In another aspect, the disclosure features a cell containing the base editor system, the guide polynucleotide, the polynucleotide, or the vector of any aspect of the disclosure delineated herein, or embodiments thereof.

In another aspect, the disclosure features a pharmaceutical composition containing the base editor system, or a component thereof, the guide polynucleotide, the polynucleotide, the vector, or the cell of any aspect of the disclosure delineated herein, or embodiments thereof.

In another aspect, the disclosure features a kit suitable for use in the method of any aspect of the disclosure delineated herein, or embodiments thereof, and containing the base editor system of, or a component thereof, the guide polynucleotide, the polynucleotide, the vector, the pharmaceutical composition, or the cell of any aspect of the disclosure delineated herein, or embodiments thereof, and a container.

In another aspect, the disclosure features a method for treating a leukemia or a lymphoma in a subject in need thereof, the method involving administering to the subject i) an immunosuppressant agent and ii) a modified allogeneic chimeric antigen receptor (CAR)-expressing T cell. The immunosuppressant agent is selected from one or more of mTOR inhibitors, calcineurin inhibitors, and glucocorticoids. The CAR is capable of specifically binding a marker expressed by a leukemia or a lymphoma cell in the subject. The modified allogeneic CAR T cell has increased resistance to the immunosuppressant agent relative to an unmodified allogeneic immune effector cell. The modified CAR T cell has been modified using a base editor system containing (a) a base editor, or a polynucleotide encoding the base editor, and (b) a guide polynucleotide, or a polynucleotide encoding the guide polynucleotide. The base editor contains an SpCas9 domain and a TadA*8.20 adenosine deaminase domain or an rAPOBEC1 domain. The guide polynucleotide contains a nucleotide sequence selected from those listed in Table 2A and directs the base editor to effect a nucleobase alteration in a polynucleotide encoding a polypeptide selected from one or more of FK506-binding protein 1A (FKBP1A), nuclear receptor subfamily 3, group C, member 1 (NR3C1), and peptidyl-prolyl isomerase A (PPIA). The nucleobase alteration reduces or eliminates activity or expression of the polypeptide in the cell.

In any aspect of the disclosure delineated herein, or embodiments thereof, the base edit reduces expression or activity of an FK506-binding protein 1A (FKBP1A), nuclear receptor subfamily 3, group C, member 1 (NR3C1), and/or peptidyl-prolyl isomerase A (PPIA) polypeptide relative to an unmodified allogeneic immune effector cell.

In any aspect of the disclosure delineated herein, or embodiments thereof, the immunosuppressant agent is selected from one or more of mTOR inhibitors, calcineurin inhibitors, and glucocorticoids.

In any aspect of the disclosure delineated herein, or embodiments thereof, the method further involves introducing the base edit to the modified allogeneic immune effector cell using a base editor system containing (i) a base editor, or a polynucleotide encoding the base editor, and (ii) a guide polynucleotide, or a polynucleotide encoding the guide polynucleotide. The base editor contains a programmable DNA binding domain and a deaminase domain. The guide polynucleotide directs the base editor to effect a nucleobase alteration in a polynucleotide encoding a polypeptide selected from one or more of FK506-binding protein 1A (FKBP1A), nuclear receptor subfamily 3, group C, member 1 (NR3C1), and peptidyl-prolyl isomerase A (PPIA).

In any aspect of the disclosure delineated herein, or embodiments thereof, the deaminase domain is an adenosine deaminase and/or a cytidine deaminase. In any aspect of the disclosure delineated herein, or embodiments thereof, the deaminase domain is a cytidine adenosine base editor (CABE). In any aspect of the disclosure delineated herein, or embodiments thereof, the deaminase domain is TadA*8.20 or rAPOBEC1, and/or the base editor is ABE8.20m or rBE4.

In any aspect of the disclosure delineated herein, or embodiments thereof, the guide polynucleotide contains at least 10 contiguous nucleotides of a spacer sequence listed in Table 2A.

In any aspect of the disclosure delineated herein, or embodiments thereof, the marker is a cluster of differentiation 19 (CD19) polypeptide. In any aspect of the disclosure delineated herein, or embodiments thereof, the marker is selected from the group consisting of CD5, CD7, CD19, CD20, CD22, CD79B, and ROR1

In any aspect of the disclosure delineated herein, or embodiments thereof, the neoplasia is a leukemia or a lymphoma. In any aspect of the disclosure delineated herein, or embodiments thereof, the neoplasia is a B-cell leukemia or a B-cell lymphoma.

In any aspect of the disclosure delineated herein, or embodiments thereof, the modified allogeneic immune effector cell shows increased proliferation, increased cytokine production, and/or increased cytotoxicity in the presence of the immunosuppressant agent relative to an unmodified allogeneic immune effector cell in the presence of the immunosuppressant agent.

In any aspect of the disclosure delineated herein, or embodiments thereof, the subject is a mammal. In embodiments, the mammal is a human.

In any aspect of the disclosure delineated herein, or embodiments thereof, the modified allogeneic immune effector cell expresses a chimeric antigen receptor capable of specifically binding a marker expressed on a target cell.

In any aspect of the disclosure delineated herein, or embodiments thereof, the nucleobase is altered with a base editing efficiency of at least about 25%, 50%, or 80%.

In any aspect of the disclosure delineated herein, or embodiments thereof, the immunosuppressant agent is selected from one or more of a rapalog, cyclosporine A, tacrolimus, dexamethasone, and/or prednisolone. In some embodiments, the rapalog is rapamycin or everolimus.

In any aspect of the disclosure delineated herein, or embodiments thereof, the modified allogeneic immune effector cell is a T cell or an NK cell. In any aspect of the disclosure delineated herein, or embodiments thereof, the modified allogeneic immune effector cell is a CD4+ or CD8+ T cell.

In any aspect of the disclosure delineated herein, or embodiments thereof, the modified allogeneic immune effector cell produces increased levels of a cytokine when activated by a target antigen in the presence of the immunosuppressant agent than the unmodified allogeneic immune effector cell. In embodiments, cytokine produced in the modified allogeneic immune effector cell is at least about 1.5-fold, 2-fold, 5-fold, or 10-fold higher than the amount produced by the unmodified allogeneic immune effector cell. In any aspect of the disclosure delineated herein, or embodiments thereof, the cytokine is tumor necrosis factor alpha (TNFa) or interferon gamma (IFNg).

In any aspect of the disclosure delineated herein, or embodiments thereof, the modified immune effector cell shows higher levels of proliferation when activated by a target antigen in the presence of the immunosuppressant agent than the unmodified allogeneic immune effector cell. In embodiments, the proliferation of the modified allogeneic immune effector cell is at least about 1.5-fold, 2-fold, 5-fold, or 10-fold higher than the proliferation of the unmodified allogeneic immune effector cells.

In any aspect of the disclosure delineated herein, or embodiments thereof, the modified allogeneic immune effector cells further contain a base edit that reduces expression of one or more polypeptides selected from one or more of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC) relative to an unmodified allogeneic immune effector cell.

In any aspect of the disclosure delineated herein, or embodiments thereof, the guide polynucleotide contains a scaffold containing the following nucleotide sequence: GUUUUAGAGCUAGAAAUAGCAAGUUAAAAUAAGGCUAGUCCGUUAUCAACUUGAAAAAGUGGCA CCGAGUCGGUGCUUUU (SEQ ID NO: 317; SpCas9 scaffold sequence), or a fragment thereof capable of binding a Cas9 polypeptide.

In any aspect of the disclosure delineated herein, or embodiments thereof, the guide polynucleotide contains a modified nucleotide.

In any aspect of the disclosure delineated herein, or embodiments thereof, the guide polynucleotide contains a sequence containing at least 10 contiguous nucleotides from the following sequence: CUCACCGUCUCCUGGGGAGA (SEQ ID NO: 553; TSBT×1538).

In any aspect of the disclosure delineated herein, or embodiments thereof, the vector is a viral vector. In any aspect of the disclosure delineated herein, or embodiments thereof, the viral vector is an AAV vector or a lentiviral vector.

In any aspect of the disclosure, or embodiments thereof, the modified allogeneic CAR-expressing T cell or modified allogeneic immune effector cell expresses functional human leukocyte antigen class I polypeptides. In any aspect of the disclosure, or embodiments thereof, the modified allogeneic CAR-expressing T cell or modified allogeneic immune effector cell expresses functional human leukocyte antigen class I polypeptides. In any aspect of the disclosure, or embodiments thereof, the modified allogeneic CAR-expressing T cell or modified allogeneic immune effector cell retains phosphorylation of the S6 ribosomal protein when contacted with rapamycin. In any aspect of the disclosure, or embodiments thereof, the modified allogeneic CAR-expressing T cell or modified allogeneic immune effector cell retains calcineurin-induced nuclear factor of activated T cells (NFAT)-driven polypeptide expression when contacted with tacrolimus. In any aspect of the disclosure, or embodiments thereof, the modified allogeneic CAR-expressing T cell or modified allogeneic immune effector cell shows proliferation, cytokine production, and/or cytotoxicity in the presence of the immunosuppressant agent that is similar to that of an allogeneic immune effector cell in the presence of the immunosuppressant agent that does not express functional human leukocyte antigen class I polypeptides.

In any aspect of the disclosure, or embodiments thereof, the method is associated with an increased reduction of leukemia or lymphoma cells in the peripheral blood, spleen, and/or bone marrow of the subject relative to a method where the subject is administered the modified allogeneic CAR-expressing T cell but not the immunosuppressant agent.

In any aspect provided herein, or embodiments thereof, the method is not a process for modifying the germline genetic identity of human beings. In any aspect provided herein, or embodiments thereof, the method is carried out in vitro or ex vivo or the cell is in vitro or ex vivo.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “beta-2 microglobulin (β2M; B2M) polypeptide” is meant a protein having at least about 85% amino acid sequence identity to UniProt Accession No. P61769, which is provided below, or a fragment thereof having immunomodulatory activity.

>sp|P61769|B2MG_HUMAN Beta-2-Microglobulin OS=Homo sapiens OX=9606 GN=B2M PE=1 SV=1

(SEQ ID NO: 429)
MSRSVALAVLALLSLSGLEAIQRTPKIQVYSRHPAENGKSNFLNCYVSG
FHPSDIEVDLLKNGERIEKVEHSDLSFSKDWSFYLLYYTEFTPTEKDEY
ACRVNHVTLSQPKIVKWDRDM.

By “beta-2-microglobulin (β2M; B2M) polynucleotide” is meant a nucleic acid molecule encoding an β2M polypeptide, as well as the introns, exons, 3′ untranslated regions, 5′ untranslated regions, and regulatory sequences associated with its expression, or fragments thereof. The beta-2-microglobulin gene encodes a serum protein associated with the major histocompatibility complex. β2M is involved in non-self-recognition by host CD8+ T cells. An exemplary β2M polynucleotide sequence is provided at GenBank Accession No. DQ217933.1, which is provided below.

>DQ217933.1 Homo sapiens Beta-2-Microglobin (β2M) Gene, Complete Cds

CATGTCATAAATGGTAAGTCCAAGAAAAATACAGGTATTCCCCCCCAAAGAAAACTGTAAAATC GACTTTTTTCTATCTGTACTGTTTTTTATTGGTTTTTAAATTGGTTTTCCAAGTGAGTAAATCA GAATCTATCTGTAATGGATTTTAAATTTAGTGTTTCTCTGTGATGTAGTAAACAAGAAACTAGA GGCAAAAATAGCCCTGTCCCTTGCTAAACTTCTAAGGCACTTTTCTAGTACAACTCAACACTAA CATTTCAGGCCTTTAGTGCCTTATATGAGTTTTTAAAAGGGGGAAAAGGGAGGGAGCAAGAGTG TCTTAACTCATACATTTAGGCATAACAATTATTCTCATATTTTAGTTATTGAGAGGGCTGGTAG AAAAACTAGGTAAATAATATTAATAATTATAGCGCTTATTAAACACTACAGAACACTTACTATG TACCAGGCATTGTGGGAGGCTCTCTCTTGTGCATTATCTCATTTCATTAGGTCCATGGAGAGTA TTGCATTTTCTTAGTTTAGGCATGGCCTCCACAATAAAGATTATCAAAAGCCTAAAAATATGTA AAAGAAACCTAGAAGTTATTTGTTGTGCTCCTTGGGGAAGCTAGGCAAATCCTTTCAACTGAAA ACCATGGTGACTTCCAAGATCTCTGCCCCTCCCCATCGCCATGGTCCACTTCCTCTTCTCACTG TTCCTCTTAGAAAAGATCTGTGGACTCCACCACCACGAAATGGCGGCACCTTATTTATGGTCAC TTTAGAGGGTAGGTTTTCTTAATGGGTCTGCCTGTCATGTTTAACGTCCTTGGCTGGGTCCAAG GCAGATGCAGTCCAAACTCTCACTAAAATTGCCGAGCCCTTTGTCTTCCAGTGTCTAAAATATT AATGTCAATGGAATCAGGCCAGAGTTTGAATTCTAGTCTCTTAGCCTTTGTTTCCCCTGTCCAT AAAATGAATGGGGGTAATTCTTTCCTCCTACAGTTTATTTATATATTCACTAATTCATTCATTC ATCCATCCATTCGTTCATTCGGTTTACTGAGTACCTACTATGTGCCAGCCCCTGTTCTAGGGTG GAAACTAAGAGAATGATGTACCTAGAGGGCGCTGGAAGCTCTAAAGCCCTAGCAGTTACTGCTT TTACTATTAGTGGTCGTTTTTTTCTCCCCCCCGCCCCCCGACAAATCAACAGAACAAAGAAAAT TACCTAAACAGCAAGGACATAGGGAGGAACTTCTTGGCACAGAACTTTCCAAACACTTTTTCCT GAAGGGATACAAGAAGCAAGAAAGGTACTCTTTCACTAGGACCTTCTCTGAGCTGTCCTCAGGA TGCTTTTGGGACTATTTTTCTTACCCAGAGAATGGAGAAACCCTGCAGGGAATTCCCAAGCTGT AGTTATAAACAGAAGTTCTCCTTCTGCTAGGTAGCATTCAAAGATCTTAATCTTCTGGGTTTCC GTTTTCTCGAATGAAAAATGCAGGTCCGAGCAGTTAACTGGCTGGGGCACCATTAGCAAGTCAC TTAGCATCTCTGGGGCCAGTCTGCAAAGCGAGGGGGCAGCCTTAATGTGCCTCCAGCCTGAAGT CCTAGAATGAGCGCCCGGTGTCCCAAGCTGGGGCGCGCACCCCAGATCGGAGGGCGCCGATGTA CAGACAGCAAACTCACCCAGTCTAGTGCATGCCTTCTTAAACATCACGAGACTCTAAGAAAAGG AAACTGAAAACGGGAAAGTCCCTCTCTCTAACCTGGCACTGCGTCGCTGGCTTGGAGACAGGTG ACGGTCCCTGCGGGCCTTGTCCTGATTGGCTGGGCACGCGTTTAATATAAGTGGAGGCGTCGCG CTGGCGGGCATTCCTGAAGCTGACAGCATTCGGGCCGAGATGTCTCGCTCCGTGGCCTTAGCTG TGCTCGCGCTACTCTCTCTTTCTGGCCTGGAGGCTATCCAGCGTGAGTCTCTCCTACCCTCCCG CTCTGGTCCTTCCTCTCCCGCTCTGCACCCTCTGTGGCCCTCGCTGTGCTCTCTCGCTCCGTGA CTTCCCTTCTCCAAGTTCTCCTTGGTGGCCCGCCGTGGGGCTAGTCCAGGGCTGGATCTCGGGG AAGCGGCGGGGTGGCCTGGGAGTGGGGAAGGGGGTGCGCACCCGGGACGCGCGCTACTTGCCCC TTTCGGCGGGGAGCAGGGGAGACCTTTGGCCTACGGCGACGGGAGGGTCGGGACAAAGTTTAGG GCGTCGATAAGCGTCAGAGCGCCGAGGTTGGGGGAGGGTTTCTCTTCCGCTCTTTCGCGGGGCC TCTGGCTCCCCCAGCGCAGCTGGAGTGGGGGACGGGTAGGCTCGTCCCAAAGGCGCGGCGCTGA GGTTTGTGAACGCGTGGAGGGGCGCTTGGGGTCTGGGGGAGGCGTCGCCCGGGTAAGCCTGTCT GCTGCGGCTCTGCTTCCCTTAGACTGGAGAGCTGTGGACTTCGTCTAGGCGCCCGCTAAGTTCG CATGTCCTAGCACCTCTGGGTCTATGTGGGGCCACACCGTGGGGAGGAAACAGCACGCGACGTT TGTAGAATGCTTGGCTGTGATACAAAGCGGTTTCGAATAATTAACTTATTTGTTCCCATCACAT GTCACTTTTAAAAAATTATAAGAACTACCCGTTATTGACATCTTTCTGTGTGCCAAGGACTTTA TGTGCTTTGCGTCATTTAATTTTGAAAACAGTTATCTTCCGCCATAGATAACTACTATGGTTAT CTTCTGCCTCTCACAGATGAAGAAACTAAGGCACCGAGATTTTAAGAAACTTAATTACACAGGG GATAAATGGCAGCAATCGAGATTGAAGTCAAGCCTAACCAGGGCTTTTGCGGGAGCGCATGCCT TTTGGCTGTAATTCGTGCATTTTTTTTTAAGAAAAACGCCTGCCTTCTGCGTGAGATTCTCCAG AGCAAACTGGGCGGCATGGGCCCTGTGGTCTTTTCGTACAGAGGGCTTCCTCTTTGGCTCTTTG CCTGGTTGTTTCCAAGATGTACTGTGCCTCTTACTTTCGGTTTTGAAAACATGAGGGGGTTGGG CGTGGTAGCTTACGCCTGTAATCCCAGCACTTAGGGAGGCCGAGGCGGGAGGATGGCTTGAGGT CCGTAGTTGAGACCAGCCTGGCCAACATGGTGAAGCCTGGTCTCTACAAAAAATAATAACAAAA ATTAGCCGGGTGTGGTGGCTCGTGCCTGTGGTCCCAGCTGCTCCGGTGGCTGAGGCGGGAGGAT CTCTTGAGCTTAGGCTTTTGAGCTATCATGGCGCCAGTGCACTCCAGCGTGGGCAACAGAGCGA GACCCTGTCTCTCAAAAAAGAAAAAAAAAAAAAAAGAAAGAGAAAAGAAAAGAAAGAAAGAAGT GAAGGTTTGTCAGTCAGGGGAGCTGTAAAACCATTAATAAAGATAATCCAAGATGGTTACCAAG ACTGTTGAGGACGCCAGAGATCTTGAGCACTTTCTAAGTACCTGGCAATACACTAAGCGCGCTC ACCTTTTCCTCTGGCAAAACATGATCGAAAGCAGAATGTTTTGATCATGAGAAAATTGCATTTA ATTTGAATACAATTTATTTACAACATAAAGGATAATGTATATATCACCACCATTACTGGTATTT GCTGGTTATGTTAGATGTCATTTTAAAAAATAACAATCTGATATTTAAAAAAAAATCTTATTTT GAAAATTTCCAAAGTAATACATGCCATGCATAGACCATTTCTGGAAGATACCACAAGAAACATG TAATGATGATTGCCTCTGAAGGTCTATTTTCCTCCTCTGACCTGTGTGTGGGTTTTGTTTTTGT TTTACTGTGGGCATAAATTAATTTTTCAGTTAAGTTTTGGAAGCTTAAATAACTCTCCAAAAGT CATAAAGCCAGTAACTGGTTGAGCCCAAATTCAAACCCAGCCTGTCTGATACTTGTCCTCTTCT TAGAAAAGATTACAGTGATGCTCTCACAAAATCTTGCCGCCTTCCCTCAAACAGAGAGTTCCAG GCAGGATGAATCTGTGCTCTGATCCCTGAGGCATTTAATATGTTCTTATTATTAGAAGCTCAGA TGCAAAGAGCTCTCTTAGCTTTTAATGTTATGAAAAAAATCAGGTCTTCATTAGATTCCCCAAT CCACCTCTTGATGGGGCTAGTAGCCTTTCCTTAATGATAGGGTGTTTCTAGAGAGATATATCTG GTCAAGGTGGCCTGGTACTCCTCCTTCTCCCCACAGCCTCCCAGACAAGGAGGAGTAGCTGCCT TTTAGTGATCATGTACCCTGAATATAAGTGTATTTAAAAGAATTTTATACACATATATTTAGTG TCAATCTGTATATTTAGTAGCACTAACACTTCTCTTCATTTTCAATGAAAAATATAGAGTTTAT AATATTTTCTTCCCACTTCCCCATGGATGGTCTAGTCATGCCTCTCATTTTGGAAAGTACTGTT TCTGAAACATTAGGCAATATATTCCCAACCTGGCTAGTTTACAGCAATCACCTGTGGATGCTAA TTAAAACGCAAATCCCACTGTCACATGCATTACTCCATTTGATCATAATGGAAAGTATGTTCTG TCCCATTTGCCATAGTCCTCACCTATCCCTGTTGTATTTTATCGGGTCCAACTCAACCATTTAA GGTATTTGCCAGCTCTTGTATGCATTTAGGTTTTGTTTCTTTGTTTTTTAGCTCATGAAATTAG GTACAAAGTCAGAGAGGGGTCTGGCATATAAAACCTCAGCAGAAATAAAGAGGTTTTGTTGTTT GGTAAGAACATACCTTGGGTTGGTTGGGCACGGTGGCTCGTGCCTGTAATCCCAACACTTTGGG AGGCCAAGGCAGGCTGATCACTTGAAGTTGGGAGTTCAAGACCAGCCTGGCCAACATGGTGAAA TCCCGTCTCTACTGAAAATACAAAAATTAACCAGGCATGGTGGTGTGTGCCTGTAGTCCCAGGA ATCACTTGAACCCAGGAGGCGGAGGTTGCAGTGAGCTGAGATCTCACCACTGCACACTGCACTC CAGCCTGGGCAATGGAATGAGATTCCATCCCAAAAAATAAAAAAATAAAAAAATAAAGAACATA CCTTGGGTTGATCCACTTAGGAACCTCAGATAATAACATCTGCCACGTATAGAGCAATTGCTAT GTCCCAGGCACTCTACTAGACACTTCATACAGTTTAGAAAATCAGATGGGTGTAGATCAAGGCA GGAGCAGGAACCAAAAAGAAAGGCATAAACATAAGAAAAAAAATGGAAGGGGTGGAAACAGAGT ACAATAACATGAGTAATTTGATGGGGGCTATTATGAACTGAGAAATGAACTTTGAAAAGTATCT TGGGGCCAAATCATGTAGACTCTTGAGTGATGTGTTAAGGAATGCTATGAGTGCTGAGAGGGCA TCAGAAGTCCTTGAGAGCCTCCAGAGAAAGGCTCTTAAAAATGCAGCGCAATCTCCAGTGACAG AAGATACTGCTAGAAATCTGCTAGAAAAAAAACAAAAAAGGCATGTATAGAGGAATTATGAGGG AAAGATACCAAGTCACGGTTTATTCTTCAAAATGGAGGTGGCTTGTTGGGAAGGTGGAAGCTCA TTTGGCCAGAGTGGAAATGGAATTGGGAGAAATCGATGACCAAATGTAAACACTTGGTGCCTGA TATAGCTTGACACCAAGTTAGCCCCAAGTGAAATACCCTGGCAATATTAATGTGTCTTTTCCCG ATATTCCTCAGGTACTCCAAAGATTCAGGTTTACTCACGTCATCCAGCAGAGAATGGAAAGTCA AATTTCCTGAATTGCTATGTGTCTGGGTTTCATCCATCCGACATTGAAGTTGACTTACTGAAGA ATGGAGAGAGAATTGAAAAAGTGGAGCATTCAGACTTGTCTTTCAGCAAGGACTGGTCTTTCTA TCTCTTGTACTACACTGAATTCACCCCCACTGAAAAAGATGAGTATGCCTGCCGTGTGAACCAT GTGACTTTGTCACAGCCCAAGATAGTTAAGTGGGGTAAGTCTTACATTCTTTTGTAAGCTGCTG AAAGTTGTGTATGAGTAGTCATATCATAAAGCTGCTTTGATATAAAAAAGGTCTATGGCCATAC TACCCTGAATGAGTCCCATCCCATCTGATATAAACAATCTGCATATTGGGATTGTCAGGGAATG TTCTTAAAGATCAGATTAGTGGCACCTGCTGAGATACTGATGCACAGCATGGTTTCTGAACCAG TAGTTTCCCTGCAGTTGAGCAGGGAGCAGCAGCAGCACTTGCACAAATACATATACACTCTTAA CACTTCTTACCTACTGGCTTCCTCTAGCTTTTGTGGCAGCTTCAGGTATATTTAGCACTGAACG AACATCTCAAGAAGGTATAGGCCTTTGTTTGTAAGTCCTGCTGTCCTAGCATCCTATAATCCTG GACTTCTCCAGTACTTTCTGGCTGGATTGGTATCTGAGGCTAGTAGGAAGGGCTTGTTCCTGCT GGGTAGCTCTAAACAATGTATTCATGGGTAGGAACAGCAGCCTATTCTGCCAGCCTTATTTCTA ACCATTTTAGACATTTGTTAGTACATGGTATTTTAAAAGTAAAACTTAATGTCTTCCTTTTTTT TCTCCACTGTCTTTTTCATAGATCGAGACATGTAAGCAGCATCATGGAGGTAAGTTTTTGACCT TGAGAAAATGTTTTTGTTTCACTGTCCTGAGGACTATTTATAGACAGCTCTAACATGATAACCC TCACTATGTGGAGAACATTGACAGAGTAACATTTTAGCAGGGAAAGAAGAATCCTACAGGGTCA TGTTCCCTTCTCCTGTGGAGTGGCATGAAGAAGGTGTATGGCCCCAGGTATGGCCATATTACTG ACCCTCTACAGAGAGGGCAAAGGAACTGCCAGTATGGTATTGCAGGATAAAGGCAGGTGGTTAC CCACATTACCTGCAAGGCTTTGATCTTTCTTCTGCCATTTCCACATTGGACATCTCTGCTGAGG AGAGAAAATGAACCACTCTTTTCCTTTGTATAATGTTGTTTTATTCTTCAGACAGAAGAGAGGA GTTATACAGCTCTGCAGACATCCCATTCCTGTATGGGGACTGTGTTTGCCTCTTAGAGGTTCCC AGGCCACTAGAGGAGATAAAGGGAAACAGATTGTTATAACTTGATATAATGATACTATAATAGA TGTAACTACAAGGAGCTCCAGAAGCAAGAGAGAGGGAGGAACTTGGACTTCTCTGCATCTTTAG TTGGAGTCCAAAGGCTTTTCAATGAAATTCTACTGCCCAGGGTACATTGATGCTGAAACCCCAT TCAAATCTCCTGTTATATTCTAGAACAGGGAATTGATTTGGGAGAGCATCAGGAAGGTGGATGA TCTGCCCAGTCACACTGTTAGTAAATTGTAGAGCCAGGACCTGAACTCTAATATAGTCATGTGT TACTTAATGACGGGGACATGTTCTGAGAAATGCTTACACAAACCTAGGTGTTGTAGCCTACTAC ACGCATAGGCTACATGGTATAGCCTATTGCTCCTAGACTACAAACCTGTACAGCCTGTTACTGT ACTGAATACTGTGGGCAGTTGTAACACAATGGTAAGTATTTGTGTATCTAAACATAGAAGTTGC AGTAAAAATATGCTATTTTAATCTTATGAGACCACTGTCATATATACAGTCCATCATTGACCAA AACATCATATCAGCATTTTTTCTTCTAAGATTTTGGGAGCACCAAAGGGATACACTAACAGGAT ATACTCTTTATAATGGGTTTGGAGAACTGTCTGCAGCTACTTCTTTTAAAAAGGTGATCTACAC AGTAGAAATTAGACAAGTTTGGTAATGAGATCTGCAATCCAAATAAAATAAATTCATTGCTAAC CTTTTTCTTTTCTTTTCAGGTTTGAAGATGCCGCATTTGGATTGGATGAATTCCAAATTCTGCT TGCTTGCTTTTTAATATTGATATGCTTATACACTTACACTTTATGCACAAAATGTAGGGTTATA ATAATGTTAACATGGACATGATCTTCTTTATAATTCTACTTTGAGTGCTGTCTCCATGTTTGAT GTATCTGAGCAGGTTGCTCCACAGGTAGCTCTAGGAGGGCTGGCAACTTAGAGGTGGGGAGCAG AGAATTCTCTTATCCAACATCAACATCTTGGTCAGATTTGAACTCTTCAATCTCTTGCACTCAA AGCTTGTTAAGATAGTTAAGCGTGCATAAGTTAACTTCCAATTTACATACTCTGCTTAGAATTT GGGGGAAAATTTAGAAATATAATTGACAGGATTATTGGAAATTTGTTATAATGAATGAAACATT TTGTCATATAAGATTCATATTTACTTCTTATACATTTGATAAAGTAAGGCATGGTTGTGGTTAA TCTGGTTTATTTTTGTTCCACAAGTTAAATAAATCATAAAACTTGATGTGTTATCTCTTATATC TCACTCCCACTATTACCCCTTTATTTTCAAACAGGGAAACAGTCTTCAAGTTCCACTTGGTAAA AAATGTGAACCCCTTGTATATAGAGTTTGGCTCACAGTGTAAAGGGCCTCAGTGATTCACATTT TCCAGATTAGGAATCTGATGCTCAAAGAAGTTAAATGGCATAGTTGGGGTGACACAGCTGTCTA GTGGGAGGCCAGCCTTCTATATTTTAGCCAGCGTTCTTTCCTGCGGGCCAGGTCATGAGGAGTA TGCAGACTCTAAGAGGGAGCAAAAGTATCTGAAGGATTTAATATTTTAGCAAGGAATAGATATA CAATCATCCCTTGGTCTCCCTGGGGGATTGGTTTCAGGACCCCTTCTTGGACACCAAATCTATG GATATTTAAGTCCCTTCTATAAAATGGTATAGTATTTGCATATAACCTATCCACATCCTCCTGT ATACTTTAAATCATTTCTAGATTACTTGTAATACCTAATACAATGTAAATGCTATGCAAATAGT TGTTATTGTTTAAGGAATAATGACAAGAAAAAAAAGTCTGTACATGCTCAGTAAAGACACAACC ATCCCTTTTTTTCCCCAGTGTTTTTGATCCATGGTTTGCTGAATCCACAGATGTGGAGCCCCTG GATACGGAAGGCCCGCTGTACTTTGAATGACAAATAACAGATTTAAA (SEQ ID NO: 430). An exemplary B2M gene sequence is provided at (SEQ ID NO: 431).

By “cluster of differentiation 3 epsilon (CD3e or CD3 epsilon) polypeptide” is meant a protein having at least about 85% amino acid sequence identity to NCBI Accession No. NP_000724.1 or a fragment thereof having immunomodulatory activity. An exemplary amino acid sequence is provided below.

>NP 000724.1 T-Cell Surface Glycoprotein CD3 Epsilon Chain Precursor [Homo sapiens]

(SEQ ID NO: 432)
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI

By “cluster of differentiation 3 epsilon (CD3e or CD3 epsilon) polynucleotide” is meant a polynucleotide encoding a CD3e polypeptide, as well as the introns, exons, 3′ untranslated regions, 5′ untranslated regions, and regulatory sequences associated with its expression, or fragments thereof. In embodiments, a CD3e polynucleotide is the genomic sequence, cDNA, mRNA, or gene associated with and/or required for CD3e expression. An exemplary CD3e nucleic acid sequence is provided below (NCBI Ref. Seq. Accession No. NM_000733.4).

>NM 000733.4 Homo sapiens CD3e Molecule (CD3E), mRNA

AGAAACCCTCCTCCCCTCCCAGCCTCAGGTGCCTGCTTCAGAAAATGAAGTAGTAAGTCTGCTG GCCTCCGCCATCTTAGTAAAGTAACAGTCCCATGAAACAAAGATGCAGTCGGGCACTCACTGGA GAGTTCTGGGCCTCTGCCTCTTATCAGTTGGCGTTTGGGGGCAAGATGGTAATGAAGAAATGGG TGGTATTACACAGACACCATATAAAGTCTCCATCTCTGGAACCACAGTAATATTGACATGCCCT CAGTATCCTGGATCTGAAATACTATGGCAACACAATGATAAAAACATAGGCGGTGATGAGGATG ATAAAAACATAGGCAGTGATGAGGATCACCTGTCACTGAAGGAATTTTCAGAATTGGAGCAAAG TGGTTATTATGTCTGCTACCCCAGAGGAAGCAAACCAGAAGATGCGAACTTTTATCTCTACCTG AGGGCAAGAGTGTGTGAGAACTGCATGGAGATGGATGTGATGTCGGTGGCCACAATTGTCATAG TGGACATCTGCATCACTGGGGGCTTGCTGCTGCTGGTTTACTACTGGAGCAAGAATAGAAAGGC CAAGGCCAAGCCTGTGACACGAGGAGCGGGTGCTGGCGGCAGGCAAAGGGGACAAAACAAGGAG AGGCCACCACCTGTTCCCAACCCAGACTATGAGCCCATCCGGAAAGGCCAGCGGGACCTGTATT CTGGCCTGAATCAGAGACGCATCTGACCCTCTGGAGAACACTGCCTCCCGCTGGCCCAGGTCTC CTCTCCAGTCCCCCTGCGACTCCCTGTTTCCTGGGCTAGTCTTGGACCCCACGAGAGAGAATCG TTCCTCAGCCTCATGGTGAACTCGCGCCCTCCAGCCTGATCCCCCGCTCCCTCCTCCCTGCCTT CTCTGCTGGTACCCAGTCCTAAAATATTGCTGCTTCCTCTTCCTTTGAAGCATCATCAGTAGTC ACACCCTCACAGCTGGCCTGCCCTCTTGCCAGGATATTTATTTGTGCTATTCACTCCCTTCCCT TTGGATGTAACTTCTCCGTTCAGTTCCCTCCTTTTCTTGCATGTAAGTIGTCCCCCATCCCAAA GTATTCCATCTACTTTTCTATCGCCGTCCCCTTTTGCAGCCCTCTCTGGGGATGGACTGGGTAA ATGTTGACAGAGGCCCTGCCCCGTTCACAGATCCTGGCCCTGAGCCAGCCCTGTGCTCCTCCCT CCCCCAACACTCCCTACCAACCCCCTAATCCCCTACTCCCTCCACCCCCCCTCCACTGTAGGCC ACTGGATGGTCATTTGCATCTCCGTAAATGTGCTCTGCTCCTCAGCTGAGAGAGAAAAAAATAA ACTGTATTTGGCTGCAA (SEQ ID NO: 433). An exemplary CD3e sequence is provided at ENSEMBL Accession No. ENSG00000198851 (SEQ ID NO: 434).

By “cluster of differentiation 3 gamma (CD3g or CD3 gamma) polypeptide” is meant a protein having at least about 85% amino acid sequence identity to NCBI Accession No. NP_000064.1 or a fragment thereof and having immunomodulatory activity. An exemplary amino acid sequence is provided below.

>NP_000064.1 T-Cell Surface Glycoprotein CD3 Gamma Chain Precursor [Homo sapiens]

(SEQ ID NO: 435)
MEQGKGLAVLILAIILLQGTLAQSIKGNHLVKVYDYQEDGSVLLTCDAE
AKNITWFKDGKMIGFLTEDKKKWNLGSNAKDPRGMYQCKGSQNKSKPLQ
VYYRMCQNCIELNAATISGFLFAEIVSIFVLAVGVYFIAGQDGVRQSRA
SDKQTLLPNDQLYQPLKDREDDQYSHLQGNQLRRN.

By “cluster of differentiation 3 gamma (CD3g or CD3 gamma) polynucleotide” is meant a polypeptide encoding a CD3g polypeptide, as well as the introns, exons, 3′ untranslated regions, 5′ untranslated regions, and regulatory sequences associated with its expression, or fragments thereof. In embodiments, a CD3g polynucleotide is the genomic sequence, cDNA, mRNA, or gene associated with and/or required for CD3g expression. An exemplary CD3g nucleic acid sequence is provided below (NCBI Ref. Seq. Accession No. NM_000073.3).

>NM 000073.3 Homo sapiens CD3g Molecule (CD3G), mRNA

AGTCTAGCTGCTGCACAGGCTGGCTGGCTGGCTGGCTGCTAAGGGCTGCTCCACGCTTTTGCCG GAGGACAGAGACTGACATGGAACAGGGGAAGGGCCTGGCTGTCCTCATCCTGGCTATCATTCTT CTTCAAGGTACTTTGGCCCAGTCAATCAAAGGAAACCACTTGGTTAAGGTGTATGACTATCAAG AAGATGGTTCGGTACTTCTGACTTGTGATGCAGAAGCCAAAAATATCACATGGTTTAAAGATGG GAAGATGATCGGCTTCCTAACTGAAGATAAAAAAAAATGGAATCTGGGAAGTAATGCCAAGGAC CCTCGAGGGATGTATCAGTGTAAAGGATCACAGAACAAGTCAAAACCACTCCAAGTGTATTACA GAATGTGTCAGAACTGCATTGAACTAAATGCAGCCACCATATCTGGCTTTCTCTTTGCTGAAAT CGTCAGCATTTTCGTCCTTGCTGTTGGGGTCTACTTCATTGCTGGACAGGATGGAGTTCGCCAG TCGAGAGCTTCAGACAAGCAGACTCTGTTGCCCAATGACCAGCTCTACCAGCCCCTCAAGGATC GAGAAGATGACCAGTACAGCCACCTTCAAGGAAACCAGTTGAGGAGGAATTGAACTCAGGACTC AGAGTAGTCCAGGTGTTCTCCTCCTATTCAGTTCCCAGAATCAAAGCAATGCATTTTGGAAAGC TCCTAGCAGAGAGACTTTCAGCCCTAAATCTAGACTCAAGGTTCCCAGAGATGACAAATGGAGA AGAAAGGCCATCAGAGCAAATTTGGGGGTTTCTCAAATAAAATAAAAATAAAAACAAATACTGT GTTTCAGAAGCGCCACCTATTGGGGAAAATTGTAAAAGAAAAATGAAAAGATCAAATAACCCCC TGGATTTGAATATAATTTTTTGTGTTGTAATTTTTATTTCGTTTTTGTATAGGTTATAATTCAC ATGGCTCAAATATTCAGTGAAAGCTCTCCCTCCACCGCCATCCCCTGCTACCCAGTGACCCTGT TGCCCTCTTCAGAGACAAATTAGTTTCTCTTTTTTTTTTTTTTTTTTTTTTTTTTGAGACAGTC TGGCTCTGTCACCCAGGCTGAAATGCAGTGGCACCATCTCGGCTCACTGCAACCTCTGCCTCCT GGGTTCAAGCGATTCTCCTGCCTCAGCCTCCCGGGCAGCTGGGATTACAGGCACACACTACCAC ACCTGGCTAATTTTTGTATTTTTAGTAGAGACAGGGTTTTGCTCTGTTGGCCAAGCTGGTCTCG AACTCCTGACCTCAAGTGATCCGCCCGCCTCAGCCTCCCAAAGTGCTGGGATTACAGGTGTGAG CCACCATGCCTGGTCTTAAAACCAGTTTCTTATATATCTCTCTGGAGGTATTCTAGGCATATAT GAGCACATTCTCAAGTACATATTATCCTCCCTTCCCCTATCTTTTAGACAAATGATATCAAACT ATACATCTTGTGAGATTATTGCATACCATTATATGAAGATACCATTATATCCTTTTTAATGCAA CCATATTGTACAAATAGACTATGATTTATTTAACCTGTTATCTATCAGTGGATATTTAAGTTGG TAGTTGGTTCCAATCTTTTGCTCTTACAACAATTCTGCAATGACTAACATTGTATAAATATCAT TTTTAAAAATAATTGCATTGAAGCATAATGTACATGCCATAAAATCCACCCATCTTAAGTGATT TCACCTGTTCTCAGAAATTTTTAGTAAATTTAACTAATTGTACAGCCATTACCATAATCCAGCT TTAGGACATTTTCTTTTTTTTCTTTTCTTTTCTTTTTTTTCTTTTTTTTTTTTTTTTGAAGTGG AATCTTGCTCTGTGGCCCAGGCTGGAGTGCAGTGGCGCGATCTCAGCTCACTGCAACCTCCACC TCCTGGGTTCAAGCGATTCTCTTGCCTTGGCCTCCCGAGTAGCTGAGACTACAGGCACATGCCA CCACGCCCAGCTCATTTTTTGTGTATTTAGTATTTGTGTATCTAGTATTTGTGTACTTAGTAGA GACAGGGTTTCACCATGTTGGCCAGGCTGGTCTCCAATTCCTGACCTCAGGCGATCCACCCGCC TTGACCTCCCAAAGTGCTGGGATTACAGGTGTGAGCCACCGCGCCAGGCCCGTAACTGTATTTT AATATAGCCATTCTATGGATTTAATATGGTATTTTATTATGGCCTTAATTTGCATTTCCCTAGA TACTAACCATGCTGAGTGTCCTGTCTTGTGTTTATTAACCATTCATATATTTTTAGTGAAATGT GTATCAAATCTTTTGCCCATTTTTAAGTTGACTTATTTGTTTGTCTTCTTACTATTGGGTTGCA TATGTTTTTGATATAAGTCCTTTATCAGATATATGATTTGGAAATATTTTCTACCAATCTGTGG TTTGTTTTTCTTAATGGTGTCTTTTGAAGTGCAAAAGGTTTGAATTTTGAAGTACATTTTATTG ATTTTTTCTTCTATATATTGTGCTTTTGGTATCATGTCTAATAAATCTTTACCAAACCCACAGT TACAAAGATTTTCTCCTGTCTTCTTTTTATACTTTTTACAGCTTTATGGTTTTAGCTCTAACAA TAAATGTGATTTTGAACATACATAAGACTATTTGTAACAAACACAAATAAATTGAATTGTTGGG CA (SEQ ID NO: 436). An exemplary CD3g sequence is provided at ENSEMBL Accession No. ENSG00000160654 (SEQ ID NO: 437).

By “B-lymphocyte antigen CD19 (CD19) polypeptide” is meant a protein having at least about 85% amino acid sequence identity to GenBank Accession No.: AAB60697.1, which is provided below, or a fragment thereof having immunomodulatory activity.

>AAB60697.1 CD19 [Homo sapiens]

(SEQ ID NO: 438)
MPPPRLLFFLLFLTPMEVRPEEPLVVKVEGEGDNAVLQCLKGTSDGPTQQLTWSRESPLKPFLK
LSLGLPGLGIHMRPLASWLFIFNVSQQMGGFYLCQPGPPSEKAWQPGWTVNVEGSGELFRWNVS
DLGGLGCGLKNRSSEGPSSPSGKLMSPKLYVWAKDRPEIWEGEPPCVPPRDSLNQSLSQDLTMA
PGSTLWLSCGVPPDSVSRGPLSWTHVHPKGPKSLLSLELKDDRPARDMWVMETGLLLPRATAQD
AGKYYCHRGNLTMSFHLEITARPVLWHWLLRTGGWKVSAVTLAYLIFCLCSLVGILHLQRALVL
RRKRKRMTDPTRRFFKVTPPPGSGPQNQYGNVLSLPTPTSGLGRAQRWAAGLGGTAPSYGNPSS
DVQADGALGSRSPPGVGPEEEEGEGYEEPDSEEDSEFYENDSNLGQDQLSQDGSGYENPEDEPL
GPEDEDSFSNAESYENEDEELTQPVARTMDFLSPHGSAWDPSREATSLGSQSYEDMRGILYAAP
QLRSIRGQPGPNHEEDADSYENMDNPDGPDPAWGGGGRMGTWSTR.

By “B-lymphocyte antigen CD19 (CD19) polynucleotide” is meant a polynucleotide encoding a CD19 polypeptide, as well as the introns, exons, 3′ untranslated regions, 5′ untranslated regions, and regulatory sequences associated with its expression, or fragments thereof. In embodiments, a CD19 polynucleotide is the genomic sequence, cDNA, mRNA, or gene associated with and/or required for CD19 expression. An exemplary CD19 nucleic acid sequence is provided below (GenBank Accession No. AH005421.2:331-421,665-931,1230-1433,1554-1829,2057-2167,2769-2817,3128-3216,3591-3704,3896-4000,4174-4242,4343-4399,4488-4544,4813-4905,5231-5322).

>AH005421.2:331-421,665-931,1230-1433,1554-1829,2057-2167,2769-2817,3128-3216,3591-3704,3896-4000,4174-4242,4343-4399,4488-4544,4813-4905,5231-5322 Homo sapiens CD19 (CD19) Gene, Complete Cds

ATGCCACCTCCTCGCCTCCTCTTCTTCCTCCTCTTCCTCACCCCCATGGAAGTCAGGCCCGAGG AACCTCTAGTGGTGAAGGTGGAAGGTGAGGGAGATAACGCTGTGCTGCAGTGCCTCAAGGGGAC CTCAGATGGCCCCACTCAGCAGCTGACCTGGTCTCGGGAGTCCCCGCTTAAACCCTTCTTAAAA CTCAGCCTGGGGCTGCCAGGCCTGGGAATCCACATGAGGCCCCTGGCATCCTGGCTTTTCATCT TCAACGTCTCTCAACAGATGGGGGGCTTCTACCTGTGCCAGCCGGGGCCCCCCTCTGAGAAGGC CTGGCAGCCTGGCTGGACAGTCAATGTGGAGGGCAGCGGGGAGCTGTTCCGGTGGAATGTTTCG GACCTAGGTGGCCTGGGCTGTGGCCTGAAGAACAGGTCCTCAGAGGGCCCCAGCTCCCCTTCCG GGAAGCTCATGAGCCCCAAGCTGTATGTGTGGGCCAAAGACCGCCCTGAGATCTGGGAGGGAGA GCCTCCGTGTGTCCCACCGAGGGACAGCCTGAACCAGAGCCTCAGCCAGGACCTCACCATGGCC CCTGGCTCCACACTCTGGCTGTCCTGTGGGGTACCCCCTGACTCTGTGTCCAGGGGCCCCCTCT CCTGGACCCATGTGCACCCCAAGGGGCCTAAGTCATTGCTGAGCCTAGAGCTGAAGGACGATCG CCCGGCCAGAGATATGTGGGTAATGGAGACGGGTCTGTTGTTGCCCCGGGCCACAGCTCAAGAC GCTGGAAAGTATTATTGTCACCGTGGCAACCTGACCATGTCATTCCACCTGGAGATCACTGCTC GGCCAGTACTATGGCACTGGCTGCTGAGGACTGGTGGCTGGAAGGTCTCAGCTGTGACTTTGGC TTATCTGATCTTCTGCCTGTGTTCCCTTGTGGGCATTCTTCATCTTCAAAGAGCCCTGGTCCTG AGGAGGAAAAGAAAGCGAATGACTGACCCCACCAGGAGATTCTTCAAAGTGACGCCTCCCCCAG GAAGCGGGCCCCAGAACCAGTACGGGAACGTGCTGTCTCTCCCCACACCCACCTCAGGCCTCGG ACGCGCCCAGCGTTGGGCCGCAGGCCTGGGGGGCACTGCCCCGTCTTATGGAAACCCGAGCAGC GACGTCCAGGCGGATGGAGCCTTGGGGTCCCGGAGCCCGCCGGGAGTGGGCCCAGAAGAAGAGG AAGGGGAGGGCTATGAGGAACCTGACAGTGAGGAGGACTCCGAGTTCTATGAGAACGACTCCAA CCTTGGGCAGGACCAGCTCTCCCAGGATGGCAGCGGCTACGAGAACCCTGAGGATGAGCCCCTG GGTCCTGAGGATGAAGACTCCTTCTCCAACGCTGAGTCTTATGAGAACGAGGATGAAGAGCTGA CCCAGCCGGTCGCCAGGACAATGGACTTCCTGAGCCCTCATGGGTCAGCCTGGGACCCCAGCCG GGAAGCAACCTCCCTGGGGTCCCAGTCCTATGAGGATATGAGAGGAATCCTGTATGCAGCCCCC CAGCTCCGCTCCATTCGGGGCCAGCCTGGACCCAATCATGAGGAAGATGCAGACTCTTATGAGA ACATGGATAATCCCGATGGGCCAGACCCAGCCTGGGGAGGAGGGGGCCGCATGGGCACCTGGAG CACCAGGTGA (SEQ ID NO: 439). An exemplary CD19 gene sequence is provided at ENSEMBL Accession No. ENSG00000177455 (SEQ ID NO: 440).

By “cluster of differentiation 20 (CD20) polypeptide” is meant a protein having at least about 85% amino acid sequence identity to NCBI Ref. Seq. Accession No. NP_690606.1, provided below, or fragment thereof and having immunomodulatory activity.

>NP_690606.1 B-Lymphocyte Antigen CD20 [Homo sapiens]

(SEQ ID NO: 441)
MTTPRNSVNGTFPAEPMKGPIAMQSGPKPLFRRMSSLVGPTQSFFMRES
KTLGAVQIMNGLFHIALGGLLMIPAGIYAPICVTVWYPLWGGIMYIISG
SLLAATEKNSRKCLVKGKMIMNSLSLFAAISGMILSIMDILNIKISHFL
KMESLNFIRAHTPYINIYNCEPANPSEKNSPSTQYCYSIQSLFLGILSV
MLIFAFFQELVIAGIVENEWKRTCSRPKSNIVLLSAEEKKEQTIEIKEE
VVGLTETSSQPKNEEDIEIIPIQEEEEEETETNFPEPPQDQESSPIEND
SSP.

By “cluster of differentiation 20 (CD20) polynucleotide” is meant a polynucleotide encoding a CD20 polypeptide, as well as the introns, exons, 3′ untranslated regions, 5′ untranslated regions, and regulatory sequences associated with its expression, or fragments thereof. In embodiments, a CD20 polynucleotide is the genomic sequence, cDNA, mRNA, or gene associated with and/or required for CD20 expression. An exemplary CD20 nucleic acid sequence is provided below (NCBI Ref. Seq. Accession No. NM_152867.2:290-1183). (SEQ ID NO: 442).

>NM_152867.2:290-1183 Homo sapiens Membrane Spanning 4-Domains A1 (MS4A1), Transcript Variant 2, mRNA

(SEQ ID NO: 443)
ATGACAACACCCAGAAATTCAGTAAATGGGACTTTCCCGGCAGAGCCAATGAAAGGCCCTATTG
CTATGCAATCTGGTCCAAAACCACTCTTCAGGAGGATGTCTTCACTGGTGGGCCCCACGCAAAG
CTTCTTCATGAGGGAATCTAAGACTTTGGGGGCTGTCCAGATTATGAATGGGCTCTTCCACATT
GCCCTGGGGGGTCTTCTGATGATCCCAGCAGGGATCTATGCACCCATCTGTGTGACTGTGTGGT
ACCCTCTCTGGGGAGGCATTATGTATATTATTTCCGGATCACTCCTGGCAGCAACGGAGAAAAA
CTCCAGGAAGTGTTTGGTCAAAGGAAAAATGATAATGAATTCATTGAGCCTCTTTGCTGCCATT
TCTGGAATGATTCTTTCAATCATGGACATACTTAATATTAAAATTTCCCATTTTTTAAAAATGG
AGAGTCTGAATTTTATTAGAGCTCACACACCATATATTAACATATACAACTGTGAACCAGCTAA
TCCCTCTGAGAAAAACTCCCCATCTACCCAATACTGTTACAGCATACAATCTCTGTTCTTGGGC
ATTTTGTCAGTGATGCTGATCTTTGCCTTCTTCCAGGAACTTGTAATAGCTGGCATCGTTGAGA
ATGAATGGAAAAGAACGTGCTCCAGACCCAAATCTAACATAGTTCTCCTGTCAGCAGAAGAAAA
AAAAGAACAGACTATTGAAATAAAAGAAGAAGTGGTTGGGCTAACTGAAACATCTTCCCAACCA
AAGAATGAAGAAGACATTGAAATTATTCCAATCCAAGAAGAGGAAGAAGAAGAAACAGAGACGA
ACTTTCCAGAACCTCCCCAAGATCAGGAATCCTCACCAATAGAAAATGACAGCTCTCCTTAA.

By “cluster of differentiation 34 (CD34) polypeptide” is meant a protein having at least about 85% amino acid sequence identity to GenBank Accession No. AAA03181.1, provided below, or fragment thereof and having immunomodulatory activity.

>AAA03181.1 CD34 [Homo sapiens]

(SEQ ID NO: 444)
MPRGWTALCLLSLLPSGFMSLDNNGTATPELPTQGTFSNVSTNVSYQET
TTPSTLGSTSLHPVSQHGNEATTNITETTVKFTSTSVITSVYGNTNSSV
QSQTSVISTVFTTPANVSTPETTLKPSLSPGNVSDLSTTSTSLATSPTK
PYTSSSPILSDIKAEIKCSGIREVKLTQGICLEQNKTSSCAEFKKDRGE
GLARVLCGEEQADADAGAQVCSLLLAQSEVRPQCLLLVLANRTEISSKL
QLMKKHQSDLKKLGILDFTEQDVASHQSYSQKTLIALVTSGALLAVLGI
TGYFLMNRRSWSPTGERLGEDPYYTENGGGQGYSSGPGTSPEAQGKASV
NRGAQKNGTGQATSRNGHSARQHVVADTEL.

By “cluster of differentiation 34 (CD34) polynucleotide” is meant a polynucleotide encoding a CD34 polypeptide, as well as the introns, exons, 3′ untranslated regions, 5′ untranslated regions, and regulatory sequences associated with its expression, or fragments thereof. In embodiments, an CD34 polynucleotide is the genomic sequence, cDNA, RNA, or gene associated with and/or required for CD34 expression. An exemplary CD34 nucleic acid sequence is provided below (GenBank Accession No. M81104.1:294-1415).

>M81104.1:294-1415 Homo sapiens CD34 mRNA, Complete Cds

(SEQ ID NO: 445)
ATGCCGCGGGGCTGGACCGCGCTTTGCTTGCTGAGTTTGCTGCCTTCTGGGTTCATGAGTCTTG
ACAACAACGGTACTGCTACCCCAGAGTTACCTACCCAGGGAACATTTTCAAATGTTTCTACAAA
TGTATCCTACCAAGAAACTACAACACCTAGTACCCTTGGAAGTACCAGCCTGCACCCTGTGTCT
CAACATGGCAATGAGGCCACAACAAACATCACAGAAACGACAGTCAAATTCACATCTACCTCTG
TGATAACCTCAGTTTATGGAAACACAAACTCTTCTGTCCAGTCACAGACCTCTGTAATCAGCAC
AGTGTTCACCACCCCAGCCAACGTTTCAACTCCAGAGACAACCTTGAAGCCTAGCCTGTCACCT
GGAAATGTTTCAGACCTTTCAACCACTAGCACTAGCCTTGCAACATCTCCCACTAAACCCTATA
CATCATCTTCTCCTATCCTAAGTGACATCAAGGCAGAAATCAAATGTTCAGGCATCAGAGAAGT
GAAATTGACTCAGGGCATCTGCCTGGAGCAAAATAAGACCTCCAGCTGTGCGGAGTTTAAGAAG
GACAGGGGAGAGGGCCTGGCCCGAGTGCTGTGTGGGGAGGAGCAGGCTGATGCTGATGCTGGGG
CCCAGGTATGCTCCCTGCTCCTTGCCCAGTCTGAGGTGAGGCCTCAGTGTCTACTGCTGGTCTT
GGCCAACAGAACAGAAATTTCCAGCAAACTCCAACTTATGAAAAAGCACCAATCTGACCTGAAA
AAGCTGGGGATCCTAGATTTCACTGAGCAAGATGTTGCAAGCCACCAGAGCTATTCCCAAAAGA
CCCTGATTGCACTGGTCACCTCGGGAGCCCTGCTGGCTGTCTTGGGCATCACTGGCTATTTCCT
GATGAATCGCCGCAGCTGGAGCCCCACAGGAGAAAGGCTGGGCGAAGACCCTTATTACACGGAA
AACGGTGGAGGCCAGGGCTATAGCTCAGGACCTGGGACCTCCCCTGAGGCTCAGGGAAAGGCCA
GTGTGAACCGAGGGGCTCAGAAAAACGGGACCGGCCAGGCCACCTCCAGAAACGGCCATTCAGC
AAGACAACACGTGGTGGCTGATACCGAATTGTGA.

By “Intercellular Adhesion Molecule 1 (ICAM1)” or “Cluster of Differentiation 54 (CD54) polypeptide” is meant a protein having at least about 85% amino acid sequence identity to GenBank Accession No. AAA52709.1, or a fragment thereof that functions in the immune system.

By “Cluster of Differentiation 54 (CD54) polynucleotide” is meant a nucleic acid molecule encoding an CD54 polypeptide, as well as the introns, exons, 3′ untranslated regions, 5′ untranslated regions, and regulatory sequences associated with its expression, or fragments thereof. An exemplary CD54 polynucleotide is provided at GenBank Accession No. J03132.1. The CD54 gene corresponds to Ensembl: ENSG00000090339.

By “Cluster of Differentiation 58 (CD58) polypeptide” is meant a protein having at least about 85% amino acid sequence identity to NCBI Reference Sequence Accession No. NP_001770.1, or a fragment thereof that functions in the immune system. CD58 and the immunobiology thereof is described in Zhang, et al. “CD58 Immunobiology at a Glance,” Frontiers in Immunology, vol. 12, article 705260 (2021), the disclosure of which is incorporated herein by reference in its entirety for all purposes.

By “Cluster of Differentiation 58 (CD58) polynucleotide” is meant a nucleic acid molecule encoding an CD58 polypeptide, as well as the introns, exons, 3′ untranslated regions, 5′ untranslated regions, and regulatory sequences associated with its expression, or fragments thereof. An exemplary CD58 polynucleotide is provided at NCBI Accession No. NM_001779.3. The CD58 gene corresponds to Ensembl: ENSG00000116815.

By “class II, major histocompatibility complex, transactivator (CIITA) polypeptide” is meant a protein having at least about 85% amino acid sequence identity to NCBI Accession No. NP_001273331.1, which is provided below, or a fragment thereof having DNA binding activity.

>NP 001273331.1 MHC Class II Transactivator Isoform 1 [Homo sapiens]

(SEQ ID NO: 446)
MRCLAPRPAGSYLSEPQGSSQCATMELGPLEGGYLELLNSDADPLCLYHFYDQMDLAGEEEIEL
YSEPDTDTINCDQFSRLLCDMEGDEETREAYANIAELDQYVFQDSQLEGLSKDIFIEHIGPDEV
IGESMEMPAEVGQKSQKRPFPEELPADLKHWKPAEPPTVVTGSLLVGPVSDCSTLPCLPLPALF
NQEPASGQMRLEKTDQIPMPFSSSSLSCLNLPEGPIQFVPTISTLPHGLWQISEAGTGVSSIFI
YHGEVPQASQVPPPSGFTVHGLPTSPDRPGSTSPFAPSATDLPSMPEPALTSRANMTEHKTSPT
QCPAAGEVSNKLPKWPEPVEQFYRSLQDTYGAEPAGPDGILVEVDLVQARLERSSSKSLERELA
TPDWAERQLAQGGLAEVLLAAKEHRRPRETRVIAVLGKAGQGKSYWAGAVSRAWACGRLPQYDF
VFSVPCHCLNRPGDAYGLQDLLFSLGPQPLVAADEVFSHILKRPDRVLLILDGFEELEAQDGFL
HSTCGPAPAEPCSLRGLLAGLFQKKLLRGCTLLLTARPRGRLVQSLSKADALFELSGFSMEQAQ
AYVMRYFESSGMTEHQDRALTLLRDRPLLLSHSHSPTLCRAVCQLSEALLELGEDAKLPSTLTG
LYVGLLGRAALDSPPGALAELAKLAWELGRRHQSTLQEDQFPSADVRTWAMAKGLVQHPPRAAE
SELAFPSFLLQCFLGALWLALSGEIKDKELPQYLALTPRKKRPYDNWLEGVPRFLAGLIFQPPA
RCLGALLGPSAAASVDRKQKVLARYLKRLQPGTLRARQLLELLHCAHEAEEAGIWQHVVQELPG
RLSFLGTRLTPPDAHVLGKALEAAGQDFSLDLRSTGICPSGLGSLVGLSCVTRFRAALSDTVAL
WESLQQHGETKLLQAAEEKFTIEPFKAKSLKDVEDLGKLVQTQRTRSSSEDTAGELPAVRDLKK
LEFALGPVSGPQAFPKLVRILTAFSSLQHLDLDALSENKIGDEGVSQLSATFPQLKSLETLNLS
QNNITDLGAYKLAEALPSLAASLLRLSLYNNCICDVGAESLARVLPDMVSLRVMDVQYNKFTAA
GAQQLAASLRRCPHVETLAMWTPTIPFSVQEHLQQQDSRISLR.

By “class II, major histocompatibility complex, transactivator (CIITA) polynucleotide” is meant a nucleic acid molecule encoding an CIITA polypeptide, as well as the introns, exons, 3′ untranslated regions, 5′ untranslated regions, and regulatory sequences associated with its expression, or fragments thereof. An exemplary CIITA polynucleotide is provided at NCBI Accession No. NM_001286402.1, which is provide below.

>NM_001286402.1 Homo sapiens Class II Major Histocompatibility Complex Transactivator (CIITA), Transcript Variant 1, mRNA

(SEQ ID NO: 447)
GGTTAGTGATGAGGCTAGTGATGAGGCTGTGTGCTTCTGAGCTGGGCATCCGAAGGCATCCTTG
GGGAAGCTGAGGGCACGAGGAGGGGCTGCCAGACTCCGGGAGCTGCTGCCTGGCTGGGATTCCT
ACACAATGCGTTGCCTGGCTCCACGCCCTGCTGGGTCCTACCTGTCAGAGCCCCAAGGCAGCTC
ACAGTGTGCCACCATGGAGTTGGGGCCCCTAGAAGGTGGCTACCTGGAGCTTCTTAACAGCGAT
GCTGACCCCCTGTGCCTCTACCACTTCTATGACCAGATGGACCTGGCTGGAGAAGAAGAGATTG
AGCTCTACTCAGAACCCGACACAGACACCATCAACTGCGACCAGTTCAGCAGGCTGTTGTGTGA
CATGGAAGGTGATGAAGAGACCAGGGAGGCTTATGCCAATATCGCGGAACTGGACCAGTATGTC
TTCCAGGACTCCCAGCTGGAGGGCCTGAGCAAGGACATTTTCATAGAGCACATAGGACCAGATG
AAGTGATCGGTGAGAGTATGGAGATGCCAGCAGAAGTTGGGCAGAAAAGTCAGAAAAGACCCTT
CCCAGAGGAGCTTCCGGCAGACCTGAAGCACTGGAAGCCAGCTGAGCCCCCCACTGTGGTGACT
GGCAGTCTCCTAGTGGGACCAGTGAGCGACTGCTCCACCCTGCCCTGCCTGCCACTGCCTGCGC
TGTTCAACCAGGAGCCAGCCTCCGGCCAGATGCGCCTGGAGAAAACCGACCAGATTCCCATGCC
TTTCTCCAGTTCCTCGTTGAGCTGCCTGAATCTCCCTGAGGGACCCATCCAGTTTGTCCCCACC
ATCTCCACTCTGCCCCATGGGCTCTGGCAAATCTCTGAGGCTGGAACAGGGGTCTCCAGTATAT
TCATCTACCATGGTGAGGTGCCCCAGGCCAGCCAAGTACCCCCTCCCAGTGGATTCACTGTCCA
CGGCCTCCCAACATCTCCAGACCGGCCAGGCTCCACCAGCCCCTTCGCTCCATCAGCCACTGAC
CTGCCCAGCATGCCTGAACCTGCCCTGACCTCCCGAGCAAACATGACAGAGCACAAGACGTCCC
CCACCCAATGCCCGGCAGCTGGAGAGGTCTCCAACAAGCTTCCAAAATGGCCTGAGCCGGTGGA
GCAGTTCTACCGCTCACTGCAGGACACGTATGGTGCCGAGCCCGCAGGCCCGGATGGCATCCTA
GTGGAGGTGGATCTGGTGCAGGCCAGGCTGGAGAGGAGCAGCAGCAAGAGCCTGGAGCGGGAAC
TGGCCACCCCGGACTGGGCAGAACGGCAGCTGGCCCAAGGAGGCCTGGCTGAGGTGCTGTTGGC
TGCCAAGGAGCACCGGCGGCCGCGTGAGACACGAGTGATTGCTGTGCTGGGCAAAGCTGGTCAG
GGCAAGAGCTATTGGGCTGGGGCAGTGAGCCGGGCCTGGGCTTGTGGCCGGCTTCCCCAGTACG
ACTTTGTCTTCTCTGTCCCCTGCCATTGCTTGAACCGTCCGGGGGATGCCTATGGCCTGCAGGA
TCTGCTCTTCTCCCTGGGCCCACAGCCACTCGTGGCGGCCGATGAGGTTTTCAGCCACATCTTG
AAGAGACCTGACCGCGTTCTGCTCATCCTAGACGGCTTCGAGGAGCTGGAAGCGCAAGATGGCT
TCCTGCACAGCACGTGCGGACCGGCACCGGCGGAGCCCTGCTCCCTCCGGGGGCTGCTGGCCGG
CCTTTTCCAGAAGAAGCTGCTCCGAGGTTGCACCCTCCTCCTCACAGCCCGGCCCCGGGGCCGC
CTGGTCCAGAGCCTGAGCAAGGCCGACGCCCTATTTGAGCTGTCCGGCTTCTCCATGGAGCAGG
CCCAGGCATACGTGATGCGCTACTTTGAGAGCTCAGGGATGACAGAGCACCAAGACAGAGCCCT
GACGCTCCTCCGGGACCGGCCACTTCTTCTCAGTCACAGCCACAGCCCTACTTTGTGCCGGGCA
GTGTGCCAGCTCTCAGAGGCCCTGCTGGAGCTTGGGGAGGACGCCAAGCTGCCCTCCACGCTCA
CGGGACTCTATGTCGGCCTGCTGGGCCGTGCAGCCCTCGACAGCCCCCCCGGGGCCCTGGCAGA
GCTGGCCAAGCTGGCCTGGGAGCTGGGCCGCAGACATCAAAGTACCCTACAGGAGGACCAGTTC
CCATCCGCAGACGTGAGGACCTGGGCGATGGCCAAAGGCTTAGTCCAACACCCACCGCGGGCCG
CAGAGTCCGAGCTGGCCTTCCCCAGCTTCCTCCTGCAATGCTTCCTGGGGGCCCTGTGGCTGGC
TCTGAGTGGCGAAATCAAGGACAAGGAGCTCCCGCAGTACCTAGCATTGACCCCAAGGAAGAAG
AGGCCCTATGACAACTGGCTGGAGGGCGTGCCACGCTTTCTGGCTGGGCTGATCTTCCAGCCTC
CCGCCCGCTGCCTGGGAGCCCTACTCGGGCCATCGGCGGCTGCCTCGGTGGACAGGAAGCAGAA
GGTGCTTGCGAGGTACCTGAAGCGGCTGCAGCCGGGGACACTGCGGGCGCGGCAGCTGCTGGAG
CTGCTGCACTGCGCCCACGAGGCCGAGGAGGCTGGAATTTGGCAGCACGTGGTACAGGAGCTCC
CCGGCCGCCTCTCTTTTCTGGGCACCCGCCTCACGCCTCCTGATGCACATGTACTGGGCAAGGC
CTTGGAGGCGGCGGGCCAAGACTTCTCCCTGGACCTCCGCAGCACTGGCATTTGCCCCTCTGGA
TTGGGGAGCCTCGTGGGACTCAGCTGTGTCACCCGTTTCAGGGCTGCCTTGAGCGACACGGTGG
CGCTGTGGGAGTCCCTGCAGCAGCATGGGGAGACCAAGCTACTTCAGGCAGCAGAGGAGAAGTT
CACCATCGAGCCTTTCAAAGCCAAGTCCCTGAAGGATGTGGAAGACCTGGGAAAGCTTGTGCAG
ACTCAGAGGACGAGAAGTTCCTCGGAAGACACAGCTGGGGAGCTCCCTGCTGTTCGGGACCTAA
AGAAACTGGAGTTTGCGCTGGGCCCTGTCTCAGGCCCCCAGGCTTTCCCCAAACTGGTGCGGAT
CCTCACGGCCTTTTCCTCCCTGCAGCATCTGGACCTGGATGCGCTGAGTGAGAACAAGATCGGG
GACGAGGGTGTCTCGCAGCTCTCAGCCACCTTCCCCCAGCTGAAGTCCTTGGAAACCCTCAATC
TGTCCCAGAACAACATCACTGACCTGGGTGCCTACAAACTCGCCGAGGCCCTGCCTTCGCTCGC
TGCATCCCTGCTCAGGCTAAGCTTGTACAATAACTGCATCTGCGACGTGGGAGCCGAGAGCTTG
GCTCGTGTGCTTCCGGACATGGTGTCCCTCCGGGTGATGGACGTCCAGTACAACAAGTTCACGG
CTGCCGGGGCCCAGCAGCTCGCTGCCAGCCTTCGGAGGTGTCCTCATGTGGAGACGCTGGCGAT
GTGGACGCCCACCATCCCATTCAGTGTCCAGGAACACCTGCAACAACAGGATTCACGGATCAGC
CTGAGATGATCCCAGCTGTGCTCTGGACAGGCATGTTCTCTGAGGACACTAACCACGCTGGACC
TTGAACTGGGTACTTGTGGACACAGCTCTTCTCCAGGCTGTATCCCATGAGCCTCAGCATCCTG
GCACCCGGCCCCTGCTGGTTCAGGGTTGGCCCCTGCCCGGCTGCGGAATGAACCACATCTTGCT
CTGCTGACAGACACAGGCCCGGCTCCAGGCTCCTTTAGCGCCCAGTTGGGTGGATGCCTGGTGG
CAGCTGCGGTCCACCCAGGAGCCCCGAGGCCTTCTCTGAAGGACATTGCGGACAGCCACGGCCA
GGCCAGAGGGAGTGACAGAGGCAGCCCCATTCTGCCTGCCCAGGCCCCTGCCACCCTGGGGAGA
AAGTACTTCTTTTTTTTTATTTTTAGACAGAGTCTCACTGTTGCCCAGGCTGGCGTGCAGTGGT
GCGATCTGGGTTCACTGCAACCTCCGCCTCTTGGGTTCAAGCGATTCTTCTGCTTCAGCCTCCC
GAGTAGCTGGGACTACAGGCACCCACCATCATGTCTGGCTAATTTTTCATTTTTAGTAGAGACA
GGGTTTTGCCATGTTGGCCAGGCTGGTCTCAAACTCTTGACCTCAGGTGATCCACCCACCTCAG
CCTCCCAAAGTGCTGGGATTACAAGCGTGAGCCACTGCACCGGGCCACAGAGAAAGTACTTCTC
CACCCTGCTCTCCGACCAGACACCTTGACAGGGCACACCGGGCACTCAGAAGACACTGATGGGC
AACCCCCAGCCTGCTAATTCCCCAGATTGCAACAGGCTGGGCTTCAGTGGCAGCTGCTTTTGTC
TATGGGACTCAATGCACTGACATTGTTGGCCAAAGCCAAAGCTAGGCCTGGCCAGATGCACCAG
CCCTTAGCAGGGAAACAGCTAATGGGACACTAATGGGGCGGTGAGAGGGGAACAGACTGGAAGC
ACAGCTTCATTTCCTGTGTCTTTTTTCACTACATTATAAATGTCTCTTTAATGTCACAGGCAGG
TCCAGGGTTTGAGTTCATACCCTGTTACCATTTTGGGGTACCCACTGCTCTGGTTATCTAATAT
GTAACAAGCCACCCCAAATCATAGTGGCTTAAAACAACACTCACATTTA.

An exemplary CIITA gene sequence is provided at (SEQ ID NO: 448).

By “FK506-binding protein 1A (FKBP1A) polypeptide” is meant a protein having at least about 85% amino acid sequence identity to GenBank Accession No. AAA35844.1, which is provided below, or a fragment thereof having cis-trans prolyl isomerase activity and/or having FK506 (tacrolimus), rapamycin, or other immunosuppressant agent binding activity.

>AAA35844.1 FK506-Binding Protein (FKBP) [Homo sapiens]

(SEQ ID NO: 449)
MGVQVETISPGDGRTFPKRGQTCVVHYTGMLEDGKKFDSSRDRNKPFKFM
LGKQEVIRGWEEGVAQMSVGQRAKLTISPDYAYGATGHPGIIPPHATLVF
DVELLKLE.

By “FK506-binding protein 1A (FKBP1A) polynucleotide” is meant a nucleic acid molecule encoding an FKBP1A polypeptide, as well as the introns, exons, 3′ untranslated regions, 5′ untranslated regions, and regulatory sequences associated with its expression, or fragments thereof. An exemplary FKBP1A polynucleotide sequence is provided at GenBank Accession No. M34539.1:79-405, which is provided below.

>M34539.1:79-405 Human FK506-Binding Protein (FKBP) mRNA, Complete Cds

ATGGGAGTGCAGGTGGAAACCATCTCCCCAGGAGACGGGCGCACCTTCCCCAAGCGCGGCCAGA CCTGCGTGGTGCACTACACCGGGATGCTTGAAGATGGAAAGAAATTTGATTCCTCCCGGGACAG AAACAAGCCCTTTAAGTTTATGCTAGGCAAGCAGGAGGTGATCCGAGGCTGGGAAGAAGGGGTT GCCCAGATGAGTGTGGGTCAGAGAGCCAAACTGACTATATCTCCAGATTATGCCTATGGTGCCA CTGGGCACCCAGGCATCATCCCACCACATGCCACTCTCGTCTTCGATGTGGAGCTTCTAAAACT GGAATGA (SEQ ID NO: 450). An exemplary FKBP1A gene sequence is provided at Ensembl Accession No. ENSG00000088832 (SEQ ID NO: 426).

By “granzyme B (GZMB) polypeptide” is meant a protein having at least about 85% amino acid sequence identity to GenBank accession No. AAA75490.1, which is provided below, or a fragment thereof having immunomodulatory activity.

>AAA75490.1 Granzyme B [Homo sapiens]

(SEQ ID NO: 451)
MQPILLLLAFLLLPRADAGEIIGGHEAKPHSRPYMAYLMIWDQKSLKRCG
GFLIQDDFVLTAAHCWGSSINVTLGAHNIKEQEPTQQFIPVKRAIPHPAY
NPKNFSNDIMLLQLERKAKRTRAVQPLRLPSNKAQVKPGQTCSVAGWGQT
APLGKHSHTLQEVKMTVQEDRKCESDLRHYYDSTIELCVGDPEIKKTSFK
GDSGGPLVCNKVAQGIVSYGRNNGMPPRACTKVSSFVHWIKKTMKRY.

By “granzyme B (GZMB) polynucleotide” is meant a nucleic acid molecule encoding an GZMB polypeptide, as well as the introns, exons, 3′ untranslated regions, 5′ untranslated regions, and regulatory sequences associated with its expression, or fragments thereof. An exemplary GZMB polynucleotide sequence is provided below (GenBank Accession No. M28879.1:1161-1215,2256-2403,2857-2992,3200-3460,4105-4248).

### >M28879.1:1161-1215,2256-2403,2857-2992,3200-3460,4105-4248 Human Granzyme B (CTLA-1) Gene, Complete Cds

ATGCAACCAATCCTGCTTCTGCTGGCCTTCCTCCTGCTGCCCAGGGCAGATGCAGGGGAGATCA TCGGGGGACATGAGGCCAAGCCCCACTCCCGCCCCTACATGGCTTATCTTATGATCTGGGATCA GAAGTCTCTGAAGAGGTGCGGTGGCTTCCTGATACAAGACGACTTCGTGCTGACAGCTGCTCAC TGTTGGGGAAGCTCCATAAATGTCACCTTGGGGGCCCACAATATCAAGGAACAGGAGCCGACCC AGCAGTTTATCCCTGTGAAAAGAGCCATCCCCCATCCAGCCTATAATCCTAAGAACTTCTCCAA TGACATCATGCTACTGCAGCTGGAGAGAAAGGCCAAGCGGACCAGAGCTGTGCAGCCCCTCAGG CTACCTAGCAACAAGGCCCAGGTGAAGCCAGGGCAGACATGCAGTGTGGCCGGCTGGGGGCAGA CGGCCCCCCTGGGAAAACACTCACACACACTACAAGAGGTGAAGATGACAGTGCAGGAAGATCG AAAGTGCGAATCTGACTTACGCCATTATTACGACAGTACCATTGAGTTGTGCGTGGGGGACCCA GAGATTAAAAAGACTTCCTTTAAGGGGGACTCTGGAGGCCCTCTTGTGTGTAACAAGGTGGCCC AGGGCATTGTCTCCTATGGACGAAACAATGGCATGCCTCCACGAGCCTGCACCAAAGTCTCAAG CTTTGTACACTGGATAAAGAAAACCATGAAACGCTACTAA (SEQ ID NO: 452). An exemplary human GZMB polynucleotide sequence is provided at Ensembl Accession No. ENSG00000100453 (SEQ ID NO: 453).

By “interferon gamma (IFN-G) polypeptide” is meant a protein having at least about 85% amino acid sequence identity to GenBank accession No. CAA44325.1, which is provided below, or a functional fragment thereof having immunomodulatory activity.

>CAA44325.1 Interferon-Gamma [Homo sapiens]

(SEQ ID NO: 454)
MQDPYVKEAENLKKYFNAGHSDVADNGTLFLGILKNWKEESDRKIMQSQI
VSFYFKLFKNFKDDQSIQKSVETIKEDMNVKFFNSNKKKRDDFEKLTNYS
VTDLNVQRKAIHELIQVMAELSPAAKTGKRKRSQMLFRGRRASQ.

By “interferon gamma (IFN-G; INFg) polynucleotide” is meant a nucleic acid molecule encoding an IFN-G polypeptide, as well as the introns, exons, 3′ untranslated regions, 5′ untranslated regions, and regulatory sequences associated with its expression, or fragments thereof. An exemplary INFg polynucleotide sequence is provided below (GenBank Accession No. X62468.1:13-447).

>X62468.1:13-447 H. sapiens mRNA for IFN-Gamma (pKC-0)

ATGCAAGACCCATATGTAAAAGAAGCAGAAAACCTTAAGAAATATTTTAATGCAGGTCATTCAG ATGTAGCGGATAATGGAACTCTTTTCTTAGGCATTTTGAAGAATTGGAAAGAGGAGAGTGACAG AAAAATAATGCAGAGCCAAATTGTCTCCTTTTACTTCAAACTTTTTAAAAACTTTAAAGATGAC CAGAGCATCCAAAAGAGTGTGGAGACCATCAAGGAAGACATGAATGTCAAGTTTTTCAATAGCA ACAAAAAGAAACGAGATGACTTCGAAAAGCTGACTAATTATTCGGTAACTGACTTGAATGTCCA ACGCAAAGCAATACATGAACTCATCCAAGTGATGGCTGAACTGTCGCCAGCAGCTAAAACAGGG AAGCGAAAAAGGAGTCAGATGCTGTTTCGAGGTCGAAGAGCATCCCAGTAA (SEQ ID NO: 455). An exemplary human IFN-G polynucleotide sequence is provided at Ensembl Accession No. ENSG00000111537 (SEQ ID NO: 456).

By “interleukin-2 (IL-2) polypeptide” is meant a protein having at least about 85% amino acid sequence identity to GenBank Accession No. CAA23827.1, provided below, or fragment thereof, and having immunomodulatory activity.

>CAA23827.1 Interleukin-2 [Homo sapiens]

(SEQ ID NO: 457)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT.

By “interleukin-2 (IL-2) polynucleotide” is meant a polynucleotide encoding an IL-2 polypeptide, as well as the introns, exons, 3′ untranslated regions, 5′ untranslated regions, and regulatory sequences associated with its expression, or fragments thereof. In embodiments, an IL-2 polynucleotide is the genomic sequence, cDNA, mRNA, or gene associated with and/or required for IL-2 expression. An exemplary IL-2 nucleic acid sequence is provided below (GenBank Accession No. V00564.1:48-509).

>V00564.1:48-509 Human mRNA Encoding Interleukin-2 (IL-2) a Lymphocyte Regulatory Molecule

(SEQ ID NO: 458)
ATGTACAGGATGCAACTCCTGTCTTGCATTGCACTAAGTCTTGCACTTGT
CACAAACAGTGCACCTACTTCAAGTTCTACAAAGAAAACACAGCTACAAC
TGGAGCATTTACTGCTGGATTTACAGATGATTTTGAATGGAATTAATAAT
TACAAGAATCCCAAACTCACCAGGATGCTCACATTTAAGTTTTACATGCC
CAAGAAGGCCACAGAACTGAAACATCTTCAGTGTCTAGAAGAAGAACTCA
AACCTCTGGAGGAAGTGCTAAATTTAGCTCAAAGCAAAAACTTTCACTTA
AGACCCAGGGACTTAATCAGCAATATCAACGTAATAGTTCTGGAACTAAA
GGGATCTGAAACAACATTCATGTGTGAATATGCTGATGAGACAGCAACCA
TTGTAGAATTTCTGAACAGATGGATTACCTTTTGTCAAAGCATCATCTCA
ACACTAACTTGA.

By “nuclear receptor subfamily 3, group C, member 1 (NR3C1) polypeptide” is meant a protein having at least about 85% amino acid sequence identity to GenBank Accession No. CAA26976.1, which is provided below, or a fragment thereof, and capable of binding a steroid drug.

>CAA26976.1 Alpha-Glucocorticoid Receptor [Homo sapiens]

(SEQ ID NO: 459)
MDSKESLTPGREENPSSVLAQERGDVMDFYKTLRGGATVKVSASSPSLAV
ASQSDSKQRRLLVDFPKGSVSNAQQPDLSKAVSLSMGLYMGETETKVMGN
DLGFPQQGQISLSSGETDLKLLEESIANLNRSTSVPENPKSSASTAVSAA
PTEKEFPKTHSDVSSEQQHLKGQTGTNGGNVKLYTTDQSTFDILQDLEFS
SGSPGKETNESPWRSDLLIDENCLLSPLAGEDDSFLLEGNSNEDCKPLIL
PDTKPKIKDNGDLVLSSPSNVTLPQVKTEKEDFIELCTPGVIKQEKLGTV
YCQASFPGANIIGNKMSAISVHGVSTSGGQMYHYDMNTASLSQQQDQKPI
FNVIPPIPVGSENWNRCQGSGDDNLTSLGTLNFPGRTVFSNGYSSPSMRP
DVSSPPSSSSTATTGPPPKLCLVCSDEASGCHYGVLTCGSCKVFFKRAVE
GQHNYLCAGRNDCIIDKIRRKNCPACRYRKCLQAGMNLEARKTKKKIKGI
QQATTGVSQETSENPGNKTIVPATLPQLTPTLVSLLEVIEPEVLYAGYDS
SVPDSTWRIMTTLNMLGGRQVIAAVKWAKAIPGFRNLHLDDQMTLLQYSW
MFLMAFALGWRSYRQSSANLLCFAPDLIINEQRMTLPCMYDQCKHMLYVS
SELHRLQVSYEEYLCMKTLLLLSSVPKDGLKSQELFDEIRMTYIKELGKA
IVKREGNSSQNWQRFYQLTKLLDSMHEVVENLLNYCFQTFLDKTMSIEFP
EMLAEIITNQIPKYSNGNIKKLLFHQK.

By “nuclear receptor subfamily 3, group C, member 1 (NR3C1) polynucleotide” is meant a nucleic acid molecule encoding an NR3C1 polypeptide, as well as the introns, exons, 3′ untranslated regions, 5′ untranslated regions, and regulatory sequences associated with its expression, or fragments thereof. An exemplary NR3C1 polynucleotide sequence is provided at

GenBank Accession No. X03225.1:133-2466, which is Provided Below.

>X03225.1:133-2466 Human mRNA for alpha-glucocorticoid receptor (clone OB7)

ATGGACTCCAAAGAATCATTAACTCCTGGTAGAGAAGAAAACCCCAGCAGTGTGCTTGCTCAGG AGAGGGGAGATGTGATGGACTTCTATAAAACCCTAAGAGGAGGAGCTACTGTGAAGGTTTCTGC GTCTTCACCCTCACTGGCTGTCGCTTCTCAATCAGACTCCAAGCAGCGAAGACTTTTGGTTGAT TTTCCAAAAGGCTCAGTAAGCAATGCGCAGCAGCCAGATCTGTCCAAAGCAGTTTCACTCTCAA TGGGACTGTATATGGGAGAGACAGAAACAAAAGTGATGGGAAATGACCTGGGATTCCCACAGCA GGGCCAAATCAGCCTTTCCTCGGGGGAAACAGACTTAAAGCTTTTGGAAGAAAGCATTGCAAAC CTCAATAGGTCGACCAGTGTTCCAGAGAACCCCAAGAGTTCAGCATCCACTGCTGTGTCTGCTG CCCCCACAGAGAAGGAGTTTCCAAAAACTCACTCTGATGTATCTTCAGAACAGCAACATTTGAA GGGCCAGACTGGCACCAACGGTGGCAATGTGAAATTGTATACCACAGACCAAAGCACCTTTGAC ATTTTGCAGGATTTGGAGTTTTCTTCTGGGTCCCCAGGTAAAGAGACGAATGAGAGTCCTTGGA GATCAGACCTGTTGATAGATGAAAACTGTTTGCTTTCTCCTCTGGCGGGAGAAGACGATTCATT CCTTTTGGAAGGAAACTCGAATGAGGACTGCAAGCCTCTCATTTTACCGGACACTAAACCCAAA ATTAAGGATAATGGAGATCTGGTTTTGTCAAGCCCCAGTAATGTAACACTGCCCCAAGTGAAAA CAGAAAAAGAAGATTTCATCGAACTCTGCACCCCTGGGGTAATTAAGCAAGAGAAACTGGGCAC AGTTTACTGTCAGGCAAGCTTTCCTGGAGCAAATATAATTGGTAATAAAATGTCTGCCATTTCT GTTCATGGTGTGAGTACCTCTGGAGGACAGATGTACCACTATGACATGAATACAGCATCCCTTT CTCAACAGCAGGATCAGAAGCCTATTTTTAATGTCATTCCACCAATTCCCGTTGGTTCCGAAAA TTGGAATAGGTGCCAAGGATCTGGAGATGACAACTTGACTTCTCTGGGGACTCTGAACTTCCCT GGTCGAACAGTTTTTTCTAATGGCTATTCAAGCCCCAGCATGAGACCAGATGTAAGCTCTCCTC CATCCAGCTCCTCAACAGCAACAACAGGACCACCTCCCAAACTCTGCCTGGTGTGCTCTGATGA AGCTTCAGGATGTCATTATGGAGTCTTAACTTGTGGAAGCTGTAAAGTTTTCTTCAAAAGAGCA GTGGAAGGACAGCACAATTACCTATGTGCTGGAAGGAATGATTGCATCATCGATAAAATTCGAA GAAAAAACTGCCCAGCATGCCGCTATCGAAAATGTCTTCAGGCTGGAATGAACCTGGAAGCTCG AAAAACAAAGAAAAAAATAAAAGGAATTCAGCAGGCCACTACAGGAGTCTCACAAGAAACCTCT GAAAATCCTGGTAACAAAACAATAGTTCCTGCAACGTTACCACAACTCACCCCTACCCTGGTGT CACTGTTGGAGGTTATTGAACCTGAAGTGTTATATGCAGGATATGATAGCTCTGTTCCAGACTC AACTTGGAGGATCATGACTACGCTCAACATGTTAGGAGGGCGGCAAGTGATTGCAGCAGTGAAA TGGGCAAAGGCAATACCAGGTTTCAGGAACTTACACCTGGATGACCAAATGACCCTACTGCAGT ACTCCTGGATGTTTCTTATGGCATTTGCTCTGGGGTGGAGATCATATAGACAATCAAGTGCAAA CCTGCTGTGTTTTGCTCCTGATCTGATTATTAATGAGCAGAGAATGACTCTACCCTGCATGTAC GACCAATGTAAACACATGCTGTATGTTTCCTCTGAGTTACACAGGCTTCAGGTATCTTATGAAG AGTATCTCTGTATGAAAACCTTACTGCTTCTCTCTTCAGTTCCTAAGGACGGTCTGAAGAGCCA AGAGCTATTTGATGAAATTAGAATGACCTACATCAAAGAGCTAGGAAAAGCCATTGTCAAGAGG GAAGGAAACTCCAGCCAGAACTGGCAGCGGTTTTATCAACTGACAAAACTCTTGGATTCTATGC ATGAAGTGGTTGAAAATCTCCTTAACTATTGCTTCCAAACATTTTTGGATAAGACCATGAGTAT TGAATTCCCCGAGATGTTAGCTGAAATCATCACCAATCAGATACCAAAATATTCAAATGGAAAT ATCAAAAAACTTCTGTTTCATCAAAAGTGA (SEQ ID NO: 460). An exemplary NR3C1 gene sequence is provided at Ensembl Accession No. ENSG00000113580 (SEQ ID NO: 427).

By “peptidyl-prolyl isomerase A (PPIA) polypeptide” is meant a protein having at least about 85% amino acid sequence identity to GenBank Accession No. CAA37039.1, which is provided below, or a fragment thereof, and having peptidyl-prolyl cis-trans isomerase activity and/or having cyclosporin binding activity.

>CAA37039.1 Peptidylprolyl Isomerase [Homo sapiens]

(SEQ ID NO: 461)
MVNPTVFFDIAVDGEPLGRVSFELFADKVPKTAENFRALSTGEKGFGYKG
SCFHRIIPGFMCQGGDFTRHNGTGGKSIYGEKFEDENFILKHTGPGILSM
ANAGPNINGSQFFICTAKTEWLDGKHVVFGKVKEGMNIVEAMERFGSRNG
KTSKKITIADCGQLE.

By “peptidyl-prolyl isomerase A (PPIA) polynucleotide” is meant a nucleic acid molecule encoding an PPIA polypeptide, as well as the introns, exons, 3′ untranslated regions, 5′ untranslated regions, and regulatory sequences associated with its expression, or fragments thereof. An exemplary PPIA polynucleotide sequence is provided at GenBank Accession No. X52851.1:1660-1728,4173-4203,4318-4406,4628-4800,6215-6350, which is provided below.

### >X52851.1:1660-1728,4173-4203,4318-4406,4628-4800,6215-6350 Human Cyclophilin Gene for Cyclophilin (EC 5.2.1.8)

(SEQ ID NO: 462)
ATGGTCAACCCCACCGTGTTCTTCGACATTGCCGTCGACGGCGAGCCCTT
GGGCCGCGTCTCCTTTGAGCTGTTTGCAGACAAGGTCCCAAAGACAGCAG
AAAATTTTCGTGCTCTGAGCACTGGAGAGAAAGGATTTGGTTATAAGGGT
TCCTGCTTTCACAGAATTATTCCAGGGTTTATGTGTCAGGGTGGTGACTT
CACACGCCATAATGGCACTGGTGGCAAGTCCATCTATGGGGAGAAATTTG
AAGATGAGAACTTCATCCTAAAGCATACGGGTCCTGGCATCTTGTCCATG
GCAAATGCTGGACCCAACACAAATGGTTCCCAGTTTTTCATCTGCACTGC
CAAGACTGAGTGGTTGGATGGCAAGCATGTGGTGTTTGGCAAAGTGAAAG
AAGGCATGAATATTGTGGAGGCCATGGAGCGCTTTGGGTCCAGGAATGGC
AAGACCAGCAAGAAGATCACCATTGCTGACTGTGGACAACTCGAATAA.

An exemplary PPIA gene sequence is provided at Ensembl Accession No. ENSG00000196262 (SEQ ID NO: 428).

By “programmed cell death 1 (PD1) polypeptide” is meant a protein having at least about 85% amino acid sequence identity to GenBank Accession No. AJS10360.1, which is provided below, or a fragment thereof, and having immunomodulatory activity.

>AJS10360.1 Programmed Cell Death 1 Protein [Homo sapiens]

(SEQ ID NO: 463)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

By “programmed cell death 1 (PD1) polynucleotide” is meant a nucleic acid molecule encoding a PD1 polypeptide, as well as the introns, exons, 3′ untranslated regions, 5′ untranslated regions, and regulatory sequences associated with its expression, or fragments thereof. An exemplary PD1 polynucleotide sequence is provided at GenBank Accession No. KJ865861.1, which is provided below.

>KJ865861.1 Homo sapiens Cell-Line G3361 Programmed Cell Death 1 Protein (PDCD1) mRNA, Complete Cds

ATGCAGATCCCACAGGCGCCCTGGCCAGTCGTCTGGGCGGTGCTACAACTGGGCTGGCGGCCAG GATGGTTCTTAGACTCCCCAGACAGGCCCTGGAACCCCCCCACCTTCTCCCCAGCCCTGCTCGT GGTGACCGAAGGGGACAACGCCACCTTCACCTGCAGCTTCTCCAACACATCGGAGAGCTTCGTG CTAAACTGGTACCGCATGAGCCCCAGCAACCAGACGGACAAGCTGGCCGCCTTCCCCGAGGACC GCAGCCAGCCCGGCCAGGACTGCCGCTTCCGTGTCACACAACTGCCCAACGGGCGTGACTTCCA CATGAGCGTGGTCAGGGCCCGGCGCAATGACAGCGGCACCTACCTCTGTGGGGCCATCTCCCTG GCCCCCAAGGCGCAGATCAAAGAGAGCCTGCGGGCAGAGCTCAGGGTGACAGAGAGAAGGGCAG AAGTGCCCACAGCCCACCCCAGCCCCTCACCCAGGCCAGCCGGCCAGTTCCAAACCCTGGTGGT TGGTGTCGTGGGCGGCCTGCTGGGCAGCCTGGTGCTGCTAGTCTGGGTCCTGGCCGTCATCTGC TCCCGGGCCGCACGAGGGACAATAGGAGCCAGGCGCACCGGCCAGCCCCTGAAGGAGGACCCCT CAGCCGTGCCTGTGTTCTCTGTGGACTATGGGGAGCTGGATTTCCAGTGGCGAGAGAAGACCCC GGAGCCCCCCGTGCCCTGTGTCCCTGAGCAGACGGAGTATGCCACCATTGTCTTTCCTAGCGGA ATGGGCACCTCATCCCCCGCCCGCAGGGGCTCAGCCGACGGCCCTCGGAGTGCCCAGCCACTGA GGCCTGAGGATGGACACTGCTCTTGGCCCCTCTGA (SEQ ID NO: 464). An exemplary PD1 polynucleotide sequence is also provided at Ensenbl accession no: ENSG00000188389 (SEQ ID NO: 465).

By “Tumor necrosis factor alpha (TNFa or TNFa) polypeptide” is meant a protein having at least about 85% amino acid sequence identity to GenBank Accession No. CAA26669.1, provided below, or a fragment thereof, and having immunomodulatory activity.

>CAA26669.1 TNF-Alpha [Homo sapiens]

(SEQ ID NO: 466)
MSTESMIRDVELAEEALPKKTGGPQGSRRCLFLSLFSFLIVAGATTLFCL
LHFGVIGPQREEFPRDLSLISPLAQAVRSSSRTPSDKPVAHVVANPQAEG
QLQWLNRRANALLANGVELRDNQLVVPSEGLYLIYSQVLFKGQGCPSTHV
LLTHTISRIAVSYQTKVNLLSAIKSPCQRETPEGAEAKPWYEPIYLGGVF
QLEKGDRLSAEINRPDYLDFAESGQVYFGIIAL.

By “Tumor necrosis factor alpha (TNFa or TNFa) polynucleotide” is meant a polynucleotide encoding a Tumor Necrosis Factor Alpha polypeptide, as well as the introns, exons, 3′ untranslated regions, 5′ untranslated regions, and regulatory sequences associated with its expression, or fragments thereof. In embodiments, a Tumor Necrosis Factor Alpha polynucleotide is the genomic sequence, cDNA, mRNA, or gene associated with and/or required for Tumor Necrosis Factor Alpha expression. An exemplary Tumor Necrosis Factor Alpha nucleic acid sequence is provided below (GenBank Accession No. X02910.1:796-981,1589-1634,1822-1869,2171-2592).

### >X02910.1:796-981,1589-1634,1822-1869,2171-2592 Human Gene for Tumor Necrosis Factor (TNF-Alpha)

ATGAGCACTGAAAGCATGATCCGGGACGTGGAGCTGGCCGAGGAGGCGCTCCCCAAGAAGACAG GGGGGCCCCAGGGCTCCAGGCGGTGCTTGTTCCTCAGCCTCTTCTCCTTCCTGATCGTGGCAGG CGCCACCACGCTCTTCTGCCTGCTGCACTTTGGAGTGATCGGCCCCCAGAGGGAAGAGTTCCCC AGGGACCTCTCTCTAATCAGCCCTCTGGCCCAGGCAGTCAGATCATCTTCTCGAACCCCGAGTG ACAAGCCTGTAGCCCATGTTGTAGCAAACCCTCAAGCTGAGGGGCAGCTCCAGTGGCTGAACCG CCGGGCCAATGCCCTCCTGGCCAATGGCGTGGAGCTGAGAGATAACCAGCTGGTGGTGCCATCA GAGGGCCTGTACCTCATCTACTCCCAGGTCCTCTTCAAGGGCCAAGGCTGCCCCTCCACCCATG TGCTCCTCACCCACACCATCAGCCGCATCGCCGTCTCCTACCAGACCAAGGTCAACCTCCTCTC TGCCATCAAGAGCCCCTGCCAGAGGGAGACCCCAGAGGGGGCTGAGGCCAAGCCCTGGTATGAG CCCATCTATCTGGGAGGGGTCTTCCAGCTGGAGAAGGGTGACCGACTCAGCGCTGAGATCAATC GGCCCGACTATCTCGACTTTGCCGAGTCTGGGCAGGTCTACTTTGGGATCATTGCCCTGTGA (SEQ ID NO: 467). An exemplary TNFa gene sequence is provided at ENSEMBL Accession No. ENSG00000232810 (SEQ ID NO: 468).

By “T cell receptor alpha chain (TRAC) polypeptide” is meant a protein having at least about 85% amino acid sequence identity to GenBank Accession No.: AAO72258.1, which is provided below, or a fragment thereof, and having immunomodulatory activity.

>AAO72258.1 T Cell Receptor Alpha Chain [Homo sapiens]

(SEQ ID NO: 469)
METLLGVSLVILWLQLARVNSQQGEEDPQALSIQEGENATMNCSYKTSIN
NLQWYRQNSGRGLVHLILIRSNEREKHSGRLRVTLDTSKKSSSLLITASR
AADTASYFCATANAGGTSYGKLTFGQGTILTVHPNIQNPDPAVYQLRDSK
SSDKSVCLFTDFDSQTNVSQSKDSDVYITDKTVLDMRSMDFKSNSAVAWS
NKSDFACANAFNNSIIPEDTFFPSPESSCDVKLVEKSFETDTNLNFQNLS
VIGFRILLLKVAGFNLLMTLRLWSS.

By “T cell receptor alpha chain (TRAC) polynucleotide” is meant a polynucleotide encoding a TRAC polypeptide, as well as the introns, exons, 3′ untranslated regions, 5′ untranslated regions, and regulatory sequences associated with its expression, or fragments thereof. In embodiments, a TRAC polynucleotide is the genomic sequence, cDNA, mRNA, or gene associated with and/or required for TRAC expression. An exemplary TRAC nucleic acid sequence is provided below (GenBank Accession No. AY247834.1).

>AY247834.1 Homo sapiens T Cell Receptor Alpha Chain (TCRA) mRNA, TCRA-AV17S1 J52 AC Allele, Complete Cds

ATGGAAACTCTCCTGGGAGTGTCTTTGGTGATTCTATGGCTTCAACTGGCTAGGGTGAACAGTC AACAGGGAGAAGAGGATCCTCAGGCCTTGAGCATCCAGGAGGGTGAAAATGCCACCATGAACTG CAGTTACAAAACTAGTATAAACAATTTACAGTGGTATAGACAAAATTCAGGTAGAGGCCTTGTC CACCTAATTTTAATACGTTCAAATGAAAGAGAGAAACACAGTGGAAGATTAAGAGTCACGCTTG ACACTTCCAAGAAAAGCAGTTCCTTGTTGATCACGGCTTCCCGGGCAGCAGACACTGCTTCTTA CTTCTGTGCTACGGCTAATGCTGGTGGTACTAGCTATGGAAAGCTGACATTTGGACAAGGGACC ATCTTGACTGTCCATCCAAATATCCAGAACCCTGACCCTGCCGTGTACCAGCTGAGAGACTCTA AATCCAGTGACAAGTCTGTCTGCCTATTCACCGATTTTGATTCTCAAACAAATGTGTCACAAAG TAAGGATTCTGATGTGTATATCACAGACAAAACTGTGCTAGACATGAGGTCTATGGACTTCAAG AGCAACAGTGCTGTGGCCTGGAGCAACAAATCTGACTTTGCATGTGCAAACGCCTTCAACAACA GCATTATTCCAGAAGACACCTTCTTCCCCAGCCCAGAAAGTTCCTGTGATGTCAAGCTGGTCGA GAAAAGCTTTGAAACAGATACGAACCTAAACTTTCAAAACCTGTCAGTGATTGGGTTCCGAATC CTCCTCCTGAAAGTGGCCGGGTTTAATCTGCTCATGACGCTGCGGCTGTGGTCCAGCTGA (SEQ ID NO: 470). An exemplary TRAC gene sequence is provided at ENSEMBL Accession No. ENSG00000277734 (SEQ ID NO: 472).

By “adenine” or “9H-Purin-6-amine” is meant a purine nucleobase with the molecular formula C₅H₅N₅, having the structure

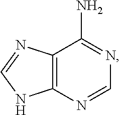

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

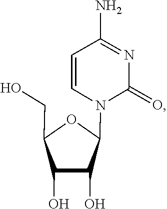

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, PCT/US2017/045381, and PCT/US2020/028568, the full contents of which are each incorporated herein by reference in their entireties for all purposes.

By “adenosine deaminase activity” is meant catalyzing the deamination of adenine or adenosine to guanine in a polynucleotide.

By “Adenosine Base Editor (ABE)” is meant a base editor comprising an adenosine deaminase.

By “Adenosine Base Editor (ABE) polynucleotide” is meant a polynucleotide encoding an ABE.

By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, such alterations are relative to the following reference sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1 In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject. By way of example and without limitation, composition administration (e.g., injection) can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route.

By “agent” is meant any cell, small molecule chemical compound, antibody, nucleic acid molecule, or polypeptide, or fragments thereof.

“Allogeneic,” as used herein, refers to cells that are genetically dissimilar from the cells of a subject to which they are to be administered. In embodiments, allogeneic cells are administered to a genetically dissimilar and/or immunologically incompatible subject.

By “alteration” is meant a change in the level, structure, or activity of an analyte, gene or polypeptide as detected by standard art known methods such as those described herein. As used herein, an alteration includes a change (e.g., increase or reduction) in expression levels. In embodiments, the increase or reduction in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant reduce, suppress, attenuate, diminish, arrest, or stabilize the development or progression of a disease.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antibody” refers to an immunoglobulin molecule that specifically binds to, or is immunologically reactive with, a particular antigen. The term “antibody” includes polyclonal, monoclonal, genetically engineered, and otherwise modified forms of antibodies, including but not limited to chimeric antibodies, humanized antibodies, heteroconjugate antibodies (e.g., bi-tri- and quad-specific antibodies, diabodies, triabodies, and tetrabodies), and antigen binding fragments of antibodies, including, for example, Fab′, F(ab′)2, Fab, Fv, rlgG, and scFv fragments. Further non-limiting examples of antibodies include nanobodies and VHH domains. Unless otherwise indicated, the term “monoclonal antibody” (mAb) is meant to include both intact molecules, as well as antibody fragments (including, for example, Fab and F(ab′)2 fragments) that are capable of specifically binding to a target protein. As used herein, the Fab and F(ab′)2 fragments refer to antibody fragments that lack the Fc fragment of an intact antibody. Examples of these antibody fragments are described herein. Typically, an antibody binds to an antigen using a combination of a variable light chain (VL) and a corresponding variable heavy chain (VH) domain.

The term “antigen-binding domain” refers to a polypeptide or fragment thereof that binds an antigen. In embodiments, two or more antigen-binding domains can function in combination to bind an antigen. In some embodiments, the antigen-binding domain is a region of an antibody.

Antibodies (immunoglobulins) comprise two heavy chains linked together by disulfide bonds, and two light chains, with each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end. The variable domain of the light chain (VL) is aligned with the variable domain of the heavy chain (VL), and the light chain constant domain (CL) is aligned with the first constant domain of the heavy chain (CH1). The variable domains of each pair of light and heavy chains form the antigen binding site. The isotype of the heavy chain (gamma, alpha, delta, epsilon or mu) determines the immunoglobulin class (IgG, IgA, IgD, IgE or IgM, respectively). The light chain is either of two isotypes (kappa (κ) or lambda (λ)) found in all antibody classes. The terms “antibody” or “antibodies” include intact antibodies, such as polyclonal antibodies or monoclonal antibodies (mAbs), as well as proteolytic portions or fragments thereof, such as the Fab or F(ab′)2 fragments, that are capable of specifically binding to a target protein. Antibodies may include chimeric antibodies; recombinant and engineered antibodies, and antigen binding fragments thereof. Exemplary functional antibody fragments comprising whole or essentially whole variable regions of both the light and heavy chains are defined as follows: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating an intact antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating an intact antibody with the enzyme pepsin, followed by reduction (two Fab′ fragments are generated per antibody molecule); and (v) F(ab′)2, a fragment of an antibody molecule containing a monovalent antigen-binding portion of an antibody molecule, obtained by treating an intact antibody with the enzyme pepsin (i.e., a dimer of Fab′ fragments held together by two disulfide bonds).

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1). Representative nucleic acid and protein sequences of base editors include those sequences having about or at least about 85% sequence identity to any base editor sequence provided in the sequence listing, such as those corresponding to SEQ ID NOs: 2-11.

By “BE4 cytidine deaminase (BE4) polypeptide,” is meant a base editor comprising a nucleic acid programmable DNA binding protein (napDNAbp) domain, a cytidine deaminase domain, and two uracil glycosylase inhibitor domains (UGIs). In embodiments, the napDNAbp is a Cas9n(D10A) polypeptide. Non-limiting examples of cytidine deaminase domains include rAPOBEC, ppAPOBEC, RrA3F, AmAPOBEC1, and SsAPOBEC3B.

By “BE4 cytidine deaminase (BE4) polynucleotide,” is meant a polynucleotide encoding a BE4 polypeptide.

By “base editing activity” is meant acting to chemically alter a base within a polynucleotide. In one embodiment, a first base is converted to a second base. In one embodiment, the base editing activity is cytidine deaminase activity, e.g., converting target C•G to T•A. In another embodiment, the base editing activity is adenosine or adenine deaminase activity, e.g., converting A•T to G•C.

By “base editing efficiency” is meant the total percent of one or more target bases in a sample that have been modified using a base editor. In some cases, the base editing efficiency is calculated as the total percent of target polynucleotides in a sample containing a modified target base. In some instances, the base editing efficiency is calculated as the total percent of target polynucleotides in a sample containing a modification to one or more (e.g., 2, 3, 4, 5, 6, 7, 8, 9 or 10) of 2, 3, 4, 5, 6, 7, 8, 9, or 10 target bases. Methods for measuring base editing efficiency for a base editor are known in the art (see, e.g., Gaudelli, et al. Nature 551:464-471 (2017), the disclosure of which is incorporated herein in its entirety for all purposes). In some cases a base editing efficiency is a median base editing efficiency calculated across 2, 3, 4, 5, 6, 7, 8, 9, 10, 15, 20, 25, 30, 35, 40, 45, 50, 60, 70, 80, 90, 100 or more target sites.

By “base editing window” for a base editor is meant bases within a target polynucleotide sequence that can be modified using the base editor. In some embodiments, the position of the nucleobases in the target polynucleotide sequence are numbered relative to a protospacer adjacent motif (PAM) for which a nucleic acid programmable DNA binding protein (napDNAbp) domain of the base editor has specificity, where base 1 corresponds to the base immediately adjacent to the PAM. In some embodiments, the position of the nucleobases in the target polynucleotide sequence are numbered relative to the 5′ or 3′ end of a spacer of a guide polynucleotide used to guide a nucleic acid programmable DNA binding protein (napDNAbp) domain of the base editor to a target site, where base 1 corresponds to the 5′ or 3′ terminal base of the spacer.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

By “calcineurin inhibitor” is meant an immunosuppressive agent capable of inhibiting the activity of calcineurin, a calcium and calmodulin dependent serine/threonine protein phosphatase. Non-limiting examples of calcineurin inhibitors include cyclosporine A, pimecrolimus, tacrolimus, and voclosporin (see Table A).

TABLE A
Representative calcineurin Inhibitors

| | CAS Number | |
|---|---|---|
| Drug Name | (IUPAC name) | Structure |
| Cyclosporine A | 59865-13-3 ((3S,6S,9S,12R,15S,18S,21S, 24S,30S,33S)-30-Ethyl-33- [(1R,2R,4E)-1-hydroxy-2- methyl-4-hexen-1-yl]- 6,9,18,24-tetraisobutyl-3,21- diisopropyl- 1,4,7,10,12,15,19,25,28- nonamethyl- 1,4,7,10,13,16,19,22,25,28,31- undecaazacyclotritriacontane- 2,5,8,11,14,17,20,23,26,29,32- undecone) | 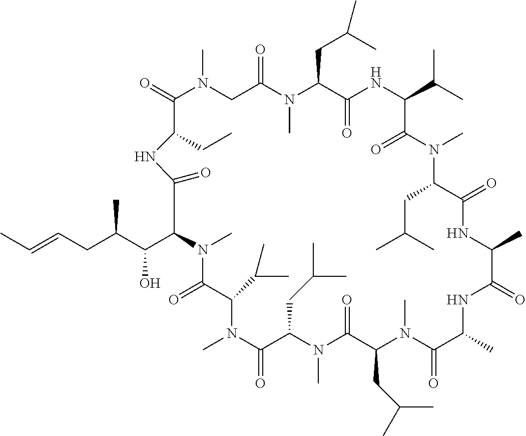 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C] |
| Pimecrolimus | 137071-32-0 ((3S,4R,5S,8R,9E,12S,14S, 15R,16S,18R,19R,26aS)-3- {(E)-2-[(1R,3R,4S)-4-chloro- 3-methoxycyclohexyl]-1- methylvinyl}-8-ethyl- 5,6,8,11,12,13,14,15,16, 17,18,19,24,25,26,26a- hexadecahydro-5,19- dihydroxy-14,16-dimethoxy- 4,10,12,18-tetramethyl-15,19- epoxy-3H-pyrido[2,1- c][1,4]oxaazacyclotricosin- 1,7,20,21(4H,23H)-tetrone) | 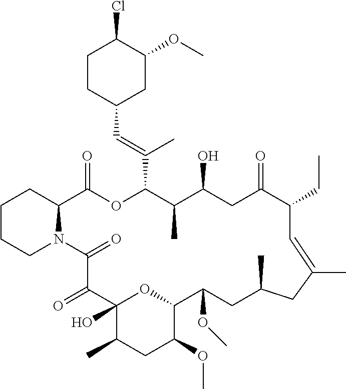 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](Cl)[C@H](OC)C3)[C@H](C)[C@@H](O)CC1=O)[C@H](C)C[C@@H]2OC] |
| Tacrolimus | 104987-11-3 ((−)- (3S,4R,5S,8R,9E,12S,14S, 15R,16S,18R,26aS)-8-allyl- 5,6,8,11,12,13,14,15,16, 17,18,19,24,25,26,26a- hexadecahydro-5,19- dihydroxy-3-{(E)-2- [(1R,3R,4R)-4-hydroxy-3- methylcyclohexyl]-1- methylvinyl}-14,16- dimethoxy-4,10,12,18- tetramethyl-15,19-epoxy-3H- pyrido[2,1- c][1,4]oxaazacyclotricosane- 1,7,20,21(4H,23H)-tetrone) | 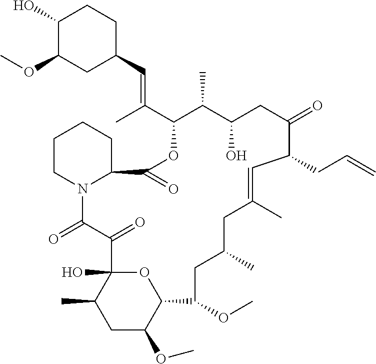 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC] |
| Voclosporin (Lupkynis) | 515814-01-4 ((3S,6S,9S,12R,15S,18S,21S, 24S,30S,33S)-30-Ethyl-33- [(1R,2R,4E)-1-hydroxy-2- methylhepta-4,6-dien-1-yl]- 1,4,7,10,12,15,19,25,28- nonamethyl-6,9,18,24- tetrakis(2-methylpropyl)- 3,21-di(propan-2-yl)- 1,4,7,10,13,16,19,22,25,28,31- undecaazacyclotritriacontane- 2,5,8,11,14,17,20,23,26,29,32- undecone) | 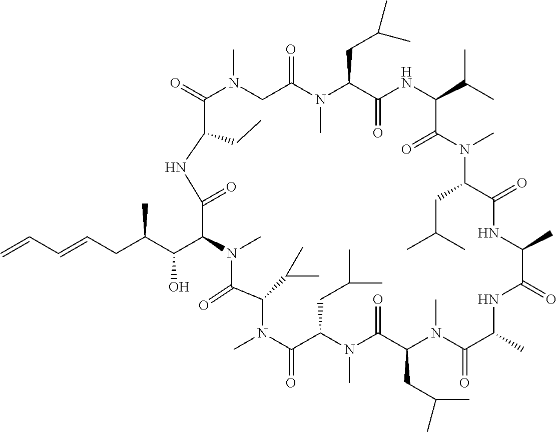 [SMILES: C=C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C] |

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

By “chimeric antigen receptor” or “CAR” is meant a synthetic or engineered receptor comprising an extracellular antigen binding domain operationally joined to one or more intracellular signaling domains where the CAR confers specificity for an antigen bound by the extracellular antigen binding domain onto an immune effector cell. In some cases, the intracellular signaling domain is a T cell signaling domain. In embodiments, the immune effector cell is a T cell, an NK cell, or a macrophage. In embodiments, the CAR is a SUPRA CAR, an anti-tag CAR, a TCR-CAR, or a TCR-like CAR (see, e.g., Guedan, et al. “Engineering and Design of Chimeric Antigen Receptors,” Methods and Clinical Development, 12:145-156 (2019); Poorebrahim, et al., “TCR-like CARs and TCR-CARs targeting neoepitopes: an emerging potential,” Cancer Gene Therapy, 28:581-589 (2021); and Minutolo, et al. “The Emergence of Universal Immune Receptor T Cell Therapy for Cancer,” Front Oncol., 9:176 (2019), the disclosures of which are incorporated herein by reference in their entireties for all purposes).

By “chimeric antigen receptor (CAR) T cell” or “CAR-T cell” is meant a T cell expressing a CAR that has antigen specificity determined by the antibody-derived targeting domain of the CAR. As used herein, “CAR-T cells” include T cells, regulatory T cells (TREG), macrophages, and NK cells. As used herein, the term “CAR-T cells” includes cells engineered to express a CAR or a T cell receptor (TCR, sometimes referred to as TCR-CARs or TCR-like CARs). Methods of making CARs (e.g., for treatment of cancer) are publicly available (see, e.g., Park et al., Trends Biotechnol., 29:550-557, 2011; Grupp et al., N Engl J Med., 368:1509-1518, 2013; Han et al., J. Hematol Oncol. 6:47, 2013; Haso et al., (2013) Blood, 121, 1165-1174; Mohseni, et al., (2020) Front. Immunol., 11, art. 1608, doi: 10.3389/fimmu.2020.01608; Eggenhuizen, et al. Int. J. Mol. Sci. (2020), 21:7015, doi: 10.3390/ijms21197015; Poorebrahim, et al., Cancer Gene Ther 28, 581-589 (2021), doi.org/10.1038/s41417-021-00307-7, PCT Pubs. WO2012/079000, WO2013/059593; and U.S. Pub. 2012/0213783, the disclosure of each of which is incorporated herein by reference herein in its entirety).

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free-OH can be maintained; and glutamine for asparagine such that a free —NH₂ can be maintained.

Amino acids generally can be grouped into classes according to the following common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA, TAG, TAA, TGA.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and x-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2 (1H)-one” is meant a purine nucleobase with the molecular formula C₄H₅N₃O, having the structure

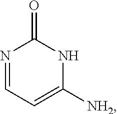

and corresponding to CAS No. 71-30-7.

By “cytidine” is meant a cytosine molecule attached to a ribose sugar via a glycosidic bond, having the structure

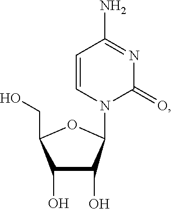

and corresponding to CAS No. 65-46-3. Its molecular formula is C₉H₁₃N₃O₅.

By “Cytidine Base Editor (CBE)” is meant a base editor comprising a cytidine deaminase.

By “Cytidine Base Editor (CBE) polynucleotide” is meant a polynucleotide encoding a CBE.

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, and PCT/US2016/058344. By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. In some embodiments, the disease is a cancer (e.g., a hematological cancer or a solid tumor). In some instances, the disease is a disease that can be treated using the base edited CAR-T cells of the disclosure. In one embodiment, the disease is a neoplasia or cancer. In some embodiments, the disease is a hematological cancer. By “hematological cancer” is meant a malignancy of immune system cells. In some embodiments, the hematological cancer is leukemia, myeloma, and/or lymphoma. Lymphomas and Leukemias are examples of “liquid cancers” or cancers present in the blood and are derived from the transformation of either a hematopoietic precursor in the bone marrow or a mature hematopoietic cell in the blood. Leukemias can be lymphoid or myeloid, and acute or chronic. In the case of myelomas, the transformed cell is a fully differentiated plasma cell that may be present as a dispersed collection of malignant cells or as a solid mass in the bone marrow. In the case of lymphomas, a transformed lymphocyte in a secondary lymphoid tissue generates a solid mass. Lymphomas are classified either Hodgkin lymphoma (HL) or non-Hodgkin lymphoma (NHL). In some instances, the hematologic cancer is a mantle cell lymphoma (MCL) or a B cell lymphoma (BCL).

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C→T activity. In another embodiment, a dual editor has A→G activity that is no more than about 10% or 20% less than C→T activity. In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity.

By “effective amount” is meant the amount of an agent (e.g., a base editor, cell, such as a CAR-T cell) as described herein, that is required to ameliorate the symptoms of a disease relative to an untreated patient or an individual without disease, i.e., a healthy individual, or is the amount of the agent sufficient to elicit a desired biological response. The effective amount of active compound(s) used to practice embodiments of the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

By “glucocorticoid” is meant a class of corticosteroids that bind to a glucocorticoid receptor. Non-limiting examples of glucocorticoids include dexamethasone and prednisolone (see Table B). Further non-limiting examples of glucocorticoids include Cortisol (hydrocortisone), Cortisone, Prednisone, Methylprednisolone, Betamethasone, Triamcinolone, Deflazacort, Fludrocortisone acetate, Deoxycorticosterone acetate, Aldosterone, and Beclometasone.

TABLE B
Representative glucocorticoids

| Drug Name | CAS Number (IUPAC name) | Structure |
|---|---|---|
| Dexamethasone | 50-02-2 ((8S,9R,10S,11S,13S,14S, 16R,17R)-9-Fluoro-11,17- dihydroxy-17-(2- hydroxyacetyl)-10,13,16- trimethyl- 6,7,8,9,10,11,12,13,14,15,16, 17-dodecahydro-3H- cyclopenta[a]phenanthren-3- one) | 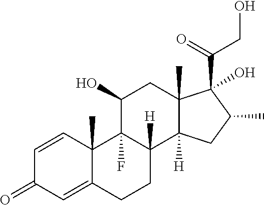 |
| Prednisolone | 50-24-8 ((11β)-11,17,21- Trihydroxypregna-1,4-diene- 3,20-dione) | 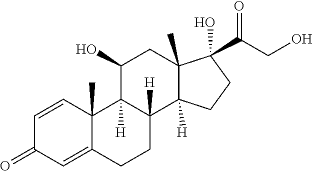 |

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

“Host versus graft disease” (HVGD) or “host-versus-graft rejection” refers to a pathological condition where the immune system of a host generates an immune response against transplanted cells of an allogeneic donor.

By “Human Leukocyte Antigen-E (HLA-E) polypeptide” is meant a protein having at least about 85% amino acid sequence identity to NCBI Accession No. NP_005507.3, or a fragment thereof, and having immunomodulatory activity. An exemplary amino acid sequence is provided below.

(SEQ ID NO: 472)

| | |
|---|---|
| 1 | mvdgtlllll sealaltqtw agshslkyfh tsvsrpgrge |
| | prfisvgyvd dtqfvrfdnd |
| 61 | aasprmvpra pwmeqegsey wdretrsard taqifrvnlr |
| | tlrgyynqse agshtlqwmh |
| 121 | gcelgpdgrf lrgyeqfayd gkdyltlned lrswtavdta |
| | aqiseqksnd aseaehqray |
| 181 | ledtcvewlh kylekgketl lhleppkthv thhpisdhea |
| | tlrcwalgfy paeitltwqq |
| 241 | dgeghtqdte lvetrpagdg tfqkwaavvv psgeeqrytc |
| | hvqheglpep vtlrwkpasq |
| 301 | ptipivgiia glvllgsvvs gavvaaviwr kkssggkggs |
| | yskaewsdsa qgseshsl. |

By “Human Leukocyte Antigen-E (HLA-E) polynucleotide” is meant a nucleic acid molecule encoding an HLA-E polypeptide, as well as the introns, exons, 3′ untranslated regions, 5′ untranslated regions, and regulatory sequences associated with its expression, or fragments thereof. An exemplary HLA-E polynucleotide is provided at NCBI Accession No. NM_005516.6, which is provided below.
1 ctcaggactc agaggctggg atcatggtag atggaaccct ccttttacte ctctcggagg
61 ccctggccct tacccagacc tggggggct cccactcctt gaagtatttc cacacttccg
121 tgtcccggcc cggccgcggg gagccccgct tcatctctgt gggctacgtg gacgacaccc
181 agttcgtgcg cttcgacaac gacgccgcga gtccgaggat ggtgccgcgg gcgccgtgga
241 tggagcagga ggggtcagag tattgggacc gggagacacg gagcgccagg gacaccgcac
301 agattttccg agtgaatctg cggacgctgc gcggctacta caatcagage gaggccgggt
361 ctcacaccct gcagtggatg catggctgcg agctggggcc cgacgggcgc ttcctccgcg
421 ggtatgaaca gttcgcctac gacggcaagg attatctcac cctgaatgag gacctgcgct
481 cctggaccgc ggtggacacg gcggctcaga tctccgagca aaagtcaaat gatgcctctg
541 aggcggagca ccagagagcc tacctggaag acacatgcgt ggagtggctc cacaaatacc
601 tggagaaggg gaaggagacg ctgcttcacc tggagccccc aaagacacac gtgactcacc
661 accccatctc tgaccatgag gccaccctga ggtgctgggc cctgggcttc taccctgcgg
721 agatcacact gacctggcag caggatgggg agggccatac ccaggacacg gagctcgtgg
781 agaccaggcc tgcaggggat ggaaccttcc agaagtgggc agctgtggtg gtgccttctg
841 gagaggagca gagatacacg tgccatgtgc agcatgaggg gctacccgag cccgtcaccc
901 tgagatggaa gccggcttcc cagcccacca tccccatcgt gggcatcatt gctggcctgg
961 ttctccttgg atctgtggtc tctggagctg tggttgctgc tgtgatatgg aggaagaaga
1021 gctcaggtgg aaaaggaggg agctactcta aggctgagtg gagcgacagt gcccaggggt
1081 ctgagtctca cagcttgtaa agcctgagac agctgccttg tgtgcgactg agatgcacag
1141 ctgccttgtg tgcgactgag atgcaggatt tectcacgcc tcccctatgt gtcttagggg
1201 actctggctt ctctttttgc aagggcctct gaatctgtct gtgtccctgt tagcacaatg
1261 tgaggaggta gagaaacagt ccacctctgt gtctaccatg acccccttcc tcacactgac
1321 ctgtgttcct tccctgttct cttttctatt aaaaataaga acctgggcag agtgcggcag
1381 ctcatgcctg taatcccagc acttagggag gccgaggagg gcagatcacg aggtcaggag
1441 atcgaaacca tcctggctaa cacggtgaaa ccccgtctct actaaaaaat acaaaaaatt
1501 agctgggcgc agaggcacgg gcctgtagtc ccagctactc aggaggcgga ggcaggagaa
1561 tggcgtcaac ccgggaggcg gaggttgcag tgagccagga ttgtgcgact gcactccagc
1621 ctgggtgaca gggtgaaacg ccatctcaaa aaataaaaat tgaaaaataa aaaaagaacc
1681 tggatctcaa tttaattttt catattcttg caatgaaatg gacttgagga agctaagatc
1741 atagctagaa atacagataa ttccacagca catctctage aaatttagcc tattcctatt
1801 ctctagccta ttccttacca cctgtaatct tgaccatata ccttggagtt gaatattgtt
1861 ttcatactgc tgtggtttga atgttccctc caacactcat gttgagactt aatccctaat
1921 gtggcaatac tgaaaggtgg ggcctttgag atgtgattgg atcgtaaggc tgtgccttca
1981 ttcatgggtt aatggattaa tgggttatca caggaatggg actggtggct ttataagaag
2041 aggaaaagag aactgagcta gcatgcccag cccacagaga gcctccacta gagtgatgct
2101 aagtggaaat gtgaggtgca gctgccacag agggccccca ccagggaaat gtctagtgtc
2161 tagtggatcc aggccacagg agagagtgcc ttgtggagcg ctgggagcag gacctgacca
2221 ccaccaggac cccagaactg tggagtcagt ggcagcatgc agcgccccct tgggaaagct
2281 ttaggcacca gcctgcaacc cattcgagca gccacgtagg ctgcacccag caaagccaca
2341 ggcacggggc tacctgaggc cttgggggcc caatccctgc tccagtgtgt ccgtgaggca
2401 gcacacgaag tcaaaagaga ttattctctt cccacagata ccttttctct cccatgaccc
2461 tttaacagca tctgcttcat tcccctcacc ttcccaggct gatctgaggt aaactttgaa
2521 gtaaaataaa agctgtgttt gagcatca (SEQ ID NO: 473). The HLA-E gene corresponds to Ensembl: ENSG00000116815.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immune cell” is meant a cell of the immune system capable of generating an immune response. Exemplary immune cells include, but are not limited to, T cells, NK cells, B cells, macrophages, hematopoietic stem cells, or precursors thereof. In embodiments, an immune cell is allogeneic to a subject to whom the cell is to be administered. In embodiments, an immune cell is from a donor and is allogeneic to a subject to which the immune cell will be administered after being modified according to the methods provided herein. The disclosure features methods for preparing modified allogeneic immune cells with improved characteristics (e.g., increased persistence in a subject) as well as the cells produced by these methods.

By “immune effector cell” is meant a lymphocyte, once activated, capable of effecting an immune response upon a target cell. In some embodiments, immune effector cells are effector T cells. In some embodiments, the effector T cell is a naïve CD8⁺ T cell, a cytotoxic T cell, a natural killer T (NKT) cell, a macrophage, or a natural killer (NK) cell. In some embodiments, immune effector cells are effector NK cells. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell).

By “immunogen encoding polynucleotide” is meant a nucleic acid molecule that encodes an immunogen.

By “immunomodulatory activity” is meant increasing, decreasing, or sustaining an immune response.

By “immunosuppressant agent” is meant an agent associated with inhibiting or preventing activity of an immune cell. In some embodiments, an immunosuppressant agent is an agent that reduces or prevents the ability of a subject's immune system to elicit an immune response to an allogeneic T- or NK-cell (e.g, a CAR-T cell) administered to the subject. Non-limiting examples of immunosuppressant agents include mTOR inhibitors (e.g., a rapalog, such as rapamycin or Everolimus), Calcineurin Inhibitors (e.g., cyclosporine A or tacrolimus), and Glucocorticoids (e.g., Dexamethasone or Prednisolone).

By “immunosuppression” is meant a reduction in or elimination of the ability of an immune cell to elicit an immune response when exposed to an immunosuppressive agent. In embodiments, the immunosuppressive agent is an immunosuppressive agent. In some cases, the immune response is antigen-dependent proliferation, cytokine production, and/or killing of a target cell.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “kill switch” refers to a polypeptide capable of mediating the killing of a cell when the polypeptide is specifically bound by an agent. In some cases, the agent is a small molecule or monoclonal antibody. In embodiments, a chimeric antigen receptor of the disclosure contains a kill switch and/or a cell of the disclosure surface-expresses a kill switch. In some cases, the agent is Rituximab. In various embodiments, the kill switch is selected from RQR1, RQR2, RQR8, RQR1G4S, RQR2G4S, RR, G4SRR, G4SRRG4S, G4SRRG4SCD8, G4SRRG4SCD28, G4SRRCD28, and QG4S, the amino acid sequences of which are listed in Table C. In various embodiments, the kill switch is fused to a chimeric antigen receptor. In some embodiments, a kill switch is expressed on the surface of a cell and is not fused to a chimeric antigen receptor.

TABLE C
Representative kill switch amino acid sequences

| Kill switch | | SEQ ID |
|---|---|---|
| name | Amino acid sequence | NO |
| RQR1 | CPYSNPSLCSGGGGSELPTQGTFSNVSTNVSPAKPTTTACPYSNPSLC | 474 |
| RQR2 | CPYSNPSLCSGGGGSELPTQGTFSNVSTNVSPAKPTTTCPYSNPSLC | 475 |
| RQR1G4S | SGGGGSCPYSNPSLCSGGGGSELPTQGTFSNVSTNVSPAKPTTTACPYSNPS | 476 |
| | LCSGGGGS | |
| RQR2G4S | SGGGGSCPYSNPSLCSGGGGSELPTQGTFSNVSTNVSPAKPTTTACPYSNPS | 476 |
| | LCSGGGGS | |
| RR | CPYSNPSLCSGGGGSPAKPTTTACPYSNPSLC | 478 |
| G4SRR | SGGGGSCPYSNPSLCSGGGGSPAKPTTTACPYSNPSLC | 479 |
| G4SRRG4S | SGGGGSCPYSNPSLCSGGGGSPAKPTTTACPYSNPSLCSGGGGS | 480 |
| G4SRRG4SCD8 | SGGGGSCPYSNPSLCSGGGGSPAKPTTTACPYSNPSLCSGGGGSPAPRPPTP | 481 |
| | APTIASQPLSLRPEACRPAAGGAVHTRGLDFACD | |
| G4SRRG4SCD28 | SGGGGSCPYSNPSLCSGGGGSPAKPTTTACPYSNPSLCSGGGGSIEVMYPPP | 482 |
| | YLDNEKSNGTIIHVKGKHLCPSPLFPGPSKP | |
| G4SRRCD28 | SGGGGSCPYSNPSLCSGGGGSPAKPTTTACPYSNPSLCSIEVMYPPPYLDNE | 483 |
| | KSNGTIIHVKGKHLCPSPLFPGPSKP | |
| QG4S | ELPTQGTFSNVSTNVSSSGGGGSGGGGSGGGGS | 484 |

The term “linker”, as used herein, refers to a molecule that links two moieties. In one embodiment, the term “linker” refers to a covalent linker (e.g., covalent bond) or a non-covalent linker.

By “marker” is meant any agent or clinical parameter having an alteration that is associated with a disease or disorder. In embodiments, the agent is a polypeptide or polynucleotide and the alteration is in expression, level, structure, or activity. In embodiments, the disease or disorder is a neoplasia, such as a hematologic cancer (e.g., a lymphoma). A non-limiting examples of a markers include CD5, CD7, CD19, CD20, CD22, CD79B, and ROR1.

By “mechanistic target of rapamycin (mTOR) inhibitor” is meant an agent that inhibits activity of the mechanistic target of rapamycin (mTOR), which is a serine/threonine-specific protein kinase belonging to the family of phosphatidinyl-3 kinase (PI3K) related kinases (PIKKs). In embodiments, the mTOR is a rapalog. Non-limiting examples of rapalogs include rapamycin (see Table D) and analogs thereof. Further non-limiting examples of rapalogs include temsirolimus (CCI-779), everolimus (RAD001), and ridaforolimus (AP-23573) (see Table D).

TABLE D
Representative mTOR Inhibitors

| Drug Name | CAS Number (IUPAC name) | Structure |
|---|---|---|
| Rapamycin | 53123-88-9 ((1R,9S,12S,15R,16E,18R,19R,21R, 23S,24E,26E,28E,30S,32S,35R)- 1,18-dihydroxy-12-{(2R)-1- [(1S,3R,4R)-4-hydroxy-3- methoxycyclohexyl]-2-propanyl}- 19,30-dimethoxy- 15,17,21,23,29,35-hexamethyl- 11,36-dioxa-4- azatricyclo[30.3.1.0~4,9~] hexatriaconta-16,24,26,28-tetraene- 2,3,10,14,20-pentone) | 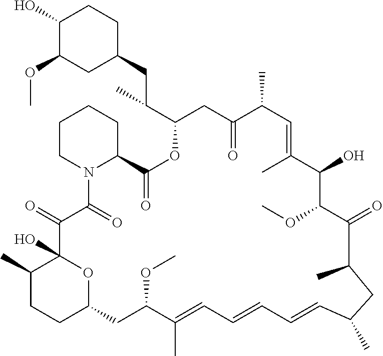 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C] |
| Temsirolimus | 162635-04-3 ((1R,2R,4S)-4-{(2R)- 2- [(3S,6R,7E,9R,10R,12R,14S,15E, 17E,19E,21S,23S,26R,27R,34aS)- 9,27-dihydroxy-10,21-dimethoxy- 6,8,12,14,20,26-hexamethyl- 1,5,11,28,29-pentaoxo- 1,4,5,6,9,10,11,12,13,14,21,22,23, 24,25,26,27,28,29,31,32,33,34,34a- tetracosahydro-3H-23,27- epoxypyrido[2,1- c][1,4]oxazacyclohentriacontin-3- yl]propyl}-2-methoxycyclohexyl 3- hydroxy-2-(hydroxymethyl)-2- methylpropanoate) | 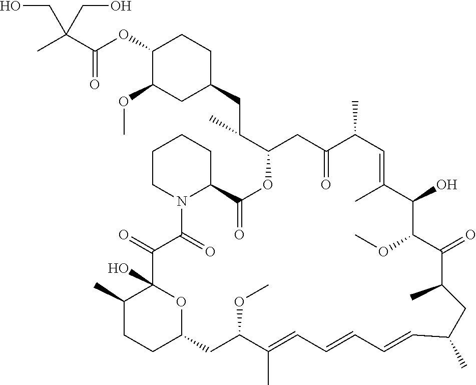 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C] |
| Everolimus (Afinitor, Zortress) | 159351-69-6 (Dihydroxy-12-[(2R)- 1-[(1S,3R,4R)-4-(2- hydroxyethoxy)-3- methoxycyclohexyl]propan-2-yl]- 19,30-dimethoxy- 15,17,21,23,29,35-hexamethyl- 11,36-dioxa-4-azatricyclo[30.3.1.0 hexatriaconta-16,24,26,28-tetraene- 2,3,10,14,20-pentone) | 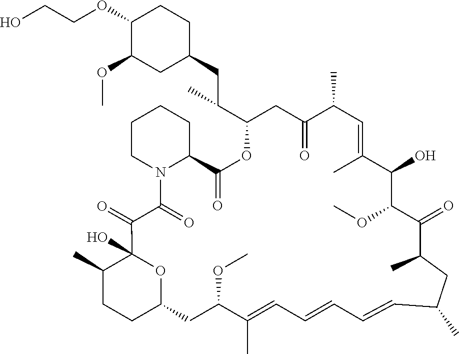 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C] |
| Ridaforolimus | 572924-54-0 ((1R,2R,4S)-4-[(2R)- 2- [(1R,9S,12S,15R,16E,18R,19R,21R, 23S,24E,26E,28Z,30S,32S,35R)- 1,18-dihydroxy-19,30-dimethoxy- 15,17,21,23,29,35-hexamethyl- 2,3,10,14,20-pentaoxo-11,36-dioxa- 4-azatricyclo [30.3.1.04,9]hexatriaconta- 16,24,26,28-tetraen-12- yl]propyl]-2-methoxycyclohexyl dimethylphosphinate) | 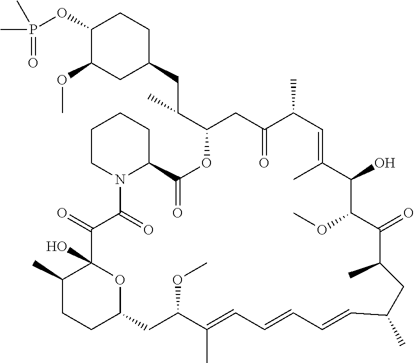 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OP(C)(C)=O)[C@H](OC)C2)CC(=O)[C@H](C)/C=C(\C)[C@@H](O)[C@@H](OC)C(=O)[C@H](C)C[C@H](C)/C=C/C=C/C=C/1C] |

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

“Neoplasia” refers to cells or tissues exhibiting abnormal growth or proliferation. The term neoplasia encompasses cancer, liquid, and solid tumors. In some embodiments, the neoplasia is a solid tumor. In other embodiments, the neoplasia is a liquid tumor. In some embodiments, the neoplasia is a hematological cancer. In some embodiments, the hematological cancer is leukemia, myeloma, and/or lymphoma. In some embodiments, the hematological cancer is a B cell cancer (e.g., a B cell lymphoma). In some embodiments, the B cell cancer is a lymphoma or a leukemia. In some cases, the leukemia comprises a pre-leukemia. In some instances, the neoplasia is a mantle cell lymphoma. In some cases, the leukemia is an acute leukemia. Acute leukemias include, for example, an acute myeloid leukemia (AML). Acute leukemias also include, for example, an acute lymphoid leukemia or an acute lymphocytic leukemia (ALL); ALL includes B-lineage ALL; T-lineage ALL; and T-cell acute lymphocytic leukemia (T-ALL).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence

(SEQ ID NO: 190)
KRTADGSEFESPKKKRKV,
(SEQ ID NO: 191)
KRPAATKKAGQAKKKK,
(SEQ ID NO: 192)
KKTELQTTNAENKTKKL,
(SEQ ID NO: 193)
KRGINDRNFWRGENGRKTR,
(SEQ ID NO: 194)
RKSGKIAAIVVKRPRK,
(SEQ ID NO: 195)
PKKKRKV,
(SEQ ID NO: 196)
MDSLLMNRRKFLYQFKNVRWAKGRRETYLC,
(SEQ ID NO: 328)
PKKKRKVEGADKRTADGSEFESPKKKRKV,
or
(SEQ ID NO: 329)
RKSGKIAAIVVKRPRKPKKKRKV.

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpfl, Cas12b/C2cl, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpfl, Cas12b/C2cl, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-231, 232-245, 254-257, 260, and 378. In some embodiments, the napDNAbp is a (CRISPR-associated system) Cas9 endonuclease, for example, Cas9 (Csnl) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9).

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

By “persistence” in the context of an allogeneic transplant is meant the continued survival of a donor cell in a host organism. In some embodiments, allogeneic cell(s) comprising one or more of the edits described herein (e.g., a base edit in a CD5, CD3e, CD3g, B2M, and/or CIITA gene, or regulatory element(s) thereof; or knockdown of a CD5, TCRαβ, B2M, and/or CIITA polypeptide) persist in a subject allogeneic to the cells at higher levels over time post-infusion than corresponding unedited allogeneic control cells. In embodiments, the percentage of edited cells (e.g., T cells, NK cells, or lymphocytes) persisting in a subject at a given time point (e.g., 7 days, 14 days, 1 month, 3 months, 6 months, 9 months, or greater than 1, 2, or 3 years is at least about 10%, 15%, 20%, 25%, 30%, 35%, 40%, 45%, 50%, 60%, 70%, 80%, 90%, 95%, 99%, or 100% greater than the level of unedited control cells at the same time point. A cell(s) modified by methods of the present disclosure are more persistent than a reference unmodified cell(s).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide, which comprises protein domains from at least two different proteins.

By “rBE4 polypeptide” is meant a polypeptide sharing at least 85% amino acid sequence identity to the below amino acid sequence and having cytidine base editor activity.

(SEQ ID NO: 485)
MSSETGPVAVDPTLRRRIEPHEFEVFFDPRELRKETCLLYEINWGGRHS
IWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQ
ESGYCWRNFVNYSPSNEAHWPRYPHLWVRLYVLELYCIILGLPPCLNIL
RRKQPQLTFFTIALQSCHYQRLPPHILWATGLKSGGSSGGSSGSETPGT
SESATPESSGGSSGGSDKKYSIGLAIGTNSVGWAVITDEYKVPSKKFKV
LGNTDRHSIKKNLIGALLFDSGETAEATRLKRTARRRYTRRKNRICYLQ
EIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDV
DKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLL
AQIGDQYADLFLAAKNLSDAILLSDILRVNTEITKAPLSASMIKRYDEH
HQDLTLLKALVRQQLPEKYKEIFFDQSKNGYAGYIDGGASQEEFYKFIK
PILEKMDGTEELLVKLNREDLLRKQRTFDNGSIPHQIHLGELHAILRRQ
EDFYPFLKDNREKIEKILTFRIPYYVGPLARGNSRFAWMTRKSEETITP
WNFEEVVDKGASAQSFIERMTNFDKNLPNEKVLPKHSLLYEYFTVYNEL
TKVKYVTEGMRKPAFLSGEQKKAIVDLLFKTNRKVTVKQLKEDYFKKIE
CFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSL
HEHIANLAGSPAIKKGILQTVKVVDELVKVMGRHKPENIVIEMARENQT
TQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGF
IKRQLVETRQITKHVAQILDSRMNTKYDENDKLIREVKVITLKSKLVSD
FRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILP
KRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGR
KRMLASAGELQKGNELALPSKYVNFLYLASHYEKLKGSPEDNEQKQLFV
EQHKHYLDEIIEQISEFSKRVILADANLDKVLSAYNKHRDKPIREQAEN
IIHLFTLTNLGAPAAFKYFDTTIDRKRYTSTKEVLDATLIHQSITGLYE
TRIDLSQLGGDSGGSGGSGGSTNLSDIIEKETGKQLVIQESILMLPEEV
EEVIGNKPESDILVHTAYDESTDENVMLLTSDAPEYKPWALVIQDSNGE
NKIKMLSGGSGGSGGSTNLSDIIEKETGKQLVIQESILMLPEEVEEVIG
NKPESDILVHTAYDESTDENVMLLTSDAPEYKPWALVIQDSNGENKIKM
LSGGSKRTADGSEFESPKKKRKVE.

By “rBE4 polynucleotide” is meant a polynucleotide encoding a rBE4 polypeptide.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a cell (e.g., a CAR-T cell) not base edited according to the methods provided herein. In other embodiments and without limitation, a reference is an untreated cell that is not subjected to a test condition, or is subjected to placebo or normal saline, medium, buffer, a control polynucleotide that does not encode a polypeptide of interest, and/or a control vector that does not harbor a polynucleotide of interest. In some cases, a reference is a healthy subject, such as a subject not having a neoplasia or a subject having a neoplasia and not treated for the neoplasia according to a method provided herein. In some embodiments, the reference is a cell lacking a nucleobase alteration and/or having an additional nucleobase alteration. The reference may be a cell that does not express one or more of the polypeptides described herein. The reference may be a subject before administration of a composition provided herein or treated according to a method provided herein and/or the subject before a change in a treatment (e.g., an alteration in dose or agent administered to the subject).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease-RNA complex. Typically, the bound

RNA(s) is referred to as a guide RNA (gRNA). In some embodiments, the RNA-programmable nuclease is the (CRISPR-associated system) Cas9 endonuclease, for example, Cas9 (Csnl) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g. a sequence with at least about 85% sequence identity to a Cas9, such as Nme2Cas9 or spCas9).

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody. By “specifically binds” is meant a nucleic acid molecule, polypeptide, polypeptide/polynucleotide complex, compound, or molecule that recognizes and binds a polypeptide and/or nucleic acid molecule of the disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced in some embodiments to form a “reconstituted” protein. In some embodiments, the split polypeptide is a nucleic acid programmable DNA binding protein (e.g., a Cas9) or a base editor.

The term “target site” refers to a nucleotide sequence or nucleobase of interest within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas 12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, reduces the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>splP14739IUNGI_BPPB2 Uracil-DNA Glycosylase Inhibitor

(SEQ ID NO: 231)
MTNLSDIIEKETGKQLVIQESILMLPEEVEEVIGNKPESDILVHTAYDE
STDENVMLLTSDAPEYKPWALVIQDSNGENKIKML.

In some embodiments, the agent inhibiting the uracil-excision repair system is a uracil stabilizing protein (USP). See, e.g., WO 2022015969 A1, incorporated herein by reference.

As used herein, the term “vector” refers to a means of introducing a nucleic acid molecule into a cell, resulting in a transformed cell. Vectors include plasmids, transposons, phages, viruses, liposomes, lipid nanoparticles, and episomes.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIGS. 1A and 1B provide a schematic diagram and flow cytometry scatter plots demonstrating that rapamycin and tacrolimus inhibited the priming and proliferation of alloreactive T cells in response to HLA class I mismatched peripheral blood mononuclear cells. FIG. 1A provides a schematic diagram summarizing the experiment carried out to obtain the data shown in FIG. 1B. Purified primary human T cells (effector cells) from a donor (Donor #1) were labeled with CellTrace™ dye and co-cultured at a 1:1 effector-to-target cell ratio with HLA-mismatched peripheral blood mononuclear cells (PBMCs) from another donor (Donor #2) that were pretreated with mitomycin C to inhibit their growth (mitomycin C inhibits cell growth). The cells were co-cultured in the presence of the immunosuppressant rapamycin (0.001 μg/mL) or tacrolimus (0.1 μg/mL) or in the presence of dimethylsulfoxide (DMSO) in place of any immunosuppressant. The immunosuppressant or DMSO was re-administered to the cultures every 48 hours. On day 7, proliferation was measured as dilution of the CellTrace™ dye assessed using flow cytometry. The numbers at the lower-left of each plot indicate the percent of total cells counted falling within the indicated region.

FIG. 2 provides a series of flow cytometry scatter plots demonstrating that NK cell-mediated lysis of HLA class-I negative T cells is attenuated in the presence of the immunosuppressant rapamycin or tacrolimus. The rightmost plots represent the ratio of HLA class-I negative (target cells) and positive T cells in the absence of any NK cells (effector cells) (i.e., a culture with an effector-to-target cell ratio (E:T) of 0:1 because the culture contained no effector cells and only the target cells). Primary human NK cells were activated in vitro with IL-2 and IL-15 cytokines for 72 hours. The activated NK cells were then cultured for 24 hours in the presence of rapamycin (10 μg/mL), tacrolimus (10 μg/mL), or a dimethylsulfoxide (DMSO) control. After the 24 hours, the NK cells were then co-cultured at a 4:1 effector-to-target ratio with an equal mixture of unedited HLA class-I positive (off-target cells) or beta-2-microglobulin (B2M) knock-out (HLA class-I deficient) T cells (target cells) in the presence or absence if the immunosuppressant rapamycin or tacrolimus. T cells co-cultured in the absence of NK cells (0:1 E:T ratio) were used as control. Following 48 hours of co-culture, the frequency of HLA class-I negative and positive T cells was assessed using flow cytometry by immunostaining for pan HLA class-I (ABC). The numbers within each plot indicate the percent of total cells counted falling within the indicated region, where the left regions correspond to HLA class-I negative cells and the right regions correspond to HLA class-I positive cells.

FIG. 3 provides a bar graph showing maximum A to G and C to T base editing within an FKBP1A polynucleotide achieved using base editor systems containing one of the guide polynucleotides indicated along the x-axis (see Table 1 for nucleotide sequences) and either an adenosine deaminase base editor (ABE) or a cytidine deaminase base editor (CBE), as indicated. The ABE was ABE8.20m and the CBE was rBE4. Primary human T cells were thawed and activated in vitro using an anti-CD3/CD28/CD2 reagent. On day 2 following contacting with the reagent, the T cells were washed and resuspended in P3 Buffer (Lonza) and plated in 96-well electroporation plates containing 2 μg ABE8.20m or rBE4 (CBE) and 2 μg of the indicated guide polynucleotide targeting the FKBP1A (FKBP prolyl isomerase 1A) polynucleotide. Plates were electroporated using a DH-102 setting on a Lonza Nucleofector™ 96-well plate unit. The electroporated cells were incubated for 3 days. Following the 3-day incubation, the cells were harvested, genomic DNA was extracted, and next-generation sequencing was carried out on the genomic DNA to determine frequencies of base pair conversion within the FKBP1A polynucleotide.

FIG. 4 provides Western blot analysis images demonstrating knock-out of FKBP1A protein expression in T cells edited using a base editor system containing the base editor ABE8.20m and a TSBT×1538 sgRNA. Primary human T cells were activated with anti-CD3/CD28/CD2 reagent. On day 2 following contacting with the reagent, the T cells were contacted with the TSBT×1538 sgRNA and ABE8.20m and subsequently incubated for approximately 5 days. Unedited cells were used as a negative control. Following the approximately 5-day incubation, the cells were harvested and cell-associated protein was extracted to be evaluated in the Western blot analysis. Protein samples from the edited and unedited cells were contacted with a rabbit anti-FKBP1A or mouse anti-GAPDH (control used to normalize protein concentrations) antibodies, followed by staining with secondary anti-rabbit (HRP) and anti-mouse (NIR) antibodies, respectively. Protein was analyzed using the Jess automated Western blot analysis device available from ProteinSimple.

FIG. 5 provides a bar graph showing maximum A to G and C to T base editing within an NR3C1 polynucleotide achieved using base editor systems containing one of the guide polynucleotides indicated along the x-axis (see Table 1 for nucleotide sequences) and either an adenosine deaminase base editor (ABE) or a cytidine deaminase base editor (CBE), as indicated. The ABE was ABE8.20m and the CBE was rBE4. Primary human T cells were thawed and activated in vitro using an anti-CD3/CD28/CD2 reagent. On day 2 following contacting with the reagent, the T cells were washed and resuspended in P3 Buffer (Lonza) and plated in 96-well electroporation plates containing 2 μg mRNA encoding ABE8.20m or rBE4 (CBE) and 2 μg of the indicated guide polynucleotide targeting the NR3C1 (nuclear receptor subfamily 3 group C member 1) polynucleotide. Plates were electroporated using a DH-102 setting on a Lonza Nucleofector™ 96-well plate unit. The electroporated cells were incubated for 3 days. Following the 3-day incubation, the cells were harvested, genomic DNA was extracted, and next-generation sequencing was carried out on the genomic DNA to determine frequencies of base pair conversion within the NR3C1 polynucleotide.

FIG. 6 provides a bar graph showing maximum A to G base editing within a PPIA polynucleotide achieved using base editor systems containing one of the guide polynucleotides indicated along the x-axis (see Table 1 for nucleotide sequences) and an adenosine deaminase base editor (ABE). The ABE was ABE8.20m. Primary human T cells were thawed and activated in vitro using an anti-CD3/CD28/CD2 reagent. On day 2 following contacting with the reagent, the T cells were washed and resuspended in P3 Buffer (Lonza) and plated in 96-well electroporation plates containing 2 μg ABE8.20m and 2 μg of the indicated guide polynucleotide targeting the PPIA (peptidylprolyl isomerase A) polynucleotide. Plates were electroporated using a DH-102 setting on a Lonza Nucleofector™ 96-well plate unit. The electroporated cells were incubated in 24-well GRex™ (Wilson Wolf) plates for 5 days. Following the 5-day incubation, the cells were harvested, genomic DNA was extracted, and next-generation sequencing was carried out on the genomic DNA to determine frequencies of base pair conversion within the PPIA polynucleotide.

FIG. 7 provides a set of overlaid flow cytometry histograms confirming knock-out of PPIA protein expression in primary human T cells edited using base editor systems containing

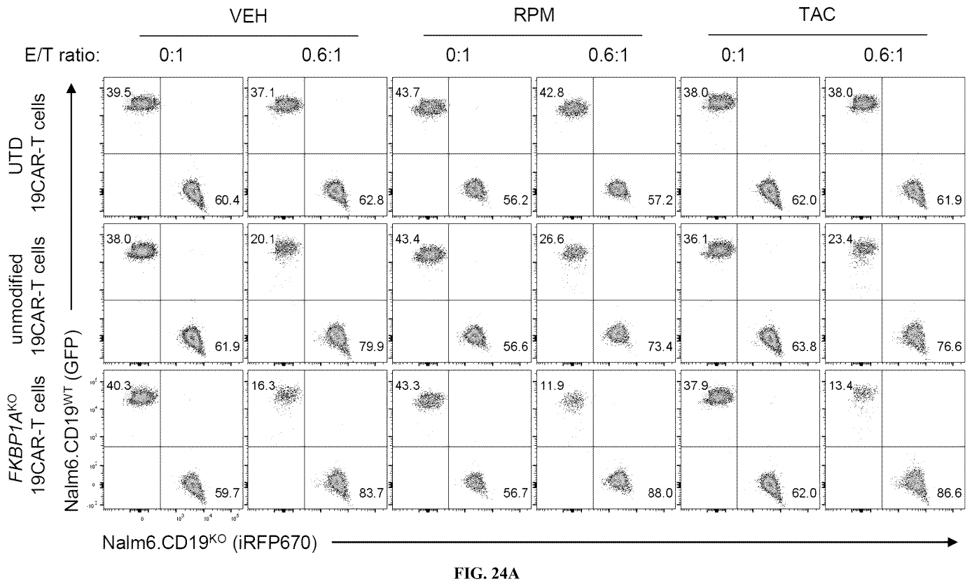

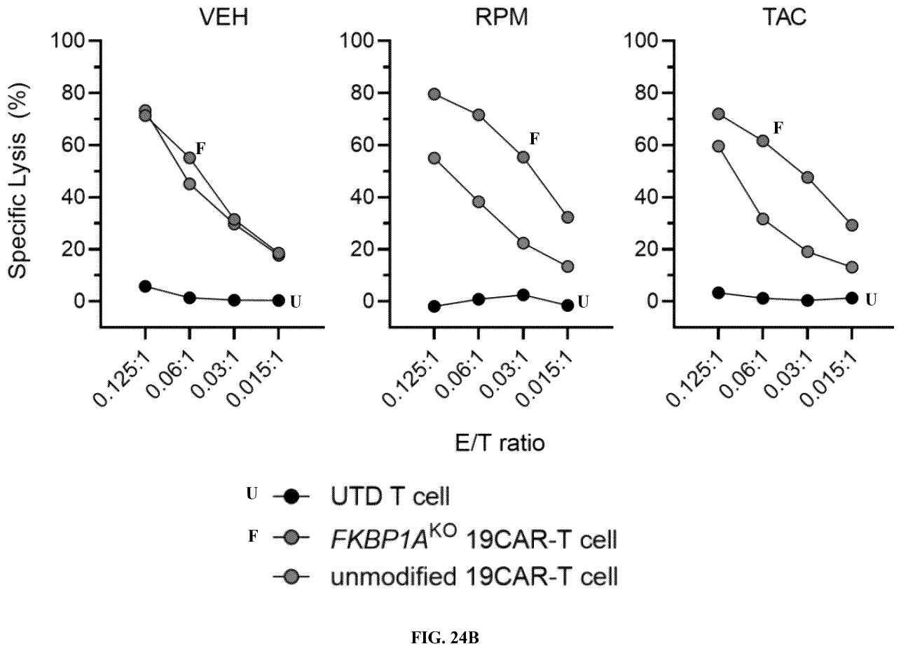

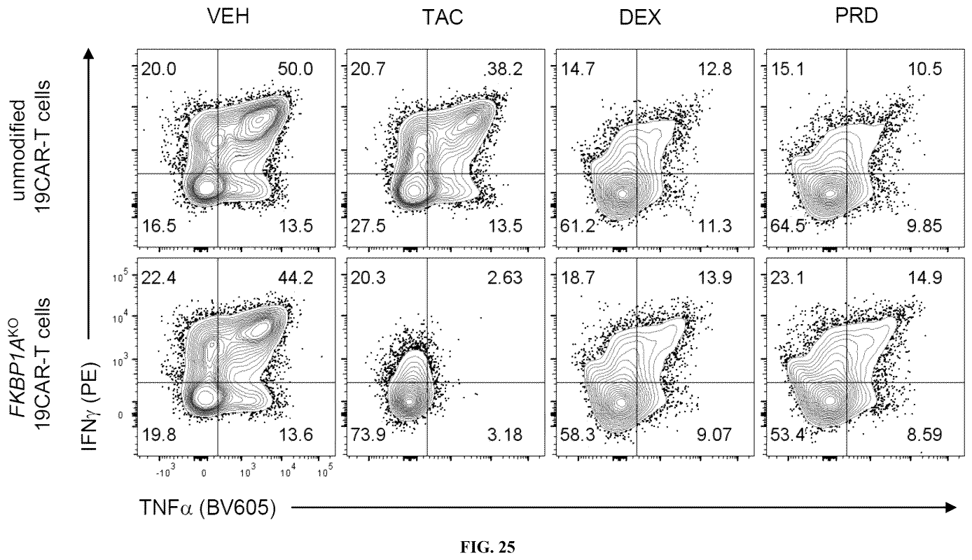

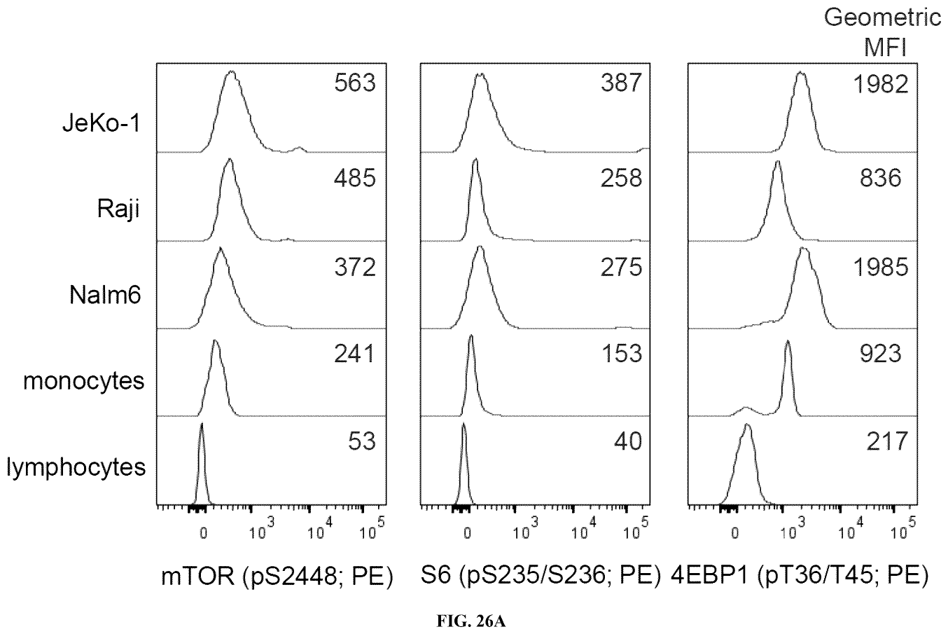

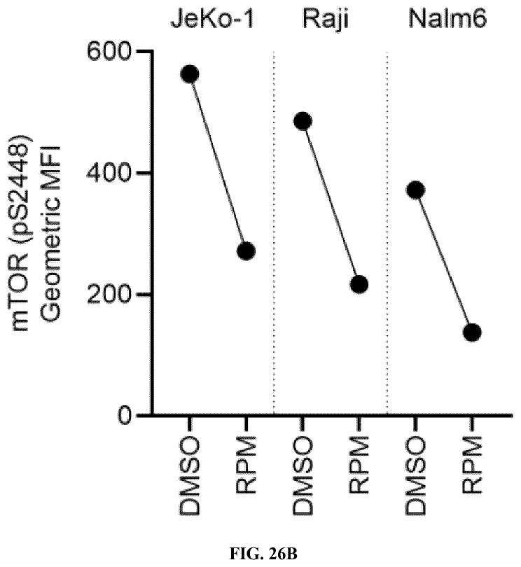

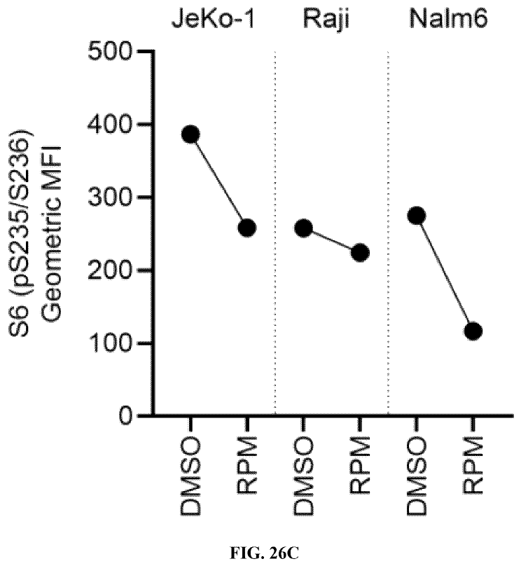

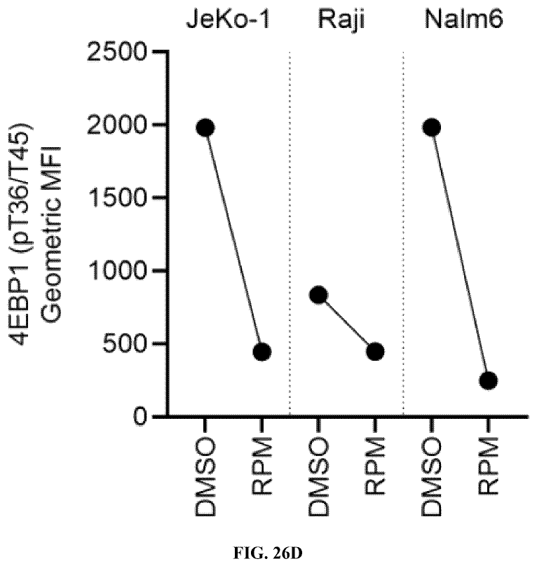

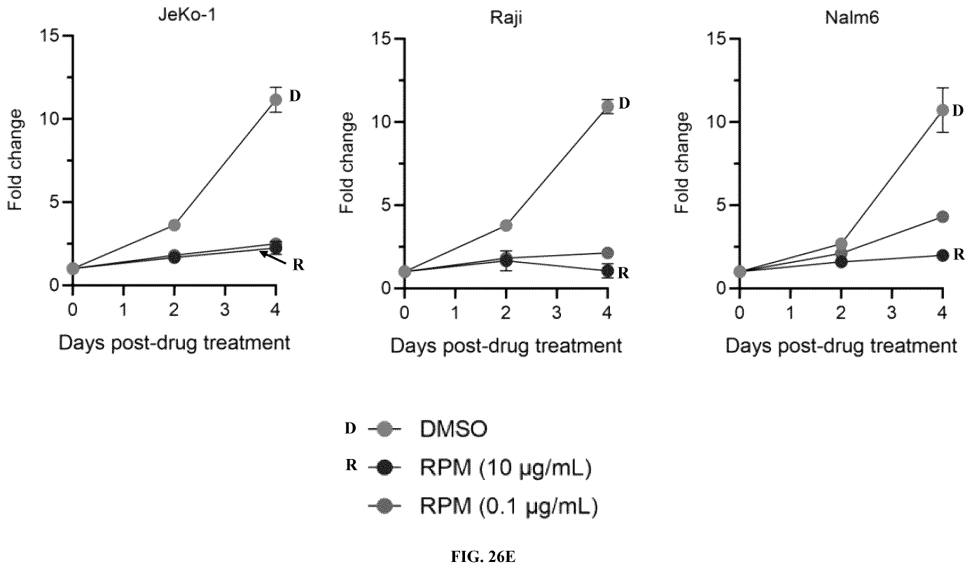

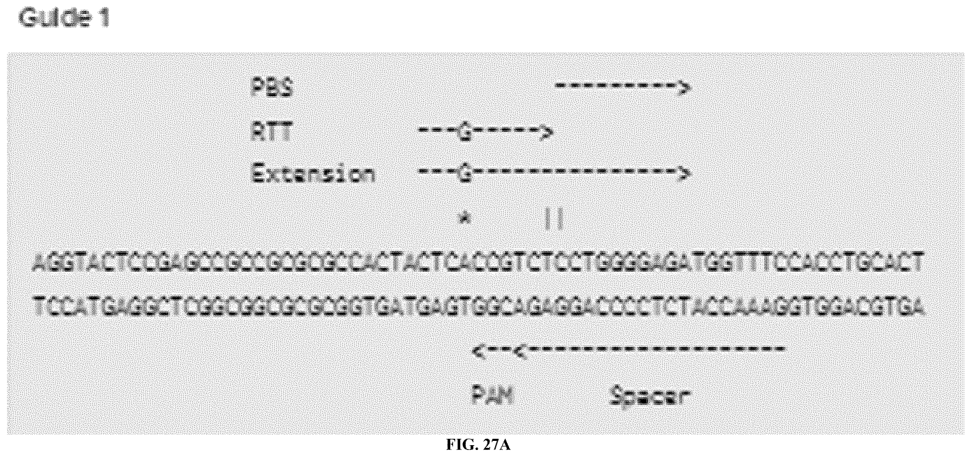

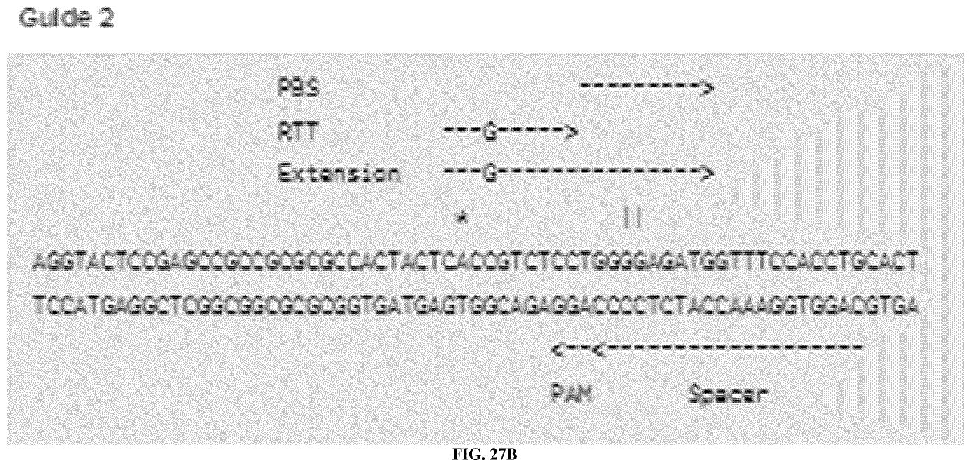

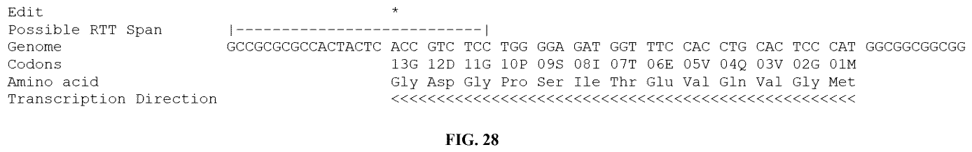

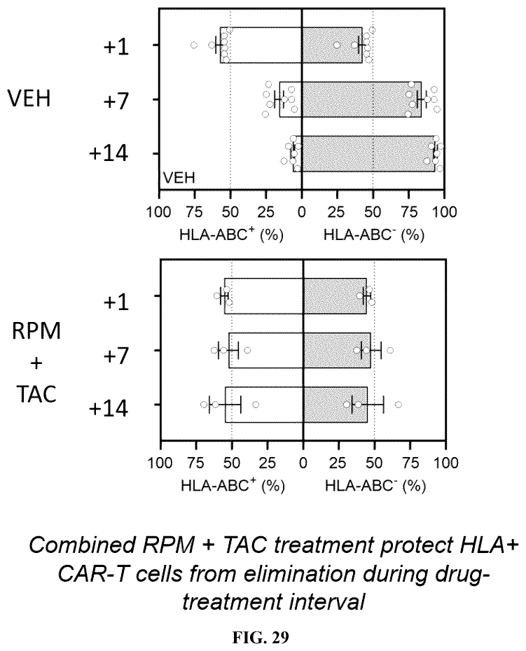

ABE8.20m and the guide polynucleotide TSBT×6143 or TSBT×6146 (see Table 1 for nucleotide sequences). Five days following being contacted with the base editor systems, the T cells were stained using LIVE/DEAD™ Fixable Near-Infra Red (NIR) stain (Thermo). The cells were then fixed and permeabilized followed by immunostaining using a primary antibody targeting PPIA (polyclonal Cyclophilin A Antibody from ProteinTech; 10720-1-AP). The cells were then contacted with a secondary antibody (polyclonal Goat anti-Rabbit IgG (H+L) Cross-Adsorbed Secondary Antibody labeled using Alexa Fluor™ 488 and available from Invitrogen; A-11008) and analyzed using flow cytometry. Unedited cells and isotype control antibody stained cells were used as controls representing high and low levels of PPIA protein, respectively. The x-axis of FIG. 7 indicates the level of PPIA protein measured. In FIG. 7, the y-axis represents cell counts normalized to the mode cell count measured for PPIA expression levels in a sample (i.e., the most frequently counted expression level was set to have a total cell count value (y-axis value) equal to 100).

FIG. 8 provides plots demonstrating that primary human anti-CD19 CAR-T cells edited using a base editor system containing the base editor ABE8.20m and the guide polynucleotide TSBT×1538 to reduce expression of FKBP1A showed improved proliferation in the presence of rapamycin relative to unedited anti-CD19 CAR-T cells. Primary human T cells were activated using an anti-CD3/CD28/CD2 reagent. Two-days after being contacted with the reagent, the T cells were edited using a base editor system containing the guide polynucleotide TSBT×1538 and the base editor ABE8.20m using electroporation, as described above. Following electroporation, both the T cells were transduced with a lentiviral vector encoding an anti-CD19 CAR (19CAR) and allowed to expand in culture for approximately 7 days before being cryopreserved for later analysis. Unedited anti-CD19 CAR-T cells were prepared as a negative control. The 19CAR-T cells were then thawed from cryopreservation and rested overnight before being stimulated using dynabeads coated with recombinant human CD19 protein-coated at a bead-to-cell ratio of 1:1. The T cells were cultured in the presence of 100 nM of the immunosuppressant rapamycin or dimethylsulfoxide (DMSO) as a control for 6 days. The total number of T cells were enumerated using a NucleoCounter NC-200™ automated cell counter.

FIG. 9 provides flow cytometry histograms demonstrating that 19CAR-T cells edited to reduce FKB1A protein expression using a base editor system containing the guide TSBT×1538 and the base editor ABE8.20m showed improved cell proliferation in response to a universal T cell activator relative to unedited 19CAR-T cells. Primary human T cells were activated using an anti-CD3/CD28/CD2 reagent. Two days after being contacted with the reagent, the T cells were edited using a base editor system containing the guide polynucleotide TSBT×1538 and the base editor ABE8.20m using the electroporation protocol described above. Both edited and unedited T cells were transduced with a lentiviral vector encoding an anti-CD19 CAR (19CAR) and allowed to expand in culture for approximately 7 days before being cryopreserved for later analysis. Unedited 19CAR-T cells were used as a control. For analysis, the 19CAR-T cells were thawed and rested for 72 hours before labeling using a CellTrace™ Far Red (CTFR) dye. After CTFR labeling, the 19CAR-T cells were stimulated using an Immunocult™ Human CD3/CD28/CD2 universal T cell activator in the presence of 100 nM of the immunosuppressant agent rapamycin or in the presence of DMSO (control) and allowed to incubate for an additional 3 days. Following the three-day incubation, the T cells were analyzed using flow cytometry to measure dilution of CTFR dye as an indicator of cell proliferation.

FIG. 10 provides flow cytometry contour plots demonstrating that CD4+ 19CAR-T cells edited to reduce FKBP1A protein expression using a base editor system containing the guide polynucleotide TSBT×1538 and the base editor ABE8.20m showed improved cytokine production levels (IFNg and TNFa) when contacted with target cells in the presence of an immunosuppressant agent relative to unedited CD4+ 19CAR-T cells. Unedited (control) and edited 19CAR-T cells were thawed from cryopreservation and rested overnight in the presence of the immunosuppressant rapamycin (RPM; 100 ng/mL) or tacrolimus (TCR; 100 ng/mL), or in the presence of DMSO (control). The following day, the 19CAR-T cells were stimulated by being contacted with CD19⁺ Jeko-1 tumor cells for 6 hours in the presence of 100 ng/ml of an immunosuppressant agent, as indicated in FIG. 10. Monensin and Brefeldin A were added to the cell cultures to inhibit protein secretion for the duration of the stimulation. After the 6 hours, the cells were stained with reagents against 19CAR, CD4, CD8, IFNg, and TNFa, and the cells were also stained using a viability stain. Fluorescence activated cell sorting (FACS) was then used to measure intracellular levels of IFNg (y-axis) and TNFa (x-axis) in CD4⁺ 19CAR-T cells. In FIG. 10, the numbers in each quadrant of each contour plot represent the percent of total cells counted that fell within the quadrant.

FIG. 11 provides flow cytometry contour plots demonstrating that CD8+ 19CAR-T cells edited to reduce FKBP1A protein expression using a base editor system containing the guide polynucleotide TSBT×1538 and the base editor ABE8.20m showed improved cytokine production levels (IFNg and TNFa) when contacted with target cells in the presence of an immunosuppressant agent relative to unedited CD8+ 19CAR-T cells. Unedited (control) and edited 19CAR-T cells were thawed from cryopreservation and rested overnight in the presence of the immunosuppressant rapamycin (RPM; 100 ng/mL) or tacrolimus (TCR; 100 ng/mL), or in the presence of DMSO (control). The following day, the 19CAR-T cells were stimulated by being contacted with CD19+Jeko-1 tumor cells for 6 hours in the presence of 100 ng/mL of an immunosuppressant agent, as indicated in FIG. 10. Monensin and Brefeldin A were added to the cell cultures to inhibit protein secretion for the duration of the stimulation. After the 6 hours, the cells were stained with reagents against 19CAR, CD4, CD8, IFNg, and TNFa, and the cells were also stained using a viability stain. Fluorescence activated cell sorting (FACS) was then used to measure intracellular levels of IFNg (y-axis) and TNFa (x-axis) in CD8⁺ 19CAR-T cells. In FIG. 10, the numbers in each quadrant of each contour plot represent the percent of total cells counted that fell within the quadrant.

FIGS. 12A and 12B provide plots demonstrating that anti-CD19 CAR (19CAR)-T cells edited to reduce FKBP1A protein expression using a base editor system containing the guide polynucleotide TSBT×1538 and the base editor ABE8.20m showed improved cytotoxicity (i.e., lysis of target CD19+ Jeko-1 tumor cells) in vitro when contacted with target cells in the presence of an immunosuppressant agent relative to unedited CD8+ 19CAR-T cells. Unedited and edited 19CAR-T cells were thawed from cryopreservation and rested overnight in the presence of the immunosuppressant rapamycin (100 nM) or tacrolimus (100 ng/ml). The following day, the 19CAR-T cells were co-cultured with green fluorescent protein-expressing (GFP⁺) Jeko-1 tumor cells at a 0.25:1 effector-to-target cell ratio. Rapamycin (100 nM) and tacrolimus (100 ng/mL) were added to the cultures at 0 hr, 48 hr, 96 hr, and 144 hr post co-culture (vertical dashed lines). Tumor associated GFP fluorescence (y-axis) was longitudinally quantified in real-time in 4-hour intervals (x-axis) by using an Incucyte™ Live-Cell Analysis System (Sartorius). Tumor cells alone were used as a control. Co-cultures were evaluated in biological triplicates. The data points in FIGS. 12A and 12B indicate mean measurements and the error bars represent one standard deviation (SD) from the mean. In FIGS. 12A and 12B, “GCU” represents “Green Calibrated Unit.”

FIGS. 13A and 13B provide plots demonstrating that anti-CD19 CAR (19CAR)-T cells edited to reduce FKBP1A protein expression using a base editor system containing the guide polynucleotide TSBT×1538 and the base editor ABE8.20m showed improved cytotoxicity (i.e., lysis of target CD19+ Jeko-1 tumor cells) in Jeko-1 tumor bearing mice administered an immunosuppressant agent relative to unedited CD8+ 19CAR-T cells. The edited 19CAR-T cells were able to eliminate Jeko-1 tumor cells from mice administered tacrolimus, whereas unedited 19CAR-T cells were unable to eliminate Jeko-1 tumor cells from mice administered tacrolimus. Immunocompromised NOD scid gamma (NSG) mice were implanted with 5E5 Jeko-1 tumor cells and the tumor cells were allowed to engraft for 6 days. After the six days of engraftment, 1E6 unedited or edited 19CAR-T cells were infused into tumor bearing mice (N=8 mice evaluated for each experimental condition) and administered daily for 2 weeks (dotted vertical lines) either a vehicle (5% PEG400+5% Tween80) or tacrolimus (10 mg/kg in 5% PEG400+5% Tween80). Tumor burden was assessed twice per week by measuring bioluminescence using an IVIS® spectral imaging instrument. In FIGS. 13A and 13B, each data point represents a measurement taken for an individual mouse.

FIGS. 14A to 14J provide schematic diagrams, flow cytometry histograms, plots, flow cytometry contour plots, and bar graphs showing that HLA-I expression modulated susceptibility of allogeneic cells to T cell or NK cell-driven rejection. FIG. 14A provides a schematic diagram showing generation of HLA-I and HLA-II deficient allogeneic T cells using base editing to knock-out (^{KO}) b2M and CIITA, respectively. FIGS. 14B and 14C provide flow cytometry histograms and a plot showing surface HLA-I/-II expression (FIG. 14B) and frequency of on-target A>G nucleotide conversion by next-generation sequencing (FIG. 14C) in T cells base-edited with b2M- and CIITA-specific sgRNAs and ABE8.20m mRNA. Symbols represent independent donors. FIGS. 14D and 14E show results from a mixed leukocyte assay as flow cytometry (i.e., fluorescence-activated cell sorting (FACS)) contour plots (FIG. 14D) and summarized data (FIG. 14E) for frequency of allogeneic b2M^{KO}CIITA^{KO} and unmodified T cells after coculture with alloreactive T cells from an HLA disparate donor. Symbols represent allogeneic T cells from 2 independent experiments in duplicate. FIG. 14F provides a plot showing results from a cytotoxicity assay. The data indicated NK cell-driven lysis of b2M^{KO} and CIITA^{KO} T cells at different E:T ratios. Symbols represent mean of 4 independent NK cell donors in duplicate. FIG. 14G provides a plot showing percent change in CD107a⁺ NK cells after stimulation with b2M^{KO} or CIITA^{KO} T cells from stimulation with unmodified T cells. Symbols indicate NK cells from 3 independent donors in duplicate. FIGS. 14H to 14J provide a schematic diagram, flow cytometry contour plots, and a plot relating to an experiment where 5×10⁶ TCR^{KO} and TCR^{KO}b2M^{KO} allogeneic CAR-T cells were coinfused into NSG-IL-15tg mice engrafted with human NK cells (blue; n=5) or NSG-IL-15tg mice (gray; n=5) (FIG. 14H). FIGS. 14I and 14J provide a flow cytometry contour plot and a plot showing frequency (FIG. 14I) and concentration (FIG. 14H) of peripheral b2M^{KO} CAR-T cells in recipient mice. In FIG. 14J, thin and thick lines indicate individual mice and mean, respectively. Statistical significance was calculated by Wilcoxon rank-sum test (FIGS. 14F and 14G). Error bars show ±s.e.m. and sample sizes indicate biologically independent animals.

FIGS. 15A to 15F provide a schematic diagram, a bubble plot, scatter plots, plots and bar graphs showing complete retention of HLA-I alleles was necessary to broadly inhibit NK cell reactivity to allogeneic cells. FIG. 15A provides a schematic diagram of allogeneic HLA-I deficient (b2M^{KO}) T cells expressing an HLA-I single chain (HLA^{SC}) molecule that inhibits NK cells by engaging cognate HLA-specific inhibitory receptor. FIG. 15B provides a bubble plot showing frequency of CD56⁺ NK cells expressing the indicated HLA-specific inhibitory receptor from 14 independent donors. FIGS. 15C to 15E provide a flow cytometry scatter plot, plots, and a bar graphs presenting data relating to an experiment where NK cells were stimulated with allogeneic HLA-I⁺ (unmodified) T cells, b2M^{KO} T cells, or b2M^{KO} T cells engineered to express one HLA^{SC}, including HLA-Bw4^{SC} (HLA-B*57), HLA-C1^{SC} (HLA-C*01:02 or *07:02), HLA-C2^{SC} (HLA-C*04:01, *05:01, *06:02 or *18:01), or HLA-E^{SC} (HLA-E*01:03). The contour plots (FIG. 15C) and summary data (FIG. 15D) indicate frequency of CD107a⁺ KIRx⁺ or NKG2A⁺ NK cells after stimulation with unmodified T cells, b2M^{KO} T cells, or b2M^{KO} T cells expressing the cognate HLA^{SC} inhibitory ligand for the corresponding NK cell subset. FIG. 15E provides a bar graph showing frequency of total CD107a⁺ NK cells after stimulation with the indicated target T cell population. FIG. 15F provides bar graphs showing results from a cytotoxicity assay. The bar graph of FIG. 15F shows frequency of NK cell-driven specific lysis after 48 hour stimulation at different E:T ratios with unmodified T cells, b2M^{KO} T cells, or b2M^{KO} T cells engineered to express the indicated HLA^{SC}. In FIGS. 15D to 15F, symbols represent aggregated data from 3 independent NK cell donors in duplicate. Bars indicate mean and error bars show ±s.e.m. Statistical significance was calculated by Wilcoxon matched-pairs signed rank test (FIG. 15D) and Kruskall-Wallace test with Dunn's test for multiple comparisons (FIGS. 15E and 15F).

FIGS. 16A to 16J provide a schematic diagram, charts, flow cytometry contour plots, bar graphs, and plots showing immunosuppressant treatment mitigated in vivo T cell-driven rejection of allogeneic HLA-I⁺ CAR-T cells. FIG. 16A provides a schematic diagram and charts showing how five independent cohorts (#1-5) of human immune system (HIS) mice were allocated into groups that received vehicle (VEH; n=25), rapamycin (RPM; n=14), or tacrolimus (TAC; n=13) daily for 2 weeks. At 1-day post-treatment, 5×10⁶ allogeneic CD4-based CAR-T cells base-edited for TCR^{KO} (HLA⁺) or TCR^{KO}b2M^{KO}CIITA^{KO} (HLA-deficient) were mixed and coinfused into recipient HLA disparate mice. T cell infusion product for Cohorts #3 an #4 included an equal amount of syngeneic HIS-mouse derived CAR-T cells. FIG. 16B provides flow cytometry contour plots showing longitudinal frequency of peripheral HLA⁺ and HLA-deficient CAR-T cells in mice treated with VEH, RPM or TAC. FIG. 16C provides a bar graph showing aggregate peripheral allogeneic HLA⁺ CAR-T cell persistence relative to HLA-deficient CAR-T cells from within individual mice during drug treatment interval. FIG. 16D provides bar graphs showing peripheral allogeneic HLA⁺ CAR-T cell persistence relative syngeneic CAR-T cells from within individual mice in Cohorts 3 and 4 during drug treatment interval. FIGS. 16E and 16F provide plots showing a correlation between percentage change in allogeneic HLA⁺ CAR-T cells from 1 to 7 days post-infusion and contemporaneous plasma concentration of RPM in Cohorts 1-4 (FIG. 16E) and TAC in Cohorts 3-4 (FIG. 16F). FIGS. 16G and 16H provide contour plots and a plot showing frequency (FIG. 16G) and summary data (FIG. 16H) for splenic allogeneic HLA⁺ and HLA-deficient CAR-T cells from individual mice treated with VEH (n=4), RPM (n=6) and TAC (n=6) in Cohorts 3 and 4. FIGS. 16I and 16J provide plots showing cumulative persistence of peripheral allogeneic HLA⁺ CAR-T cells during drug treatment interval (1 to 15 days post-infusion) (FIG. 16I) and post-drug treatment interruption (22 to 42 days post-infusion) (FIG. 16J). For all data, symbols and sample sizes indicate biologically independent animals. Bars and lines represent mean and error bars show ±s.e.m. Statistical significance was calculated by Kruskall-Wallace test with Dunn's test for multiple comparisons (FIGS. 16C and 16D), Spearman correlation (FIGS. 16E and 16F), and Wilcoxon matched-pairs signed rank test (FIGS. 16H to 16J). AUC, area under the curve. r, coefficient of correlation.

FIGS. 17A to 17I provide plots, flow cytometry contour plots, and bar graphs showing disruption of FKBP1A in T cells conferred in vitro functional resistance to immunosuppression by rapamycin and tacrolimus. FIG. 17A provides a plot showing frequency of maximum on-target A>G nucleotide conversion by NGS in T cells base-edited with TSBT×1538 sgRNA and ABE8.20m mRNA (FKBP1A^{KO}). Symbols indicate independent donors. FIGS. 17B and 17C provide flow cytometry contour plots (FIG. 17B) and a plot of summary data (FIG. 17D) showing frequency of phosphorylated S6 protein in unmodified and FKBP1A^{KO} T cells after treatment with rapamycin (RPM) or vehicle (VEH; DMSO) control. FIGS. 17D and 17E provide flow cytometry contour plots (FIG. 17D) and a plot of summary data (FIG. 17F) showing frequency of GFP expression in unmodified or FKBP1A^{KO} T cells that expressed an NFAT-GFP reporter after treatment with tacrolimus (TAC) or VEH. FIG. 17F provides a bar graph showing percentage change in total CD19-specific CAR-T cells (19CAR) counts 1-week post-treatment with RPM or TAC relative to VEH. Symbols represent 3 independent donors in duplicate. FIGS. 17G and 17H provide flow cytometry contour plots and a bar graph showing data from an experiment where intracellular cytokine expression was measured in unmodified and FKBP1A^{KO} 19CAR-T cells after stimulation with JeKo-1 tumor cells in the presence of RPM, TAC or VEH. The flow cytometry contour plots of FIG. 17G show frequency of 19CAR-T cells expressing IFNg and TNFa, and the summary data plot of FIG. 17H shows percentage change in cytokine expression in RPM- and TAC-treated conditions relative to VEH. FIG. 17I provides plots showing results from an IncuCyte® Live-Cell Analysis System cytotoxicity assay. Tumor burden was quantified as green calibrated units (GCUs) derived from the fluorescence intensity of GFP⁺ JeKo-1 tumors that were cultured in triplicate with either untransduced (UTD) T cells, unmodified 19CAR-T cells, or FKBP1A^{KO} 19CAR-T cells at a 0.25:1 ratio. The solid lines represents mean GCU from images taken every 4 hours, dotted lines show ±s.e.m., and vertical lines indicates redosing with VEH, RPM or TAC. In FIGS. 17C, E, and H, Symbols represent 2 independent donors in duplicate. For all data, lines and bars represent mean and error bars show ±s.e.m.

FIGS. 18A to 18G provide a schematic diagram, a chart, images, plots, and bar graphs showing FKBP1A^{KO} 19CAR-T cells retained in vivo anti-tumor function in the presence of Tacrolimus and Rapamycin. FIG. 18A provides a schematic diagram and chart of drug-treatment of a tumor-bearing mouse model relating to FIGS. 18B to 18E. 5×10⁵ JeKo-1.Luc cells were transplanted into recipient NSG mice (female; aged 6-8 weeks; n=72), then 7 days after mice initiated VEH (n=24), RPM (n=24) or TAC (n=24) treatment daily for 2 weeks. One day later, mice from each drug-treatment group were infused with either 1×10⁶ untransduced (UTD) T cells (n=8 per group), unmodified CD19-specific CAR-T cells (19CAR) (n=8 per group), or FKBP1A^{KO} 19CAR-T cells (n=8 per group). FIG. 18B provides representative longitudinal bioluminescent flux imaging of JeKo-1. Luc bearing NSG mice treated with TAC and UTD, 19CAR, or FKBP1A^{KO} 19CAR-T cells. FIG. 18C provides plots showing longitudinal tumor burden (flux p/s) of T cell-treated mice that received VEH or TAC. FIG. 18D provides a bar graph showing cumulative tumor burden of T cell-treated mice during drug-treatment interval that received VEH or TAC. FIG. 18E provides a plot showing longitudinal tumor burden of T cell-treated mice that received VEH or RPM. FIGS. 18F and 18G present data relating to an experiment where female NSG mice were implanted with 5×10⁵ JeKo-1.FKBP1A^{KO}.Luc cells and 6 days later initiated VEH (n=30) or RPM (n=30) treatment daily for 2 weeks. One day later, mice from each drug-treatment arm were infused with either 1×10⁶ UTD T cells (n=10 per group), unmodified 19CAR-T cells (n=10 per group), or FKBP1A^{KO} 19CAR-T cells (n=10 per group). FIG. 18F provides plots showing longitudinal tumor burden of T cell-treated mice that received VEH or RPM. FIG. 18G provides a bar graph showing cumulative tumor burden of T cell-treated mice during drug-treatment interval that received VEH or RPM. For all data, symbols and bars reflect means and error bars show ±s.e.m., except FIGS. 18D and 18G where symbols represent individual mice. Statistical significance was calculated by Kruskall-Wallace test with Dunn's test for multiple comparisons (FIGS. 18D and 18G). AUC, area under the curve.

FIGS. 19A to 19J provide a schematic diagram, a chart, bar graphs, and flow cytometry contour plots showing FKBP1A^{KO} 19CAR-T cells with concomitant tacrolimus treatment induced B cell aplasia in immunocompetent mice. FIG. 19A provides a schematic diagram and a chart showing how four independent cohorts (#6-9) of huNCG human immune system (HIS) mice were evenly distributed into 4 groups that received VEH and UTD T cells (Group 1; n=16), VEH and HLA⁺ 19CAR-T cells (Group 2; n=16), TAC and HLA⁺ FKBP1A^{KO} 19CAR-T cells (Group 3; n=15), or VEH and HLA-deficient 19CAR-T cells (Group 4; n=16). FIGS. 19B and 19C provide a bar graph of cell concentration (FIG. 19B) and flow cytometry contour plots (FIG. 19C) for peripheral CD19⁺ B cells 6 days post-T cell infusion from mice in Groups 1-4. FIGS. 19D and 19E provide bar graphs showing total CD19⁺ B cells from individual mouse splenic (FIG. 19D) and bone marrow (FIG. 19E) tissue 10 days post-T cell infusion in Groups 1-4. FIG. 19F provides a bar graph showing geometric median fluorescent intensity (MFI) of CD19 expression on residual peripheral CD22⁺ B cells 6 days post-T cell infusion from mice in Groups 1-4. FIG. 19G provides a bar graph showing concentration of peripheral CD22⁺CD19^{dim} B cells 6 days post-T cell infusion from mice in Groups 1-4. FIG. 19H provides flow cytometry contour plots showing frequency of splenic 19CAR-T cells from mice in Groups 2-4 10 days post-T cell infusion. FIGS. 19I and 19J provide bar graphs showing concentration (FIG. 19I) and total splenic (FIG. 19J) 19CAR-T cells from mice in Groups 2-4 10 days post-T cell infusion. For all data, bars reflect mean, error bars show ±s.e.m, and symbols indicate biologically independent animals. Statistical significance was calculated by Wilcoxon rank sum test (FIGS. 19B, 19D, and 19E) and Kruskall-Wallace test with Dunn's test for multiple comparisons (FIGS. 19F, 19G, 19I, and 19J).

FIGS. 20A to 20E provide plots, a schematic diagram, and a flow cytometry scatter plot showing human immune system (HIS) mice under-reconstituted human NK cells and necessitated IL-15 treatment to eliminate HLA-deficient T cells. FIG. 20A provides a plot showing frequency of human CD56⁺ NK cells of CD45⁺ cells in PBMCs from human donors (n=10), BLT-NSG mice (Cohort A, n=18; Cohort B, n=15; Cohort C, n=18) and huCD34⁺ NCG mice (Cohort D, n=17; Cohort E, n=12; Cohort F, n=24; Cohort G, n=12). FIGS. 20B to 20E relate to an experiment where HIS mice were treated every 2-3 days with recombinant human IL-15 (2.5 mg) or PBS for 6 total injections. Mice were then coinfused 1:1 ratio with allogeneic CD4-based CAR-T cells that were base-edited for TCRKO (HLA+) or TCRKOb2MKOCIITAKO (HLA−). FIG. 20B provides a schematic diagram of in vivo study design. FIG. 20C provides flow cytometry scatter plots showing frequency of peripheral allogeneic HLA⁺ and HLA− CAR-T cells 4 days post-infusion in PBS- and IL-15-treated mice. FIG. 20D provides a plot showing concentration of peripheral allogeneic HLA+ and HLA− CAR-T cells in PBS-treated mice 4 days post-infusion. FIG. 20E provides a plot showing concentration of peripheral allogeneic HLA⁺ and HLA− CAR-T cells in IL-15-treated mice at 1 and 7 days post-infusion. For all data, symbols indicate biologically independent samples. Statistical significance was calculated by Wilcoxon matched-pairs signed rank test (FIG. 20E).

FIGS. 21A to 21C provide flow cytometry scatter plots and plots showing Rapamycin and Tacrolimus inhibited in vitro priming of alloreactive T cells. Human CD3-depleted PBMCs served as allogeneic target cells to prime CellTrace Violet labeled CD3⁺ T cells from an HLA disparate donor. CD3⁺ T cells were cultured alone (unstimulated) or in the presence of allogeneic CD3⁻ PBMCs with DMSO, or rapamycin (RPM) or tacrolimus (TAC) at different concentrations. FIG. 21A provides flow cytometry scatter plots showing frequency of dividing alloreactive CD8⁺ and CD4⁺ T cells 7 days post-coculture. FIGS. 21B and 21C provide plots showing frequency of dividing alloreactive CD8⁺ and CD4⁺ T cells at 5 (FIG. 21B) and 7 (FIG. 21C) days post-coculture. Symbols represent replicates, bars indicate mean and error bars show ±s.e.m.

FIGS. 22A to 22C provide a schematic diagram and flow cytometry scatter plots showing CD4-based CAR-T cell generation and ex vivo identification by flow cytometry. FIG. 22A provides a schematic diagram of lentiviral constructs used to generate CD4-based CAR-T cells. CD4-based CAR (4CAR) consists of the CD4 extracellular domain (ECD) fused to the CD8a hinge (H) and transmembrane (TM) regions along with the intracellular 4-1BB and CD3z activating domains. 4CAR was separated by an intervening T2A self-cleaving peptide to a molecular tag comprising GFP or truncated EGFR, NGFR or CD19. FIG. 22B provides flow cytometry scatter plots showing frequency of transduced T cells expressing molecular tag incorporated into the 4CAR lentiviral construct. FIG. 22C provides a schematic diagram showing a representative flow cytometry gating strategy to identify HLA+ and HLA-deficient 4CAR-T cells from whole blood.

FIGS. 23A to 23D provide plots showing FKBP1AKO 19CAR-T cells (effectors) exhibited in vitro anti-tumor cytotoxic activity in the presence of rapamycin and tacrolimus. Anti-tumor cytotoxicity activity was evaluated using an IncuCyte® Live-Cell Analysis System cytotoxicity assay. Tumor burden was quantified as Green Calibrated Units (GCU) derived from the fluorescence intensity of GFP⁺ JeKo-1 tumors (targets) cultured in triplicate with effector T cells either untransduced (UTD) T cells, unmodified 19CAR-T cells, or FKBP1AKO 19CAR-T cells. FIGS. 23A and 23B provide plots showing longitudinal tumor burden at 1:1 (FIG. 23A) and 0.125:1 (FIG. 23B) effector-to-target (E/T) ratios treated with vehicle (VEH; DMSO), rapamycin (RPM) or tacrolimus (TAC). FIGS. 23C and 23D provide plots showing longitudinal tumor burden at 1:1 (FIG. 23C) and 0.125:1 (FIG. 23D) E/T ratios treated with VEH or combination RPM and TAC. Data in FIGS. 23A and 23B and FIGS. 23C and 23D were generated using independent T cell donors. Bold lines indicate mean GCU from images taken every 4 hours, dotted lines show ±s.e.m., and vertical lines indicate redosing with VEH, RPM and/or TAC.

FIGS. 24A and 24B provide flow cytometry scatter plots and plots showing FKBP1AKO 19CAR-T cells (effectors) exhibited in vitro anti-tumor cytotoxic activity in the presence of immunosuppressants using a VITAL killing assay (see, e.g., Hermans, et al., J. Immunol Methods, 285:25-40 (2003), the disclosure of which is incorporated by reference in its entirety for all purposes). FIG. 24A provides flow cytometry scatter plots showing frequency of residual on-target Nalm6.CD19WT.GFP⁺ tumor cells and off-target Nalm6.CD19KO.iRFP670⁺ tumor cells (targets) at 0:1 and 0.6:1 effector-to-target (E/T) ratio with untransduced (UTD) T cells, unmodified 19CAR-T cells, or FKBP1AKO 19CAR-T cells at 48 hours post-culture. Cultures were treated with vehicle (VEH; DMSO) control, rapamycin (RPM) or tacrolimus (TAC) at the start of the assay. FIG. 24B provides plots showing frequency of specific lysis at the indicated E/T ratios in VEH-, RPM- and TAC-treated conditions 48 hours post-culture. Symbols represent mean from conditions set-up in duplicate.

FIG. 25 provides flow cytometry contour plots showing FKBP1AKO 19CAR-T cells were sensitive to dexamethasone and prednisone immunosuppression. Unmodified and FKBP1AKO 19CAR-T cells were stimulated with JeKo-1 tumor cells in the presence of vehicle (VEH; DMSO) control, tacrolimus (TAC), dexamethasone (DEX), prednisone (PRD) and then analyzed for intracellular production of cytokines. The flow cytometry contour plots show frequency of IFNg and TNFa producing 19CAR-T cells

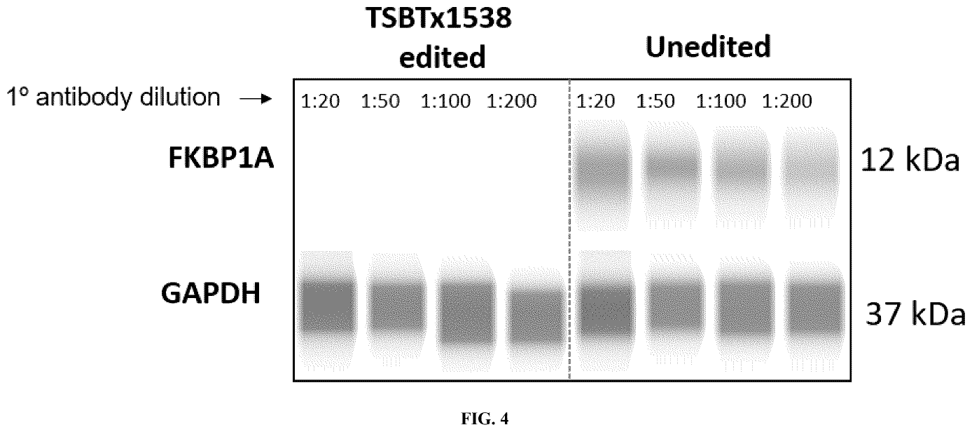

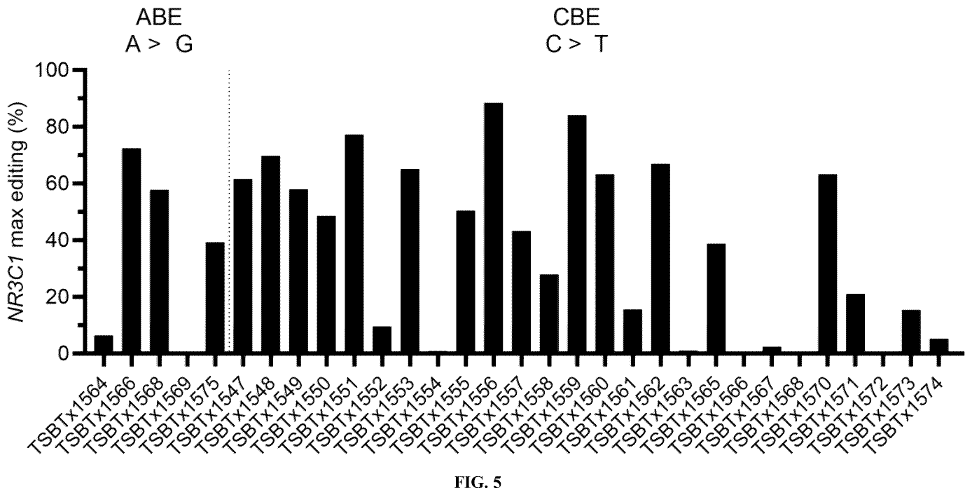

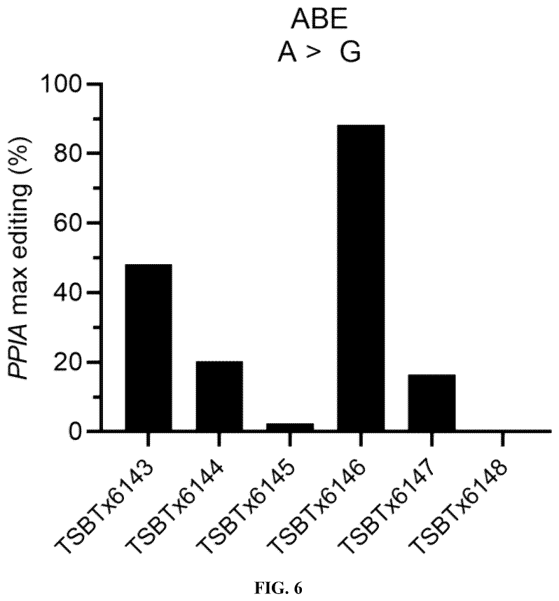

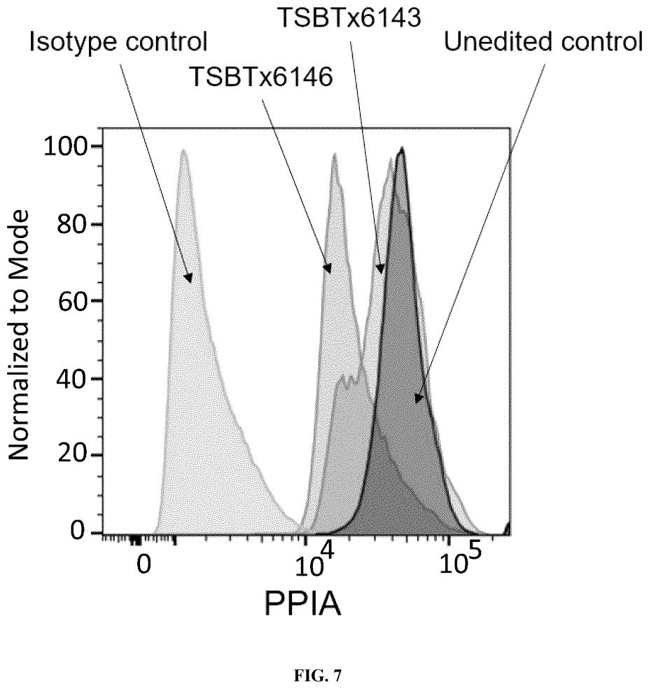

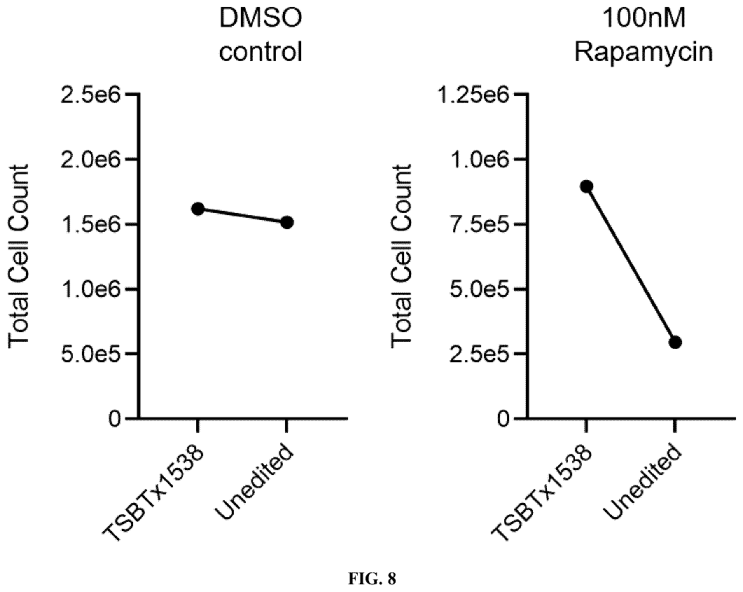

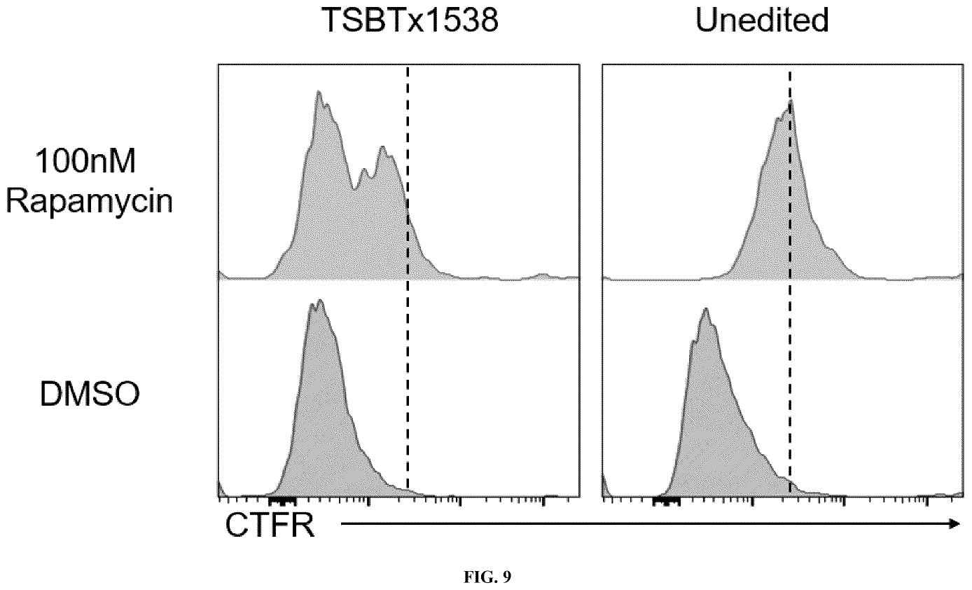

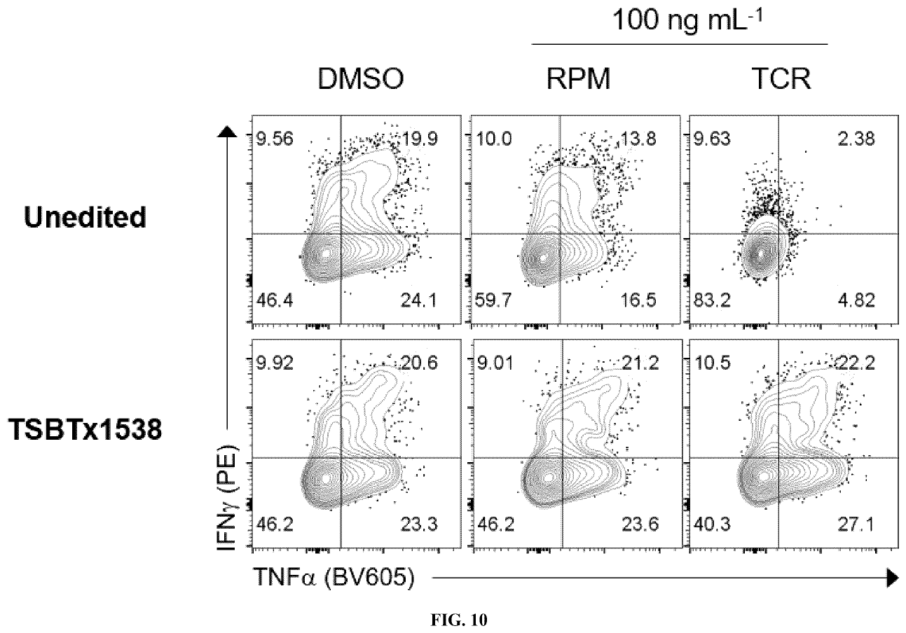

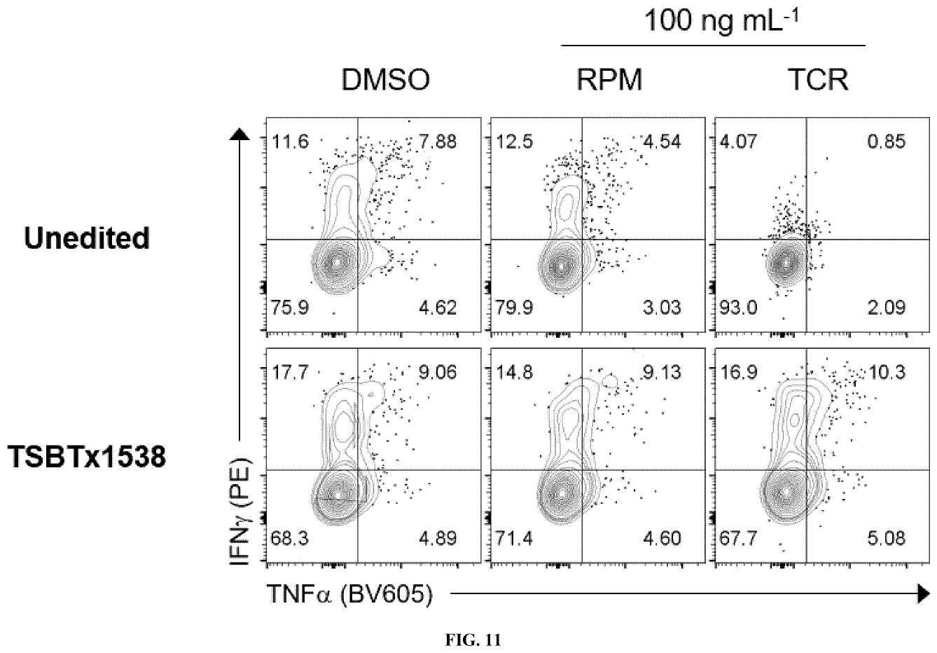

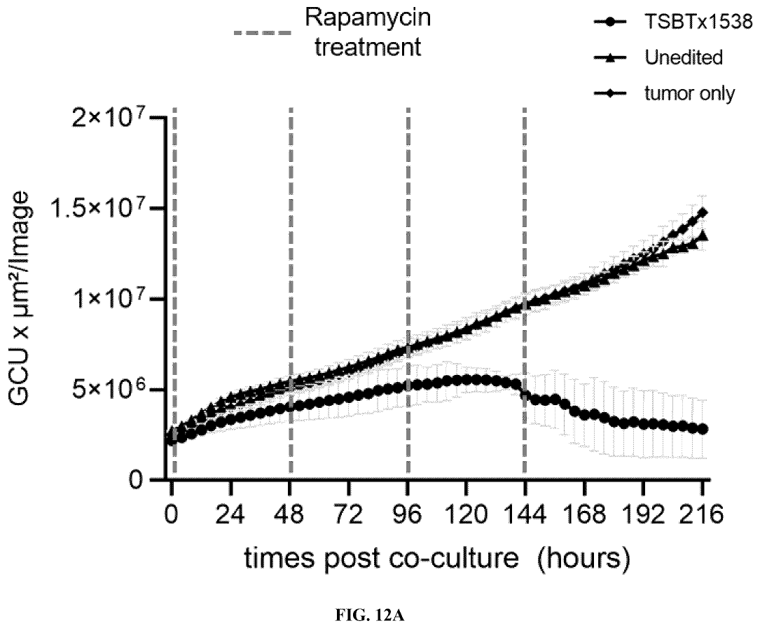

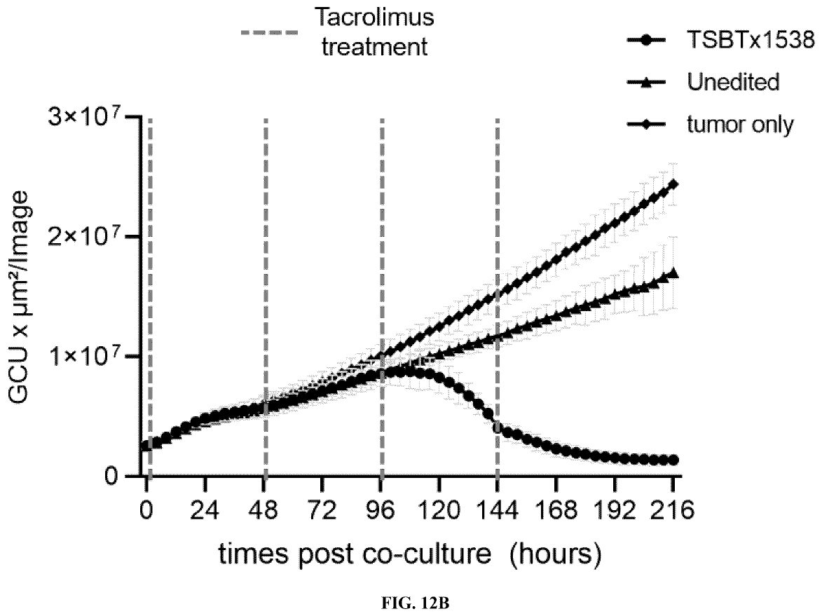

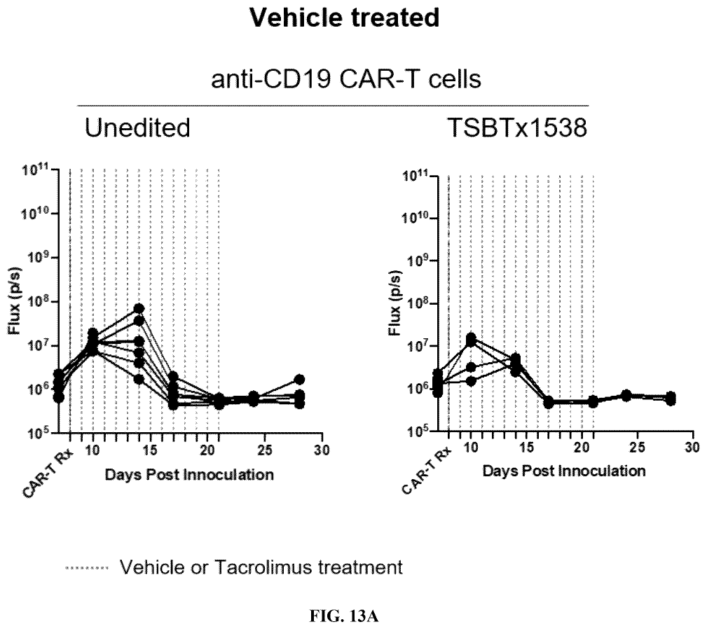

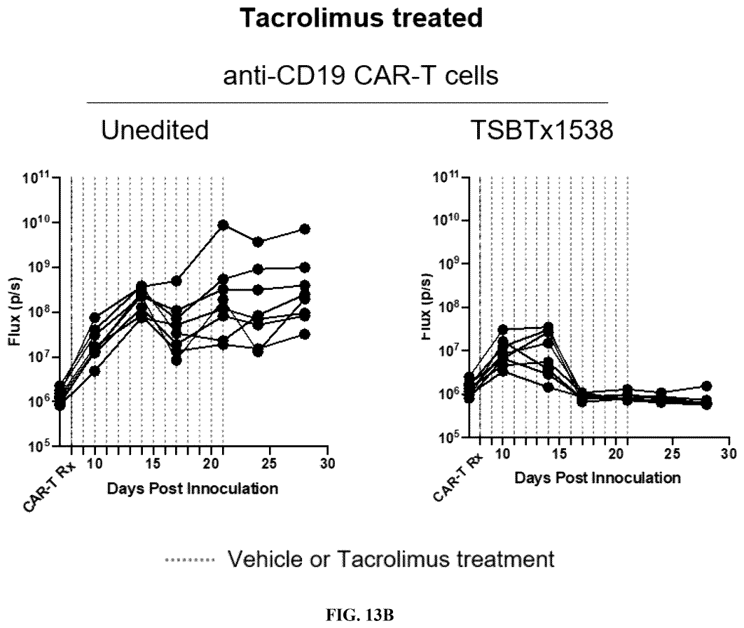

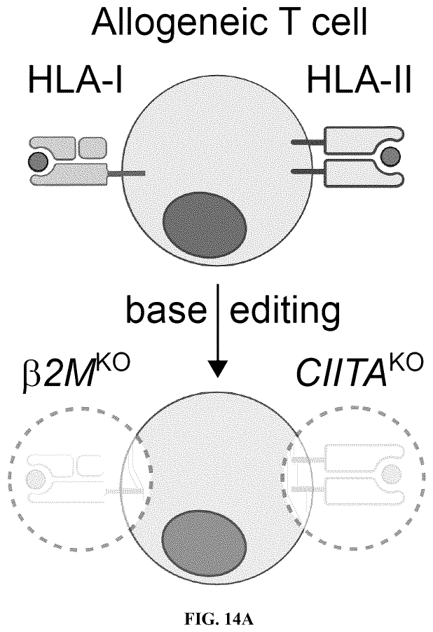

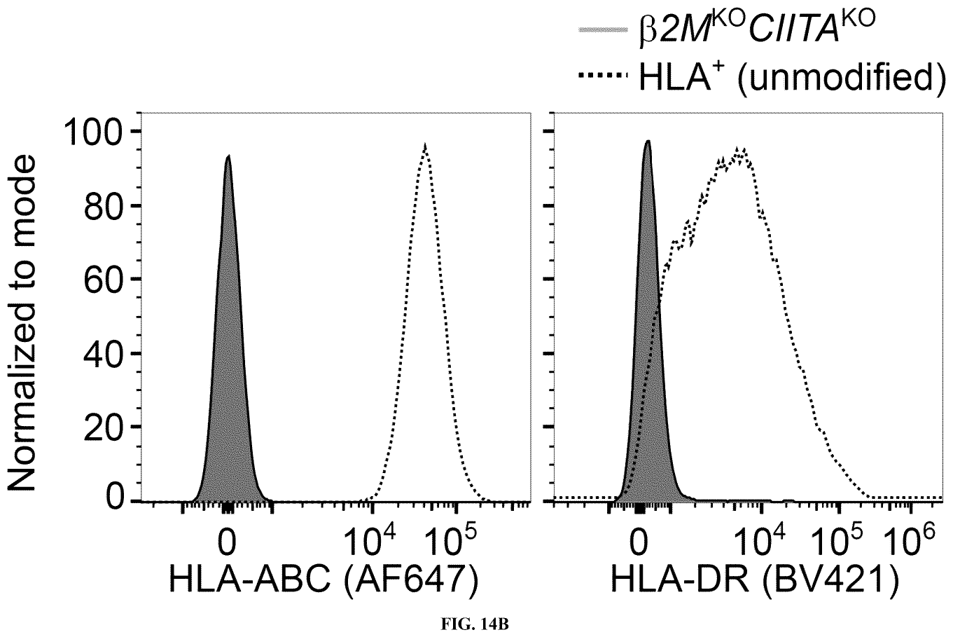

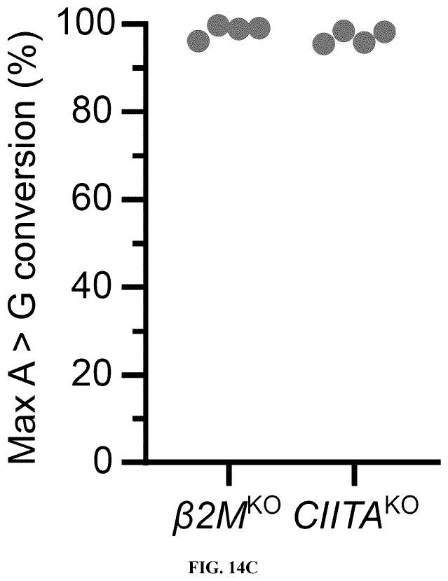

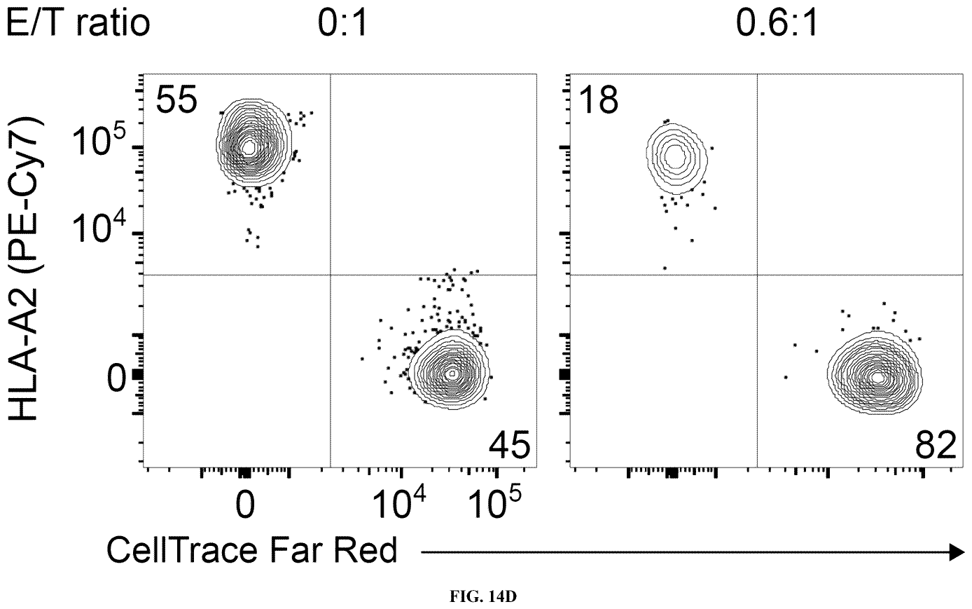

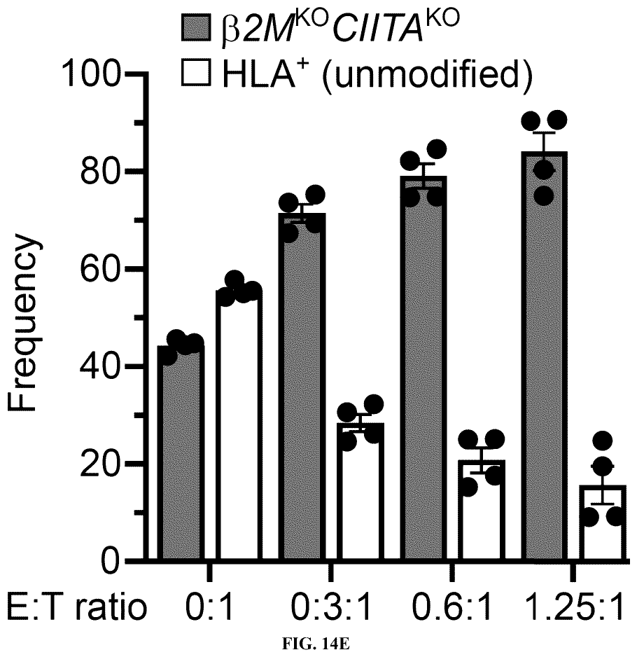

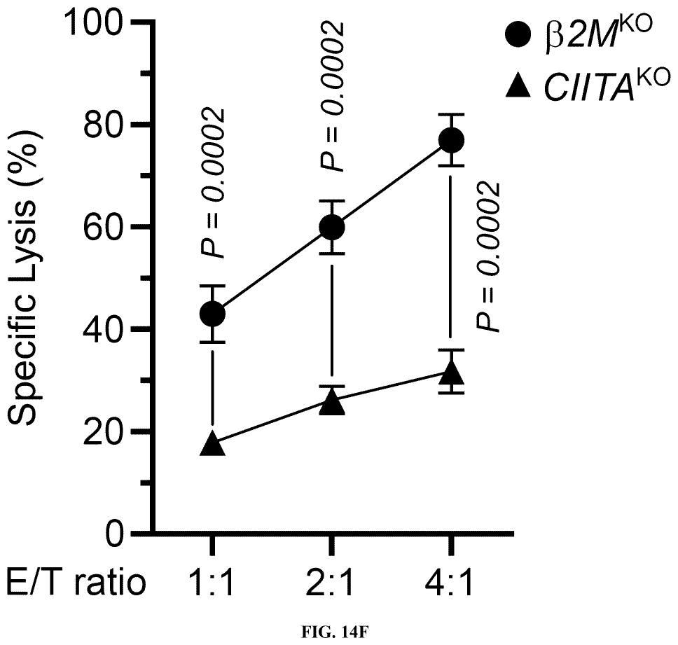

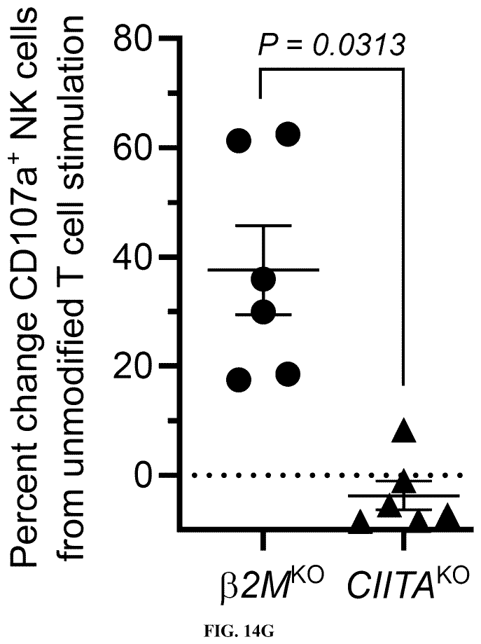

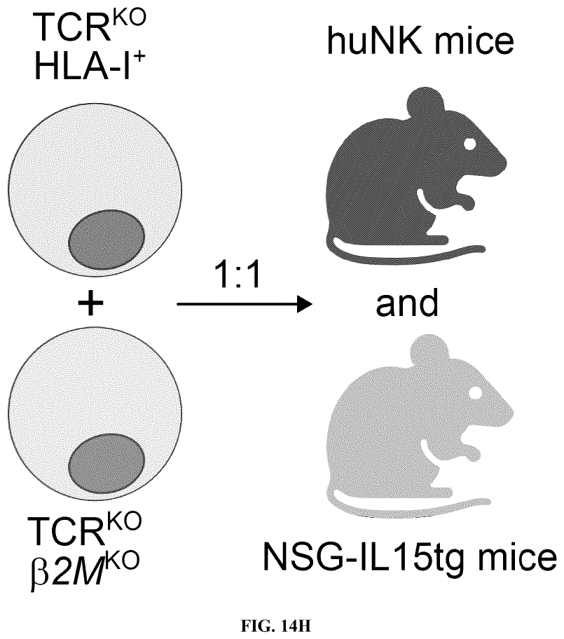

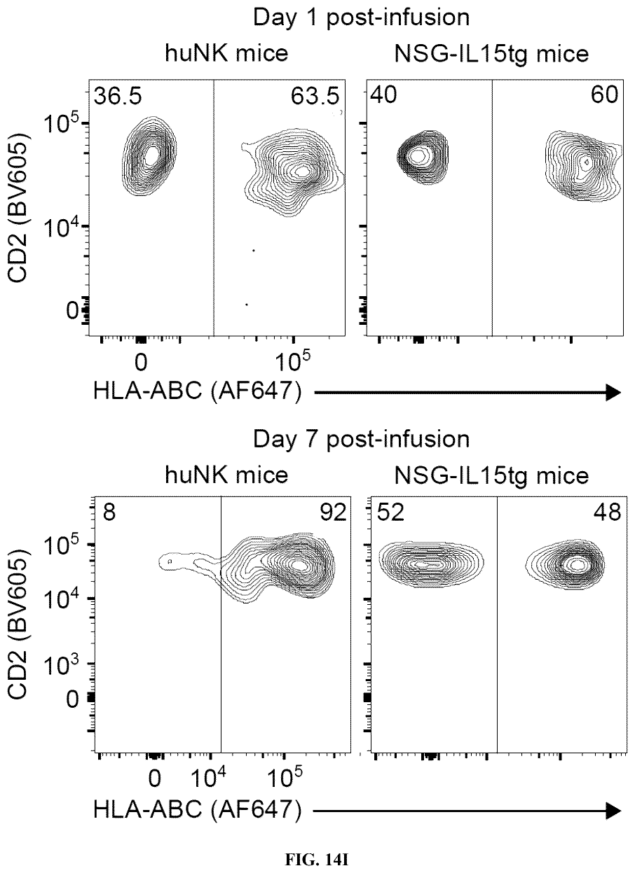

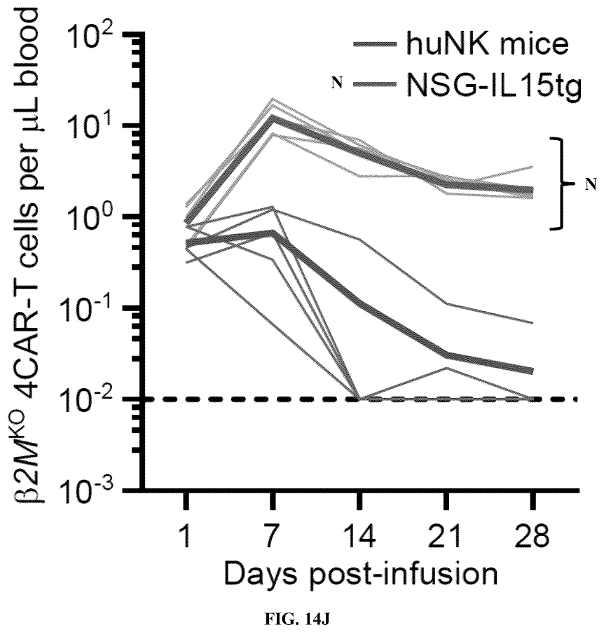

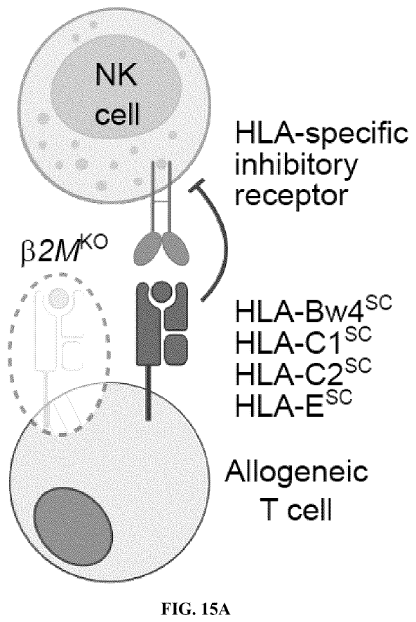

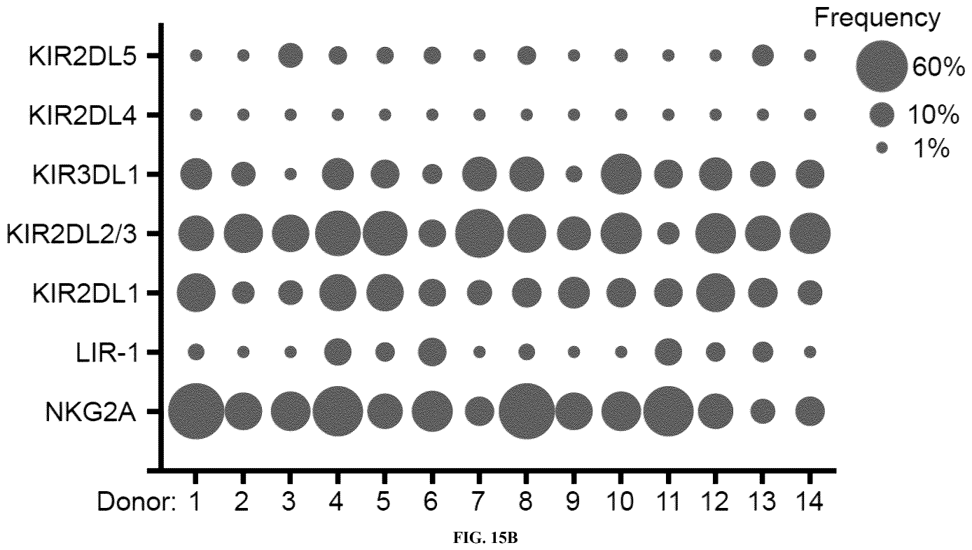

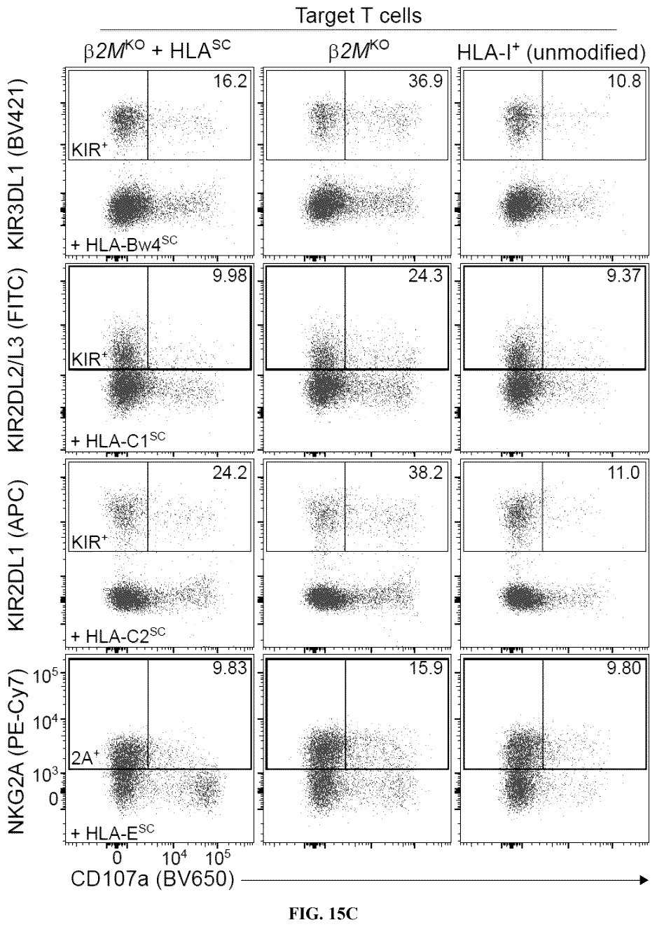

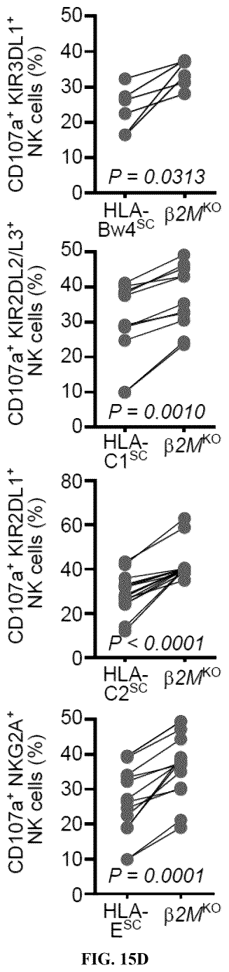

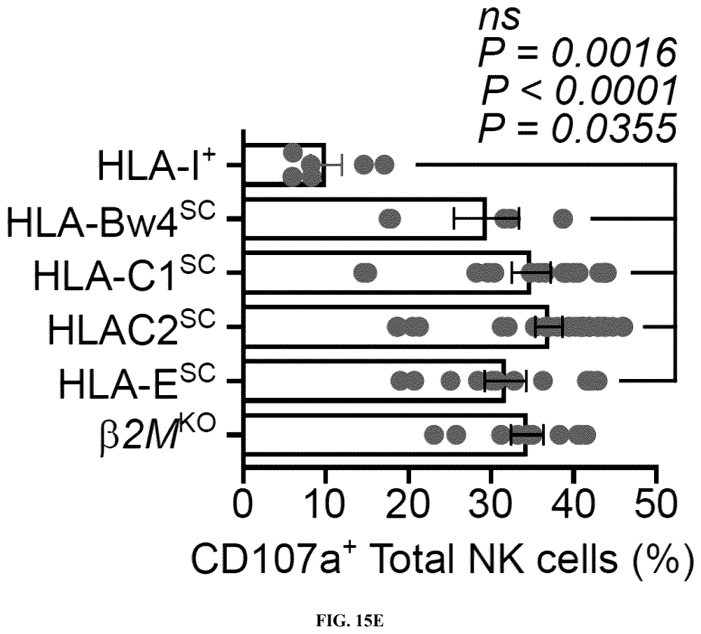

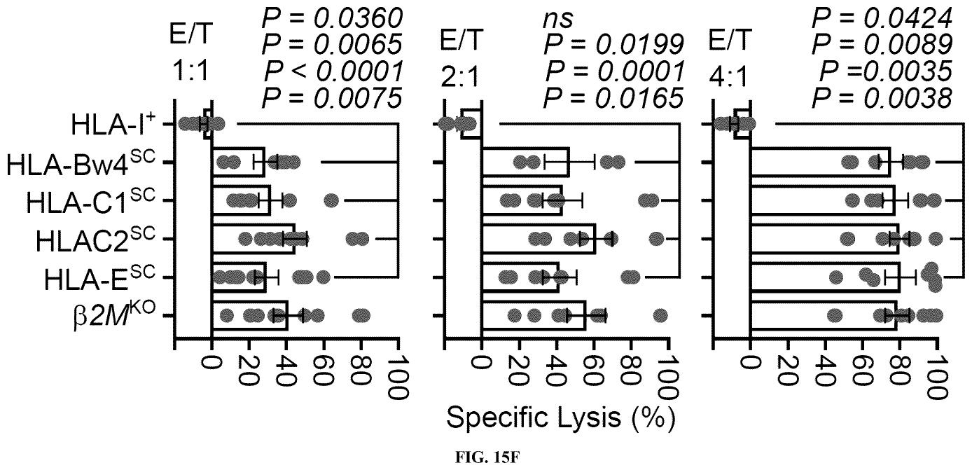

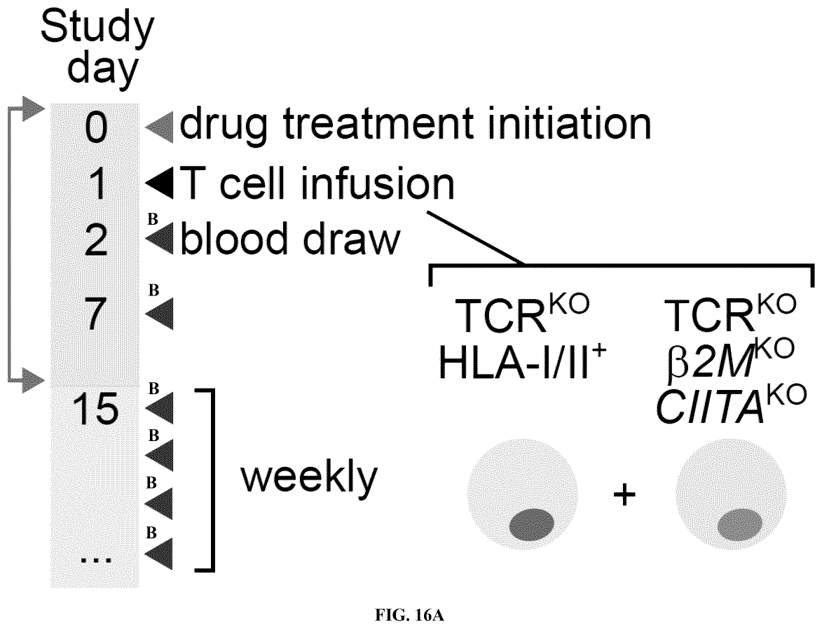

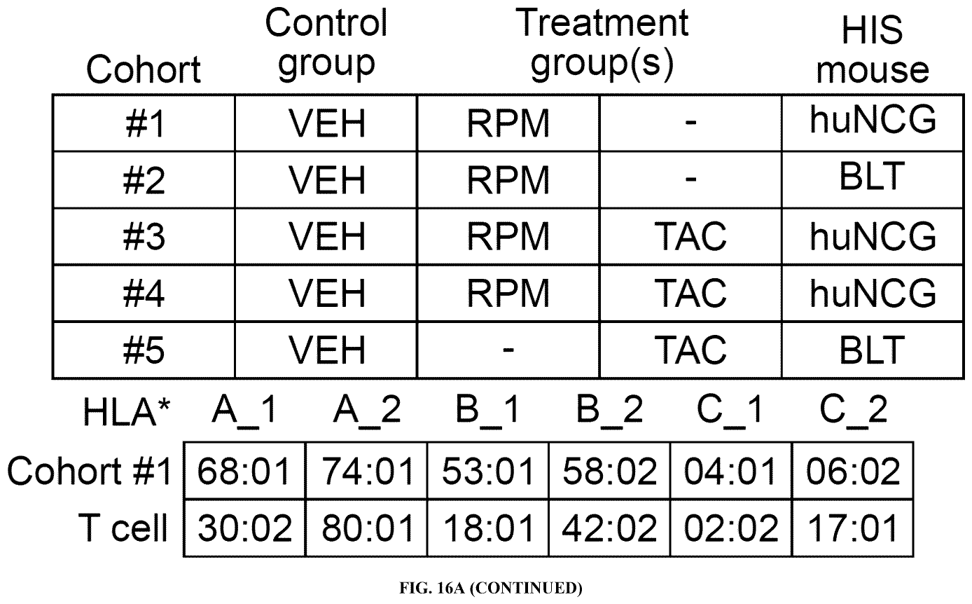

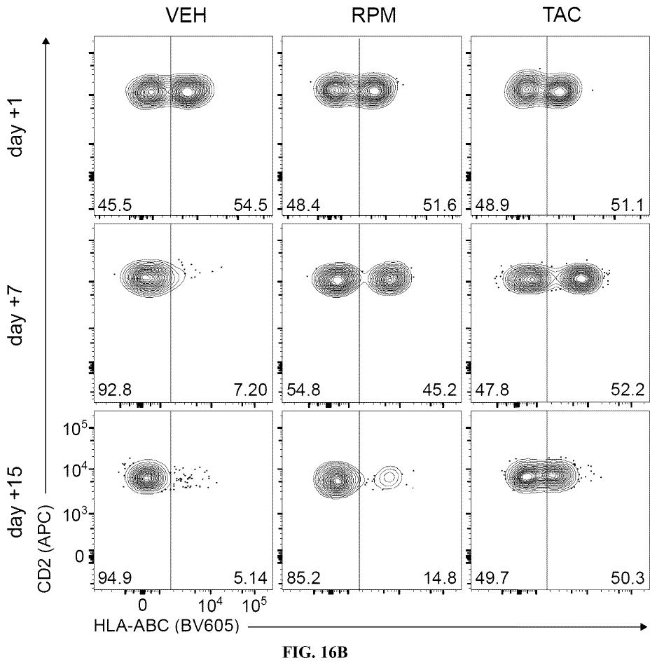

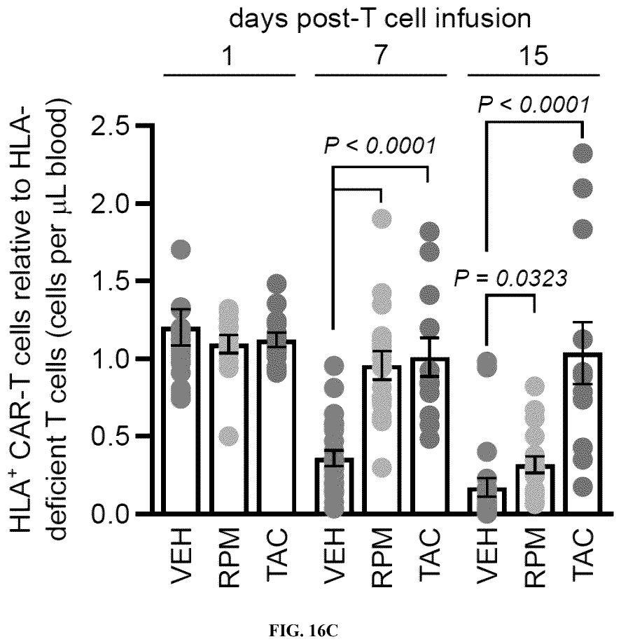

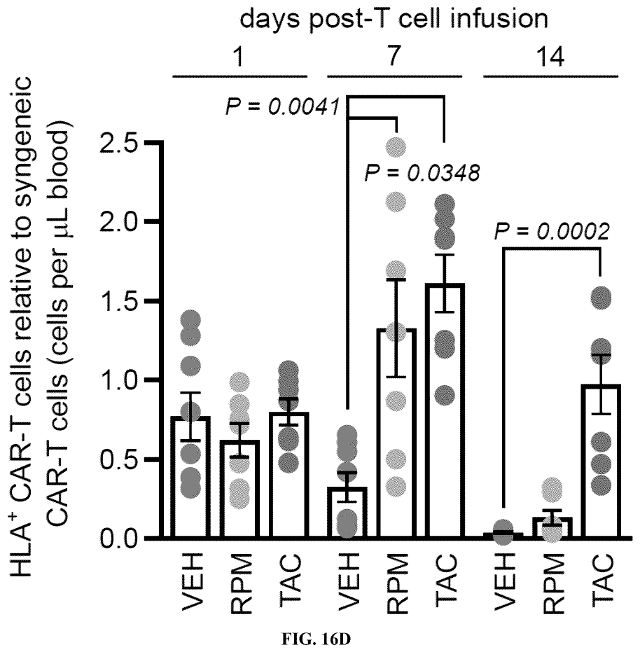

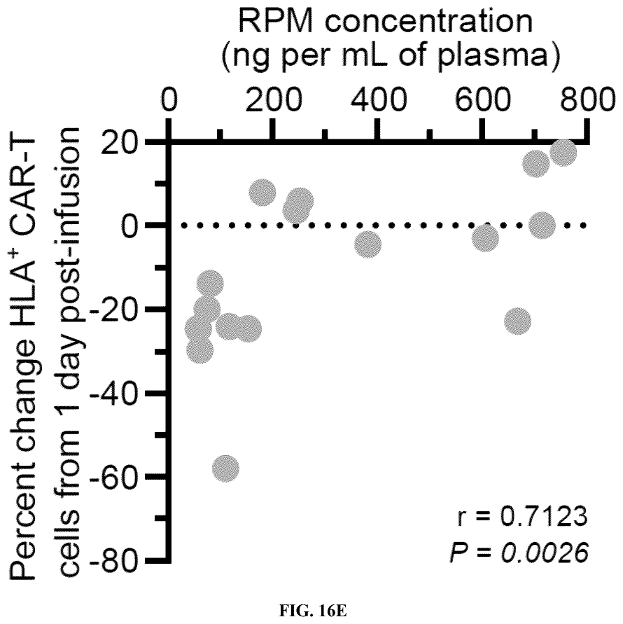

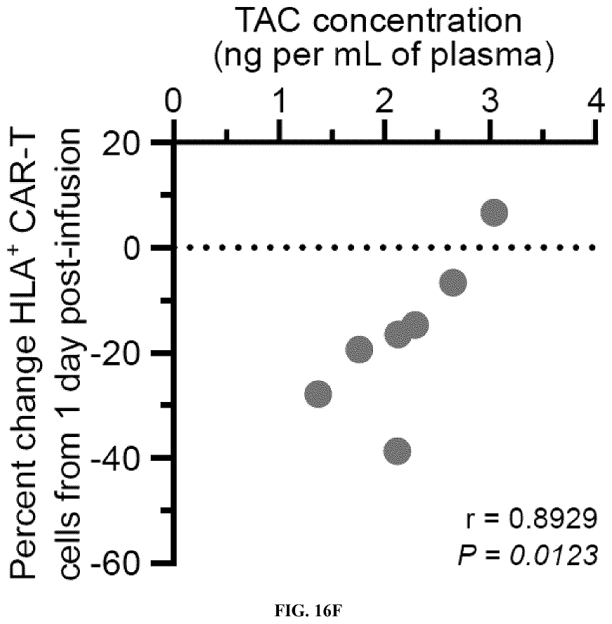

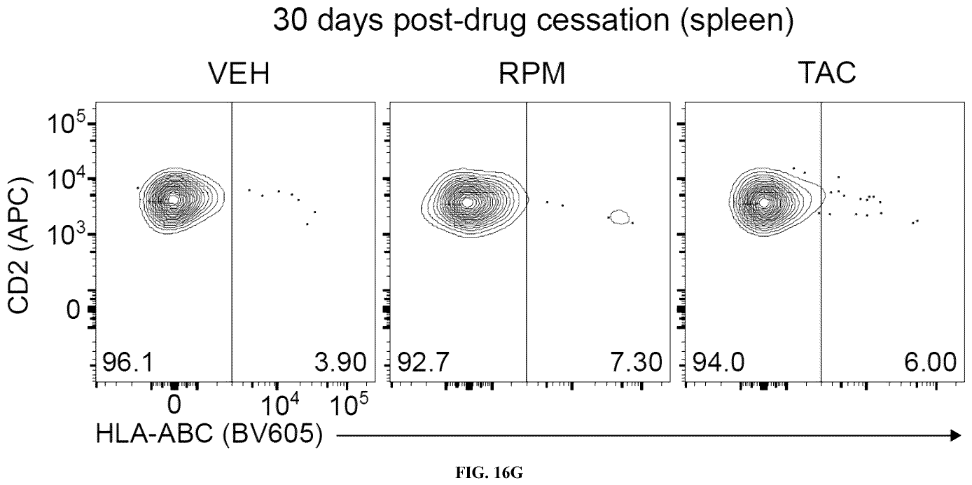

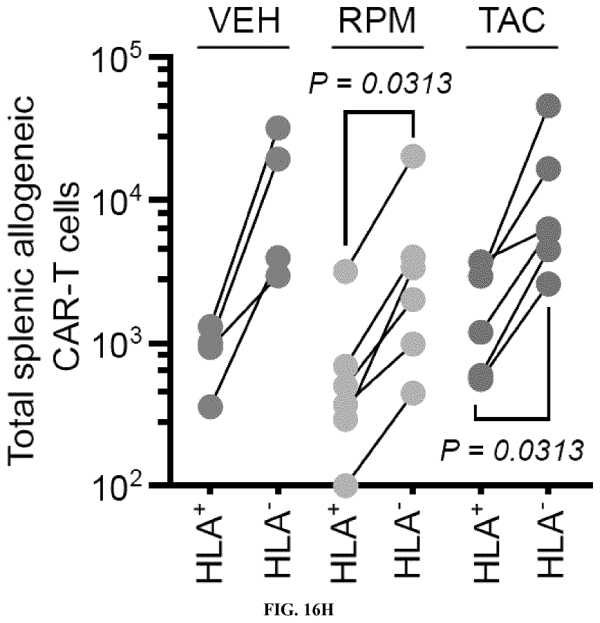

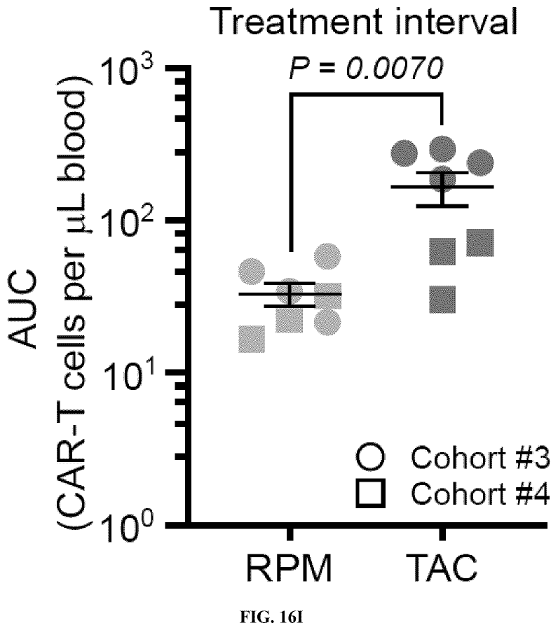

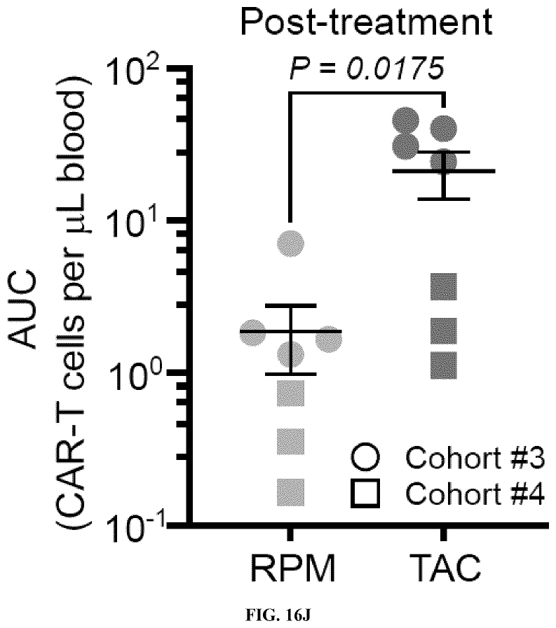

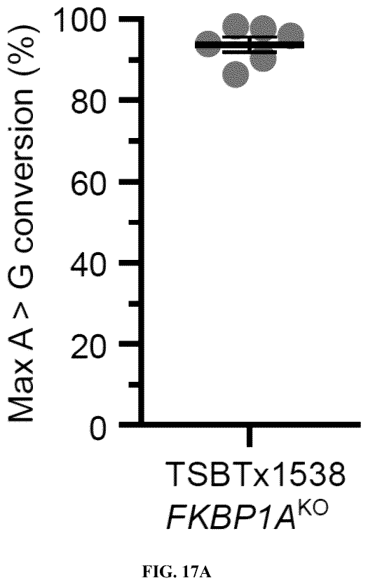

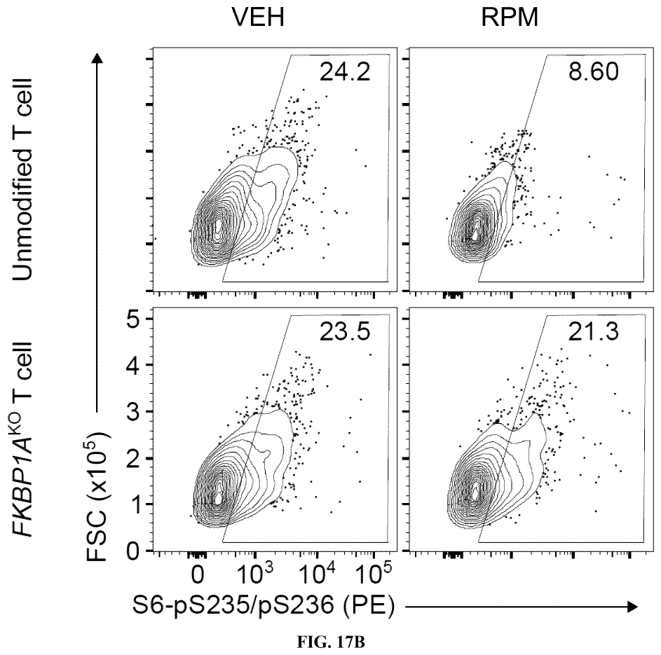

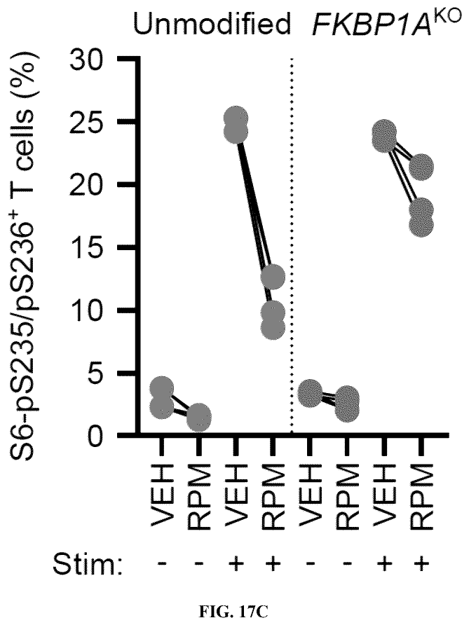

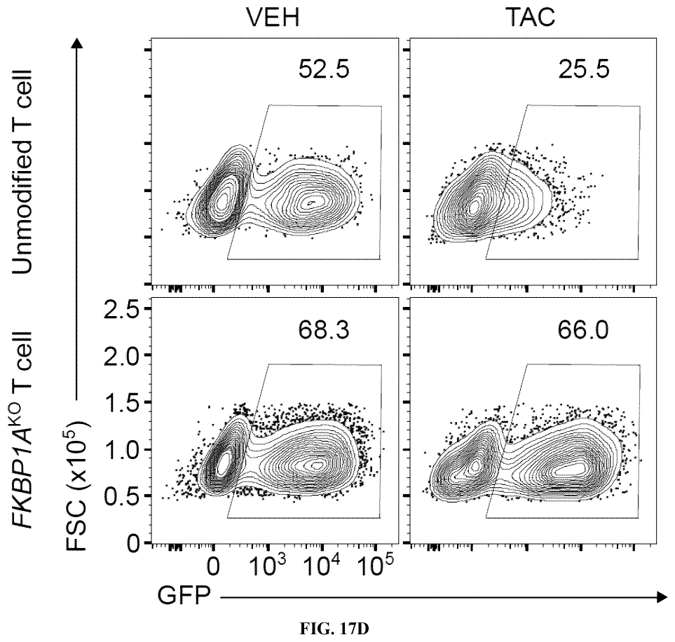

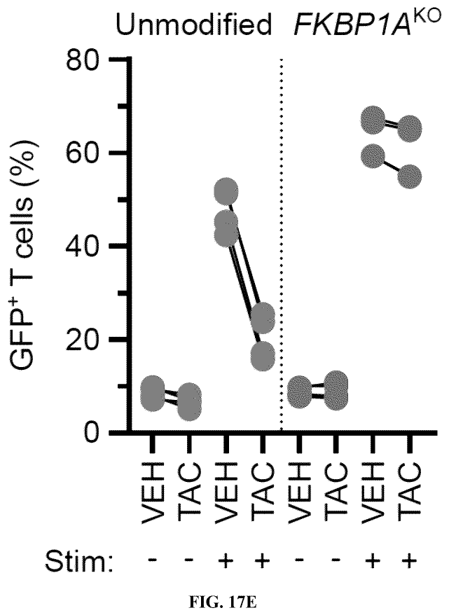

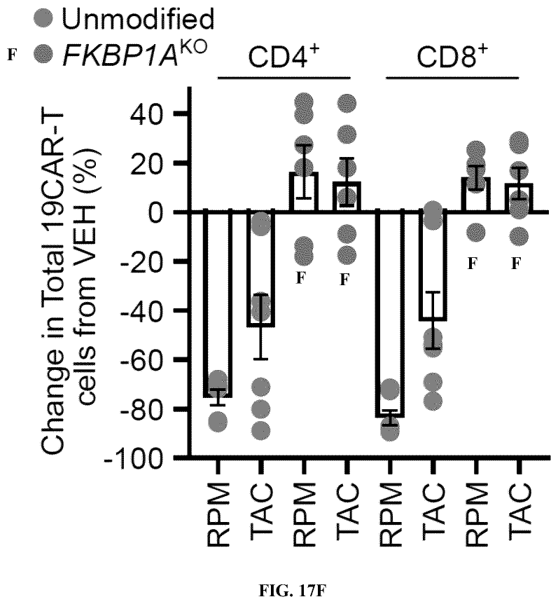

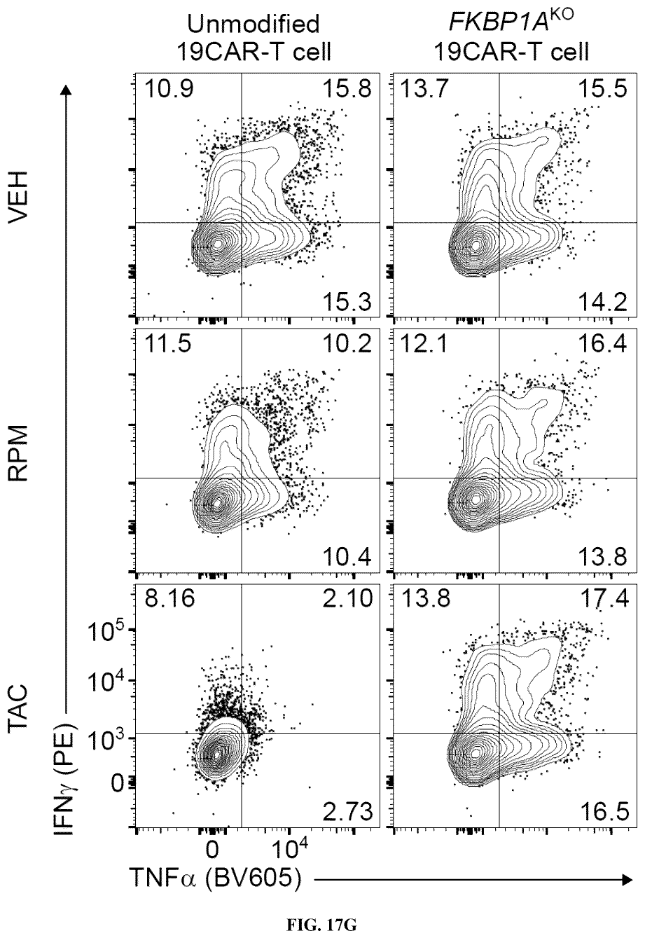

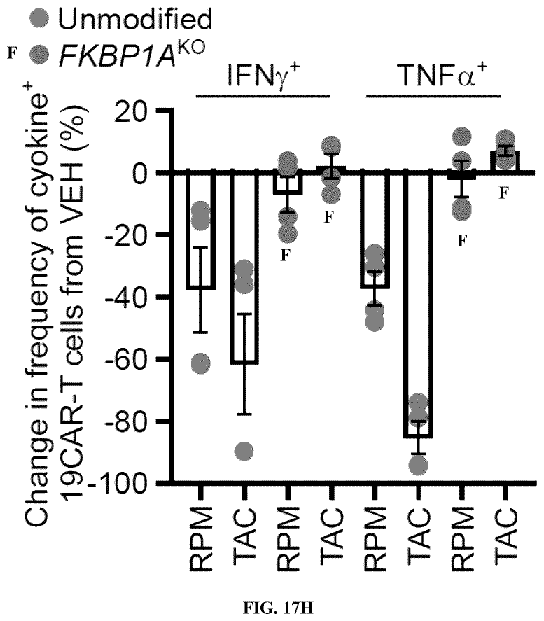

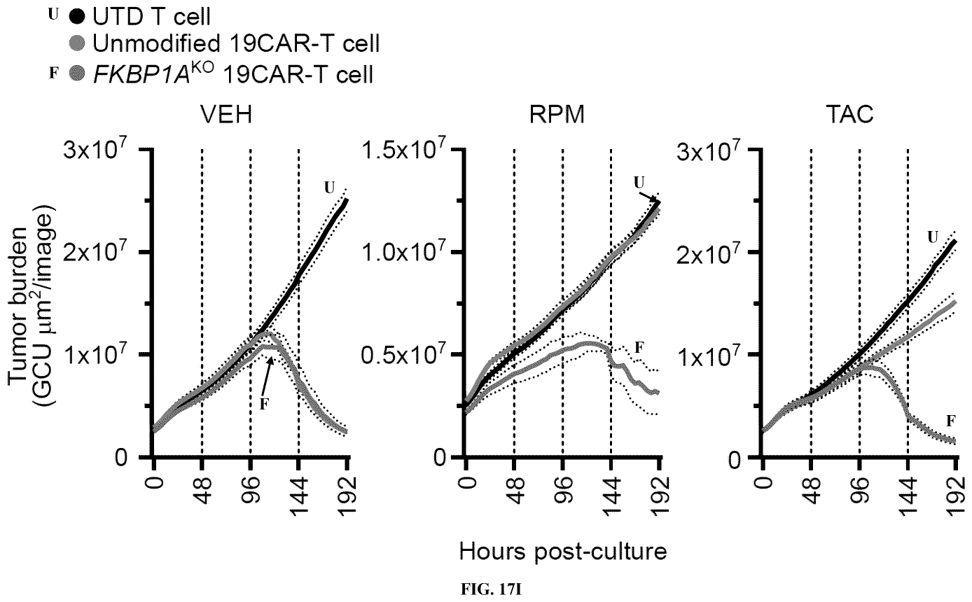

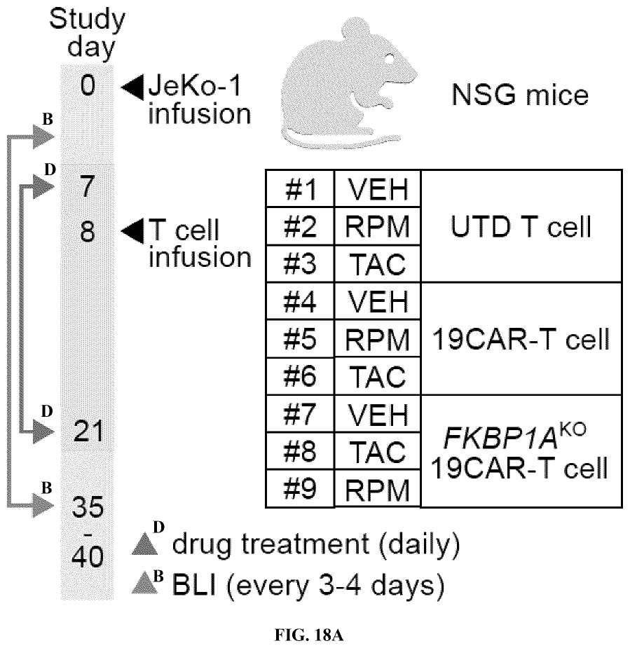

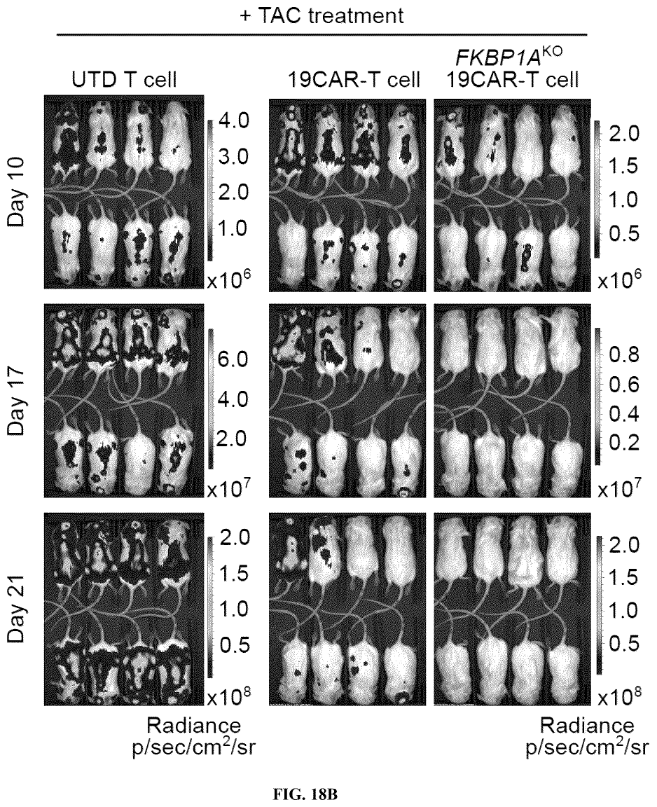

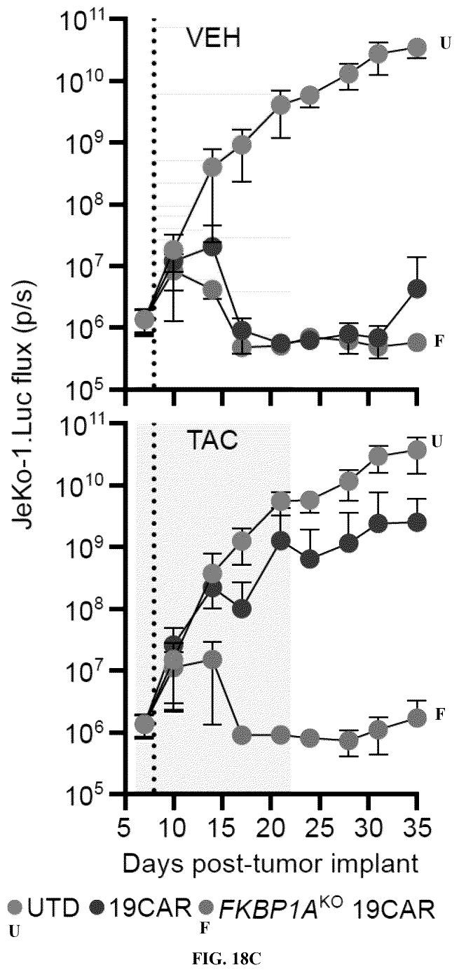

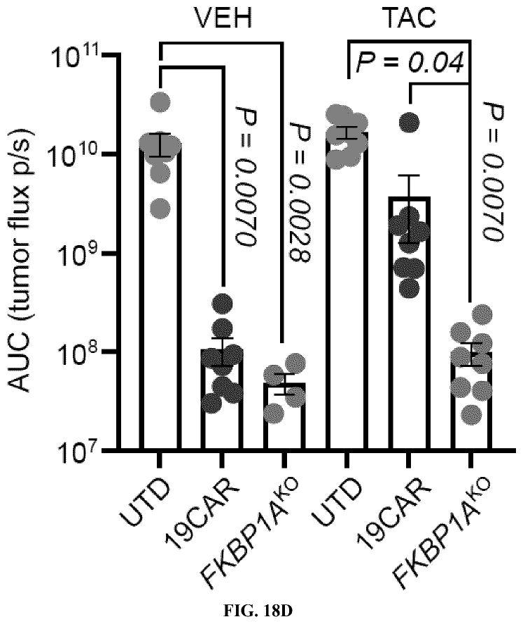

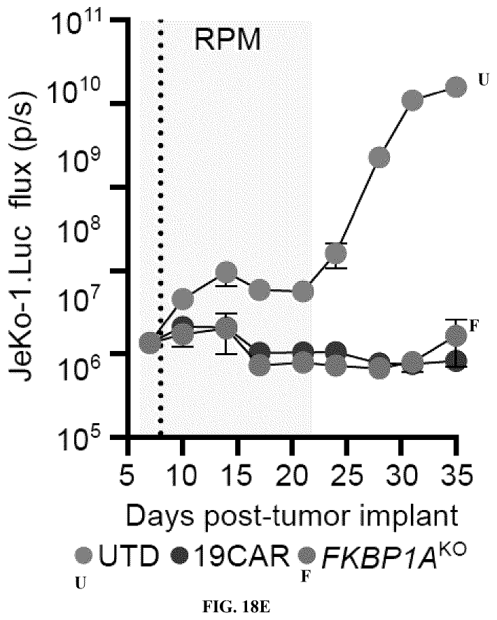

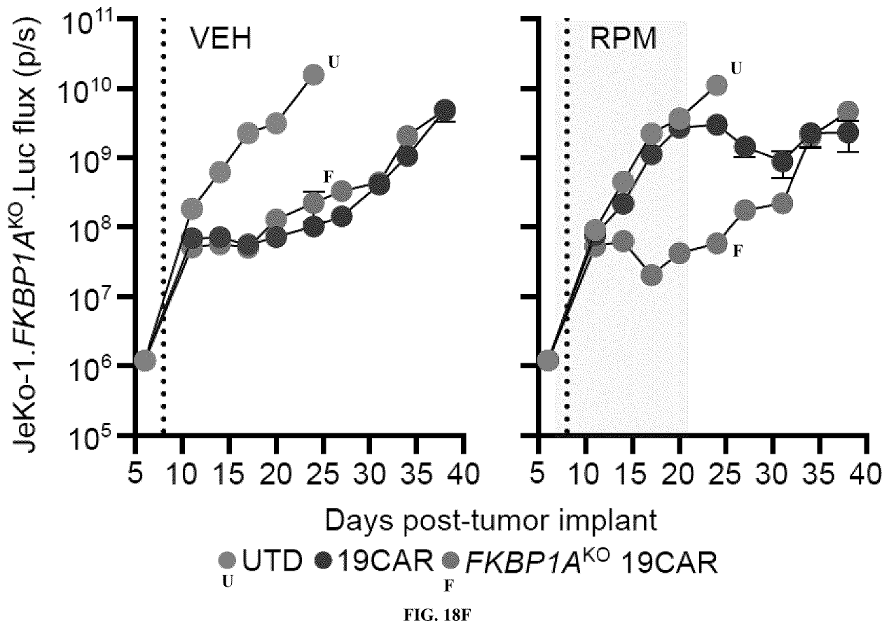

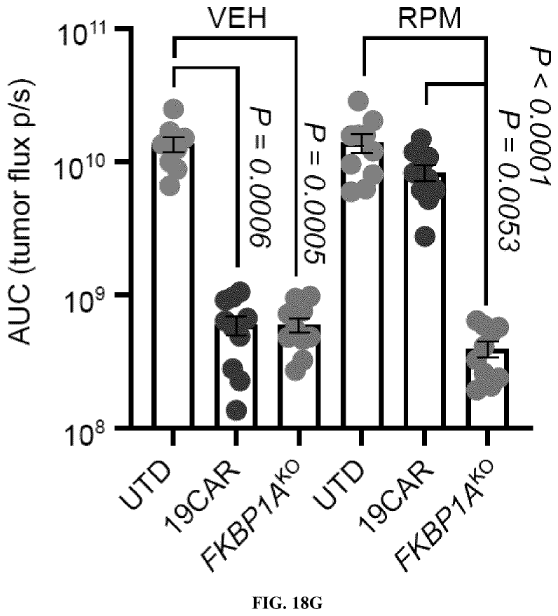

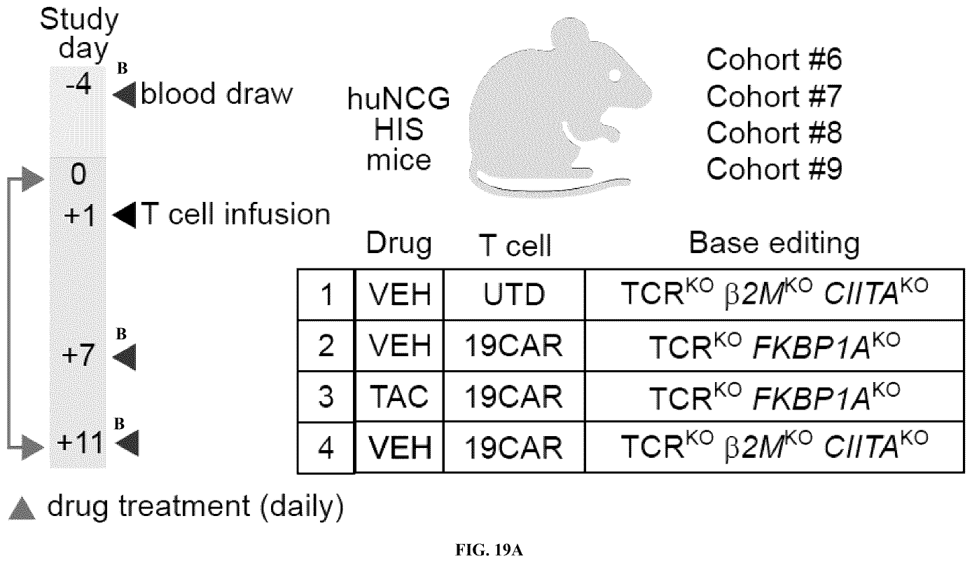

FIGS. 26A to 26E provide histograms and plots showing malignant B cell lines were sensitive to Rapamycin treatment in vitro. FIG. 26A provides histograms showing geometric median fluorescent intensity (MFI) of phosphorylated mTOR (pS2448), S6 (pS235/S2346) and 4EBP1 (pT36/T45) in JeKo-1, Raji and Nalm6 cell lines, as well as primary human monocytes and bulk lymphocytes. FIGS. 26B to 26D relate to an experiment where JeKo-1, Raji and Nalm6 cells were treated with DMSO or Rapamycin (RPM) and then analyzed for phosphorylation level of mTOR (FIG. 26B), S6 (FIG. 26C) and 4EBP1 (FIG. 26D). FIG. 26E provides plots showing JeKo-1, Raji and Nalm6 cell growth kinetics after treatment with DMSO or RPM at different concentrations. Symbols indicate mean and error bars show ±s.e.m.

FIGS. 27A and 27B provide schematic diagrams describing how the different regions of the prime editing guide RNA (pegRNA) sequences of Table 10 correspond to regions of the FKBP1A gene. FIG. 27A provides a schematic diagram showing how components of the pegRNAs containing Spacer 1 of Example 11 correspond to different regions of the FKBP1A gene. FIG. 27B provides a schematic diagram showing how components of the pegRNAs containing Spacer 2 of Example 11 correspond to different regions of the FKBP1A gene. In the pegRNA molecules, the spacer sequence remains constant (i.e., is Spacer 1 or Spacer 2), but the length of the reverse transcriptase template (RTT) and/or primer binding sequence (PBS) varies between the different pegRNA molecules of Table 10. In a cell, the spacers of the pegRNA molecules bind the forward strand (upper sequence) depicted in each of FIGS. 27A and 27B), and the “extension” containing the RTT and PBS binds the reverse strand (lower sequence). The protospacer adjacent motif was CGG or AGG and the reverse strand was nicked on the reverse strand between the nucleotides indicated by the two pipes (i.e., |). In FIGS. 27A and 27B, the star (*) indicates the location of the nucleobase targeted for editing. The nucleotide sequence depicted in FIG. 27A corresponds to SEQ ID NO: 775 and the nucleotide sequence depicted in FIG. 27B corresponds to SEQ ID NO: 775.

FIG. 28 provides a schematic diagram describing how the reverse transcriptase template (RTT) region of the prime editing guide RNA (pegRNA) sequences of Table 10 corresponds to regions of the FKBP1A gene. In FIG. 28, the term “Edit” indicates the nucleobase targeted for editing, as indicated by a “*,” the term “Possible RTT Span” indicates the nucleotides that may correspond to the RTT (see also FIGS. 27A and 27B), “Genome” indicates FKBP1A gene sequence, “Codons” indicates the position number of the corresponding codons and the encoded amino acids, “Amino acid” indicates the amino acid sequence encoded by the codons indicated in the gene sequence, and “Transcription Direction” indicates the direction of transcription by the reverse transcriptase during primer editing. The sequences depicted in FIG. 28 in order of occurrence, from top-to-bottom, correspond to SEQ ID NOs: 776-777.

FIG. 29 provides combined bar graphs (left bar graphs relate to cells expressing human leukocyte antigen A (HLA-A), HLA-B, and HLA-C (HLA-ABC+), and right bar graphs relate to cells base edited to knock out expression of HLA-A, HLA-B, and HLA-C (HLA-ABC−)) demonstrating that combined treatment with rapamycin and tacrolimus protected HLA-mismatched 4CAR-T cells from allorejection by recipient humanized mice. The mice were administered 5 million HLA-positive 4CAR-T cells that were base edited to knock-out (KO) T cell receptor (TCR) expression and 5 million HLA-negative 4CAR-T cells that were base-edited to KO TCR expression, beta-2-microglobulin (B2M) expression and class-II transcriptional activator (CIITA) expression. The bar graphs of FIG. 29 depict the percent of total 4CAR-T cells in peripheral blood that were HLA-positive (HLA-ABC+) or HLA-negative (HLA-ABC-) at 1, 7 and 14 days post-infusion from mice treated with vehicle (VEH) or the combination of rapamycin and tacrolimus (RPM+TAC). Unfilled circles represent individual mice, bars indicate mean, and error bars indicate +/−SEM.

### DETAILED DESCRIPTION

The disclosure features modified immune effector cells (e.g., T or NK cells) having increased resistance to inhibition by immunosuppressant agents relative to unmodified immune effector cells, compositions containing the same, and methods for use thereof.

The disclosure is based, at least in part, on the discovery that immune cells, such as T cells (e.g., CAR-T cells) or NK cells, can be modified through the use of base editor systems (e.g., those systems provided herein) to reduce or eliminate expression and/or activity of a polypeptide selected from one or more of FKBP1A, NR3C1, or PPIA to reduce susceptibility of the modified cells to an immunosuppressive agent. For example, it was found that cells modified to reduce or eliminate expression of FKBP1A showed reduced susceptibility to the immunosuppressants rapamycin and tacrolimus (e.g., increased proliferation, improved cytokine production, and improved cytotoxicity in the presence of the immunosuppressant agents).

Accordingly, the disclosure provides modified immune cells (e.g., CAR-T cells) with reduced or undetectable expression of FKBP1A, NR3C1, and/or PPIA and reduced susceptibility to immunosuppression by an immunosuppressive agent. The disclosure further provides methods for treatment of a neoplasia, where the methods involve administering to a subject a chimeric antigen receptor (CAR) T cell with reduced or undetectable expression of FKBP1A, NR3C1, and/or PPIA, and administering to the subject an immunosuppressant agent. Co-administration of the immunosuppressant agent (e.g., as part of an immunosuppression therapy) can advantageously inhibit rejection of the edited CAR T cells by a patient's immune system while having a reduced or negligible inhibitory effect on the edited CAR T cells themselves. Administration of the immunosuppressant agent dampens the function of the host immune system thereby inhibiting the generation of an effective alloreactive immune response.

### Immunosuppressant Agents

Immunosuppressant agents are therapeutic agents used to reduce an immune response in a subject. Such agents include, but are not limited to, mTOR inhibitors (e.g., a rapalog, such as rapamycin or Everolimus), Calcineurin Inhibitors (e.g., cyclosporine A or tacrolimus), and Glucocorticoids (e.g., Dexamethasone or Prednisolone). Immunosuppressant agents are often used to inhibit rejection of transplanted cells (e.g., allogeneic cells) obtained from a donor by the host's immune system. Immunosuppressant agents can reduce the proliferation of immune effector cells, reduce the cytotoxicity of immune effector cells, and/or reduce cytokine release. This immunosuppressive effect is mediated, for example, by binding of the immunosuppressant agent to a protein (e.g., FKBP1A, NR3C1, PPIA). For example, an NR3C1 polypeptide is capable of binding a steroid drug. Tacrolimus is capable of binding FKBP1A, and PPIA is capable of binding a glucocorticoid. When FKBP1A, PPIA, or NR3C1 expression is reduced or eliminated in an immune effector cell (T cell, NK cell) it renders the cell resistant to the effects of the immunosuppressant agent. In embodiments, an edited immune effector cell shows a 10%, 20%, 30%, 40%, 50%, 60%, 70%, 80%, 90%, 95%, 99% or greater resistance to the immunosuppressant agent (e.g., resistance to reductions in proliferation, cytotoxicity, and/or cytokine release caused by the agent as compared to a control cell). Resistance may be measured by assaying the immune effector cell's cytotoxicity, cytokine release or proliferation in the presence of the agent relative to the effects of the agent on an unedited immune effector cell.

### Car-T Cell Therapies

The present disclosure provides immune cells (e.g., T- or NK-cells) modified using nucleobase editors and/or nucleases described herein. The modified immune cells may express chimeric antigen receptors (CARs) (e.g., CAR-T cells). Modification of immune cells to express a chimeric antigen receptor can enhance an immune cell's immunoreactive activity, where the chimeric antigen receptor has an affinity for an epitope on an antigen, and where the antigen is associated with an altered fitness of an organism. For example, the chimeric antigen receptor can have an affinity for an epitope on a protein expressed in a diseased cell. Because the CAR-T cells can act independently of major histocompatibility complex (MHC), activated CAR-T cells can kill the diseased cell expressing the antigen. The direct action of the CAR-T cell evades defensive mechanisms that have evolved in response to MHC presentation of antigens to immune cells.

The modified immune cells and methods provided herein address known limitations of CAR-T therapy and represent a promising development towards the next generation of precision cell-based therapies.

In embodiments, one or more genes are modified in an immune effector cell so that the cell has a reduced level of, reduced activity of, lacks, or has virtually undetectable levels of FKBP1A, NR3C1, and/or PPIA. In embodiments, the immune effector cells are genetically modified to knock-out expression of FKBP1A, NR3C1, and/or PPIA. In some cases, the immune effector cells are genetically modified to reduce the activity of a FKBP1A, NR3C1, and/or PPIA polypeptide (e.g., through the introduction of a missense mutation to a codon encoding an amino acid in a ligand or DNA binding domain). In embodiments, one or more genes are modified in an immune effector cell so that the cell has a reduced level of, lacks, or has virtually undetectable levels of 1, 2, or all of the following polypeptides: FKBP1A, NR3C1, and/or PPIA. In embodiments, one or more genes are modified in an immune effector cell so that the cell has a reduced level of, lacks, or have virtually undetectable levels 1, 2, 3, 4, 5, or all of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and/or T cell receptor constant region (TRAC). In embodiments, one or more genes are modified in an immune effector cell so that the cell has a reduced level of, lacks, or have virtually undetectable levels of FKBP1A, NR3C1, and/or PPIA, and of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and/or T cell receptor constant region (TRAC).

In embodiments, the modified immune effector cells have increased resistance to immunosuppression by one or more of glucocorticoids (e.g., dexamethasone or prednisolone), calcineurin inhibitors (e.g., cyclosporine A or tacrolimus), and mTOR inhibitors (e.g., a rapalog, such as rapamycin or everolimus).

In embodiments, the modified immune effector cells of the disclosure activated by an antigen produce cytokines in the presence of an immunosuppressive agent at a level that is about or at least about 1.5-fold, 2-fold, 3-fold, 4-fold, 5-fold, 10-fold, 15-fold, 20-fold, 25-fold, 30-fold, 35-fold, 40-fold, 45-fold, or 50-fold greater than a level produced by unmodified immune effector cells under similar conditions. Non-limiting examples of cytokines include granzyme B, tumor necrosis factor alpha (TNFa), and interferon gamma (IFNg). In some cases, the modified immune effector cells of the disclosure activated by an antigen show levels of proliferation in the presence of an immunosuppressive agent that is about or at least about 1.5-fold, 2-fold, 3-fold, 4-fold, 5-fold, 10-fold, 15-fold, 20-fold, 25-fold, 30-fold, 35-fold, 40-fold, 45-fold, or 50-fold greater than levels of proliferation for unmodified immune effector cells under similar conditions.

In embodiments, one or more genes are modified in an immune effector cell so that the cell has a reduced level of, lacks, or have virtually undetectable levels of FKBP1A, NR3C1, and/or PPIA, and/or one or more of the following polypeptides relative to an unmodified immune cell: B cell leukemia/lymphoma 11b (Bcl11b); B cell leukemia/lymphoma 2 related protein A1d (Bcl2a1d); B cell leukemia/lymphoma 6 (Bcl6); butyrophilin-like 6 (Btnl6); CD151 antigen (Cd151); chemokine (C—C motif) receptor 7 (Ccr7); discs large MAGUK scaffold protein 5 (Dlg5); erythropoietin (Epo); G protein-coupled receptor 18 (Gpr18); interferon alpha 15 (Ifna15); interleukin 6 signal transducer (Il6st); interleukin 7 receptor (Il7r); Janus kinase 3 (Jak3); membrane associated ring-CH-type finger 7 (Marchf7); NCK associated protein 1 like (Nckap1l); phospholipase A2, group IIF (Pla2g2f); runt related transcription factor 3 (Runx3); Signal-regulatory protein beta 1B (Sirpb1b); transforming growth factor, beta 1 (Tgfb1); tumor necrosis factor (ligand) superfamily, member 14 (Tnfsf14); tumor necrosis factor (ligand) superfamily, member 18 (Tnfsf18); tumor necrosis factor (ligand) superfamily, member 8 (Tnfsf8); zinc finger CCCH type containing 8 (Zc3 h8); (Rac family small GTPase 2); (Slc4a1); 5-azacytidine induced gene 2 (Azi2); a disintegrin and metalloprotease domain 17 (Adam 17); a disintegrin and metalloprotease domain 8 (Adam8); Acetyl-CoA Acetyltransferase 1 (ACAT1); ACLY; adapter related protein complex 3 beta 1 subunit (Ap3b1); adapter related protein complex 3 delta 1 subunit (Ap3d1); adenosine A2a receptor (Adora2a); adenosine deaminase (Ada); adenosine kinase (Adk); adenosine regulating molecule 1 (Adrm1); advanced glycosylation end product-specific receptor (Ager) allograft inflammatory factor 1 (Aif1); AKT1; AKT2; amyloid beta (A4) precursor protein-binding family B member 1 interacting protein (Apbb1ip); ankyrin repeat and LEM domain (Ankle1); annecin A1 (Anxa1); arginase liver (Arg 1); arginase type II (Arg 2); AtPase Cu++ transporting, alpha polypeptide (Atp7a); autoimmune regulator (Aire); autophagy related 5 (Atg5); AXL; B and T Lymphocyte Associated (BTLA); B and T lymphocyte associated (Btla); B cell leukemia/lymphoma 10 (Bcl10); B cell leukemia/lymphoma 11a (Bcl11a); B cell leukemia/lymphoma 2 (Bcl2); B cell leukemia/lymphoma 3 (Bcl3); basic leucine zipper transcription factor, ATF-like (Batf); BCL2-associated X protein (Bax); BCL2L11; beta 2 microglobulin (B2m); BL2-associated agonist of cell death (Bad); BLIMP1; Bloom syndrome, RecQ like helicase (Blm); Bmi1 polycomb ring finger oncogene (Bmi1); Bone morphogenic protein 4 (Bmp4); Braf transforming gene (Braf); butyrophilin, subfamily 2, member A1 (Btn2a1); butyrophilin, subfamily 2, member A2 (Btn2a2); butyrophilin-like 1 (Btnl1); butyrophilin-like 2 (Btnl2); c-abl oncogene 1 (Abl1); c-abl oncogene 2 (Abl2); cadherin-like 26 (Cdh26); calcium channel, voltage dependent, beta 4 subunit (Cacnb4); CAMK2D; capping protein regulator and myosin 1 linker 2 (Carmil2); carcinoembryonic antigen-related cell adhesion molecule (Ceacam1); Casitas B-lineage lymphoma b (Cblb); CASP8; Caspase 3 (Casp3); caspase recruitment domain family member 11 (Card11); catenin (cadherin associated protein), beta 1 (Ctnnb1); caveolin 1 (Cav1); CBL-B; CCAAT/enhancer binding protein (C/EBP), beta (Cebpb); CCR10; CCR4; CCR5; CCR6; CCR9; CD103; CD11a; CD122; CD123; CD127; CD130; CD132; CD160 antigen (Cd160); CD161; CD19; CD1d1 antigen (Cd1d1); CD1d2 antigen (CD1d2); CD2 antigen (CD2); CD209e antigen (Cd209e); CD23; CD244 molecule A (Cd244a); CD24a antigen (Cd24a); CD27 antigen (CD27); CD274 antigen (Cd274); CD276 antigen (Cd276); CD28 antigen (Cd28); CD3 delta; CD3 epsilon; CD3 gamma; CD30; CD300A molecule (Cd300a); CD33; CD38; CD4 antigen (Cd4); CD40 ligand (Cd40lg); CD44 antigen (Cd44); CD46 antigen, complement regulatory protein (Cd46); CD47 antigen (Rh-related antigen, integrin-associated signal transducer) (Cd47); CD48 antigen (Cd48); CD5 antigen (Cd5); CD52; CD58; CD59b antigen (Cd59b); CD6 antigen (Cd6); CD69; CD7; CD70; CD74 antigen (Cd74); CD8; CD8 antigen (Cd8); CD80 antigen (Cd80); CD81 antigen (Cd81); CD82; CD83 antigen (Cd83); CD86; CD86 antigen (Cd86); CD8A; CD96; CD99; CDK4; CDK8; CDKN1B; chemokine (C motif) ligand 1 (Xcl1); chemokine (C—C motif) ligand 19 (Ccl19); chemokine (C—C motif) ligand 2 (Ccl2); chemokine (C—C motif) ligand 20 (Ccl20); chemokine (C—C motif) ligand 5 (Ccl5); chemokine (C—C motif) receptor 2 (Ccr2); chemokine (C—C motif) receptor 6 (Ccr6); chemokine (C—C motif) receptor 9 (Ccr9); chemokine (C—X—C motif) ligand 12 (Cxcl12); chemokine (C—X—C motif) receptor (Cxcr4); Chitinase 3 Like 1 (Chi31l); cholinergic receptor, nicotinic, alpha polypeptide 7 (Chrna7); chromodomain helicase DNA binding protein 7 (Chd7); CLA; Class II Major Histocompatibility Complex Transactivator (CIITA); cleft lip and palate associated transmembrane protein 1 (Clptm1); Cluster of Differentiation 123 (CD123); Cluster of Differentiation 3 (CD3); Cluster of Differentiation 33 (CD33); Cluster of Differentiation 52 (CD52); Cluster of Differentiation 7 (CD7); Cluster of Differentiation 96 (CD96); coagulation factor II (thrombin) receptor-like 1 (F2rl1); coil-coil domain containing 88B (Ccdc88b); core-binding factor beta (Cbfb); coronin, actin binding protein 1A (Coro1a); coxsackie virus and adenovirus receptor (Cxadr); CS-1; CSF2CSK; c-src tyrosine kinase (Csk); C-type lectin domain family 2, member i (Clec2i); C-type lectin domain family 4, member a2 (Clec4a2); C-type lectin domain family 4, member d (Clec4d); C-type lectin domain family 4, member e (Clec4e); C-type lectin domain family 4, member f (Clec4f); C-type lectin domain family 4, member g (Clec4g); CUL3; CXCR3; cyclic GMP-AMP synthase (Cgas); cyclin D3 (Ccnd3); cyclin dependent kinase inhibitor 2A (Cdkn2a); cyclin-dependent kinase (Cdk6); CYLD lysine 63 deubiquitinase (Cyld); cysteine-rich protein 3 (Crip3); cytidine 5′-triphosphate synthase (Ctps); Cytochrome P450 Family 11 Subfamily A Member 1 (Cyp11a1); cytochrome P450, family 26, subfamily b, polypeptide (Cyp26b1); Cytokine Inducible SH2 Containing Protein (CISH); cytotoxic T lymphocyte-associated protein 2 alpha (Ctla2a); Cytotoxic T-Lymphocyte Associated Protein 4 (CTLA-4); DCK; dedicator of cytokinesis 2 (Dock2); dedicator of cytokinesis 8 (Dock8); delta like canonical Notch ligand 4 (Dll4); deltex 1, E3 ubiquitin ligase (Dtx1); deoxyhypusine synthase (Dhps); DGKA; DGKZ; DHX37; dicer 1, ribonuclease type III (Dicer1); dipeptidylpeptidase 4 (Dpp4); discs large MAGUK scaffold protein 1 (Dlg1); DnaJ heat shock protein family (Hsp40) member A3 (Dnaja3); dolichyl-di-phosphooligosaccharide-protein glycotransferase (Ddost); double homeobox B-like 1 (Duxbl1); drosha, ribonuclease type III (Drosha); dual specificity phosphatase 10 (Dusp10); dual specificity phosphatase 22 (Dusp22); dual specificity phosphatase 3 (Dusp3); E74-like factor 4 (Elf4); early growth response 1 (Egr1); early growth response 3 (Egr3); ELOB (TCEB2); ENTPD1 (CD39); eomesodermin (Eomes); Eph receptor B4 (Ephb4); Eph receptor B6 (Ephb6); ephrin B1 (Efnb1); ephrin B2 (Efnb2); ephrin B3 (Efnb3); Epstein-Barr virus induced gene 3 (Ebi3); erb-b2 receptor tyrosine kinase (Erbb2); eukaryotic translation initiation factor 2 alpha kinase 4 (Eif2ak4); FADD; family with sequence similarity 49, member B (Fam49b); Fanconi anemia, complementation group A (Fanca); Fanconi anemia, complementation group D2 (Fancd2); Fas (TNF receptor superfamily member 6) (Fas); Fas (TNFRSF6)-associated via death domain (Fadd); Fas Cell Surface Death Receptor (FAS); Fc receptor, IgE, high affinity I, gamma polypeptide (Fcer1g); fibrinogen-like protein 1 (Fgl1); fibrinogen-like protein 2 (Fgl2); FK506 binding protein 1a (Fkbp1a); FK506 binding protein 1b ((Fkbp1b); flotillin 2 (Flot2); FMS-like tyrosine kinase (Flt3); forkhead box J1 (Foxj1); forkhead box N1 (Foxn1); forkhead box P1 (Foxp1); forkhead box P3 (Foxp3); frizzled class receptor 5 (Fzd5); frizzled class receptor 7 (Fzd7); frizzled class receptor 8 (Fzd8); fucosyltransferase 7 (Fut7); Fyn proto-oncogene (Fyn); gap junction protein, alpha 1 (Gja1); GATA binding protein 3 (GATA3); GCN2 kinase (IDO pathway); gelsolin (Gsn); GLI-Kruppel family member GLI3 (Gli3); glycerol-3-phosphate acyltransferase, mitochondrial (Gpam); growth arrest and DNA-damage-inducible 45 gamma (Gadd45g); GTPase, IMAP family member 1 (Gimap1); H1TET2; H2.0-like homeobox (Hlx); haematopoietic 1 (hem1); HCLS1 binding protein 3 (Hs1bp3); heat shock 105 kDa/110 kDa protein 1 (Hsph1); heat shock protein 1 (chaperonin) (Hspd1); heat shock protein 90, alpha (cytosolic), class A member 1 (Hsp90aa1); hematopoietic SH2 domain containing (Hsh2d); hepatitis A virus cellular receptor 2 (Haver2); hes family bHLH transcription factor 1 (Hes1); histocompatibility 2, class II antigen A, alpha (H2-Aa); histocompatibility 2, class II antigen A, beta 1 (H2-Ab1); histocompatibility 2, class II, locus dMa (H2-dMa); histocompatibility 2, M region locus 3 (H3-M3); histocompatibility 2, O region alpha locus (H2-Oa); histocompatibility 2, T region locus 23 (H2-T23); HLA-DR; homeostatic iron regulator (Hfe); icos ligand (Icosl); IKAROS family zinc finger 1 (Ikzf1); IL10; IL10RA; IL2 inducible T cell kinase (Itk); IL6R; Indian hedgehog (Ihh); indoleamine 2,3-dioxygenase 1 (Ido1); inducible T cell co-stimulator (Icos); inositol 1,4,5-trisphosphate 3-kinase B (Itpkb); insulin II (Ins2); insulin-like growth factor 1 (Igf1); insulin-like growth factor 2 (Igf2); insulin-like growth factor binding protein 2 (Igfbp2); integrin alpha L (Itgal); integrin alpha M (Itgam); integrin alpha V (Itgav); integrin alpha X (Itgax); integrin beta 2 (Itgb2); integrin, alpha D (Itgad); intercellular adhesion molecule 1 (Icam1); interferon (alpha and beta) receptor 1 (Ifnar1); interferon alpha 1 (Ifna1); interferon alpha 11 (Ifna11); interferon alpha 12 (Ifna12); interferon alpha 13 (Ifna13); interferon alpha 14 (Ifna14); interferon alpha 16 (Ifna16); interferon alpha 2 (Ifna2); interferon alpha 4 (Ifna4); interferon alpha 5 (Ifna5); interferon alpha 6 (Ifna6); interferon alpha 7 (Ifna7); interferon alpha 9 (Ifna9); interferon alpha B (Ifnab); interferon beta 1 (Ifnb1); interferon gamma (IFNg); interferon kappa (Ifnk); interferon regulatory factor 1 (Irf1); interferon regulatory factor 4 (Irf4); interferon zeta (Ifnz); interleukin 1 beta (Il1b; interleukin 1 family, member 8 (Il1f8); interleukin 1 receptor-like 2 (Il1rl2); interleukin 12 receptor, beta1 (Il12rb1); interleukin 12a (Il12a); interleukin 12b (Il12b); interleukin 15 (Il15); interleukin 18 (Il18); interleukin 18 receptor 1 (Il18r1); interleukin 2 (Il2); interleukin 2 receptor, alpha chain (Il2ra); interleukin 2 receptor, gamma chain (Il2rg); interleukin 20 receptor beta (Il20rb); interleukin 21 (Il21); interleukin 23, alpha subunit p19 (Il23a); interleukin 27 (Il27); interleukin 4 (Il4); interleukin 4 receptor, alpha (Il4ra); interleukin 6 (Il6); interleukin 7 (Il7); IRF8; itchy, E3 ubiquitin protein ligase (Itch); jagged 2 (Jag2); jumonji domain containing 6 (Jmjd6); JUNB; junction adhesion molecule like 9 (Jam9); K (lysine) acetyltransferase 2A (Kat2a); KDEL (Lys-Asp-Glu-Leu) endoplasmic reticulum protein retention receptor 1 (Kdelr1); KIT proto-oncogene receptor tyrosine kinase (Kit); LAG-3; LAIR-1 (CD305); LDHA; lectin, galactose binding, soluble 1 (Lgals1); lectin, galactose binding, soluble 3 (Lgals3); lectin, galactose binding, soluble 8 (Lgals8); lectin, galactose binding, soluble 9 (Lgals9); leptin (Lep); leptin receptor (Lepr); leucine rich repeat containing 32 (Lrrc32); leukocyte immunoglobulin-like receptor, subfamily B, member 4A (Lilrb4a); LFNG O-fucosylpeptide 3-beta-N-acetylglucosaminyltransferase (Lfng); LIF; ligase IV, DNA, ATP-dependent (Lig4); LIM domain only 1 (Lmo1); limb region 1 like (Lmbrl); linker for activation of T cells (Lat); lymphocyte antigen 9 (Ly9); lymphocyte cytosolic protein 1 (Lcp1); lymphocyte protein tyrosine kinase (Lck); lymphocyte transmembrane adaptor 1 (Lax1); lymphocyte-activation gene 3 (Lag3); lymphoid enhancer binding factor 1 (Lef1); LYN; lysyl oxidase-like 3 (Lox13); MAD1 mitotic arrest deficient 1-like 1 (Mad1l1); MALTI paracaspase (Malt1); MAP4K4; MAPK14; MCJ; mechanistic target of rapamycin kinase (Mtor); MEF2D; Methylation-Controlled J Protein (MCJ); methyltransferase like 3 (Mettl3); MGAT5; MHC I like leukocyte 2 (Mill2); midkine (Mdk); mitogen-activated protein kinase 8 interacting protein 1 (Mapk8ip10); moesin (Msn); myelin protein zero-like 2 (Mpzl2); myeloblastosis oncogene (Myb); myosin, heavy polypeptide 9, non-muscle (Myh9); Nedd4 family interacting protein 1 (Ndfip1); neural precursor cell expressed, developmentally down-regulated 4 (Nedd4); NFATc1; NFATC2; NFATC4; NFKB activating protein (Nkap); nicastrin (Ncstn); NK2 homeobox 3 (Nkx2-3); NLR family, CARD domain containing 3 (Nlrc3); NLR family, pyrin domain containing 3 (Nlrp3); non-catalytic region of tyrosine kinase adaptor protein 1 (Nck1); non-catalytic region of tyrosine kinase adaptor protein 2 (Nck2); non-homologous end joining factor 1 (Nhej1); non-SMC condensin II complex, subunit H2 (Ncaph2); Notch-regulated ankyrin repeat protein (Nrarp); NT5E (CD73); nuclear factor of activated T cells, cytoplasmic, calcineurin dependent (Nfatc3); nuclear factor of kappa light polypeptide gene enhancer in B cells inhibitor, delta (Nfkbid); nuclear receptor co-repressor 1 (Ncor1); Nuclear Receptor Subfamily 4 Group A Member 1 (NR4A1); Nuclear Receptor Subfamily 4 Group A Member 2 (NR4A2); Nuclear Receptor Subfamily 4 Group A Member 3 (NR4A3); ODC1; OTU domain containing 5 (Otud5); OTULINL (FAM105A); paired box 1 (Pax1); PDCD1 (PD1; PD-1); PDIA3; pellino 1 (Peli1); peroxiredoxin 2 (Prdx2); PHD1 (EGLN2); PHD2 (EGLN1); PHD3 (EGLN3); phosphodiesterase 5A, cGMP-specific (Pde5a); phosphoinositide-3-kinase regulatory subunit (Pik3r6); phospholipase A2, group IIA (Pla2g2a); phospholipase A2, group IID (Pla2g2d); phospholipase A2, group IIE (Pla2g2e); phosphoprotein associated with glycosphingolipid microdomains 1 (Pag1); PIK3CD; PIKFYVE; POZ (BTB) and AT hook containing zinc finger 1 (Patz1); PPARa; PPARd; PR domain containing 1, with ZNF domain (Prdm1); presenilin 1 (Psen1); presenilin 2 (Psen2); PRKACA; PRKC, apoptosis, WT1, regulator (Pawr); programmed cell death 1 ligand 2 (Pdcd1lg2); prosaposin (Psap); prostaglandin E receptor 4 (subtype EP4) (Ptger4); protein kinase C, theta 2 (Prkcq); protein kinase C, zeta (Prkcz); protein kinase, cAMP dependent regulatory, type I, alpha (Prkar1a); protein kinase, DNA activated, catalytic polypeptide (Prkdc); protein phosphatase 3, catalytic subunit, beta isoform (Ppp3cb); protein tyrosine phosphatase, non-receptor type 2 (Ptpn2); protein tyrosine phosphatase, non-receptor type 22 (lymphoid) (Ptpn22); protein tyrosine phosphatase, non-receptor type 6 (Ptpn6); protein tyrosine phosphatase, receptor type, C (Ptprc); PTEN; PTPN11; purine-nucleoside phosphorylase (Pnp); purinergic receptor P2X, ligand-gated ion channel, 7 (P2rx7); PVR Related Immunoglobulin Domain Containing (PVRIG; CD112R); PYD and CARD domain containing 7 (Pycard); RAB27A, member RAS oncogene family (Rab27a); RAB29, member RAS oncogene family (Rab29); radical S-adenosyl methionine domain containing 2 (Rsad2); RAR-related orphan receptor alpha (Rora); RAR-related orphan receptor gamma (Ror); RAS guanyl releasing protein 1 (Rasgrp1); ras homolog family member A (Rhoa); ras homolog family member H (Rhoh); RAS protein activator like 3 (Rasal3); RASA2; receptor (TNFRSF)-interacting serine-threonine kinase 2 (Ripk2); recombination activating gene 1 (Rag1); recombination activating gene 2 (Rag2); Regulatory Factor X Associated Ankyrin Containing Protein (RFXANK); RHO family interacting cell polarization regulator 2 (Ripor2); ribosomal protein L22 (Rpl 22); ribosomal protein S6 (Rps6); RING CCCH (C3H) domains 1 (Rc3 h1); ring finger and CCCH-type zinc finger domains 2 (Rc3 h2); RNF2; runt related transcription factor 1 (Runx1); runt related transcription factor 2 (Runx2); SAM and SH3 domain containing 3 (Sash3); schlafen 1; Selectin P Ligand/P-Selectin Glycoprotein Ligand-1 (SELPG/PSGL1) polypeptide; selenoprotein K (Selenok); sema domain immunoglobulin domain (Ig), transmembrane domain (TM) and short cytoplasmic domain, (semaphorin) 4A (Sema4a); serine/threonine kinase 11 (Stk11); SH3 domain containing ring finger 1 (Sh3rf1); SHP1; sialophorin (Spn); SIGLEC15; signal transducer and activator of transcription 3 (Stat3); signal transducer and activator of transcription 5A (Stat5A); signal transducer and activator of transcription 5B (Stat5B); signal-regulatory protein alpha (Sirpa); Signal-regulatory protein beta 1A (Sirpb1a); Signal-regulatory protein beta 1C (Sirpb1c); SLA; SLAM family member 6 (Slamf6); SLAMF7; SMAD family member 3 (Smad3); SMAD family member 7 (Smad7); SMARCA4; solute carrier family 11 (proton-coupled divalent metal ion transporters), member 1 (Slc11a1); solute carrier family 4 (anion exchanger), member 1; solute carrier family 46, member 2 (Slc46a2); sonic hedgehog (Shh); SOS Ras/Rac guanine nucleotide exchange factor 1 (Sos1); SOS Ras/Rac guanine nucleotide exchange factor 2 (Sos2); special AT-rich sequence binding protein 1 (Satb1); spleen tyrosine kinase (Syk); Sprouty RTK Signaling Antagonist 1 (Spry1); Sprouty RTK Signaling Antagonist 2 (Spry2); squamous cell carcinoma antigen recognized by T cells (Sart1); src homology 2 domain-containing transforming protein B (Shb); Src-like-adaptor 2 (Sla2); SRY (sex determining region Y)-box 4 (Sox4); STK4; suppression inducing transmembrane adaptor 1 (Sit1); suppressor of cytokine signaling 1 (Socs1); suppressor of cytokine signaling 5 (Socs5); suppressor of cytokine signaling 6 (Socs6); surfactant associated protein D (Sftpd); SUV39; syndecan 4 (Sdc4); syntaxin 11 (Stx11); T Cell Immunoglobulin Mucin 3 (Tim-3); T cell immunoreceptor with Ig and ITIM domains (Tigit); T cell receptor alpha joining 18 (Traj18); T Cell Receptor Beta Constant 1 (TRBC1); T Cell Receptor Beta Constant 2 (TRBC2); T cell, immune regulator 1, ATPase, H+ transporting, lysosomal V0 protein A3 (Tcirg1); T cell-interacting, activating receptor on myeloid cells 1 (Tarm1); T-box 21 (Tbx21); TCR; TCR alpha; TCR beta; TCR complex gene sequence; Tet Methylcytosine Dioxygenase 2 (TET2); TGFbRII; TGFbRII (TGFBR2); three prime repair exonuclease 1 (Trex1); thymocyte selection associated (Themis); thymus cell antigen 1, theta (Thy1); TMEM222; TNF receptor-associated factor 6 (Traf6); TNFAIP3; TNFRSF10B; TNFRSF8 (CD30); TOX; TOX2; TRAC; transformation related protein 53 (Trp53); Transforming Growth Factor Beta Receptor II (TGFbRII); transforming growth factor, beta receptor II (Tgfbr2); transmembrane 131 like (Tmem1311); transmembrane protein 98 (Tmem98); triggering receptor expressed on myeloid cells-like 2 (Trem12); TSC complex subunit 1 (Tsc1); tumor necrosis factor (ligand) superfamily, member 11 (Tnfsf11); tumor necrosis factor (ligand) superfamily, member 13b (Tnfsf13b); tumor necrosis factor (ligand) superfamily, member 4 (Tnfsf4); tumor necrosis factor (ligand) superfamily, member 9 (Tnfsf9); tumor necrosis factor receptor superfamily, member 13c (Tnfrsf13c); tumor necrosis factor receptor superfamily, member 4 (Tnfrsf4); tumor necrosis factor, alpha-induced protein 8-like 2 (Tnfa1p8l2); twisted gastrulation BMP signaling modulator 1 (Twsg1); UBASH3A; vanin 1 (Vnn1); vascular cell adhesion molecule 1 (Vcam1); VHL; v-maf musculoaponeurotic fibrosarcoma oncogene family, protein B (avian) (Mafb); V-set and immunoglobulin domain containing 4 (Vsig4); V-Set Immunoregulatory Receptor (VISTA); WD repeat and FYVE domain containing 4 (Wdfy4); wingless-type MMTV integration site family, member 1 (Wnt1); wingless-type MMTV integration site family, member 4 (Wnt4); WNT signaling pathway regulator (Apc); WW domain containing E3 ubiquitin protein ligase 1 (Wwp1); XBP1; YAP1; ZAP70; ZC3H12A; zfp35; zinc finger and BTB domain containing 1 (Zbtb1); zinc finger and BTB domain containing 7B (Zbtb7B); zinc finger CCCH type containing 12A (Zc3h12a); zinc finger CCCH type containing 12D (Zc3h12d); zinc finger E-box binding homeobox 1 (Zeb1); zinc finger protein 36, C3H type (Zfp36); zinc finger protein 36, C3H type-like 1 (Zfp36L1); zinc finger protein 36, C3H type-like 2 (Zfp36L2); and zinc finger protein 683 (Zfp683).

Immune cells and/or immune effector cells can be isolated or purified from a sample collected from a subject/donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD4⁺ is used as a marker to select T cells. In one embodiment, CD8⁺ is used as a marker to select T cells.

In another embodiment, the present disclosure provides T cells that have targeted gene knock-outs at the TCR constant region (TRAC), which is responsible for TCRαβ surface expression. TCRαβ-deficient CAR-T cells are compatible with allogeneic immunotherapy (Qasim et al., Sci. Transl. Med. 9, eaaj2013 (2017); Valton et al., Mol Ther. 2015 September; 23 (9): 1507-1518). If desired, residual TCRαβ T cells are removed using CliniMACS magnetic bead depletion to minimize the risk of GVHD. In another embodiment, the present disclosure provides donor T cells selected ex vivo to recognize minor histocompatibility antigens expressed on recipient hematopoietic cells, thereby minimizing the risk of graft-versus-host disease (GVHD), which is the main cause of morbidity and mortality after transplantation (Warren et al., Blood 2010; 115(19):3869-3878).

Another technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4, CD8, CD28, CD45) and corresponding gating strategy. In one embodiment, a CD4 gating strategy is employed. In one embodiment, a CD8 gating strategy is employed. In one embodiment, a CD4 and CD8 gating strategy is employed. In some embodiments, a gating strategy for other markers specific to an immune effector cell is employed instead of, or in combination with, the CD4 and/or CD8 gating strategy.

In embodiments, the immune effector cells contemplated in the present disclosure are effector T cells. In some embodiments, the effector T cell is a naïve CD8+ T cell, a cytotoxic T cell, a natural killer T (NKT) cell, or a natural killer (NK) cell. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, immune effector cells are effector NK cells. In some embodiments, the immune effector cell is any other subset of T cells. The modified immune effector cell may express, in addition to the chimeric antigen receptor (CAR), an exogenous cytokine, a different chimeric receptor, or any other agent that would enhance immune effector cell signaling or function. For example, co-expression of the chimeric antigen receptor and a cytokine may enhance the CAR-T cell's ability to lyse a target cell.

Provided herein are also polynucleotides that encode the chimeric antigen receptors (CARs) described herein. In some embodiments, the nucleic acid molecule is isolated or purified. Delivery of the nucleic acid molecules ex vivo can be accomplished using methods known in the art. For example, immune cells obtained from a subject may be transformed with a nucleic acid vector encoding the chimeric antigen receptor. The vector may then be used to transform recipient immune cells so that these cells will then express the chimeric antigen receptor. Efficient means of transforming immune cells include transfection and transduction. Such methods are well known in the art. For example, applicable methods for delivery the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

Some aspects of the present disclosure provide for immune cells comprising a chimeric antigen receptor (CAR) and an altered endogenous gene that provides resistance to development of an exhausted phenotype after repeated or continuous exposure to an antigen and/or increased persistence, resistance to fratricide, enhances immune cell function, resistance to immunosuppression or inhibition, or a combination thereof. In some embodiments, the altered endogenous gene may be created by base editing. In some embodiments, the base editing may reduce or attenuate the gene expression. In some embodiments, the base editing may reduce or attenuate the gene activation. In some embodiments, the base editing may reduce or attenuate the functionality of the gene product. In some other embodiments, the base editing may activate or enhance the gene expression. In some embodiments, the base editing may increase the functionality of the gene product. In some embodiments, the altered endogenous gene may be modified or edited in a start codon, an exon, an intron, a splice acceptor site, a splice donor site, an exon-intron injunction, or a regulatory element thereof. The modification may be edit to a single nucleobase in a gene or a regulatory element thereof. The modification may be in a exon, more than one exons, an intron, or more than one introns, or a combination thereof. The modification may be in an open reading frame of a gene. The modification may be in an untranslated region of the gene, for example, a 3′-UTR or a 5′-UTR. In some embodiments, the modification is in a regulatory element of an endogenous gene. In some embodiments, the modification is in a promoter, an enhancer, an operator, a silencer, an insulator, a terminator, a transcription initiation sequence, a translation initiation sequence (e.g., a Kozak sequence), or any combination thereof.

Immune effector cells expressing an endogenous immune cell receptor and a chimeric antigen receptor (CAR) may recognize and attack host cells, a circumstance termed graft versus host disease (GVHD). The alpha component of the immune cell receptor complex is encoded by the TRAC gene, and in some embodiments, this gene is edited such that the alpha subunit of the TCR complex is nonfunctional or absent. Because this subunit is necessary for endogenous immune cell signaling, editing this gene can reduce the risk of graft versus host disease caused by allogeneic immune cells.

In some embodiments, editing of genes to provide resistance to development of an exhausted phenotype after repeated or continuous exposure to an antigen, increased persistence, fratricide resistance, enhance the function of the immune cell or to reduce immunosuppression or inhibition can occur in the immune cell before the cell is transformed to express a chimeric antigen receptor (CAR). In other aspects, editing of genes to provide resistance to development of an exhausted phenotype after repeated or continuous exposure to an antigen, increase persistence, provide fratricide resistance, enhance the function of the immune cell or to reduce immunosuppression or inhibition can occur in a CAR-T cell, i.e., after the immune cell has been transformed to express a chimeric antigen receptor (CAR).

In some embodiments, the immune cell may comprise one or more edited genes, one or more regulatory elements thereof, or combinations thereof, wherein expression of the edited gene is either knocked out or knocked down. In some embodiments, the immune cell may comprise one or more edited genes, one or more regulatory elements thereof, or combinations thereof, wherein expression of the edited gene is increased. In some embodiments, the immune cell may comprise a chimeric antigen receptor (CAR) and one or more edited genes, one or more regulatory elements thereof, or combinations thereof, wherein expression of the edited gene is either knocked out or knocked down. In some embodiments, the immune cell may comprise a chimeric antigen receptor (CAR) and one or more edited genes, one or more regulatory elements thereof, or combinations thereof, wherein expression of the edited gene is increased.

In some embodiments, the CAR-T cells have reduced (e.g., a negative alteration of at least 10%, 25%, 50%, 75%, or 100%) or inactivated surface HLA class-I expression as compared to a similar CAR-T cell lacking one or more edited genes as described herein. In some embodiments, the CAR-T cells have resistance to development of an exhausted phenotype after repeated or continuous exposure to an antigen as compared to a similar CAR-T cell lacking one or more edited genes as described herein. In some embodiments, the CAR-T cells have increased persistence as compared to a similar CAR-T cell lacking one or more edited genes as described herein. In some embodiments, the CAR-T cells have increased fratricide resistance as compared to a similar CAR-T cell lacking one or more edited genes as described herein. In some embodiments, the CAR-T cells have reduced immunogenicity as compared to a similar CAR-T cell lacking one or more edited genes as described herein. In some embodiments, the CAR-T cells have lower activation threshold as compared to a similar CAR-T lacking one or more edited genes as described herein. In some embodiments, the CAR-T cells have increased anti-neoplasia activity as compared to a similar CAR-T cell lacking one or more edited genes as described herein. In some embodiments, the CAR-T cells have increased T- and/or NK-cell resistance as compared to a similar CAR-T cell lacking one or more edited genes as described herein. The one or more genes may be edited by base editing. In some embodiments the one or more genes are directed to components of the peptide loading complex (PLC) or regulatory components thereof. In some embodiments the one or more genes may be selected from a group consisting of: β2M, TAP1, TAP2, Tapasin, and CD58. In some embodiments, the one or more genes may be selected from the group consisting of FKBP1A, NR3C1, and PPIA. In some embodiments, the one or more genes are selected from the group consisting of B2M, CD3e, CD3g, CIITA, PD1, and TRAC. In embodiments, the gene corresponds to an antigen targeted by a CAR expressed by the cell. In some the genes may be edited by base editing and or using a nuclease (e.g., Cas12b). In some cases, the one or more genes are selected from CD58, CD115, CD48, MICA, MICB, Nectin-2, ULBP, β2M, TAP1, TAP2, TAPBP, PDIA3, NLRC5, HLA-A, HLA-B, and/or HLA-C. In some embodiments, one or more additional genes may be edited using a base editor or nuclease. In some embodiments, the one or more additional genes may be selected from TRAC and CIITA. In some embodiments, the one or more additional genes edited may be selected from HLA-E, HLA-G, PD-L1, and CD47. In some embodiments, one or more of β2M, TAP1, TAP2, Tapasin, and/or CD58 are edited in combination with edits in each of HLA-E, HLA-G, PD-L1, and CD47.

In some embodiments, the one or more genes are selected from CD5, CD7, CD19, B2M, CD3γ, CIITA, CD3ε, and PD1. In some embodiments, the CAR-T cells contain modifications in genes encoding one or more of CD5, CD7, CD19, B2M, CD3γ, CIITA, CD38, and PD1. In some embodiments, the CAR-T cells have reduced or undetectable expression of one or more of CD5, CD7, CD19, B2M, CD3γ, CIITA, CD3ε, and PD1 relative to a wild type or unedited T cell.

In some embodiments, an immune cell comprises a chimeric antigen receptor and one or more edited genes, a regulatory element thereof, or combinations thereof. An edited gene may be an immune response regulation gene, an immunogenic gene, a checkpoint inhibitor gene, a gene involved in immune responses, a cell surface marker, e.g., a T cell surface marker, or any combination thereof. In some embodiments, an immune cell comprises a chimeric antigen receptor and an edited gene that is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof. In some embodiments, the edited gene may be a checkpoint inhibitor gene, for example, such as a PD1 gene, a PDC1 gene, or a member related to or regulating the pathway of their formation or activation.

In some embodiments, provided herein is an immune cell with an edited gene in the peptide loading complex (PLC) or a regulatory element thereof, such that the immune cell does not express or expresses at reduced levels surface HLA class-I peptides. In some embodiments, provided herein is an immune cell with an edited gene in the peptide loading complex (PLC) or a regulatory element thereof, such that the immune cell has increased persistence. In some embodiments, the immune cell comprises an edited gene in the peptide loading complex (PLC) or a regulatory element thereof, and additionally, at least one edited gene.

In some embodiments, provided herein is an immune cell (e.g., T- or NK-cell) with an edited β2M gene, such that the immune cell does not express an endogenous functional Beta-2-microglobulin. In some embodiments, provided herein is an immune cell with an edited β2M gene, such that the immune cell does not express or expresses at reduced levels surface HLA class-I peptides. In some embodiments, provided herein is an immune cell with an edited β2M gene, such that the immune cell has increased persistence. In some embodiments, the immune cell comprises an edited β2M gene, and additionally, at least one edited gene.

In some embodiments, provided herein is an immune cell (e.g., T- or NK-cell) with an edited TAP1 gene, such that the immune cell does not express an endogenous functional TAP1. In some embodiments, provided herein is an immune cell with an edited TAP1 gene, such that the immune cell does not express or expresses at reduced levels surface HLA class-I peptides. In some embodiments, provided herein is an immune cell with an edited TAP1 gene, such that the immune cell has increased persistence. In some embodiments, the immune cell comprises an edited TAP1 gene, and additionally, at least one edited gene.

In some embodiments, provided herein is an immune cell (e.g., T- or NK-cell) with an edited TAP2 gene, such that the immune cell does not express an endogenous functional TAP2. In some embodiments, provided herein is an immune cell with an edited TAP2 gene, such that the immune cell does not express or expresses at reduced levels surface HLA class-I peptides. In some embodiments, provided herein is an immune cell with an edited TAP2 gene, such that the immune cell has increased persistence. In some embodiments, the immune cell comprises an edited TAP2 gene, and additionally, at least one edited gene.

In some embodiments, provided herein is an immune cell (e.g., T- or NK-cell) with edited TAP1 and TAP2 genes, such that the immune cell does not express endogenous functional TAP1 and TAP2. In some embodiments, provided herein is an immune cell with edited TAP1 and TAP 2 genes, such that the immune cell does not express or expresses at reduced levels surface HLA class-I peptides. In some embodiments, provided herein is an immune cell with an edited TAP1 and TAP2 gene, such that the immune cell has increased persistence. In some embodiments, the immune cell comprises an edited TAP1 and TAP2 gene, and additionally, at least one edited gene.

In some embodiments, provided herein is an immune cell (e.g., T- or NK-cell) with an edited Tapasin gene, such that the immune cell does not express an endogenous functional Tapasin. In some embodiments, provided herein is an immune cell with an edited Tapasin gene, such that the immune cell does not express or expresses at reduced levels surface HLA class-I peptides. In some embodiments, provided herein is an immune cell with an edited Tapasin gene, such that the immune cell has increased persistence. In some embodiments, the immune cell comprises an edited Tapasin gene, and additionally, at least one edited gene.

In some embodiments, provided herein is an immune cell (e.g., T- or NK-cell) with an edited CD58 gene, such that the immune cell does not express an endogenous functional CD58. In some embodiments, provided herein is an immune cell with an edited CD58 gene, such that the immune cell has increased persistence. In some embodiments, the immune cell comprises an edited CD58 gene, and additionally, at least one edited gene.

In some embodiments, provided herein is an immune cell (e.g., T- or NK-cell) with an edited CD54 gene, such that the immune cell does not express an endogenous functional CD54. In some embodiments, provided herein is an immune cell with an edited CD54 gene, such that the immune cell has increased persistence. In some embodiments, the immune cell comprises an edited CD54 gene, and additionally, at least one edited gene.

In some embodiments, each edited gene may comprise a single base edit. In some embodiments, each edited gene may comprise multiple base edits at different regions of the gene. In some embodiments, a single modification event (such as electroporation), may introduce one or more gene edits. In some embodiments at least one, two, three, four, five, six, seven, eight, nine, ten, eleven, twelve, thirteen, fourteen, fifteen, sixteen, seventeen, eighteen, nineteen, twenty or more edits may be introduced in one or more genes simultaneously. In some embodiments, an immune cell, including but not limited to any immune cell comprising an edited gene selected from any of the aforementioned gene edits, can be edited to generate mutations in other genes that enhance the CAR-T's function or reduce immunosuppression or inhibition of the cell.

### Chimeric Antigen Receptors and Car-T Cells

The disclosure provides immune cells modified using nucleobase editors described herein and that express chimeric antigen receptors (CARs). Modification of immune cells to express a chimeric antigen receptor can enhance an immune cell's immunoreactive activity, wherein the chimeric antigen receptor has an affinity for an epitope on an antigen, wherein the antigen is associated with an altered fitness of an organism. For example, the chimeric antigen receptor can have an affinity for an epitope on a protein expressed in a neoplastic cell. Because the CAR-T cells can act independently of major histocompatibility complex (MHC), activated CAR-T cells can kill the neoplastic cell expressing the antigen. The direct action of the CAR-T cell evades neoplastic cell defensive mechanisms that have evolved in response to MHC presentation of antigens to immune cells. Exemplary chimeric antigen receptors, modified immune cells, and methods for preparing the same are described in PCT Applications No. PCT/US2020/013964, PCT/US2020/052822, PCT/US2020/018178, PCT/US2021/52035, and PCT/US2022/075021, or in Hardke-Wolenski, et al., Biomedicines 10:1493 (2022), the disclosures of which are incorporated herein by reference in their entirety for all purposes.

However, target antigens associated with neoplastic cells may also be expressed on healthy immune cells. Accordingly, activated CAR-T cells not only kill neoplastic cells expressing the target antigen but also healthy immune cells that also express the target antigen. To prevent this fratricide or self-killing of immune cells, the disclosure provides a CAR-T that has been modified using nucleobase editors to reduce or eliminate the expression of a target antigen (e.g., CD19) to provide fratricide resistance. In some embodiments, the disclosure provides a fratricide resistant modified immune effector cell that expresses a chimeric antigen receptor to target a neoplastic cell.

Some embodiments comprise autologous immune cell immunotherapy, wherein immune cells are obtained from a subject having a disease or altered fitness characterized by cancerous or otherwise altered cells expressing a surface marker. The obtained immune cells are genetically modified to express a chimeric antigen receptor and are effectively redirected against specific antigens. Thus, in some embodiments, immune cells are obtained from a subject in need of CAR-T immunotherapy. In some embodiments, these autologous immune cells are cultured and modified shortly after they are obtained from the subject. In other embodiments, the autologous cells are obtained and then stored for future use. This practice may be advisable for individuals who may be undergoing parallel treatment that will diminish immune cell counts in the future. In allogeneic immune cell immunotherapy, immune cells can be obtained from a donor other than the subject who will be receiving treatment. In some embodiments, immune cells are obtained from a healthy subject or donor and are genetically modified to express a chimeric antigen receptor and are effectively redirected against specific antigens. The immune cells, after modification to express a chimeric antigen receptor, are administered to a subject for treating a disease, such as a neoplasia (e.g., B-, T-, or NK-cell malignancy). In some embodiments, immune cells to be modified to express a chimeric antigen receptor can be obtained from pre-existing stock cultures of immune cells.

Immune cells and/or immune effector cells can be isolated or purified from a sample collected from a subject or a donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD4⁺ is used as a marker to select T cells. In one embodiment, CD8⁺ is used as a marker to select T cells.

In another embodiment, the disclosure provides T cells that have targeted gene knockouts at the TCR constant region (TRAC), which is responsible for TCRαβ surface expression. TCRαβ-deficient CAR-T cells are compatible with allogeneic immunotherapy (Qasim et al., Sci. Transl. Med. 9, eaaj2013 (2017); Valton et al., Mol Ther. 2015 September; 23(9): 1507-1518). If desired, residual TCRαβ T cells are removed using CliniMACS magnetic bead depletion to minimize the risk of GVHD. In another embodiment, the disclosure provides donor T cells selected ex vivo to recognize minor histocompatibility antigens expressed on recipient hematopoietic cells, thereby minimizing the risk of graft-versus-host disease (GVHD), which is the main cause of morbidity and mortality after transplantation (Warren et al., Blood 2010; 115(19):3869-3878). Another technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4, CD8, CD28, CD45) and corresponding gating strategy. In one embodiment, a CD4 gating strategy is employed. In one embodiment, a CD8 gating strategy is employed. In one embodiment, a CD4 and CD8 gating strategy is employed. In some embodiments, a gating strategy for other markers specific to an immune effector cell is employed instead of, or in combination with, the CD4 and/or CD8 gating strategy.

The immune effector cells contemplated in the disclosure include effector T cells. In some embodiments, the effector T cell is a naïve CD8⁺ T cell, a cytotoxic T cell, a natural killer T (NKT) cell, or a natural killer (NK) cell. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, immune effector cells are effector NK cells. In some embodiments, the immune effector cell is any other subset of T cells. The modified immune effector cell may express, in addition to the chimeric antigen receptor, an exogenous cytokine, a different chimeric receptor, or any other agent that would enhance immune effector cell signaling or function. For example, co-expression of the chimeric antigen receptor and a cytokine may enhance the CAR-T cell's ability to lyse a target cell.

Chimeric antigen receptors as contemplated in the present disclosure comprise an extracellular binding domain, a transmembrane domain, and an intracellular domain. Binding of an antigen to the extracellular binding domain can activate the CAR-T cell and generate an effector response, which includes CAR-T cell proliferation, cytokine production, and other processes that lead to the death, inactivation, and/or neutralization of the antigen expressing cell. In some embodiments of the present disclosure, the chimeric antigen receptor further comprises a linker. In some embodiments, the linker is a (GGGGS)ₙ linker (SEQ ID NO: 172). In some embodiments, the linker is a (GGGGS)₃ linker (SEQ ID NO: 486). In some embodiments, a CAR of the present disclosure includes a leader peptide sequence (e.g., N-terminal to the antigen binding domain). An exemplary leader peptide amino acid sequence is:

(SEQ ID NO: 487
METDTLLLWVLLLWVPGSTG.

In various embodiments, the CAR-T specifically targets a cluster of differentiation 19 (CD19) polypeptide. In some embodiments, the CAR-T specifically targets CD5, CD7, CD19, CD20, CD22, CD79B, or ROR1.

Provided herein are also nucleic acids that encode the chimeric antigen receptors described herein. In some embodiments, the nucleic acid is isolated or purified. Delivery of the nucleic acids ex vivo can be accomplished using methods known in the art. For example, immune cells obtained from a subject may be transformed with a nucleic acid vector encoding the chimeric antigen receptor. The vector may then be used to transform recipient immune cells so that these cells will then express the chimeric antigen receptor. Efficient means of transforming immune cells include transfection and transduction. Such methods are well known in the art. For example, applicable methods for delivery the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

Some aspects of the present disclosure provide for immune cells comprising a chimeric antigen and an altered endogenous gene that provides a reduced tendency relative to unedited CAR-T cells to develop an exhausted phenotype after being stimulated by multiple antigen exposures or continuous exposure to an antigen, resistance to fratricide, enhances immune cell function, resistance to immunosuppression or inhibition, or a combination thereof. In some embodiments, the altered endogenous gene may be created by base editing. In some embodiments, the base editing may reduce or attenuate the gene expression. In some embodiments, the base editing may reduce or attenuate the gene activation. In some embodiments, the base editing may reduce or attenuate the functionality of the gene product. In some other embodiments, the base editing may activate or enhance the gene expression. In some embodiments, the base editing may increase the functionality of the gene product. In some embodiments, the altered endogenous gene may be modified or edited in an exon, an intron, an exon-intron injunction, or a regulatory element thereof. The modification may be edit to a single nucleobase in a gene or a regulatory element thereof. The modification may be in a exon, more than one exons, a start codon, a splice acceptor site, a splice donor site, an intron, or more than one introns, or a combination thereof. The modification may be in an open reading frame of a gene. The modification may be in an untranslated region of the gene, for example, a 3′-UTR or a 5′5′-UTR. In some embodiments, the modification is in a regulatory element of an endogenous gene. In some embodiments, the modification is in a promoter, an enhancer, an operator, a silencer, an insulator, a terminator, a transcription initiation sequence, a translation initiation sequence (e.g., a Kozak sequence), or any combination thereof.

Allogeneic immune cells expressing an endogenous immune cell receptor as well as a chimeric antigen receptor may recognize and attack host cells, a circumstance termed graft versus host disease (GVHD). The alpha component of the immune cell receptor complex is encoded by the TRAC gene, and in some embodiments, this gene is edited such that the alpha subunit of the TCR complex is nonfunctional or absent. Because this subunit is necessary for endogenous immune cell signaling, editing this gene can reduce the risk of graft versus host disease caused by allogeneic immune cells.

In some embodiments, editing of genes to provide a reduced tendency relative to unedited CAR-T cells to develop an exhausted phenotype after being stimulated by multiple antigen exposures or continuous exposure to an antigen, fratricide resistance, enhance the function of the immune cell or to reduce immunosuppression or inhibition can occur in the immune cell before the cell is transformed to express a chimeric antigen receptor. In other aspects, editing of genes to provide a reduced tendency relative to unedited CAR-T cells to develop an exhausted phenotype after being stimulated by multiple antigen exposures or continuous exposure to an antigen, fratricide resistance, enhance the function of the immune cell or to reduce immunosuppression or inhibition can occur in a CAR-T cell, i.e., after the immune cell has been transformed to express a chimeric antigen receptor.

In some embodiments, the immune cell may comprise a chimeric antigen receptor (CAR) and one or more edited genes (e.g., those genes listed herein), one or more regulatory elements thereof, or combinations thereof, wherein expression of the edited gene is either knocked out or knocked down. In some embodiments, the CAR-T cells have a reduced tendency to develop an exhausted phenotype after being stimulated by multiple antigen exposures or continuous exposure to an antigen as compared to a similar reference CAR-T cell not having the one or more edited genes as described herein. In some embodiments, the CAR-T cells have increased fratricide resistance as compared to a similar reference CAR-T cell not having the one or more edited genes as described herein. In some embodiments, the CAR-T cells have reduced immunogenicity as compared to a similar CAR-T cell but without further having the one or more edited genes as described herein. In some embodiments, the CAR-T cells have lower activation threshold as compared to a similar reference CAR-T not having the one or more edited genes as described herein. In some embodiments, the CAR-T cells have increased anti-neoplasia activity as compared to a similar reference CAR-T cell not having the one or more edited genes as described herein. The one or more genes may be edited by base editing.

In some embodiments, an immune cell comprises a chimeric antigen receptor and one or more edited genes, a regulatory element thereof, or combinations thereof. An edited gene may be an immune response regulation gene, an immunogenic gene, a checkpoint inhibitor gene, a gene involved in immune responses, a cell surface marker, e.g., a T cell surface marker, or any combination thereof. In some embodiments, an immune cell comprises a chimeric antigen receptor and an edited gene that is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof. In some embodiments, the edited gene may be a checkpoint inhibitor gene, such as a PD-1 gene, or a member related to or regulating the pathway of their formation or activation.

In some embodiments, provided herein is an immune cell with an edited gene (e.g., CD5, CD7, CD19, CD3e, CD3g, B2M, and/or CIITa), such that the immune cell does not express an endogenous functional polypeptide encoded by the gene. In some embodiments, provided herein is a CAR-T cell with an edited gene, such that the CAR-T cell exhibits reduced or negligible expression or no expression of endogenous polypeptide encoded by the gene. In embodiments, the gene encodes CD5, CD7, CD19, CD3e, CD3g, B2M, and/or CIITa.

In some embodiments, each edited gene may comprise a single base edit. In some embodiments, each edited gene may comprise multiple base edits at different regions of the gene. In some embodiments, a single modification event (such as electroporation), may introduce one or more gene edits. In some embodiments at least one, two, three, four, five, six, seven, eight, nine, ten, eleven, twelve, thirteen, fourteen, fifteen, sixteen, seventeen, eighteen, nineteen, twenty or more edits may be introduced in one or more genes simultaneously. In some embodiments, an immune cell, including but not limited to any immune cell comprising an edited gene selected from any of the aforementioned gene edits, can be edited to generate mutations in other genes that enhance the CAR-T's function or reduce immunosuppression or inhibition of the cell.

### Extracellular Binding Domain

The chimeric antigen receptors of the disclosure include an extracellular binding domain. The extracellular binding domain of a chimeric antigen receptor contemplated herein comprises an amino acid sequence of an antibody, or an antigen binding fragment thereof, that has an affinity for a specific antigen. In some embodiments, the antigen is a cluster of differentiation 19 (CD19) polypeptide, or a fragment thereof.

In some embodiments the chimeric antigen receptor comprises an amino acid sequence of an antibody. In some embodiments, the chimeric antigen receptor comprises the amino acid sequence of an antigen binding fragment of an antibody. The antibody (or fragment thereof) portion of the extracellular binding domain recognizes and binds to an epitope of an antigen. In some embodiments, the antibody fragment portion of a chimeric antigen receptor is a single chain variable fragment (scFv). An scFv comprises the light and variable fragments of a monoclonal antibody. In other embodiments, the antibody fragment portion of a chimeric antigen receptor is a multichain variable fragment, which comprises more than one extracellular binding domains and therefore bind to more than one antigen simultaneously. In a multiple chain variable fragment embodiment, a hinge region may separate the different variable fragments, providing necessary spatial arrangement and flexibility.

In some embodiments, the extracellular binding domain is an anti-CD19 scFv. In some cases, the extracellular binding domain is an anti-CD5, anti-CD7, anti-CD19, anti-CD20, anti-CD22, anti-CD79B, or anti-ROR1 scFv.

In other embodiments, the antibody portion of a chimeric antigen receptor comprises at least one heavy chain and at least one light chain. In some embodiments, the antibody portion of a chimeric antigen receptor comprises two heavy chains, joined by disulfide bridges and two light chains, wherein the light chains are each joined to one of the heavy chains by disulfide bridges. In some embodiments, the light chain comprises a constant region and a variable region. Complementarity determining regions residing in the variable region of an antibody are responsible for the antibody's affinity for a particular antigen. Thus, antibodies that recognize different antigens comprise different complementarity determining regions. Complementarity determining regions reside in the variable domains of the extracellular binding domain, and variable domains (i.e., the variable heavy and variable light) can be linked with a linker or, in some embodiments, with disulfide bridges. In some embodiments, the variable heavy chain and variable light chain are linked by a (GGGGS)ₙ linker (SEQ ID NO: 172), wherein the n is an integer from 1 to 10. In some embodiments, the linker is a (GGGGS)₃ linker (SEQ ID NO: 486).

In some embodiments, the antigen recognized and bound by the extracellular domain is a protein or peptide, a nucleic acid, a lipid, or a polysaccharide. Antigens can be heterologous, such as those expressed in a pathogenic bacteria or virus. Antigens can also be synthetic; for example, some individuals have extreme allergies to synthetic latex and exposure to this antigen can result in an extreme immune reaction. In some embodiments, the antigen is autologous, and is expressed on a diseased or otherwise altered cell.

For example, in some embodiments, the antigen is expressed in a neoplastic cell. In some embodiments, the neoplastic cell is a malignant T-, B-, or NK-cell. In some embodiments, the malignant T-, B-, or NK-cell is a malignant precursor T-, B-, or NK-cell. In some embodiments, the malignant T-, B-, or NK-cell is a malignant mature T-, B-, or NK-cell. Nonlimiting examples of neoplasia include B cell lymphoma, mantle cell lymphoma, T-cell acute lymphoblastic leukemia (T-ALL), mycosis fungoides (MF), Sézary syndrome (SS), Peripheral T/NK-cell lymphoma, Anaplastic large cell lymphoma ALK+, Primary cutaneous T-cell lymphoma, T-cell large granular lymphocytic leukemia, Angioimmunoblastic T/NK-cell lymphoma, Hepatosplenic T-cell lymphoma, Primary cutaneous CD30+lymphoproliferative disorders, Extranodal NK/T-cell lymphoma, Adult T-cell leukemia/lymphoma, T-cell prolymphocytic leukemia, Subcutaneous panniculitis-like T-cell lymphoma, Primary cutaneous gamma-delta T-cell lymphoma, Aggressive NK-cell leukemia, and Enteropathy-associated T-cell lymphoma.

Antibody-antigen interactions are noncovalent interactions resulting from hydrogen bonding, electrostatic or hydrophobic interactions, or from van der Waals forces. The affinity of extracellular binding domain of the chimeric antigen receptor for an antigen can be calculated with the following formula:

$${K}_{A} = [ Antibody - Antigen ] / [ Antibody ] [ Antigen ] , wherein$$

$$[ Ab ] = molar concentration of unoccupied binding sites on the antibody ;$$

$$[ Ag ] = molar concentration of unoccupied binding sites on the antigen ; and$$

$$[ Ab - Ag ] = molar concentration of the antibody - antigen complex .$$

The antibody-antigen interaction can also be characterized based on the dissociation of the antigen from the antibody. The dissociation constant (K_{D}) is the ratio of the association rate to the dissociation rate and is inversely proportional to the affinity constant. Thus, K_{D}=1/K_{A}. Those skilled in the art will be familiar with these concepts and will know that traditional methods, such as ELISA assays, can be used to calculate these constants.

### Transmembrane Domain

The chimeric antigen receptors of the disclosure include a transmembrane domain. The transmembrane domain of the chimeric antigen receptors described herein spans the CAR-T cell's lipid bilayer cellular membrane and separates the extracellular binding domain and the intracellular signaling domain. In some embodiments, this domain is derived from other receptors having a transmembrane domain, while in other embodiments, this domain is synthetic. In some embodiments, the transmembrane domain may be derived from a non-human transmembrane domain and, in some embodiments, humanized. By “humanized” is meant having the sequence of the nucleic acid encoding the transmembrane domain optimized such that it is more reliably or efficiently expressed in a human subject. In some embodiments, the transmembrane domain is derived from another transmembrane protein expressed in a human immune effector cell. Examples of such proteins include, but are not limited to, subunits of the T cell receptor (TCR) complex, PD1, or any of the Cluster of Differentiation proteins, or other proteins, that are expressed in the immune effector cell and that have a transmembrane domain. In some embodiments, the transmembrane domain will be synthetic, and such sequences will comprise many hydrophobic residues.

Transmembrane domains for use in the disclosed CARs can include at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, the transmembrane domain is derived from CD4, CD8α, CD28 and CD3ζ.

The chimeric antigen receptor is designed, in some embodiments, to comprise a spacer between the transmembrane domain and the extracellular domain, the intracellular domain, or both. Such spacers can be 1, 2, 3, 4, 5, 6, 7, 8, 9, 10, 11, 12, 13, 14, 15, 16, 17, 18, 19, or 20 amino acids in length. In some embodiments, the spacer can be 20, 30, 40, 50, 60, 70, 80, 90, or 100 amino acids in length. In still other embodiments the spacer can be between 100 and 500 amino acids in length. The spacer can be any polypeptide that links one domain to another and are used to position such linked domains to enhance or optimize chimeric antigen receptor function.

### Intracellular Signaling Domain

The chimeric antigen receptors of the disclosure include an intracellular signaling domain. The intracellular signaling domain is the intracellular portion of a protein expressed in a T cell that transduces a T cell effector function signal (e.g., an activation signal) and directs the T cell to perform a specialized function. T cell activation can be induced by a number of factors, including binding of cognate antigen to the T cell receptor on the surface of T cells and binding of cognate ligand to costimulatory molecules on the surface of the T cell. A T cell co-stimulatory molecule is a cognate binding partner on a T cell that specifically binds with a co-stimulatory ligand, thereby mediating a co-stimulatory response by the T cell, such as, but not limited to, proliferation. Co-stimulatory molecules include but are not limited to an MHC class I molecule. Activation of a T cell leads to immune response, Such as T cell proliferation and differentiation (see, e.g., Smith-Garvin et al., Annu. Rev. Immunol., 27:591-619, 2009). Exemplary T cell signaling domains are known in the art. Non-limiting examples include the CD35, CD8, CD28, CD27, CD154, GITR (TNFRSF18), CD134 (OX40), and CD137 (4-1BB) signaling domains.

The intracellular signaling domain of the chimeric antigen receptor contemplated herein comprises a primary signaling domain. In some embodiments, the chimeric antigen receptor comprises the primary signaling domain and a secondary, or co-stimulatory, signaling domain.

In some embodiments, the primary signaling domain comprises one or more immunoreceptor tyrosine-based activation motifs, or ITAMs. In some embodiments, the primary signaling domain comprises more than one ITAM. ITAMs incorporated into the chimeric antigen receptor may be derived from ITAMs from other cellular receptors. In some embodiments, the primary signaling domain comprising an ITAM may be derived from subunits of the TCR complex, such as CD3γ, CD3ε, CD3ζ, or CD3δ. In some embodiments, the primary signaling domain comprising an ITAM may be derived from FcRγ, FcRβ, CD5, CD22, CD79a, CD79b, or CD66d.

In some embodiments, the primary signaling domain is selected from the group consisting of CD8, CD28, CD134 (OX40), CD137 (4-1BB), and CD3ζ.

In some embodiments, the secondary, or co-stimulatory, signaling domain is derived from CD2, CD4, CD5, CD8α, CD28, CD83, CD134, CD137 (4-1BB), ICOS, or CD154, or a combination thereof. In some embodiments, the co-signaling domain is a cytoplasmic domain.

In some embodiments, the CAR comprises one or more signaling domains. In some embodiments, the CAR comprises a combination of signaling domains.

### Editing of Target Genes in Immune Cells

In some embodiments, provided herein is an immune cell with at least one modification in an endogenous gene or regulatory elements thereof. In some embodiments, the immune cell may comprise a further modification in at least one, two, three, four, five, six, seven, eight, nine, ten, eleven, twelve, thirteen, fourteen, fifteen, sixteen, seventeen, eighteen, nineteen, twenty or more endogenous genes or regulatory elements thereof. In some embodiments, the at least one modification is a single nucleobase modification. In some embodiments, the at least one modification is by base editing. The base editing may be positioned at any suitable position of the gene, or in a regulatory element of the gene. Thus, it may be appreciated that a single base editing at a start codon, for example, can completely abolish the expression of the gene. In some embodiments, the base editing may be performed at a site within an exon. In some embodiments, the base editing may be performed at a site on more than one exons. In some embodiments, the base editing may be performed at a start codon. In some embodiments, the base editing may be performed at a splice acceptor site. In some embodiments, the base editing may be performed at a splice donor site. In some embodiments, the base editing may be performed at any exon of the multiple exons in a gene. In some embodiments, base editing may introduce a premature STOP codon into an exon, resulting in either lack of a translated product or in a truncated that may be misfolded and thereby eliminated by degradation, or may produce an unstable mRNA that is readily degraded. In some embodiments, the immune cell is a T cell. In some embodiments, the immune cell is a CAR-T cell. In some embodiments, the immune cell is a NK cell.

In some embodiments, the immune cell is modified using prime editing. Methods for editing polynucleotide sequences using prime editing are well known in the art (see, e.g., Petrova I O, Smirnikhina S A. The Development, Optimization and Future of Prime Editing. Int J Mol Sci. 2023 Dec. 1; 24(23):17045. doi: 10.3390/ijms242317045, the disclosure of which is incorporated herein in its entirety by reference for all purposes). In some embodiments, the cell is modified using a CRISPR/Cas system. In some cases, expression of a gene in the cell may be disrupted through introduction of an insertion/deletion (indel) mutation to the gene using, e.g., a nuclease, such as a Cas12b or Cas9 protein, or through insertion of a heterologous polynucleotide sequence into the gene, such as through the use of a transposon or a CRISPR/Cas system.

In some embodiments, an edited gene may be an immune response regulation gene, an immunogenic gene, a checkpoint inhibitor gene, a gene involved in immune responses, a cell surface marker, e.g., a T cell surface marker, or any combination thereof. In some embodiments, the edited gene is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof. In some embodiments, the edited gene may be a checkpoint inhibitor gene.

In some embodiments, the gene is selected from those genes listed herein. Further non-limiting examples of genes that may be edited include those listed in any one of PCT Applications No. PCT/US2020/013964, PCT/US2020/052822, PCT/US2020/018178, PCT/US2021/52035, and PCT/US2022/075021, the disclosures of which are incorporated herein by reference in their entirety for all purposes.

In some embodiments, the editing of the endogenous gene reduces expression of the gene. In some embodiments, the editing of the endogenous gene reduces expression of the gene by at least 50% as compared to a control cell without the modification. In some embodiments, the editing of the endogenous gene reduces expression of the gene by at least 60% as compared to a control cell without the modification. In some embodiments, the editing of the endogenous gene reduces expression of the gene by at least 70% as compared to a control cell without the modification. In some embodiments, the editing of the endogenous gene reduces expression of the gene by at least 80% as compared to a control cell without the modification. In some embodiments, the editing of the endogenous gene reduces expression of the gene by at least 90% as compared to a control cell without the modification. In some embodiments, the editing of the endogenous gene reduces expression of the gene by at least 100% as compared to a control cell without the modification. In some embodiments, the editing of the endogenous gene eliminates gene expression.

In some embodiments, base editing may be performed on an intron. For example, base editing may be performed on an intron. In some embodiments, the base editing may be performed at a site within an intron. In some embodiments, the base editing may be performed at a site one or more introns. In some embodiments, the base editing may be performed at any exon of the multiple introns in a gene. In some embodiments, one or more base editing may be performed on an exon, an intron or any combination of exons and introns.

In some embodiments, the modification or base edit may be within a promoter site. In some embodiments, the base edit may be introduced within an alternative promoter site. In some embodiments, the base edit may be in a 5′ regulatory element, such as an enhancer. In some embodiment, base editing may be introduced to disrupt the binding site of a nucleic acid binding protein. Exemplary nucleic acid binding proteins may be a polymerase, nuclease, gyrase, topoisomerase, methylase or methyl transferase, transcription factors, enhancer, PABP, zinc finger proteins, among many others.

In some embodiments, base editing may be used for splice disruption to silence target protein expression. In some embodiments, base editing may generate a splice acceptor-splice donor (SA-SD) site. Targeted base editing generating a SA-SD, or at a SA-SD site can result in reduced expression of a gene. In some embodiments, base editors (e.g., ABE, CBE, CABE) are used to target dinucleotide motifs that constitute splice acceptor and splice donor sites, which are the first and last two nucleotides of each intron. In some embodiments, splice disruption is achieved with an adenosine base editor (ABE). In some embodiments, splice disruption is achieved with a cytidine base editor (CBE). In some embodiments, base editors (e.g., CBE, CABE) are used to edit exons by creating STOP codons.

In some embodiments, provided herein is an immune cell with at least one modification in one or more endogenous genes. In some embodiments, the immune cell may have at least one modification in one, two, three, four, five, six, seven, eight, nine, ten, eleven, twelve, thirteen, fourteen, fifteen, sixteen, seventeen, eighteen, nineteen, twenty or more endogenous genes. In some embodiments, the modification generates a premature stop codon in the endogenous genes. In some embodiments, the STOP codon silences target protein expression. In some embodiments, the modification is a single base modification. In some embodiments, the modification is generated by base editing. The premature stop codon may be generated in an exon, an intron, or an untranslated region. In some embodiments, base editing may be used to introduce more than one STOP codon, in one or more alternative reading frames. In some embodiments, the stop codon is generated by a adenosine base editor (ABE). In some embodiments, the stop codon is generated by a cytidine base editor (CBE). In some embodiments, the CBE generates any one of the following edits (shown in underlined font) to generate a STOP codon: CAG→TAG; CAA→TAA; CGA→TGA; TGG→TGA; TGG→TAG; or TGG→TAA.

In some embodiments, modification/base edits may be introduced at a 3′-UTR, for example, in a poly adenylation (poly-A) site. In some embodiments, base editing may be performed on a 5′-UTR region.

### Molecular Switches

In various embodiments, an immune cell (e.g., a CAR-T cell) of the disclosure expresses a molecular switch alternatively referred to as a “kill switch,” “suicide switch,” or “safety switch.” A kill switch is activated by a pharmaceutical agent (e.g., an antibody). When a kill switch is activated, the kill switch mediates killing of the cell expressing the kill switch. For example, in an embodiment, a kill switch expressed on the surface of a cell mediates the induction of complement-mediated killing of the cell in the presence of a monoclonal antibody (e.g., Rituximab). In some cases, a kill switch binds Rituximab.

Non-limiting examples of molecular switches include RQR1, RQR2, RQR3, RQR4, RQR8, RQR1G4S, RQR2G4S, RR, G4SRR, G4SRRG4S, G4SRRG4SCD8, G4SRRG4SCD28, G4SRRCD28, and QG4S, the amino acid sequences of which are listed in Table C, where each of “R” (e.g., CPYSNPSLC (SEQ ID NO: 488) or PAKPTTTACPYSNPSLC (SEQ ID NO: 489)), “R1” (e.g., PAKPTTTACPYSNPSLC (SEQ ID NO: 489)), “R2” (e.g., PAKPTTTACPYSNPSLC (SEQ ID NO: 489) or PAKPTTTCPYSNPSLC (SEQ ID NO: 490)), “R3,” “R4,” and “R8” represents a Rituximab-binding epitope, “Q” is a QBEnd10-binding epitope (e.g., ELPTQGTFSNVSTNVS), “G4S” represents a linker (e.g., GGGGS (SEQ ID NO: 172) or SGGGGS (SEQ ID NO: 491)), “CD8” represents a CD8 stalk domain (e.g., PAPRPPTPAPTIASQPLSLRPEACRPAAGGAVHTRGLDFACD (SEQ ID NO: 492)), and “CD28” represents a CD28 stalk domain (IEVMYPPPYLDNEKSNGTIIHVKGKHLCPSPLFPGPSKP (SEQ ID NO: 492)). In embodiments, the Rituximab-binding epitope is derived from CD20 and the QBEnd10-binding epitope is derived from CD34. Further non-limiting examples of molecular switches (e.g., “kill switches”), components thereof (e.g., Rituximab-binding or QBEnd10-binding epitopes), polypeptides (e.g., chimeric antigen receptors) comprising the molecular switches, and methods for use thereof suitable for use in embodiments of the disclosure include those polypeptides described in Patent Application Publications No. WO 2013/153391 or US 2018/0002435, and/or in Moghanloo, et al., Translational Oncology 14:101070 (2021), the disclosures of each of which are incorporated herein by reference in their entireties by reference for all purposes. In some cases, the kill switch is fused at the C-terminus or N-terminus thereof to a transmembrane domain (e.g., a CD8a transmembrane domain).

In various embodiments, the methods of the disclosure involve using a QBEnd10-binding epitope or other epitope derived from CD34 as a marker for use in cell sorting. A non-limiting example of a commercially available system for such cell sorting is the Miltenyi CD34 cliniMACS system. Cells binding QBEnd10 may be sorted by any method known in the art, such as a fluorescence activated cell sorting (FACS) based method.

Kill switches are genetically encoded elements integrated into CAR-T cells that allow the elimination of the introduced T cells in case of unexpected toxicities. The kill switch may be anchored to the surface of a cell. For example, in some cases, the kill switch is anchored to the surface of a cell by being fused to a transmembrane domain or by being fused to a membrane-bound protein, such as a chimeric antigen receptor (CAR). Kill switches are activated by being contacted with a pharmaceutical agent. These genes include inducible caspase 9 (iC9), truncated EGFR (tEGFR or EGFRt), herpes simplex virus thymidine kinase (HSV-TK), and CD20.

iCasp9

iCasp9 (inducible caspase 9) is a pro-apoptotic kill switch made by the fusion of a mutant FKBP12, a receptor for the immunosuppressant drug FK506, to a modified human caspase 9 using a flexible SGGGS-linker (SEQ ID NO: 750). The mutant FKBP12 moiety allows a small molecular chemical inducer of dimerization (CID) (AP1903/AP20187) to attach to it while it cannot bind to the wild-type FKBP12. The modified caspase 9 is a truncated protein without the physiological dimerization domain or caspase recruitment domain (CARD) to minimize basal signaling. Conditional intravenous administration of a CID (such as AP1903) produces crosslinking of the drug-binding domains of this chimeric protein that results in the dimerization of caspase 9, and thereby activates the downstream executioner caspase3 molecules, leading to apoptosis of the cells expressing the fusion protein. In-vitro and in-vivo experiments show that this safety switch can cause apoptosis of approximately 99% of donor T cells using a 10 nM dose of AP1903.

### HSV-TK

The Thymidine kinase (TK) derived from Herpes simplex viruses-1 (HSV-1) (HSV-TK), which has been probably evolved distinctly from TK1, the cell-cycle dependent cytosolic TK, can phosphorylate thymidine, various other pyrimidines, and also pyrimidine and purine analogs. Tri-phosphorylated nucleoside analogs are cytotoxic because they interfere with DNA synthesis. Several pro-drugs for the HSV-TK system have been evaluated, including ganciclovir (GCV), acyclovir (ACV), and brivudin (BVDU) and among them, GCV was found to be the most effective pro-drug for this system. By conditional administration of GCV, HSV-TK catalyzes the phosphorylation of GCV that produces a toxic GCV-triphosphate resulting in competitive inhibition of guanosine incorporation with subsequent inhibition of DNA synthesis and cellular death.

### CD20 and EGFRt Kill Switches

Another kill switch involves expression of a targetable component, such as a well-known surface antigen such as CD20 or the truncated epidermal growth factor receptor (EGFRt) in a cell. This approach allows a selective cell removal through the complement-dependent cytotoxicity (CDC) and antibody-dependent cell-mediated cytotoxicity (ADCC) after administration of a specific monoclonal antibody. Rituximab has been used as a clinically approved monoclonal antibody for CD20 and cetuximab for EGFRt. If an antibody has poor bio-distribution, poor tissue penetration, and/or has limited CDC/ADCC (complement dependent cytotoxicity/antibody-dependent cell-mediated cytotoxicity) capacity in a patient, these issues may be addressed by creating anti-idiotype CARs recognizing CD19-specific CARs or synthesizing a short peptide epitope (E-tag) in the extracellular domain of the CAR and using an anti-E-tag CAR in order to omit the anti-tumor CARs.

### Human Leukocyte Antigen (HLA) Single-Chain Trimers and Dimers (HLA^{SC} Molecules)

In various embodiments, an immune cell (e.g., a CAR-T cell) of the disclosure expresses a human leukocyte antigen (HLA) (e.g., HLA-E or HLA-G) single-chain trimer (SCT) and/or single-chain dimer (SCD). The single-chain trimer or single-chain dimer can be secreted or membrane-bound. In embodiments, a single-chain HLA dimer contains a cognate peptide (cPep) and an HLA domain (e.g., HLA-E or HLA-G). In embodiments, a single-chain HLA trimer contains a B2M domain, an HLA domain (e.g., HLA-E or HLA-G), and a cognate peptide. In some cases, the HLA single-chain trimer or dimer contains a transmembrane domain. In other cases, the HLA single-chain trimer or dimer does not contain any transmembrane domain.

HLA-E and HLA-G single-chain dimers and single-chain dimers can improve the ability of immune cells to evade NK cells. The SCTs and SCDs bind to the NKG2A inhibitory receptor of natural killer (NK) cells, thereby inhibiting the NK cells and preventing lysis of the immune cells thereby. In embodiments, the SCTs and SCDs allow immune cells expressing the same to resist allogeneic rejection mechanisms of a host subject.

Non-limiting examples of HLA single-chain trimers include polypeptides with the following elements, from N-terminus to C-terminus:
a) a cPep, at least a fragment of an HLA-G polypeptide, and at least a fragment of a β2M polypeptide;
b) at least a fragment of a β2M polypeptide, a cPep, and at least a fragment of an HLA-G polypeptide;
c) a cPep, at least a fragment of a β2M polypeptide, and at least a fragment of an HLA-G polypeptide; or
d) fragment of an HLA-G polypeptide, a cPep, and at least at least a fragment of a β2M polypeptide.

Non-limiting examples of HLA single-chain trimers and single-chain dimers include polypeptides with about or at least about 85%, 90%, 95%, 96%, 97%, 98%, 99%, or 100% sequence identity to those single-chain HLA trimers and dimers listed in Table A below:

TABLE E
Secreted HLA-E and HLA-G constructs. In Table A, the HLA domain is indicated
by underlined text, linkers are indicated by bold text, signal peptides (e.g., the signal
peptide of HLA-G, IL-2, or β2M) are indicated by italicized text, cognate peptides are
indicated by bold underlined text, an HLA-G5 intron tail is indicated by double-underlined
text, and the β2M domain is indicated by bold, italic, underlined text.
Construct: BTx_CM118

| | |
|---|---|
| HLA-G5 | MVVMAPRTLFLLLSGALTLTETWAGSHSMRYFSAAVSRPGRGEPRFIAM |
| isoform | GYVDDTQFVRFDSDSACPRMEPRAPWVEQEGPEYWEEETRNTKAHAQTD |
| | RMNLQTLRGYYNQSEASSHTLQWMIGCDLGSDGRLLRGYEQYAYDGKDY |
| | LALNEDLRSWTAADTAAQISKRKCEAANVAEQRRAYLEGTCVEWLHRYL |
| | ENGKEMLQRADPPKTHVTHHPVFDYEATLRCWALGFYPAEIILTWQRDG |
| | EDQTQDVELVETRPAGDGTFQKWAAVVVPSGEEQRYTCHVQHEGLPEPL |
| | MLRWSKEGDGGIMSVRESRSLSEDL (SEQ ID NO: 493) |

Construct: BTx_CM119

| | |
|---|---|
| HLA-G5 | MYRMQLLSCIALSLALVTNSGSHSMRYFSAAVSRPGRGEPRFIAMGYVD |
| + | DTQFVRFDSDSACPRMEPRAPWVEQEGPEYWEEETRNTKAHAQTDRMNL |
| IL-2 signal peptide | QTLRGYYNQSEASSHTLQWMIGCDLGSDGRLLRGYEQYAYDGKDYLALN |
| | EDLRSWTAADTAAQISKRKCEAANVAEQRRAYLEGTCVEWLHRYLENGK |
| | EMLQRADPPKTHVTHHPVFDYEATLRCWALGFYPAEIILTWQRDGEDQT |
| | QDVELVETRPAGDGTFQKWAAVVVPSGEEQRYTCHVQHEGLPEPLMLRW |
| | SKEGDGGIMSVRESRSLSEDL (SEQ ID NO: 494) |

Construct: BTx_CM120

| | |
|---|---|
| HLA-G5 | MYRMQLLSCIALSLALVTNSIQRTPKIQVYSRHPAENGKSNFLNCYVSG |
| Single chain trimer | FHPSDIEVDLLKNGERIEKVEHSDLSFSKDWSFYLLYYTEFTPTEKDEY |
| + | ACRVNHVTLSQPKIVKWDRDMGGGGSGGGGSGGGGSRIIPRHLQLGGGG |
| IL-2 signal peptide | SGGGGSGGGGSGGGGSGSHSMRYFSAAVSRPGRGEPRFIAMGYVDDTQF |
| | VRFDSDSACPRMEPRAPWVEQEGPEYWEEETRNTKAHAQTDRMNLQTLR |
| | GYYNQSEASSHTLQWMIGCDLGSDGRLLRGYEQYAYDGKDYLALNEDLR |
| | SWTAADTAAQISKRKCEAANVAEQRRAYLEGTCVEWLHRYLENGKEMLQ |
| | RADPPKTHVTHHPVFDYEATLRCWALGFYPAEIILTWQRDGEDQTQDVE |
| | LVETRPAGDGTFQKWAAVVVPSGEEQRYTCHVQHEGLPEPLMLRWSKEG |
| | DGGIMSVRESRSLSEDL (SEQ ID NO: 495) |

Construct: BTx_CM193

| | |
|---|---|
| HLA-E(ΔTM) | MSRSVALAVLALLSLSGLEAVMAPRTLFLGGGGSGGGGSGGGGSIQRTP |
| Single chain trimer | KIQVYSRHPAENGKSNFLNCYVSGFHPSDIEVDLLINGERIEKVEHSDL |
| + | SFSKDWSFYLLYYTEFTPTEKDEYACRVNHVTLSQPKIVKWDRDMGGGG |
| HLA-G5 intron tail | SGGGGSGGGGSGGGGSGSHSLKYFHTSVSRPGRGEPRFISVGYVDDTQF |
| | VRFDNDAASPRMVPRAPWMEQEGSEYWDRETRSARDTAQIFRVNLRTLR |
| | GYYNQSEAGSHTLQWMHGCELGPDGRFLRGYEQFAYDGKDYLTLNEDLR |
| | SWTAVDTAAQISEQKSNDASEAEHQRAYLEDTCVEWLHKYLEKGKETLL |
| | HLEPPKTHVTHHPISDHEATLRCWALGFYPAEITLTWQQDGEGHTQDTE |
| | LVETRPAGDGTFQKWAAVVVPSGEEQRYTCHVQHEGLPEPVTLRWSKEG |
| | DGGIMSVRESRSLSEDL (SEQ ID NO: 496) |

Construct: BTx_CM211

| | |
|---|---|
| HLA-E(ΔTM) | MSRSVALAVLALLSLSGLEAVMAPRTLFLGGSGGGASGGGSHSLKYFHT |
| ß2M (C-term) | SVSRPGRGEPRFISVGYVDDTQFVRFDNDAASPRMVPRAPWMEQEGSEY |
| Single chain trimer | WDRETRSARDTAQIFRVNLRTLRGYYNQSEAGSHTLQWMHGCELGPDGR |
| | FLRGYEQFAYDGKDYLTLNEDLRSWTAVDTAAQISEQKSNDASEAEHQR |
| | AYLEDTCVEWLHKYLEKGKETLLHLEPPKTHVTHHPISDHEATLRCWAL |
| | GFYPAEITLTWQQDGEGHTQDTELVETRPAGDGTFQKWAAVVVPSGEEQ |
| | RYTCHVQHEGLPEP+ VTLRWGGGGSGGGGSGGGGSIQRTPKIQVYSRHPA |
| | ENGKSNFLNCYVSGFHPSDIEVDLLKNGERIEKVEHSDLSFSKDWSFYL |
| | LYYTEFTPTEKDEYACRVNHVTLSQPKIVKWDRDM (SEQ ID NO: 497) |

Construct: BTx_CM212

| | |
|---|---|
| HLA-E(ΔTM) | MSRSVALAVLALLSLSGLEAVMAPRTLFLGGSGGGASGGGSHSLKYFHT |
| Single chain dimer | SVSRPGRGEPRFISVGYVDDTQFVRFDNDAASPRMVPRAPWMEQEGSEY |
| + | WDRETRSARDTAQIFRVNLRTLRGYYNQSEAGSHTLQWMHGCELGPDGR |
| HLA-G5 intron tail | FLRGYEQFAYDGKDYLTLNEDLRSWTAVDTAAQISEQKSNDASEAEHQR |
| | AYLEDTCVEWLHKYLEKGKETLLHLEPPKTHVTHHPISDHEATLRCWAL |
| | GFYPAEITLTWQQDGEGHTQDTELVETRPAGDGTFQKWAAVVVPSGEEQ |
| | RYTCHVQHEGLPEPVTLRWSKEGDGGIMSVRESRSLSEDL |
| | (SEQ ID NO: 498) |

Construct: BTx_CM213

| | |
|---|---|
| HLA-E(ΔTM) | MSRSVALAVLALLSLSGLEAVMAPRTLFLGGSGGGASGGGSHSLKYFHT |
| Single chain dimer | SVSRPGRGEPRFISVGYVDDTQFVRFDNDAASPRMVPRAPWMEQEGSEY |
| | WDRETRSARDTAQIFRVNLRTLRGYYNQSEAGSHTLQWMHGCELGPDGR |
| | FLRGYEQFAYDGKDYLTLNEDLRSWTAVDTAAQISEQKSNDASEAEHQR |
| | AYLEDTCVEWLHKYLEKGKETLLHLEPPKTHVTHHPISDHEATLRCWAL |
| | GFYPAEITLTWQQDGEGHTQDTELVETRPAGDGTFQKWAAVVVPSGEEQ |
| | RYTCHVQHEGLPEPVTLRW (SEQ ID NO: 499) |

A non-limiting examples of a membrane-bound HLA single-chain trimers include polypeptides containing an amino acid sequence with about or at least about 85%, 90%, 95%, 96%, 97%, 98%, 99%, or 100% sequence identity to the below amino acid sequence:

(SEQ ID NO: 500; BTx_CM188)
MSRSVALAVLALLSLSGLEAVMAPRTLFLGGGGSGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSLKYFHTSVSRPGRGEPRFISVGYVDDTQF
VRFDNDAASPRMVPRAPWMEQEGSEYWDRETRSARDTAQIFRVNLRTLR
GYYNQSEAGSHTLQWMHGCELGPDGRFLRGYEQFAYDGKDYLTLNEDLR
SWTAVDTAAQISEQKSNDASEAEHQRAYLEDTCVEWLHKYLEKGKETLL
HLEPPKTHVTHHPISDHEATLRCWALGFYPAEITLTWQQDGEGHTQDTE
LVETRPAGDGTFQKWAAVVVPSGEEQRYTCHVQHEGLPEPVTLRWKPAS
QPTIPIMALIVLGGVAGLLLFIGLGIFFCVRC.

In the above sequence, which corresponds to an HLA-E SCT (CD4TM) polypeptide, the HLA-E heavy chain domain is indicated by underlined text, linkers are indicated by bold text, the signal peptide is indicated by italicized text, the cognate peptide (alternatively, “peptide for presentation”) is indicated by bold underlined text, the β2M domain is indicated by bold, italic, underlined text, the CD4 transmembrane domain is indicated by plain text, and the CD4 truncated intracellular domain is indicated by double-underlined text.

### Editing of Target Genes

To produce the gene edits described herein, cells are contacted with one or more guide RNAs and a nucleobase editor polypeptide comprising a nucleic acid programmable DNA binding protein (napDNAbp) and a cytidine deaminase or adenosine deaminase or comprising one or more deaminases with cytidine deaminase and/or adenosine deaminase activity (e.g., a “dual deaminase” which has cytidine and adenosine deaminase activity). In some embodiments, cells to be edited are contacted with at least one nucleic acid, where the at least one nucleic acid encodes one or more guide RNAs and a nucleobase editor polypeptide containing a nucleic acid programmable DNA binding protein (napDNAbp) and a deaminase. In some embodiments, the gRNA comprises nucleotide analogs. In some instances, the gRNA is added directly to a cell. These nucleotide analogs can inhibit degradation of the gRNA from cellular processes. Tables 1, 2A, and 2B provide representative sequences to be used for gRNAs. In some embodiments, the gene edits described herein are introduced to a polynucleotide using prime editing. Methods for editing polynucleotide sequences using prime editing are well known in the art (see, e.g., Petrova I O, Smirnikhina S A. The Development, Optimization and Future of Prime Editing. Int J Mol Sci. 2023 Dec. 1; 24(23):17045. doi: 10.3390/ijms242317045, the disclosure of which is incorporated herein in its entirety by reference for all purposes). In some embodiments, the gene edits described herein are introduced to a polynucleotide using a CRISPR/Cas system. In some cases, expression of a gene may be disrupted through introduction of an insertion/deletion (indel) mutation to the gene using, e.g., a nuclease, such as a Cas 12b or Cas9 protein, or through insertion of a heterologous polynucleotide sequence into the gene, such as through the use of a transposon or a CRISPR/Cas system.

Variants of the spacer sequences provided herein comprising 1, 2, 3, 4, or 5 nucleobase alterations are contemplated. For example, variation of a target polynucleotide sequence within a population (e.g., single nucleotide polymorphisms) may require said alterations to a spacer sequence to allow the spacer to better bind a variant of a target sequence in a subject.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

In embodiments, a guide RNA of the disclosure contains a scaffold. Non-limiting examples of scaffold nucleotide sequences include the following:

GUUUUAGAGCUAGAAAUAGCAAGUUAAAAUAAGGCUAGUCCGUUAUCAA
CUUGAAAAAGUGGCACCGAGUCGGUGCUUUU
(SEQ ID NO: 371; SpCas9 scaffold sequence);
GUUCUGUCUUUUGGUCAGGACAACCGUCUAGCUAUAAGUGCUGCAGGGU
GUGAGAAACUCCUAUUGCUGGACGAUGUCUCUUACGAGGCAUUAGCAC
(SEQ ID NO: 778; Cas12b scaffold sequence).

Exemplary guide RNA sequences are provided in Tables 1, 2A, and 2B below.

TABLE 1
Exemplary guide polynucleotide sequences.

| Guide | Alternative | | SEQ |
|---|---|---|---|
| Name | Name | Guide Polynucleotide Sequence | ID NO |
| TSBTx | Guide_210 | CUCACCGUCUCCUGGGGAGAGUUUUAGAGCUAGAAAUAGCAAG | 501 |
| 1538 | | UUAAAAUAAGGCUAGUCCGUUAUCAACUUGAAAAAGUGGCACC | |
| | | GAGUCGGUGCUUUU | |
| TSBTx | Guide_214 | ACCGGUGUAGUGCACCACGCGUUUUAGAGCUAGAAAUAGCAAG | 502 |
| 1542 | | UUAAAAUAAGGCUAGUCCGUUAUCAACUUGAAAAAGUGGCACC | |
| | | GAGUCGGUGCUUUU | |
| TSBTx | Guide_217 | UACCCAAGAACAGGGAGCUAGUUUUAGAGCUAGAAAUAGCAAG | 503 |
| 1545 | | UUAAAAUAAGGCUAGUCCGUUAUCAACUUGAAAAAGUGGCACC | |
| | | GAGUCGGUGCUUUU | |
| TSBTx | Guide_218 | CCCCAACAGAUCUGCCAUGGGUUUUAGAGCUAGAAAUAGCAAG | 504 |
| 1546 | | UUAAAAUAAGGCUAGUCCGUUAUCAACUUGAAAAAGUGGCACC | |
| | | GAGUCGGUGCUUUU | |
| TSBTx | Guide_253 | UUCACAGGGAUGCUUGAAGAGUUUUAGAGCUAGAAAUAGCAAG | 505 |
| 1581 | | UUAAAAUAAGGCUAGUCCGUUAUCAACUUGAAAAAGUGGCACC | |
| | | GAGUCGGUGCUUUU | |
| TSBTx | Guide_254 | CGCCGCCAUGGGAGUGCAGGGUUUUAGAGCUAGAAAUAGCAAG | 506 |
| 1582 | | UUAAAAUAAGGCUAGUCCGUUAUCAACUUGAAAAAGUGGCACC | |
| | | GAGUCGGUGCUUUU | |
| TSBTx | Guide_209 | CAGGUGGAAACCAUCUCCCCGUUUUAGAGCUAGAAAUAGCAAG | 507 |
| 1537 | | UUAAAAUAAGGCUAGUCCGUUAUCAACUUGAAAAAGUGGCACC | |
| | | GAGUCGGUGCUUUU | |
| TSBTx | Guide_211 | GCGCCCUGAGGAGACAGAGAGUUUUAGAGCUAGAAAUAGCAAG | 508 |
| 1539 | | UUAAAAUAAGGCUAGUCCGUUAUCAACUUGAAAAAGUGGCACC | |
| | | GAGUCGGUGCUUUU | |
| TSBTx | Guide_212 | CGCCCUGAGGAGACAGAGACGUUUUAGAGCUAGAAAUAGCAAG | 509 |
| 1540 | | UUAAAAUAAGGCUAGUCCGUUAUCAACUUGAAAAAGUGGCACC | |
| | | GAGUCGGUGCUUUU | |
| TSBTx | Guide_213 | CAAGCGCGGCCAGACCUGCGGUUUUAGAGCUAGAAAUAGCAAG | 510 |
| 1541 | | UUAAAAUAAGGCUAGUCCGUUAUCAACUUGAAAAAGUGGCACC | |
| | | GAGUCGGUGCUUUU | |
| TSBTx | Guide_215 | ACUCAUCUGUGAAAAGAACAGUUUUAGAGCUAGAAAUAGCAAG | 511 |
| 1543 | | UUAAAAUAAGGCUAGUCCGUUAUCAACUUGAAAAAGUGGCACC | |
| | | GAGUCGGUGCUUUU | |
| TSBTx | Guide_216 | CGAUGUGGAGCUUCUAAAACGUUUUAGAGCUAGAAAUAGCAAG | 512 |
| 1544 | | UUAAAAUAAGGCUAGUCCGUUAUCAACUUGAAAAAGUGGCACC | |
| | | GAGUCGGUGCUUUU | |
| TSBTx | Guide_249 | AGCAGGAGGUGAUCCGAGGCGUUUUAGAGCUAGAAAUAGCAAG | 513 |
| 1577 | | UUAAAAUAAGGCUAGUCCGUUAUCAACUUGAAAAAGUGGCACC | |
| | | GAGUCGGUGCUUUU | |
| TSBTx | Guide_250 | GCAGGAGGUGAUCCGAGGCUGUUUUAGAGCUAGAAAUAGCAAG | 514 |
| 1578 | | UUAAAAUAAGGCUAGUCCGUUAUCAACUUGAAAAAGUGGCACC | |
| | | GAGUCGGUGCUUUU | |
| TSBTx | Guide_251 | GUGAUCCGAGGCUGGGAAGAGUUUUAGAGCUAGAAAUAGCAAG | 515 |
| 1579 | | UUAAAAUAAGGCUAGUCCGUUAUCAACUUGAAAAAGUGGCACC | |
| | | GAGUCGGUGCUUUU | |
| TSBTx | Guide_252 | GAUCCGAGGCUGGGAAGAAGGUUUUAGAGCUAGAAAUAGCAAG | 516 |
| 1580 | | UUAAAAUAAGGCUAGUCCGUUAUCAACUUGAAAAAGUGGCACC | |
| | | GAGUCGGUGCUUUU | |
| TSBTx | Guide_236 | AGGUUUCAGGAACUUACACCGUUUUAGAGCUAGAAAUAGCAAG | 517 |
| 1564 | | UUAAAAUAAGGCUAGUCCGUUAUCAACUUGAAAAAGUGGCACC | |
| | | GAGUCGGUGCUUUU | |
| TSBTx | Guide_238 | UACUCAUUAAUAAUCAGAUCGUUUUAGAGCUAGAAAUAGCAAG | 518 |
| 1566 | | UUAAAAUAAGGCUAGUCCGUUAUCAACUUGAAAAAGUGGCACC | |
| | | GAGUCGGUGCUUUU | |
| TSBTx | Guide_240 | ACCUGAAGAGAGAAGCAGUAGUUUUAGAGCUAGAAAUAGCAAG | 519 |
| 1568 | | UUAAAAUAAGGCUAGUCCGUUAUCAACUUGAAAAAGUGGCACC | |
| | | GAGUCGGUGCUUUU | |
| TSBTx | Guide_241 | AACCUUUUAGUUCCUAAGGAGUUUUAGAGCUAGAAAUAGCAAG | 520 |
| 1569 | | UUAAAAUAAGGCUAGUCCGUUAUCAACUUGAAAAAGUGGCACC | |
| | | GAGUCGGUGCUUUU | |
| TSBTx | Guide_247 | UUCCUUUAUAGAAUGUUAUGGUUUUAGAGCUAGAAAUAGCAAG | 521 |
| 1575 | | UUAAAAUAAGGCUAGUCCGUUAUCAACUUGAAAAAGUGGCACC | |
| | | GAGUCGGUGCUUUU | |
| TSBTx | Guide_219 | CUCCAAGCAGCGAAGACUUUGUUUUAGAGCUAGAAAUAGCAAG | 522 |
| 1547 | | UUAAAAUAAGGCUAGUCCGUUAUCAACUUGAAAAAGUGGCACC | |
| | | GAGUCGGUGCUUUU | |
| TSBTx | Guide_220 | GGGCCAAAUCAGCCUUUCCUGUUUUAGAGCUAGAAAUAGCAAG | 523 |
| 1548 | | UUAAAAUAAGGCUAGUCCGUUAUCAACUUGAAAAAGUGGCACC | |
| | | GAGUCGGUGCUUUU | |
| TSBTx | Guide_221 | GGCCAAAUCAGCCUUUCCUCGUUUUAGAGCUAGAAAUAGCAAG | 524 |
| 1549 | | UUAAAAUAAGGCUAGUCCGUUAUCAACUUGAAAAAGUGGCACC | |
| | | GAGUCGGUGCUUUU | |
| TSBTx | Guide_222 | GCCAAAUCAGCCUUUCCUCGGUUUUAGAGCUAGAAAUAGCAAG | 525 |
| 1550 | | UUAAAAUAAGGCUAGUCCGUUAUCAACUUGAAAAAGUGGCACC | |
| | | GAGUCGGUGCUUUU | |
| TSBTx | Guide_223 | CCAAAUCAGCCUUUCCUCGGGUUUUAGAGCUAGAAAUAGCAAG | 526 |
| 1551 | | UUAAAAUAAGGCUAGUCCGUUAUCAACUUGAAAAAGUGGCACC | |
| | | GAGUCGGUGCUUUU | |
| TSBTx | Guide_224 | CAACAUUUGAAGGGCCAGACGUUUUAGAGCUAGAAAUAGCAAG | 527 |
| 1552 | | UUAAAAUAAGGCUAGUCCGUUAUCAACUUGAAAAAGUGGCACC | |
| | | GAGUCGGUGCUUUU | |
| TSBTx | Guide_225 | GGCCAGACUGGCACCAACGGGUUUUAGAGCUAGAAAUAGCAAG | 528 |
| 1553 | | UUAAAAUAAGGCUAGUCCGUUAUCAACUUGAAAAAGUGGCACC | |
| | | GAGUCGGUGCUUUU | |
| TSBTx | Guide_226 | CAGGAUUUGGAGUUUUCUUCGUUUUAGAGCUAGAAAUAGCAAG | 529 |
| 1554 | | UUAAAAUAAGGCUAGUCCGUUAUCAACUUGAAAAAGUGGCACC | |
| | | GAGUCGGUGCUUUU | |
| TSBTx | Guide_227 | UACUGUCAGGCAAGCUUUCCGUUUUAGAGCUAGAAAUAGCAAG | 530 |
| 1555 | | UUAAAAUAAGGCUAGUCCGUUAUCAACUUGAAAAAGUGGCACC | |
| | | GAGUCGGUGCUUUU | |
| TSBTx | Guide_228 | GCUGUCCUAUAUGGAAUAAAGUUUUAGAGCUAGAAAUAGCAAG | 531 |
| 1556 | | UUAAAAUAAGGCUAGUCCGUUAUCAACUUGAAAAAGUGGCACC | |
| | | GAGUCGGUGCUUUU | |
| TSBTx | Guide_229 | CAGCACAAUUACCUAUGUGCGUUUUAGAGCUAGAAAUAGCAAG | 532 |
| 1557 | | UUAAAAUAAGGCUAGUCCGUUAUCAACUUGAAAAAGUGGCACC | |
| | | GAGUCGGUGCUUUU | |
| TSBTx | Guide_230 | CCGCUAUCGAAAAUGUCUUCGUUUUAGAGCUAGAAAUAGCAAG | 533 |
| 1558 | | UUAAAAUAAGGCUAGUCCGUUAUCAACUUGAAAAAGUGGCACC | |
| | | GAGUCGGUGCUUUU | |
| TSBTx | Guide_231 | UCUUCAGGCUGGAAUGAACCGUUUUAGAGCUAGAAAUAGCAAG | 534 |
| 1559 | | UUAAAAUAAGGCUAGUCCGUUAUCAACUUGAAAAAGUGGCACC | |
| | | GAGUCGGUGCUUUU | |
| TSBTx | Guide_232 | GGAAUUCAGCAGGCCACUACGUUUUAGAGCUAGAAAUAGCAAG | 535 |
| 1560 | | UUAAAAUAAGGCUAGUCCGUUAUCAACUUGAAAAAGUGGCACC | |
| | | GAGUCGGUGCUUUU | |
| TSBTx | Guide_233 | CAAGAAACCUCUGAAAAUCCGUUUUAGAGCUAGAAAUAGCAAG | 536 |
| 1561 | | UUAAAAUAAGGCUAGUCCGUUAUCAACUUGAAAAAGUGGCACC | |
| | | GAGUCGGUGCUUUU | |
| TSBTx | Guide_234 | ACCACAACUCACCCCUACCCGUUUUAGAGCUAGAAAUAGCAAG | 537 |
| 1562 | | UUAAAAUAAGGCUAGUCCGUUAUCAACUUGAAAAAGUGGCACC | |
| | | GAGUCGGUGCUUUU | |
| TSBTx | Guide_235 | ACCUGAAUUAAGAGAAAUAAGUUUUAGAGCUAGAAAUAGCAAG | 538 |
| 1563 | | UUAAAAUAAGGCUAGUCCGUUAUCAACUUGAAAAAGUGGCACC | |
| | | GAGUCGGUGCUUUU | |
| TSBTx | Guide_237 | UGACCCUACUGCAGUACUCCGUUUUAGAGCUAGAAAUAGCAAG | 539 |
| 1565 | | UUAAAAUAAGGCUAGUCCGUUAUCAACUUGAAAAAGUGGCACC | |
| | | GAGUCGGUGCUUUU | |
| TSBTx | Guide_239 | GCCUGUGAAAGAUAAAUAGCGUUUUAGAGCUAGAAAUAGCAAG | 540 |
| 1567 | | UUAAAAUAAGGCUAGUCCGUUAUCAACUUGAAAAAGUGGCACC | |
| | | GAGUCGGUGCUUUU | |
| TSBTx | Guide_242 | ACUCCAGCCAGAACUGGCAGGUUUUAGAGCUAGAAAUAGCAAG | 541 |
| 1570 | | UUAAAAUAAGGCUAGUCCGUUAUCAACUUGAAAAAGUGGCACC | |
| | | GAGUCGGUGCUUUU | |
| TSBTx | Guide_243 | UUAUCAACUGACAAAACUCUGUUUUAGAGCUAGAAAUAGCAAG | 542 |
| 1571 | | UUAAAAUAAGGCUAGUCCGUUAUCAACUUGAAAAAGUGGCACC | |
| | | GAGUCGGUGCUUUU | |
| TSBTx | Guide_244 | ACCACCUGCAAGAGAAGAUAGUUUUAGAGCUAGAAAUAGCAAG | 543 |
| 1572 | | UUAAAAUAAGGCUAGUCCGUUAUCAACUUGAAAAAGUGGCACC | |
| | | GAGUCGGUGCUUUU | |
| TSBTx | Guide_245 | CUAUUGCUUCCAAACAUUUUGUUUUAGAGCUAGAAAUAGCAAG | 544 |
| 1573 | | UUAAAAUAAGGCUAGUCCGUUAUCAACUUGAAAAAGUGGCACC | |
| | | GAGUCGGUGCUUUU | |
| TSBTx | Guide_246 | CAGAUACCAAAAUAUUCAAAGUUUUAGAGCUAGAAAUAGCAAG | 545 |
| 1574 | | UUAAAAUAAGGCUAGUCCGUUAUCAACUUGAAAAAGUGGCACC | |
| | | GAGUCGGUGCUUUU | |
| TSBTx | Guide_248 | UCAGGAGAGGGGAGAUGUGAGUUUUAGAGCUAGAAAUAGCAAG | 546 |
| 1576 | | UUAAAAUAAGGCUAGUCCGUUAUCAACUUGAAAAAGUGGCACC | |
| | | GAGUCGGUGCUUUU | |
| TSBTx | AYgRNA366 | GCCCGACCUCAAAGGAGACGGUUUUAGAGCUAGAAAUAGCAAG | 547 |
| 6143 | | UUAAAAUAAGGCUAGUCCGUUAUCAACUUGAAAAAGUGGCACC | |
| | | GAGUCGGUGCUUUU | |
| TSBTx | AYgRNA367 | CUUACAGCUGUUUGCAGACAGUUUUAGAGCUAGAAAUAGCAAG | 548 |
| 6144 | | UUAAAAUAAGGCUAGUCCGUUAUCAACUUGAAAAAGUGGCACC | |
| | | GAGUCGGUGCUUUU | |
| TSBTx | AYgRNA368 | AUGGACCAACCUGCUGUCUUGUUUUAGAGCUAGAAAUAGCAAG | 549 |
| 6145 | | UUAAAAUAAGGCUAGUCCGUUAUCAACUUGAAAAAGUGGCACC | |
| | | GAGUCGGUGCUUUU | |
| TSBTx | AYgRNA369 | CGUACCUGACACAUAAACCCGUUUUAGAGCUAGAAAUAGCAAG | 550 |
| 6146 | | UUAAAAUAAGGCUAGUCCGUUAUCAACUUGAAAAAGUGGCACC | |
| | | GAGUCGGUGCUUUU | |
| TSBTx | AYgRNA370 | GUACCCUUACCACUCAGUCUGUUUUAGAGCUAGAAAUAGCAAG | 551 |
| 6147 | | UUAAAAUAAGGCUAGUCCGUUAUCAACUUGAAAAAGUGGCACC | |
| | | GAGUCGGUGCUUUU | |
| TSBTx | AYgRNA371 | UCUUCCUUUUCAAGGUUGGAGUUUUAGAGCUAGAAAUAGCAAG | 552 |
| 6148 | | UUAAAAUAAGGCUAGUCCGUUAUCAACUUGAAAAAGUGGCACC | |
| | | GAGUCGGUGCUUUU | |

TABLE 2A
Exemplary spacer sequences. The term “PAM″ means “protospacer adjacent motif.”

| Guide | Alternative | | SEQ | | Target | Compatible |
|---|---|---|---|---|---|---|
| Name | Name | Spacer | ID NO | PAM | Gene | editor(s) |
| TSBTx | Guide_210 | CUCACCGUCUCCUGGGG | 553 | TGG | FKBP1A | ABE (e.g., |
| 1538 | | AGA | | | | ABE8.20m)/ |
| | | | | | | CBE (e.g., |
| | | | | | | rBE4) |
| TSBTx | Guide_214 | ACCGGUGUAGUGCACCA | 554 | AGG | FKBP1A | ABE (e.g., |
| 1542 | | CGC | | | | ABE8.20m)/ |
| | | | | | | CBE (e.g., |
| | | | | | | rBE4) |
| TSBTx | Guide_217 | UACCCAAGAACAGGGAG | 555 | AGG | FKBPIA | ABE (e.g., |
| 1545 | | CUA | | | | ABE8.20m)/ |
| | | | | | | CBE (e.g., |
| | | | | | | rBE4) |
| TSBTx | Guide_218 | CCCCAACAGAUCUGCCA | 556 | AGG | FKBP1A | ABE (e.g., |
| 1546 | | UGG | | | | ABE8.20m) |
| TSBTx | Guide_253 | UUCACAGGGAUGCUUGA | 557 | TGG | FKBP1A | ABE (e.g., |
| 1581 | | AGA | | | | ABE8.20m) |
| TSBTx | Guide_254 | CGCCGCCAUGGGAGUGC | 558 | TGG | FKBP1A | ABE (e.g., |
| 1582 | | AGG | | | | ABE8.20m) |
| TSBTx | Guide_209 | CAGGUGGAAACCAUCUC | 559 | AGG | FKBP1A | CBE (e.g., |
| 1537 | | CCC | | | | rBE4) |
| TSBTx | Guide_211 | GCGCCCUGAGGAGACAG | 560 | CGG | FKBP1A | CBE (e.g., |
| 1539 | | AGA | | | | rBE4) |
| TSBTx | Guide_212 | CGCCCUGAGGAGACAGA | 561 | GGG | FKBP1A | CBE (e.g., |
| 1540 | | GAC | | | | rBE4) |
| TSBTx | Guide_213 | CAAGCGCGGCCAGACCU | 562 | TGG | FKBP1A | CBE (e.g., |
| 1541 | | GCG | | | | rBE4) |
| TSBTx | Guide_215 | ACUCAUCUGUGAAAAGA | 563 | AGG | FKBPIA | CBE (e.g., |
| 1543 | | ACA | | | | rBE4) |
| TSBTx | Guide_216 | CGAUGUGGAGCUUCUAA | 564 | TGG | FKBP1A | CBE (e.g., |
| 1544 | | AAC | | | | rBE4) |
| TSBTx | Guide_249 | AGCAGGAGGUGAUCCGA | 565 | TGG | FKBPIA | CBE (e.g., |
| 1577 | | GGC | | | | rBE4) |
| TSBTx | Guide_250 | GCAGGAGGUGAUCCGAG | 566 | GGG | FKBP1A | CBE (e.g., |
| 1578 | | GCU | | | | rBE4) |
| TSBTx | Guide_251 | GUGAUCCGAGGCUGGGA | 567 | AGG | FKBP1A | CBE (e.g., |
| 1579 | | AGA | | | | rBE4) |
| TSBTx | Guide_252 | GAUCCGAGGCUGGGAAG | 568 | GGG | FKBP1A | CBE (e.g., |
| 1580 | | AAG | | | | rBE4) |
| TSBTx | Guide_236 | AGGUUUCAGGAACUUAC | 569 | TGG | NR3C1 | ABE |
| 1564 | | ACC | | | | |
| TSBTx | Guide_238 | UACUCAUUAAUAAUCAG | 570 | AGG | NR3C1 | ABE/CBE |
| 1566 | | AUC | | | | |
| TSBTx | Guide_240 | ACCUGAAGAGAGAAGCA | 571 | AGG | NR3C1 | ABE/CBE |
| 1568 | | GUA | | | | |
| TSBTx | Guide_241 | AACCUUUUAGUUCCUAA | 572 | CGG | NR3C1 | ABE |
| 1569 | | GGA | | | | |
| TSBTx | Guide_247 | UUCCUUUAUAGAAUGUU | 573 | TGG | NR3C1 | ABE |
| 1575 | | AUG | | | | |
| TSBTx | Guide_219 | CUCCAAGCAGCGAAGAC | 574 | TGG | NR3C1 | CBE |
| 1547 | | UUU | | | | |
| TSBTx | Guide_220 | GGGCCAAAUCAGCCUUU | 575 | CGG | NR3C1 | CBE |
| 1548 | | CCU | | | | |
| TSBTx | Guide_221 | GGCCAAAUCAGCCUUUC | 576 | GGG | NR3C1 | CBE |
| 1549 | | CUC | | | | |
| TSBTx | Guide_222 | GCCAAAUCAGCCUUUCC | 577 | GGG | NR3C1 | CBE |
| 1550 | | UCG | | | | |
| TSBTx | Guide_223 | CCAAAUCAGCCUUUCCU | 578 | GGG | NR3C1 | CBE |
| 1551 | | CGG | | | | |
| TSBTx | Guide_224 | CAACAUUUGAAGGGCCA | 579 | TGG | NR3C1 | CBE |
| 1552 | | GAC | | | | |
| TSBTx | Guide_225 | GGCCAGACUGGCACCAA | 580 | TGG | NR3C1 | CBE |
| 1553 | | CGG | | | | |
| TSBTx | Guide_226 | CAGGAUUUGGAGUUUUC | 581 | TGG | NR3C1 | CBE |
| 1554 | | UUC | | | | |
| TSBTx | Guide_227 | UACUGUCAGGCAAGCUU | 582 | TGG | NR3C1 | CBE |
| 1555 | | UCC | | | | |
| TSBTx | Guide_228 | GCUGUCCUAUAUGGAAU | 583 | AGG | NR3C1 | CBE |
| 1556 | | AAA | | | | |
| TSBTx | Guide_229 | CAGCACAAUUACCUAUG | 584 | TGG | NR3C1 | CBE |
| 1557 | | UGC | | | | |
| TSBTx | Guide_230 | CCGCUAUCGAAAAUGUC | 585 | AGG | NR3C1 | CBE |
| 1558 | | UUC | | | | |
| TSBTx | Guide_231 | UCUUCAGGCUGGAAUGA | 586 | TGG | NR3C1 | CBE |
| 1559 | | ACC | | | | |
| TSBTx | Guide_232 | GGAAUUCAGCAGGCCAC | 587 | AGG | NR3C1 | CBE |
| 1560 | | UAC | | | | |
| TSBTx | Guide_233 | CAAGAAACCUCUGAAAA | 588 | TGG | NR3C1 | CBE |
| 1561 | | UCC | | | | |
| TSBTx | Guide_234 | ACCACAACUCACCCCUA | 589 | TGG | NR3C1 | CBE |
| 1562 | | CCC | | | | |
| TSBTx | Guide_235 | ACCUGAAUUAAGAGAAA | 590 | AGG | NR3C1 | CBE |
| 1563 | | UAA | | | | |
| TSBTx | Guide_237 | UGACCCUACUGCAGUAC | 591 | TGG | NR3C1 | CBE |
| 1565 | | UCC | | | | |
| TSBTx | Guide_239 | GCCUGUGAAAGAUAAAU | 592 | GGC | NR3C1 | CBE |
| 1567 | | AGC | | | | |
| TSBTx | Guide_242 | ACUCCAGCCAGAACUGG | 593 | CGG | NR3C1 | CBE |
| 1570 | | CAG | | | | |
| TSBTx | Guide_243 | UUAUCAACUGACAAAAC | 594 | TGG | NR3C1 | CBE |
| 1571 | | UCU | | | | |
| TSBTx | Guide_244 | ACCACCUGCAAGAGAAG | 595 | TGG | NR3C1 | CBE |
| 1572 | | AUA | | | | |
| TSBTx | Guide_245 | CUAUUGCUUCCAAACAU | 596 | TGG | NR3C1 | CBE |
| 1573 | | UUU | | | | |
| TSBTx | Guide_246 | CAGAUACCAAAAUAUUC | 597 | TGG | NR3C1 | CBE |
| 1574 | | AAA | | | | |
| TSBTx | Guide_248 | UCAGGAGAGGGGAGAUG | 598 | TGG | NR3C1 | CBE |
| 1576 | | UGA | | | | |
| TSBTx | AYgRNA366 | GCCCGACCUCAAAGGAG | 599 | CGG | PPIA | ABE |
| 6143 | | ACG | | | | |
| TSBTx | AYgRNA367 | CUUACAGCUGUUUGCAG | 600 | AGG | PPIA | ABE |
| 6144 | | ACA | | | | |
| TSBTx | AYgRNA368 | AUGGACCAACCUGCUGU | 601 | TGG | PPIA | ABE |
| 6145 | | CUU | | | | |
| TSBTx | AYgRNA369 | CGUACCUGACACAUAAA | 602 | TGG | PPIA | ABE |
| 6146 | | CCC | | | | |
| TSBTx | AYgRNA370 | GUACCCUUACCACUCAG | 603 | TGG | PPIA | ABE |
| 6147 | | UCU | | | | |
| TSBTx | AYgRNA371 | UCUUCCUUUUCAAGGUU | 604 | TGG | PPIA | ABE |
| 6148 | | GGA | | | | |
| IMM_ | — | CCACCUGCACUCCCAUG | 779 | GTTT | FKBP1A | Cas12b |
| 288 | | GCGGC | | | | |
| IMM_ | — | CUUUCCAUCUUCAAGCA | 780 | ATTT | FKBP1A | Cas12b |
| 289 | | UCCCU | | | | |
| IMM_ | — | CUGUCCCGGGAGGAAUC | 781 | GTTT | FKBP1A | Cas12b |
| 290 | | AAAUU | | | | |
| IMM_ | — | GAUUCCUCCCGGGACAG | 782 | ATTT | FKBP1A | Cas12b |
| 291 | | AAACA | | | | |
| IMM_ | — | CUCCCGGGACAGAAACA | 783 | ATTC | FKBPIA | Cas12b |
| 292 | | AGCCC | | | | |
| IMM_ | — | AUGCUAGGCAAGCAGGA | 784 | GTTT | FKBP1A | Cas12b |
| 293 | | GGUGA | | | | |
| IMM_ | — | GGCUCUCUGACCCACAC | 785 | GTTT | FKBPIA | Cas12b |
| 294 | | UCAUC | | | | |
| IMM_ | — | UGCCUAUGGUGCCACUG | 786 | ATTA | FKBP1A | Cas12b |
| 295 | | GGCAC | | | | |
| IMM_ | — | UAGAAGCUCCACAUCGA | 787 | GTTT | FKBP1A | Cas12b |
| 296 | | AGACG | | | | |
| IMM_ | — | CAGUUUUAGAAGCUCCA | 788 | ATTC | FKBP1A | Cas12b |
| 297 | | CAUCG | | | | |
| IMM_ | — | CUGUCAUUCCAGUUUUA | 789 | ATTC | FKBPIA | Cas12b |
| 298 | | GAAGC | | | | |
| IMM_ | — | CCUUACCCAAGAACAGG | 790 | ATTT | FKBP1A | Cas12b |
| 299 | | GAGCU | | | | |
| IMM_ | — | AUGUGCACAUGUCUGGA | 791 | ATTC | FKBP1A | Cas12b |
| 300 | | GGCAC | | | | |
| IMM_ | — | CACCUGCACUCCCAUGG | 792 | TTTC | FKBP1A | Cas12b |
| 301 | | CGGCG | | | | |
| IMM_ | — | CAUCUUCAAGCAUCCCU | 793 | TTTC | FKBP1A | Cas12b |
| 302 | | GUGAA | | | | |
| IMM_ | — | AUUCCUCCCGGGACAGA | 794 | TTTG | FKBP1A | Cas12b |
| 303 | | AACAA | | | | |
| IMM_ | — | UUUCCAUCUUCAAGCAU | 795 | TTTC | FKBP1A | Cas12b |
| 304 | | CCCUG | | | | |
| IMM_ | — | UGUCCCGGGAGGAAUCA | 796 | TTTO | FKBP1A | Cas12b |
| 305 | | AAUUU | | | | |
| IMM_ | — | AGUUUAUGCUAGGCAAG | 797 | TTTA | FKBP1A | Cas12b |
| 306 | | CAGGA | | | | |
| IMM_ | — | UGCUAGGCAAGCAGGAG | 798 | TTTA | FKBP1A | Cas12b |
| 307 | | GUGAU | | | | |
| IMM_ | — | GCUCUCUGACCCACACU | 799 | TTTG | FKBP1A | Cas12b |
| 308 | | CAUCU | | | | |
| IMM_ | — | GAAGCUCCACAUCGAAG | 800 | TTTA | FKBPIA | Cas12b |
| 309 | | ACGAG | | | | |
| IMM_ | — | AGAAGCUCCACAUCGAA | 801 | TTTT | FKBP1A | Cas12b |
| 310 | | GACGA | | | | |
| IMM_ | — | CUUACCCAAGAACAGGG | 802 | TTTC | FKBP1A | Cas12b |
| 311 | | AGCUA | | | | |

TABLE 2B
Further exemplary spacer sequences.

| Polypeptide | | | |
|---|---|---|---|
| endoded by target | | | |
| polynucleotide | | | |
| (guide name in | | | Compatible |
| parenthesis) | Spacer | SEQ ID NO | base editor(s) |
| B2M | AAGUCAACUUCAAUGUCGGA | 605 | CBE, ABE |
| B2M | ACAAAGUCACAUGGUUCACA | 606 | CBE, ABE |
| B2M | ACAGCCCAAGAUAGUUAAGU | 607 | CBE |
| B2M | ACCCAGACACAUAGCAAUUC | 608 | CBE, ABE |
| B2M | ACUCACGCUGGAUAGCCUCC | 609 | CBE(e.g., |
| (TSBTx845) | | | rBE4), ABE |
| B2M | ACUUGUCUUUCAGCAAGGAC | 610 | CBE, ABE |
| B2M | AGUCACAUGGUUCACACGGC | 611 | CBE, ABE |
| B2M | AUACUCAUCUUUUUCAGUGG | 612 | CBE, ABE |
| B2M | CACAGCCCAAGAUAGUUAAG | 613 | CBE |
| B2M | CAGCCCAAGAUAGUUAAGUG | 614 | CBE |
| B2M | CAGUAAGUCAACUUCAAUGU | 615 | CBE, ABE |
| B2M | CAUACUCAUCUUUUUCAGUG | 616 | CBE, ABE |
| B2M | CGCGAGCACAGCUAAGGCCA | 617 | CBE, ABE |
| B2M | CGUGAGUAAACCUGAAUCUU | 618 | CBE |
| B2M | CUCACGCUGGAUAGCCUCC | 619 | CBE, ABE |
| B2M | CUCAGGUACUCCAAAGAUUC | 620 | |
| B2M | CUUACCCCACUUAACUAUCU | 621 | CBE, ABE |
| (TSBTx760) | | | (e.g., |
| | | | ABE8.20m) |
| B2M | GAAGUUGACUUACUGAAGAA | 622 | CBE, ABE |
| B2M | GAGUAGCGCGAGCACAGCUA | 623 | CBE, ABE |
| B2M | GCAUACUCAUCUUUUUCAGU | 624 | CBE, ABE |
| B2M | GCUACUCUCUCUUUCUGGCC | 625 | CBE, ABE |
| B2M | GGCAUACUCAUCUUUUUCAG | 626 | CBE, ABE |
| B2M | UACCCCACUUAACUAUCU | 627 | |
| B2M | UCACGCUGGAUAGCCUCC | 628 | CBE, ABE |
| B2M | UCGAUCUAUGAAAAAGACAG | 629 | ABE |
| B2M | UGGAGAGAGAAUUGAAAAAG | 630 | CBE, ABE |
| B2M | UGGAGUACCUGAGGAAUAUC | 631 | |
| B2M | UGGGCUGUGACAAAGUCACA | 632 | CBE, ABE |
| B2M | UUACCCCACUUAACUAUCUU | 633 | CBE, ABE |
| B2M | UUCAGACUUGUCUUUCAGCA | 634 | CBE, ABE |
| B2M | UUUGACUUUCCAUUCUCUGC | 635 | CBE, ABE |
| CD3e | ACACACUGUGGGGGGUGGGG | 636 | |
| CD3e | ACACAGACACGUGAGUUUAU | 637 | |
| CD3e | ACACUGUGGGGGGUGGGGUG | 638 | |
| CD3e | ACUCACCUGAUAAGAGGCAG | 639 | |
| CD3e | CACACUGUGGGGGGUGGGGU | 640 | |
| CD3e | CCGACUGCAUCUUUGUUUCA | 641 | |
| CD3e | CGUUACCUCAUAGUCUGGGU | 642 | |
| CD3e | CUGGAUUACCUCUUGCCCUC | 643 | ABE8.20m |
| (TSBTx4073) | | | |
| CD3e | GUUACCUCAUAGUCUGGGUU | 644 | |
| CD3e | UACCACCUGAAAAUGAAAAA | 645 | |
| CD3e | UAUAUGCUGGGGAGAAAGAA | 646 | |
| CD3e | UUAUAUGCUGGGGAGAAAGA | 647 | |
| CD3e | UUGUCCUGCGGAGGAAGGAG | 648 | |
| CD3e | UUUGUCCUGCGGAGGAAGGA | 649 | |
| CD3e | UUUUGUCCUGCGGAGGAAGG | 650 | |
| CD3g | ACAUACUUCUGUAAUACACU | 651 | |
| CD3g | ACCUUCAAGGAAACCAGUUG | 652 | |
| CD3g | CAUGGAACAGGGGAAGGGCC | 653 | |
| CD3g | CUCUUCCAUUGGGUACAUAA | 654 | |
| CD3g | CUUCAAGGUAAGGGCCUACU | 655 | |
| CD3g | GACCAGUACAGCCACCUUCA | 656 | |
| CD3g | UCUCCUACCUUUGAUUGACU | 657 | |
| CD3g | UGACCAGCUCUACCAGGUAA | 658 | |
| CD3g | UGACUAUCAAGAAGAUGGUU | 659 | |
| CD3g | UGGCCCAGUCAAUCAAAGGU | 660 | |
| CD3g | UUCUCCUACCUUUGAUUGAC | 661 | |
| CD3g | UUUAAACCAUGUGAUAUUUU | 662 | |
| CD7 | CCCUACCUGUCACCAGGACC | 663 | |
| CD19 | CCAGACCUCACCAUGGCCCC | 751 | |
| (TSBTx3773) | | | |
| CIITA | AAAGGCACUGCAAGAGACAA | 664 | |
| CIITA | ACACUCACUCCAUCACCCGG | 665 | |
| CIITA | ACAUCAAAGUACCCUACAGG | 666 | |
| CIITA | ACCGGCUCUGCAAAGGCCAG | 667 | |
| CIITA | ACCUCCCGAGCAAACAUGAC | 668 | |
| CIITA | ACUGGACCAGUAUGUCUUCC | 669 | |
| CIITA | AGACUCAGAGGUGAGAGGAG | 670 | |
| CIITA | AGCCAAGUACCCCCUCCCAG | 671 | |
| CIITA | AGCCCCAAGGUAAAAAGGCC | 672 | |
| CIITA | AGCCUAGGAGGCAAAGAGCA | 673 | |
| CIITA | AGGCCAUUUUGGAAGCUUGU | 674 | |
| CIITA | AGGCUGCAGGUGGAAUCAGA | 675 | |
| CIITA | CACAUCCUGCAAGGGGGGAU | 676 | |
| CIITA | CACUCACCUUAGCCUGAGCA | 677 | ABE8.20m/ |
| (TSBTx763) | | | rBE4 |
| CIITA | CACUCACUCCAUCACCCGGA | 678 | |
| CIITA | CACUCACUUGAGGGUUUCCA | 679 | |
| CIITA | CAGACAUCAAAGUACCCUAC | 680 | |
| CIITA | CAGACUGCGGGGACACAGUG | 681 | |
| CIITA | CAGCUCACAGUGUGCCACCA | 682 | |
| CIITA | CCACAUCCUGCAAGGGGGGA | 683 | |
| CIITA | CCACUCACCUUAGCCUGAGC | 684 | |
| CIITA | CCCACCCAAUGCCCGGCAGC | 685 | |
| CIITA | CCCCCAGGCUUUCCCCAAAC | 686 | |
| CIITA | CCUCCUGCAAUGCUUCCUGG | 687 | |
| CIITA | CCUGGUCCAGAGCCUGAGCA | 688 | |
| CIITA | CCUUACCUGUCAUGUUUGCU | 689 | |
| CIITA | CGCCCAGGUCCUCACGUCUG | 690 | |
| CIITA | CGUCCAGUACAACAAGUUCA | 691 | |
| CIITA | CUCUGGCAAAUCUCUGAGGC | 692 | |
| CIITA | CUGCCAAAUUCCAGCCUCCU | 693 | |
| CIITA | CUGGUCAGGGCAAGAGCUAU | 694 | |
| CIITA | CUUAGUCCAACACCCACCGC | 695 | |
| CIITA | CUUCCCCCAGCUGAAGUCCU | 696 | |
| CIITA | GAACGGCAGCUGGCCCAAGG | 697 | |
| CIITA | GACACGAGUGAUUGCUGUGC | 698 | |
| CIITA | GACCAGAUUCCCAGUAUGUU | 699 | |
| CIITA | GAGCCAGCCACAGGGCCCCC | 700 | |
| CIITA | GAGCCCCAAGGUAAAAAGGC | 701 | |
| CIITA | GCGUCCACAUCCUGCAAGGG | 702 | |
| CIITA | GGAAGCAGAAGGUGCUUGCG | 703 | |
| CIITA | GGCCCAAGGAGGCCUGGCUG | 704 | |
| CIITA | GGCGGGCCAAGACUUCUCCC | 705 | |
| CIITA | GGCGUCCACAUCCUGCAAGG | 706 | |
| CIITA | GGCUGCAGCCGGGGACACUG | 707 | |
| CIITA | GGGCCCACAGCCACUCGUGG | 708 | |
| CIITA | GGGCGUCCACAUCCUGCAAG | 709 | |
| CIITA | GUACAAGCUGUCGGAAACAG | 710 | |
| CIITA | UAACAUACUGGGAAUCUGGU | 711 | |
| CIITA | UAUGACCAGAUGGACCUGGC | 712 | |
| CIITA | UCACUCCAGAUGCUGCAGGG | 713 | |
| CIITA | UGCUCUGGAGAUGGAGAAGC | 714 | |
| CIITA | UGGGCGUCCACAUCCUGCAA | 715 | |
| CIITA | UGGUGCAGGCCAGGCUGGAG | 716 | |
| CIITA | UGUCUUCCAGGACUCCCAGC | 717 | |
| CIITA | UUCAACCAGGAGCCAGCCUC | 718 | |
| CIITA | UUCCAGAAGAAGCUGCUCCG | 719 | |
| CIITA | UUUUACCUUGGGGCUCUGAC | 720 | |
| PD1 | ACGACUGGCCAGGGCGCCUG | 721 | |
| PD1 | CACCUACCUAAGAACCAUCC | 722 | |
| PD1 | GGACCCAGACUAGCAGCACC | 723 | |
| PD1 | GGAGUCUGAGAGAUGGAGAG | 724 | |
| PD1 | GGGGUUCCAGGGCCUGUCUG | 725 | |
| PD1 | CCUCCUUCUUUGAGGAGAAA | 726 | |
| PD1 | CCAGUGGCGAGAGAAGACCC | 727 | |
| PD1 | UGCCCAGCCACUGAGGCCUG | 728 | |
| PD1 | CGACUGGCCAGGGCGCCUGU | 729 | |
| PD1 | ACCGCCCAGACGACUGGCCA | 730 | |
| PD1 | CACCGCCCAGACGACUGGCC | 731 | |
| PD1 | UUCUCUCUGGAAGGGCACAA | 732 | |
| PD1 | CUACAACUGGGCUGGCGGCC | 733 | |
| PD1 | GACGUUACCUCGUGCGGCCC | 734 | |
| PD1 | CAGCAACCAGACGGACAAGC | 735 | |
| PD1 | CCUGCAGAGAAACACACUUG | 736 | |
| PD1 | CAGUUCCAAACCCUGGUGGU | 737 | |
| PD1 | GUGUCACACAACUGCCCAAC | 738 | |
| PD1 | AGCCGGCCAGUUCCAAACCC | 739 | |
| PD1 | CGGCCAGUUCCAAACCCUGG | 740 | |
| PD1 | UCCCUGCAGAGAAACACACU | 741 | |
| PD1 | GAGACUCACCAGGGGCUGGC | 742 | |
| TRAC | CCAGCCAAGUACGUAAGUAG | 743 | |
| TRAC | CUGGAUAUCUGUGGGACAAG | 744 | |
| TRAC | CUUACCUGGGCUGGGGAAGA | 745 | |
| TRAC | GCUACAAACAAGCUCAUCUU | 746 | |
| TRAC | UUCAAAACCUGUCAGUGAUU | 747 | |
| TRAC | UUCGUAUCUGUAAAACCAAG | 748 | rBE4 |
| (TSBTx754) | | | |
| TRAC | UUUCAAAACCUGUCAGUGAU | 749 | |

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain may comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2cl (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that may comprise an amino acid change such as a deletion, insertion, substitution, variant, mutation, fusion, chimera, or any combination thereof. In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 may comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152 (5): 1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein may comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.
N is A, C, T, or G; and V is A, C, or G.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-LRKIQK | NGTN |
| spCas9-LRVSQK | NGTN |
| spCas9-LRVSQL | NGTN |
| Cpf1 | 5′ (TTTV) |
| SpyMac | 5′-NAA-3′ |

In some embodiments, the PAM is NGC. In some embodiments, the NGC PAM is recognized by a Cas9 variant. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218).

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556 (7699), 57-63; Miller et al., “Continuous evolution of SpCas9 variants compatible with non-G PAMs” Nat. Biotechnol., 2020 April; 38 (4): 471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein may comprise a deaminase flanked by an N-terminal fragment and a C-terminal fragment of a Cas9 or Cas12 (e.g., Cas12b/C2c1), polypeptide

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes may comprise more than one deaminase. The fusion protein or complex may comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein may comprise a linker between the deaminase and the napDNAbp polypeptide. The linker can be a peptide or a non-peptide linker. For example, the linker can be an XTEN, (GGGS)n (SEQ ID NO: 246), SGGSSGGS (SEQ ID NO: 330), (GGGGS)n (SEQ ID NO: 247), (G)n, (EAAAK)n (SEQ ID NO: 248), (GGS)n, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2c1, or a functional fragment thereof capable of associating with a nucleic acid (e.g., a gRNA) that guides the Cas 12 to a specific nucleic acid sequence. The Cas12 polypeptide can be a variant Cas12 polypeptide. In other embodiments, the N- or C-terminal fragments of the Cas 12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or

(SEQ ID NO: 253)
GGCTCTTCTGGATCTGAAACACCTGGCACAAGCGAGAGCGCCACCCCTG
AGAGCTCTGGC.

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor may comprise all or a portion (e.g., a functional portion) of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant (TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | I | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
TadA*8 Adenosine Deaminase Variants. Residue positions in the E. coli TadA
variant (TadA*) are indicated. Alterations are referenced to TadA*7.10 (first row).

| | 2 | 3 | 4 | 5 | 7 | 8 | 8 | 10 | 10 | 12 | 14 | 14 | 15 | 15 | 15 | 15 | 15 | |
|---|---|---|---|---|---|---|---|---|---|---|---|---|---|---|---|---|---|---|
| | 3 | 6 | 8 | 1 | 6 | 2 | 4 | 6 | 8 | 3 | 6 | 7 | 2 | 4 | 5 | 6 | 7 | 166 |
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
TadA*9 Adenosine Deaminase Variants. Alterations are referenced
to TadA*7.10. Additional details of TadA*9 adenosine
deaminases are described in International PCT Application
No. PCT/US2020/049975, which is incorporated herein by
reference in its entirety for all purposes.

| TadA*9 | |
|---|---|
| Description | Alterations |
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F_I76Y_V82S_Y123H_Y147R_Q154R |
| TadA*9.30 | I76Y_V82T_Y123H_Y147R_Q154R |
| TadA*9.31 | N38G_I76Y_V82S_Y123H_Y147R_Q154R |
| TadA*9.32 | N38G_I76Y_V82T_Y123H_Y147R_Q154R |
| TadA*9.33 | R23H_I76Y_V82S_Y123H_Y147R_Q154R |
| TadA*9.34 | P54C_I76Y_V82S_Y123H_Y147R_Q154R |
| TadA*9.35 | R21N_I76Y_V82S_Y123H_Y147R_Q154R |
| TadA*9.36 | I76Y_V82S_Y123H_D138M_Y147R_Q154R |
| TadA*9.37 | Y72S_I76Y_V82S_Y123H_Y147R_Q154R |
| TadA*9.38 | E25F_I76Y_V82S_Y123H_Y147R_Q154R |
| TadA*9.39 | I76Y_V82T_Y123H_Y147R_Q154R |
| TadA*9.40 | N38G_I76Y_V82S_Y123H_Y147R_Q154R |
| TadA*9.41 | N38G_I76Y_V82T_Y123H_Y147R_Q154R |
| TadA*9.42 | R23H_I76Y_V82S_Y123H_Y147R_Q154R |
| TadA*9.43 | P54C_I76Y_V82S_Y123H_Y147R_Q154R |
| TadA*9.44 | R21N_I76Y_V82S_Y123H_Y147R_Q154R |
| TadA*9.45 | I76Y_V82S_Y123H_D138M_Y147R_Q154R |
| TadA*9.46 | Y72S_I76Y_V82S_Y123H_Y147R_Q154R |
| TadA*9.47 | N72K_V82S, Y123H, Y147R, Q154R |
| TadA*9.48 | Q71M_V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K_V82S, Y123H, Y147R, Q154R |
| TadA*9.54 | Q71M_V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

In some embodiments, the adenosine deaminase comprises a TadA*8.20 adenosine deaminase variant further comprising an F149Y amino acid alteration. In some embodiments, the adenosine deaminase comprises a TadA*8.20 adenosine deaminase variant further comprising the amino acid alterations R147D, F149Y, T166I, and D167N (TadA*8.10+). In some embodiments, the adenosine deaminase comprises a TadA*8.20 adenosine deaminase variant further comprising the amino acid alterations S82T and F149Y (TadA*9v1). In some embodiments, the adenosine deaminase comprises a TadA*8.20 adenosine deaminase variant further comprising the amino acid alterations Y147D, F149Y, T166I, D167N and S82T (TadA*9v2).

In some embodiments, the adenosine deaminase comprises one or more of MII, MIS, S2A, S2E, S2H, S2R, S2L, E3L, V4D, V4E, V4M, V4K, V4S, V4T, V4A, E5K, F6S, F6G, F6H, F6Y, F6I, F6E, S7K, H8E, H8Y, H8H, H8Q, H8E, H8G, H8S, E9Y, E9K, E9V, E9E, Y10F, Y10W, Y10Y, M12S, M12L, M12R, M12W, R13H, R13I, R13Y, R13R, R13G, R13S, H14N, A15D, A15V, A15L, A15H, T17T, T17A, T17W, T17L, T17F, T17R, T17S, L18A, L18E, L18N, L18L, L18S, A19N, A19H, A19K, A19A, A19D, A19G, A19M, R21N, K20K, K20A, K20R, K20E, K20G, K20C, K20Q R21A, R21R, R21N, R21Y, R21C G22P, A22W, A22R, W23D, R23H, W23G, W23Q, W23L, W23R, W23H W23D W23M, W23W, W23I, D24E, D24G, D24W, D24D, D24R, E25F, E25M, E25D, E25A, E25G, E25R, E25E, E25H E25V, E25S, E25Y, R26D, R26E, R26G, R26N, R26Q, R26C, R26L, R26K, R26W, R26C, R26P, R26R, R26A, R26H, E27E, E27Q, E27H, E27C, E27G, E27K, E27S, E27P, E27R, E27L, E27V, E27D, V28V, V28A, V28C, V28G, V28P, V28S, V28T, P29V, P29P, P29A, P29G, P29K, P29L, V30V, V30I, V30L, V30F, V30G, V30A, V30M, L34S, L34V, L34L, L34M, L34W, L34G, H36E, H36V, L36H, H36L, H36N, N37N, N37H, N37R, N37T, N37S, N38G, N38R, N38N, N38E, V40I, W45A, W45W, W45R, W45L, W45N, N46N, N46M, N46P, N46G, N46L, N46R, N46V, R46W, R46F, R46Q, R46M, R47A, R47Q, R47F, R47K, R47P, R47W, R47M, R47R, R47G, R47S, R47V, R47H, P48T, P48L, P48A, P48I, P48S, P48R, P48K, P48D, P48E, P48H, P48G, P48P, P48N, 149G, I49H, I49V, I49F, I49H, I49I, I49M, I49N, I49K, I49Q, I49T, G50L, G50S, G50R, G50G, R51H, R51L, R51N, L51W, R51Y, R51G, R51V, R51R, H52D, H52Y, H52I, H52H, D53D, D53E, D53G, D53P, P54C, P54T, P54P, P54E, A55H, T55A, T55I, T55V, T55G, T55T, A56A, A56H, A56W, A56E, A56S, H57P, H57A, H57H, H57N, A58G, A58E, A58A, A58R, E59A, E59G, E59I, E59Q, E59W, E59E, E59T, E59H, E59P, M61A, M61I, M61L, M61V, M61P, M61G, M61I, L63S, L63V, L63T, L63R, L63H, L63A, R64A, R64Q, R64R, R64D, Q65V, Q65H, Q65G, Q65P, Q65F, Q65Q, Q65R, G66V, G66E, G66T, G66G, G66C, G67G, G67W, G67I, G67A, G67D, G67L, G67V, L68Q, L68M, L68V, L68H, L68L, L68G, V69A, V69M, V69V, M70V, M70L, E70A, M70A, M70M, M70E, M70T, M70v, Q71M, Q71N, Q71L, Q71R, Q71Q, Q71I, N72A, N72K, N72S, N72D, N72Y, N72N, N72H, N72G, N72M, Y73G, Y731, Y73K, Y73R, Y73S, Y73Y, Y73H, Y73A, R74A, R74Q, R74G, R74K, R74L, R74N, R74G, R74K, R74R, I76H, I76R, I76W, I76Y, I76V, I76Q, I76L, I76D, I76F, I76I, I76N, I76T, I76Y, D77G, D77D, D77A, D77Q, A78Y, A78T, A78G, A78A, A78I, T79M, T79R, T79L, T79T, L80M, L80Y, L80I, L80V, L80L, Y81D, Y81V, Y81Y, Y81M, V82A, V82S, V82G, V82T, V82V, V82Q, V82Y, T83L, T83F, T83T, T83N, L84E, L84F, L84Y, L84I, L84L, L84M, L84A, L84T, L84S, E85K, E85G, E85P, E85S, E85E, E85F, E85V, E85R, P86T, P86C, P86P, P86L, P86N, P86K, P86H, C87M, C87I, C87S, C87N, C87P, S87C, S87L, S87V, V88A, V88M, V88V, V88T, V88E, V88D, V88S, C90S, C90P, C90A, C90T, C90M, A91A, A91G, A91S, A91V, A91T, A91C, A91L, G92T, G92M, G92A, G92Y, G92G, A93I, A93C, A93M, A93V, A93A, M94M, M94T, M94A, M94V, M94L, M94I, M94H, I95S, I95G, I95L, I95H, I95V, H96A, H96L, H96R, H96S, H96H, H96N, H96E, S97C, S97G, S97I, S97M, S97R, S97S, S97P, R98K, R98I, R98N, R98Q, R98G, R98H, R98C, R98L, R98R, G100R, G100V, G100K, G100A, G100S, G100M, G100I, R101V, R101R, R101S, R101C, V102A, V102F, V102I, V102V, D103A, V103A, V103G, V103F, V103V, F104G, D104N, F104V, F104I, F104L, F104A, F104F, F104R, G105V, G105W, G105G, G105M, G105A, A106T, V106Q, V106F, V106W, V106M, A106A, A106Q, A106F, A106G, A106W, A106M, A106V, A106R, A106L, A106S, A106B, A106I, R107C, R107G, R107P, R107K, R107A, R107N, R107W, R107H, R107S, R107R, R107F, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, D108E, D108T, D108R, D108D, A109H, A109K, A109R, A109S, A109T, A109V, A109A, A109D, K110G, K110H, K110I, K110R, K110T, K110K, K110A, K1101, T111A, T111G, T111H, T111R, T111T, T111K, G112A, G112G, G112H, G112T, G112R, A113N, A114G, A114H, A114V, A114C, A114S, A114A, G115S, G115G, G115M, G115L, G115A, G115F, L117M, L117L, L117W, L117A, L117S, L117N, L117V, M118D, M118G, M118K, M118N, M118V, M118M, M118L, M118R, D119L, D119N, D119S, D119V, D119D, V120H, V120L, V120V, V120T, V120A, V120E, V120G, V120D, L121D, L121M, L121N, L121K, L121L, H122H, H122N, H122P, H122R, H122S, H122Y, H122G, H122T, H122L, H123C, H123G, H123P, H123V, H123Y, Y123H, H123Y, H123H, P124P, P124H, P124A, P124Y, P124D, P124G, P124I, P124L, P124W, G125H, G125I, G125A, G125M, G125K, G125G, G125P, M126D, M126H, M126K, M126I, M126N, M1260, M126S, M126Y, M126M, M126G, N127H, N127S, N127D, N127K, N127R, N127N, N127I, N127P, N127M, H128R, H128N, H128L, H128H, R129H, R129Q, R129V, R129I, R129E, R129V, R129R, R129M, R129P, V130R, V130V, V130E, V130D, E131E, E131I, E131V, E131K, I132I, I132F, I132T, I132L, I132V, I132E, T133V, T133E, T133G, T133K, T133T, T133A, T133H, T133F, T133I, E134A, E134E, E134G, E134I, E134H, E134K, E134T, G135G, G135V, G135I, G135P, G135E, 1136G, I136L, 1136T, 1136I, 1137A, 1137D, 1137E, L137M, 1137S, L137L, L1371, A138D, A138E, A138G, S138A, A138N, A138S, A138T, A138V, A138Y, A138A, A138M, A138L, D139E, D139I, D139C, D139L, D139M, D139D, D139G, D139H, D139A, E140A, E140C, E140L, E140R, E140K, E140E, E140D, C141S, C141A, C141C, C141V, C141E, A142N, A142D, A142G, A142A, A142L, A142S, A142T, A142N, A142S, A142V, A142E, A142C, A143D, A143E, A143G, A143D, A143G, A143E, A143L, A143W, A143M, A143S, A143Q, A143R, A143A, A143I, L144S, L144L, L144T, L144A, L145A, L145F, L145G, L145D, L145L, L145C, L145E, L145s, C146R, S146A, S146C, S146D, S146F, S146R, S146T, S146D, S146G, S146S, S146L, D147D, D147L, D147F, D147G, D147Y, Y147T, Y147R, Y147D, D147R, D147Y, D147A, D147T, D147H, D147F, D147U, D147V, D1471, D147C, F148L, F148F, F148R, F148Y, F148A, F148T, F149C, F149M, F149R, F149Y, F149N, F149F, F149A, F149T, F149V R150R, R150M, R150D, R150F, M151F, M151P, M151R, M151V, M151M, M151E, R152C, R152F, R152H, R152P, R152R, R152P, R152Q, R152M, R1520, R153C, R153Q, R153R, R153V, R153E, R153A, R153P, Q154E, Q154H, Q154M, Q154R, Q154L, Q154S, Q154V, Q154Q, Q154F, Q154I, Q154A, Q154K, E155F, E155G, E155I, E155K, E155P, E155V, E155D, E155E, E155L, E155Q, I156V, I156A, I156I, I156L, I156F, I156D, I156K, I156N, I156R, I156Y, E157A, E157F, E157I, E157P, E157T, E157V, N157K, K157N, K157V, K157P, K157I, K157F, K157F, K157T, K157A, K157S, K157R, A158Q, A158K, A158V, A158A, A158D, A158S, A158T, A158N, Q159S, Q159Q, Q159A, Q159F, Q159K, Q159L, Q159N, K160A, K160S, K160E, K160K, K160N, K160F, K160Q, K161T, K161K, K161R, K161I, K161A, K16IN, K161Q, K161S, K161T, A162D, A162Q, R162H, R162P, A162S, A162A, A162N, A162M, A162K, Q163G, Q163S, Q163Q, Q163A, Q163H, Q163N, Q163R, S164F, S164S, S164Q, S164I, S164R, S164Y, S165S, S165P, S165Q, S165A, S165D, S165I, S165T, S165Y, T166T, T166Q, T166E, T166S, T166D, T166K, T166I, T166N, T166P, T166R, D167S D167D, D167I, D167G, D167T, D167A and/or D167N mutation in a TadA reference sequence (e.g., TadA*7.10,ecTadA, or TadA8e), and any alternative mutation at the corresponding position, or one or more corresponding mutations in another adenosine deaminase. Additional mutations are described in U.S. Patent Application Publication No. 2022/0307003 A1 U.S. Pat. No. 11,155,803, and International Patent Application Publications No. WO 2023/288304 A2, PCT/CN2022/143408, WO 2018/027078 A1, WO 2021/158921 A1 and WO 2023/034959 A2, the disclosures of which are incorporated herein by reference in their entirety for all purposes.

In some embodiments, the disclosure provides TadA variants comprising a V82T, Y147T, and/or a Q154S mutation. In some embodiments, the disclosure provides TadA variants comprising a V82T, Y147T, and/or a Q154S mutation. In some embodiments, the disclosure provides TadA*8.8 further comprising a V82T mutation. In some embodiments, the disclosure provides TadA*8.8 further comprising a V82T, a Y147T, and a Q154S mutation. In some embodiments, the disclosure provides TadA*8.17 further comprising a V82T mutation. In some embodiments, the disclosure provides TadA*8.17 further comprising a V82T, a Y147T, and a Q154S mutation. In some embodiments, the disclosure provides TadA*8.20 further comprising a V82T mutation. In some embodiments, the disclosure provides TadA*8.20 further comprising a V82T, a Y147T, and a Q154S mutation.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA- | R | V | A | T | D | H | Y | F | T | D |
| | 7.10 | | | | | | | | | | |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the disclosure comprise as a heterodimer of a wild-type TadA (TadA(wt)) linked to a TadA*. Throughout the present disclosure, an adenosine deaminase base editor that comprises a single TadA* domain and a TadA(wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA(wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A•T to G•C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C: G to a T: A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor may comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor may comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can reduce or prevent off-target effects.

In some embodiments, an APOBEC deaminase incorporated into a base editor may comprise one or more mutations selected from the group consisting of H121R, H122R, R126A, R126E, R118A, W90A, W90Y, and R132E of rAPOBEC1 (SEQ ID NO: 21); D316R, D317R, R320A, R320E, R313A, W285A, W285Y, and R326E of hAPOBEC3G; and any alternative mutation at the corresponding position, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the disclosure comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs)” or “cytosine base editors derived from TadA* (CBE-Ts),” and their corresponding deaminase domains may be referred to as “TadA* acting on DNA cytosine (T_{AD}C)” domains. In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, an adenosine deaminase variant of the disclosure is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some instances, the adenosine deaminase variant comprises one or more alterations that increase cytosine deaminase activity (e.g., at least about 10-fold, 20-fold, 30-fold, 40-fold, 50-fold, 60-fold, 70-fold or more increase) relative to the activity of a reference adenosine deaminase and comprise undetectable adenosine deaminase activity or adenosine deaminase activity that is less than 30%, 20%, 10%, or 5% of that of a reference adenosine deaminase. In some embodiments, the reference adenosine deaminase is TadA*8.20 or TadA*8.19.

In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, I49K, 149M, 149N, 149Q, I49T, G67W, I76H, I76R, I76W, Y76H, Y76R, Y76W, F84A, F84M, H96N, G100A, G100K, T111H, G112H, A114C, G115M, M118L, H122G, H122R, H122T, N127I, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising an amino acid alteration or combination of amino acid alterations selected from those listed in any of Tables 6A-6F.

The residue identity of exemplary adenosine deaminase variants that are capable of deaminating adenine and/or cytidine in a target polynucleotide (e.g., DNA) is provided in Tables 6A-6F below. Further examples of adenosine deaminase variants include the following variants of 1.17 (see Table 6A): 1.17+E27H; 1.17+E27K; 1.17+E27S; 1.17+E27S+I49K; 1.17+E27G; 1.17+I49N; 1.17+E27G+I49N; and 1.17+E27Q. In some embodiments, any of the amino acid alterations provided herein are substituted with a conservative amino acid. Additional mutations known in the art can be further added to any of the adenosine deaminase variants provided herein.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6A
Adenosine Deaminase Variants. Mutations are indicated with reference to
TadA*8.20. “S” indicates “Surface,” and “NAS” indicates “Near Active Site.”

| | location in structure | | | | | | | | | | |
|---|---|---|---|---|---|---|---|---|---|---|---|
| | N/A | S h1 | S h1 | S h1 | NAS | NAS | NAS | NAS | | S | |
| | Amino Acid No. (*START Met is AA#1) | | | | | | | | | | |
| | 2 | 8 | 13 | 17 | 27 | 47 | 48 | 49 | 67 | 76 | 77 |
| TadA*8.20 | S | H | R | T | E | R | A | I | G | Y | D |
| TadA*8.19 | | | | | | | | | | I | |
| 1.1 | | | | | H | | | | | I | |
| 1.2 | | | | | H | | | K | | I | |
| 1.3 | | | | | S | | | K | | I | |
| 1.4 | | | | | S | | | K | | I | |
| 1.5 | | | | | K | | | | | | |
| 1.6 | | | | | K | | | | | | |
| 1.7 | | | | | H | | | | | I | |
| 1.8 | | | | | S | | | K | W | | |
| 1.9 | | | | | | | | T | M | | |
| 1.10 | | | | | C | | | | | I | |
| 1.11 | | | G | | Q | | | | | | |
| 1.12 | | | | A | H | | | M | | I | |
| 1.13 | | | | | | | | Q | | I | |
| 1.14 | H | | | | | | | K | | I | |
| 1.15 | | | | | | S | | | | | |
| 1.16 | | Q | | | | | | Q | | I | |
| 1.17 | | | | A | | | G | | | | |
| 1.18 | | | | | G | | | | | | |
| 1.19 | | | | | G | | | N | | | |
| 1.20 | | | | | G | | | | | | G |

Adenosine Deaminase Variants. Mutations are indicated with reference to TadA*8.20.
“I” indicates “Internal,” “S” indicates “Surface,” and “NAS” indicates “Near Active Site.”

| | location in structure | | | | | | | | | | | |
|---|---|---|---|---|---|---|---|---|---|---|---|---|
| | I | NAS | | NAS | S | | S | S | S | | S | |
| | Amino Acid No. (*START Met is AA#1) | | | | | | | | | | | |
| | 82 | 84 | 96 | 107 | 112 | 115 | 118 | 119 | 127 | 142 | 162 | 165 |
| TadA*8.20 | S | F | H | R | G | G | M | D | N | A | A | S |
| TadA*8.19 | | | | | | | | | | | | |
| 1.1 | | M | | | | | | | | | | |
| 1.2 | | | | | | | | | | | | |
| 1.3 | | | | | | | | | | | | |
| 1.4 | | | | | | | | | | | N | |
| 1.5 | | | | | | | | | | | | |
| 1.6 | | | | | | | | N | | | | |
| 1.7 | | | | | | | | | | | | |
| 1.8 | | | | | | | | | | | | |
| 1.9 | | | N | | | | | | | | | |
| 1.10 | | | | | | | | N | | | | |
| 1.11 | | | | | | | | | K | | | |
| 1.12 | | | | | | | L | | | | | |
| 1.13 | | | | | | M | | | | | | |
| 1.14 | | | | | H | | | | | | | |
| 1.15 | | | | C | | | | | | | | |
| 1.16 | | | | | | | | | | | | |
| 1.17 | T | | | | | | | | | E | | |
| 1.18 | | | | | | | | | | | | |
| 1.19 | | | | | | | | | | | | |
| 1.20 | | | | | | | | | | | | P |

TABLE 6B
Adenosine deaminase variants. Mutations are indicated with
reference to TadA*8.20.

| | Position No. | | | | | | | | | |
|---|---|---|---|---|---|---|---|---|---|---|
| | 27 | 29 | 30 | 49 | 82 | 84 | 107 | 112 | 115 | 142 |
| | TadA*8.20 | | | | | | | | | |
| | E | P | V | I | S | F | R | G | G | A |
| | Alterations Evaluated | | | | | | | | | |
| | G/S/H | G/A/K | I/L/F | K | T | L/A | C | H | M | E |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | C | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | E |
| S1.54 | S | | | K | T | L | | | M | E |
| S1.55 | S | | | K | T | L | C | H | M | E |
| S1.56 | S | | I | K | T | L | | | | |
| S1.57 | S | | I | K | T | L | C | | | |
| S1.58 | S | | I | K | T | L | | H | | |
| S1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | I | K | T | L | | H | | E |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | E |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | E |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | C | | | E |
| S1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | C | | M | |
| S1.173 | | A | | | T | | C | | | E |
| S1.174 | | A | | | T | | | H | | E |
| S1.175 | | A | | | T | | | | M | F |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| S1.180 | S | | I | | T | | | | M | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 6C
Adenosine deaminse variants. Mutations are indicated with reference to variant 1.2 (Table 6A).

| | | Residue identity (START Met is amino acid #1) | | | | | | | | |
|---|---|---|---|---|---|---|---|---|---|---|
| Variant Name | Alternative Variant Names | 4 | 6 | 17 | 23 | 76 | 77 | 100 | 111 | 114 |
| Reference | 1.2 (see Table 6A) | V | F | T | R | I | D | G | T | A |
| TadAC2.1 | pDKL-135; 2.1 | K | | | | | | | | C |
| TadAC2.2 | pDKL-136; 2.2 | K | | | | | G | | | |
| TadAC2.3 | pDKL-137; 2.3 | | Y | | | | | A | | |
| TadAC2.4 | pDKL-138; 2.4 | T | | | | R | | | | |
| TadAC2.5 | pDKL-139; 2.5 | | Y | | | W | | | | |
| TadAC2.6 | pDKL-140; 2.6 | | Y | | | | | | | |
| TadAC2.7 | pDKL-141; 2.7 | | Y | | | | | | | C |
| TadAC2.8 | pDKL-142; 2.8 | | Y | | | | | | | |
| TadAC2.9 | pDKL-143; 2.9 | K | | | | M | | | | |
| TadAC2.10 | pDKL-144; 2.10 | | G | | | R | | K | | |
| TadAC2.11 | pDKL-145; 2.11 | | H | | | | | | | |
| TadAC2.12 | pDKL-146; 2.12 | | | | | | | | | C |
| TadAC2.13 | pDKL-147; 2.13 | | Y | | | H | | | | |
| TadAC2.14 | pDKL-148; 2.14 | | | | | | | | | |
| TadAC2.15 | pDKL-149; 2.15 | | | | Q | R | | | | |
| TadAC2.16 | pDKL-150; 2.16 | | | | | H | | | | |
| TadAC2.17 | pDKL-151; 2.17 | | Y | | | | | | H | |
| TadAC2.18 | pDKL-152; 2.18 | | | | | W | | | | |
| TadAC2.19 | pDKL-153; 2.19 | | | | | | | | H | |
| TadAC2.20 | pDKL-154; 2.20 | | | | | | | | | |
| TadAC2.21 | pDKL-155; 2.21 | | Y | | | R | | | | |
| TadAC2.22 | pDKL-156; 2.22 | | | W | | H | | | | |
| TadAC2.23 | pDKL-157; 2.23 | S | | | | Y | | | | |
| TadAC2.24 | pDKL-158; 2.24 | | | | | | | | | |
| | | Residue identity (START Met is amino acid #1) | | | | | | | | |
| Variant Name | Alternative Variant Names | 119 | 122 | 127 | 143 | 147 | 158 | 159 | 162 | 166 |
| Reference | 1.2 (see Table 6A) | D | H | N | A | R | A | Q | A | T |
| TadAC2.1 | pDKL-135; 2.1 | | | | | | | | | |
| TadAC2.2 | pDKL-136; 2.2 | | | | | | | | | |
| TadAC2.3 | pDKL-137; 2.3 | | R | | | | | | | |
| TadAC2.4 | pDKL-138; 2.4 | | G | | | | | | | |
| TadAC2.5 | pDKL-139; 2.5 | | | | | | | | | |
| TadAC2.6 | pDKL-140; 2.6 | N | | | | | | | | |
| TadAC2.7 | pDKL-141; 2.7 | | | | | | | | | |
| TadAC2.8 | pDKL-142; 2.8 | | | | | | | | | |
| TadAC2.9 | pDKL-143; 2.9 | | T | | | | | | | |
| TadAC2.10 | pDKL-144; 2.10 | | | | | | | | | |
| TadAC2.11 | pDKL-145; 2.11 | | N | | | | | | | |
| TadAC2.12 | pDKL-146; 2.12 | | | | | | | | | |
| TadAC2.13 | pDKL-147; 2.13 | | R | | | | | | | I |
| TadAC2.14 | pDKL-148; 2.14 | | | P | | | | | | |
| TadAC2.15 | pDKL-149; 2.15 | | | | | | | | | |
| TadAC2.16 | pDKL-150; 2.16 | | R | | | | V | | | |
| TadAC2.17 | pDKL-151; 2.17 | | | | | | | | | |
| TadAC2.18 | pDKL-152; 2.18 | | | | | | | | | |
| TadAC2.19 | pDKL-153; 2.19 | | G | | | | | | C | |
| TadAC2.20 | pDKL-154; 2.20 | | | | E | | | | | |
| TadAC2.21 | pDKL-155; 2.21 | | | | | | | | | |
| TadAC2.22 | pDKL-156; 2.22 | | G | | | | V | | | |
| TadAC2.23 | pDKL-157; 2.23 | | | | E | | | S | | |
| TadAC2.24 | pDKL-158; 2.24 | | | I | | | | | Q | |

TABLE 6D
Adenosine deaminase variants. Mutations are indicated with reference to

| AA Positions | 6 | 27 | 49 | 76 | 77 | 82 | 107 | 112 | 114 | 115 | 119 | 122 | 127 | 142 | 143 |
|---|---|---|---|---|---|---|---|---|---|---|---|---|---|---|---|
| TadA*8.20 | F | E | I | Y | D | S | R | G | A | G | D | H | N | A | A |
| S1.154 | F | H | K | Y | D | T | C | H | | M | | | | E | |
| Alterations | Y | | | W | G | | | | C | | N | G | P | | E |
| from Table 6C | | | | | | | | | | | | | | | |
| S2.1 | Y | H | K | W | | T | C | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| S2.5 | Y | H | K | | | T | C | H | | M | | G | | | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | G | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | G | T | C | H | | M | | | | A | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| S2.41 | Y | H | K | W | | T | C | H | | M | | G | P | E | |
| S2.42 | Y | H | K | W | | T | C | H | C | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | W | G | T | C | H | C | M | | | P | E | |
| S2.48 | Y | H | K | W | G | T | C | H | C | M | | | | E | E |
| $2.49 | Y | H | K | W | G | T | C | H | C | M | | | | A | E |
| S2.50 | Y | H | K | W | G | T | C | H | C | M | N | G | | E | |
| S2.51 | Y | H | K | W | G | T | C | H | C | M | N | | P | E | |
| S2.52 | Y | H | K | W | G | T | C | H | C | M | | G | P | E | |
| S2.53 | Y | H | K | W | | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6E
Hybrid constructs. Mutations are indicated with reference to TadA*7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| TadA*7.10 | I | V | A | T | D | H | Y | Y | F | Q | T | D |
| TadA*8e | | | S | R | N | N | | D | Y | | I | N |
| TadA*8.20 | Y | S | | | | | H | R | | R | | |
| TadA*8.17 | | S | | | | | | | | R | | |
| pNMG-B878 | Y | S | | | | | H | D | | R | | |
| pNMG-B879 | Y | S | | | | | H | R | Y | R | | |
| pNMG-B880 | Y | S | | | | | H | R | | R | I | |
| pNMG-B881 | Y | S | | | | | H | R | | R | | N |
| pNMG-B882 | Y | S | | | | | H | D | Y | R | I | N |
| pNMG-B883 | Y | S | | R | N | | H | R | | R | | |
| pNMG-B884 | Y | S | S | R | N | N | H | R | | R | | |
| pNMG-B885 | Y | S | S | | | | H | R | | R | | |
| pNMG-B886 | Y | S | | R | | | H | R | | R | | |
| pNMG-B887 | Y | S | | | N | | H | R | | R | | |
| pNMG-B888 | Y | S | | | | N | H | R | | R | | |
| pNMG-B889 | Y | S | S | R | | | H | R | | R | | |
| pNMG-B890 | Y | S | | | N | N | H | R | | R | | |
| TadA*7.10 | I | V | A | T | D | H | Y | Y | F | Q | T | D |
| TadA*8e | | | S | R | N | N | | D | Y | | I | N |
| TadA*8.20 | Y | S | | | | | H | R | | R | | |
| TadA*8.17 | | S | | | | | | | | R | | |
| pNMG-B891 | Y | S | S | R | N | N | H | D | Y | R | I | N |

TABLE 6F
Base editor variants. Mutations are indicated with reference to TadA*8.19/8.20.

| AA positions: | 17 | 27 | 48 | 49 | 76 | 82 | 84 | 118 | 142 | 147 | 149 | 166 | 167 |
|---|---|---|---|---|---|---|---|---|---|---|---|---|---|
| ABE8.19m/8.20m | T | E | A | I | Y/I | S | F | M | A | Y | F | T | D |
| 1.1 + 8e(B879) | | H | | | I | | M | | | | Y | | |
| 1.2 + 8e(B879) | | H | | K | I | | | | | | Y | | |
| 1.12 + 8e(B879) | A | H | | M | I | | | L | | | Y | | |
| 1.17 + 8e(B879) | A | | G | | | T | | | E | | Y | | |
| 1.18 + 8e(B879) | | G | | | | | | | | | Y | | |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide may comprise a CRISPR RNA (crRNA) and a trans-activating CRISPR RNA (tracrRNA) or may comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 425. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides may comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and may be separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications. Such modifications can increase base editing ˜2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide may comprise one or more modifications to provide a nucleic acid with a new or enhanced feature. A guide polynucleotide may comprise a nucleic acid affinity tag. A guide polynucleotide may comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNa can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR[PAATKKAGQA]KKKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV.

In some embodiments, any of the fusion proteins or complexes provided herein comprise an NLS comprising the amino acid sequence EGADKRTADGSEFESPKKKRKV (amino acids 8 to 29 of SEQ ID NO 328). In some embodiments, any of the adenosine base editors provided herein comprise the amino acid sequence EGADKRTADGSEFESPKKKRKV (amino acids 8 to 29 of SEQ ID NO: 328). In some embodiments, the NLS is at a C-terminal portion of the adenosine base editor. In some embodiments, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some cases, a base editor is expressed in a cell in trans with a UGI polypeptide. In some embodiments, cellular DNA repair response to the presence of U: G heteroduplex DNA can be responsible for a reduction in nucleobase editing efficiency in cells. In such embodiments, uracil DNA glycosylase (UDG) can catalyze removal of U from DNA in cells, which can initiate base excision repair (BER), mostly resulting in reversion of the U: G pair to a C: G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH₂) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voß, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure may comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. coli TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G+ Y147D + F149Y + |
| | | Q154S + N157K + D167N |
| ABE-827m | MSP827 | monomer_TadA*7.10 + L36H + I76Y + V82G + Y147T + |
| | | Q154S + N157K |
| ABE-828m | MSP828 | monomer_TadA*7.10 + I76Y + V82G + Y147D + F149Y + |
| | | Q154S + D167N |
| ABE-829m | MSP829 | monomer_TadA*7.10 + L36H + I76Y + V82G + Y147D + |
| | | F149Y + Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + |
| | | Y147T + Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + |
| | | Y147D + F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS) n (SEQ ID NO: 246), (GGGGS)n (SEQ ID NO: 247), and (G) n to more rigid linkers of the form (EAAAK) n (SEQ ID NO: 248), (SGGS)n (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGS
PTSTEEGTSTEPSEGSAPGTSTEPSEGSAPGTSESATPESGPGSEPATS
GGSGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSGGS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEE
GTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.
Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure may comprise any domain, feature or amino acid sequence which facilitates the editing of a target polynucleotide sequence.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the disclosure advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations: unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The disclosure provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, I95%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein may comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### Expression of Polypeptides in a Host Cell

Polypeptides of the present disclosure may be expressed in virtually any host cell of interest, including mammalian cells (e.g., human cells). In some embodiments, the host cell is an immune cell (e.g., T-, or NK-cell). In some embodiments, the host cell is an allogeneic immune cell (e.g., T- or NK-cell). In some embodiments, the host cell is a CAR-T cell.

An expression vector containing a DNA encoding a nucleic acid sequence-recognizing module and/or a nucleic acid base converting enzyme can be produced, for example, by linking the DNA to the downstream of a promoter in a suitable expression vector.

In some embodiments, the nucleic acid sequence is inserted into the genome of the cell (e.g., T cell or NK cell) by introducing a vector, for example, a viral or non-viral vector, comprising the nucleic acid. Examples of viral vectors include, but are not limited to, adeno-associated viral (AAV) vectors, retroviral vectors or lentiviral vectors. In some embodiments, the lentiviral vector is an integrase-deficient lentiviral vector. In some embodiments, the nucleic acid sequence is inserted into the genome of the cell (e.g., T cell) via non-viral delivery. In non-viral delivery methods, the nucleic acid can be naked DNA, or in a non-viral plasmid or vector.

Regarding the promoter to be used, any promoter appropriate for a host to be used for gene expression can be used. For example, when the host is an animal cell, an SRα promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK), MND (a synthetic promoter that contains the U3 region of a modified MoMuLV LTR with myeloproliferative sarcoma virus enhancer) promoter, and the like can be used. Of these, CMV promoter, SR.alpha. promoter and the like.

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors. Non-limiting examples of viral vectors include lentivirus (e.g., HIV and FIV-based vectors), Adenovirus (e.g., AD100), rabies virus (see, e.g., U.S. Patent Application Publication No. US 2022/0290164 A1, the disclosure of which is incorporated herein by reference in its entirety for all purposes), retrovirus (e.g., Maloney murine leukemia virus, MML-V), herpesvirus vectors (e.g., HSV-2), and Adeno-associated viruses (AAVs), or other plasmid or viral vector types.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas9 nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient homology directed repair (HDR) with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (ssDNA) donors.

In some embodiments, a heterologous polynucleotide may be inserted into the genome of a cell using a transposable element such as a transposon, as described, for example, in Tipanee, et al. Human Gene Therapy, November 2017, 1087-1104, DOI: 10.1089/hum.2017.128. Transposable elements are divided into two categories: retrotransposons and DNA transposons. Transposable elements can alter the genome of the host cells through insertions, duplications, deletions, and translocations. Retrotransposons are described as mobile elements that employ an RNA intermediate that is first reverse transcribed into a complementary single-stranded (c) DNA strand by a reverse transcriptase encoded by the retrotransposon. Subsequently, the single-stranded DNA is converted into a double-stranded DNA that then integrates into the host genome. This so-called “replicative mechanism” yields several new copies of retrotransposons expanding throughout the target genome over evolutionary time. Retrotransposons are categorized into many subtypes according to the DNA sequences of the long terminal repeats and its open reading frames. Retrotransposons were employed to enable transgene integration into the target cell DNA, in some cases relying on adenoviral delivery. Alternatively, DNA transposons translocate via a “non-replicative mechanism,” whereby two Terminal Inverted Repeats (TIRs) are recognized and cleaved by a transposase enzyme, releasing the cognate DNA transposons with free DNA ends. The excised DNA transposons then integrate into a new genomic region where target sites are recognized and cut by the same transposase. This cut-and-paste mechanism usually duplicates DNA target sites upon insertion, leaving target site duplications (TSDs). Non-limiting examples of transposons include the Sleeping Beauty (SB) transposon, the piggyBac (PB) transposon, and Tol2 transposable elements. In various embodiments, a transposase is used to edit a polynucleotide so as to disrupt expression of a polypeptide (e.g., an FKBP1A polypeptide) encoded by the polynucleotide. For example, a transposase may be used in some embodiments to insert a heterologous polynucleotide within a polynucleotide encoding a polypeptide (e.g., an FKBP1A gene) to disrupt expression of the polypeptide encoded by the polynucleotide. In some instances, a transposon may be used to introduce an insertion or deletion mutation to a polynucleotide, thereby disrupting expression of a polypeptide encoded by the polynucleotide.

### Inteins

Inteins (intervening protein) are auto-processing domains found in a variety of diverse organisms, which carry out a process known as protein splicing.

Non-limiting examples of inteins include any intein or intein-pair known in the art, which include a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138(7):2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377 and 389-424. Inteins suitable for use in embodiments of the present disclosure and methods for use thereof are described in U.S. Pat. No. 10,526,401, International Patent Application Publication No. WO 2013/045632 or WO 2020/051561, and in U.S. Patent Application Publication No. US 2020/0055900, the full disclosures of which are incorporated herein by reference in their entireties by reference for all purposes.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]—C. In some embodiments, an intein-C is fused to the N-terminus of the C-terminal portion of the split Cas9, i.e., to form a structure of N-[intein-C]—[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. Methods for designing and using inteins are known in the art and described, for example by WO2014004336, WO2017132580, WO2013045632A1, US20150344549, and US20180127780, each of which is incorporated herein by reference in their entirety.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, referenced to SEQ ID NO: 197.

### PHARMACEUTICAL COMPOSITIONS

In some aspects, the present disclosure provides a pharmaceutical composition comprising any of the cells, polynucleotides, vectors, base editors, base editor systems, guide polynucleotides, fusion proteins, complexes, or the fusion protein-guide polynucleotide complexes described herein.

The pharmaceutical compositions of the present disclosure can be prepared in accordance with known techniques. See, e.g., Remington, The Science and Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present disclosure provide methods of treating a subject in need, the method comprising administering to a subject in need an effective therapeutic amount of a pharmaceutical composition as described herein. More specifically, the methods of treatment include administering to a subject in need thereof one or more pharmaceutical compositions comprising one or more cells having at least one edited gene. In other embodiments, the methods of the disclosure comprise expressing or introducing into a cell a base editor polypeptide and one or more guide RNAs capable of targeting a nucleic acid molecule encoding at least one polypeptide.

In various embodiments, the methods of the disclosure involve co-administering to a subject in need of treatment for a neoplasia allogeneic immune effector cells modified according to the methods provided herein and an immunosuppressive agent. Administration of the immunosuppressive agent can have the advantage of reducing or eliminating rejection of the allogeneic immune effector cells by the subject's immune system (e.g., T cells or NK cells). In some embodiments, the method is a treatment for a neoplasia (e.g., a lymphoma or a leukemia, such as a B cell lymphoma). If the cells are used for treating a neoplasia, it can be advantageous for the allogeneic immune effector cells (e.g., NK cells or T cells) to express a chimeric antigen receptor (CAR) capable of binding a marker associated with the neoplasia (e.g., a CD19 polypeptide). In embodiments, the modified immune effector cells have reduced expression or activity of an FKBP1A, NR3C1, or PPIA polypeptide relative to unmodified allogeneic immune effector cells. The modified immune effector cells have increased resistance to immunosuppression by the immunosuppressive agent relative to unmodified immune effector cells. Non-limiting examples of immunosuppressive agents include mTOR inhibitors (e.g., a rapalog, such as rapamycin or everolimus), calcineurin inhibitors (e.g., cyclosporine A or tacrolimus), and glucocorticoids (e.g., dexamethasone or prednisolone). In embodiments, modified allogeneic immune effector cells (e.g., CAR-T or CAR-NK cells) show increased killing of neoplastic cells in the subject administered the immunosuppressive agent relative to unmodified allogeneic immune effector cells.

The immunosuppressive agent may be administered to the subject about 1, 2, 3, 4, 5, 6, 7, 8, 9, or 10 times every 1, 2, 3, 4, 5, 6, 7, 8, 9, 10, 11, 12, 13, 14 days or every month following an initial administration of cells of the disclosure. The immunosuppressive agent may be administered to a subject before, after, or at the same time as the modified immune cells of the disclosure. In some cases, the immunosuppressive agent is administered or first administered within 1 hr, 2 hr, 3 hr, 4 hr, 5 hr, 6 hr, 12 hr, 1 day, 2 days, 3 days, 4 days, 5 days, 6 days, 7 days, or 1 month, before or after an administration (e.g., first administration) of the modified immune cells of the disclosure to a subject.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

### Kits

The disclosure provides kits for the treatment of a neoplasia (e.g., a lymphoma) in a subject where the kit comprises a chimeric antigen receptor (CAR)-expressing immune effector cell comprising an edit to make it resistant to an immunosuppressive agent. In some embodiments, the kit further comprises an immunosuppressive reagent (mTOR inhibitors (e.g., a rapalog, such as rapamycin or Everolimus), Calcineurin Inhibitors (e.g., cyclosporine A or tacrolimus), and Glucocorticoids (e.g., Dexamethasone or Prednisolone). In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the kit comprises an edited cell and instructions regarding the use of such cell.

The kits may further comprise written instructions for using a base editor, base editor system and/or edited cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Immunosuppressant Agents (e.g., Rapamycin, Tacrolimus) Inhibited T Cell and NK Cell-Mediated Alloresponse In Vitro

Experiments were undertaken to demonstrate that immunosuppressant agents (e.g., rapamycin and tacrolimus) inhibit T cell and NK cell mediated alloresponse in vitro.

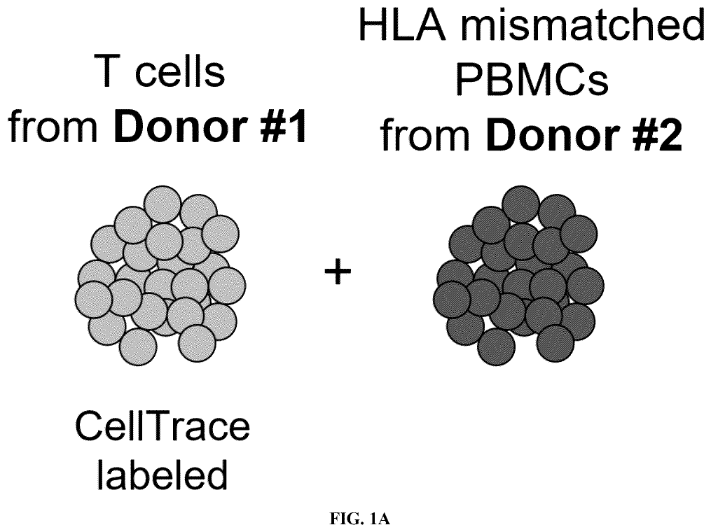

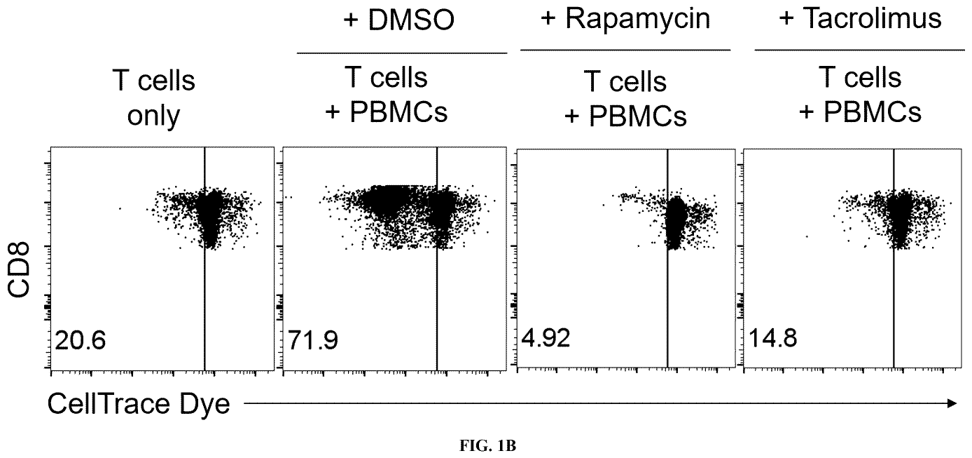

First, human leukocyte antigen (HLA) mismatched peripheral blood mononuclear cells (PBMCs) from a donor were co-cultured in the presence and absence of an immunosuppressant agents with T cells from a different donor that were labeled using a CellTrace dye for measuring cell proliferation. The peripheral blood mononuclear cells (PBMC) cells were treated with mitomycin C prior to being co-cultured to prevent proliferation of the PBMC cells. It was observed (FIGS. 1A and 1B) that the immunosuppressant agents (rapamycin and tacrolimus) hindered proliferation of the T cells.

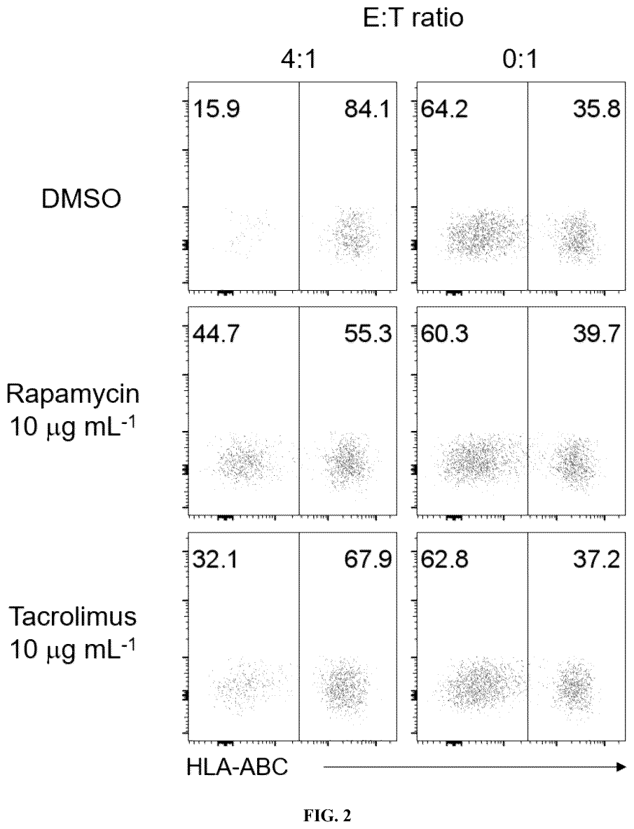

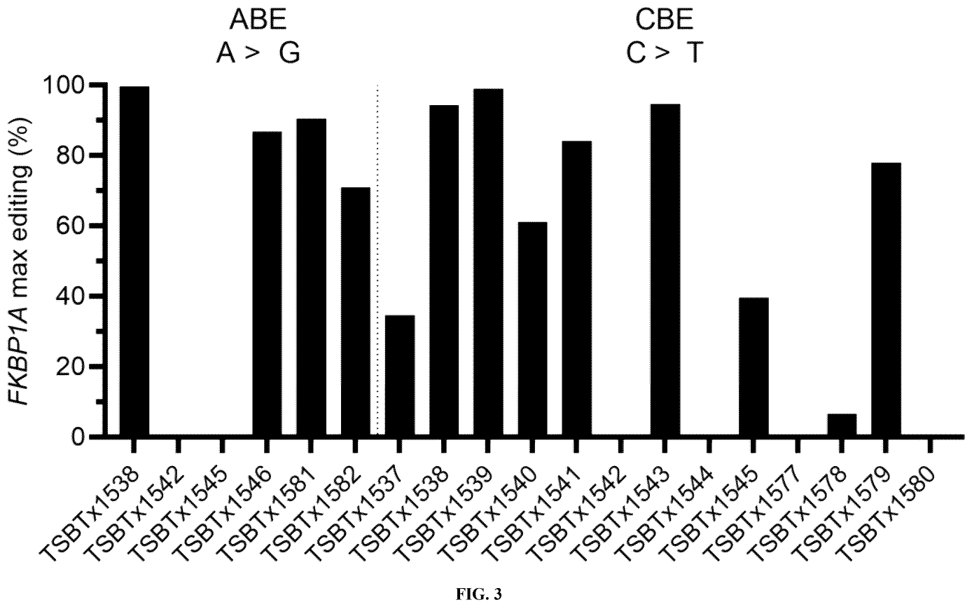

Next, NK cells were co-cultured with a population of cells containing both HLA class-I positive and HLA class-I negative cells in the presence and absence of immunosuppressant agents. Cytotoxicity of the NK cells (i.e., killing of the HLA class-I negative cells) was reduced by the immunosuppressant agents (FIG. 2).

### Example 2: Base Editing of T Cells to Reduce or Knock-Out Expression of NR3C1

Given that immunosuppressants can hinder the proliferation and/or cytotoxicity of T cells, experiments were undertaken to identify base editor systems suitable for editing T cells to reduce or eliminate expression of NR3C1 so that the cells would become resistant to suppression by immunosuppressive agents, such as glucocorticoids.

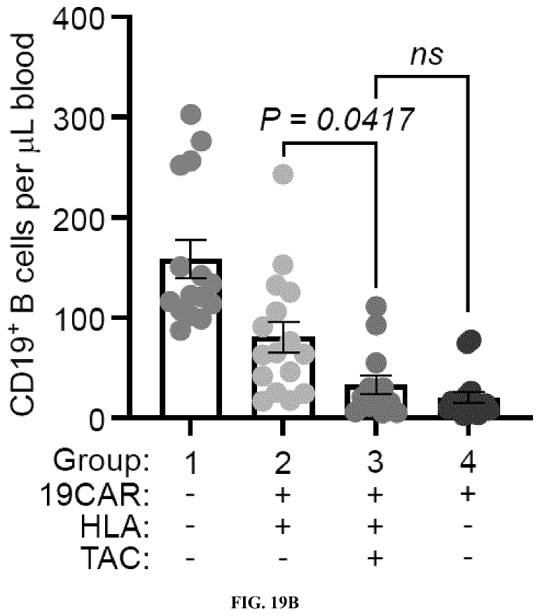

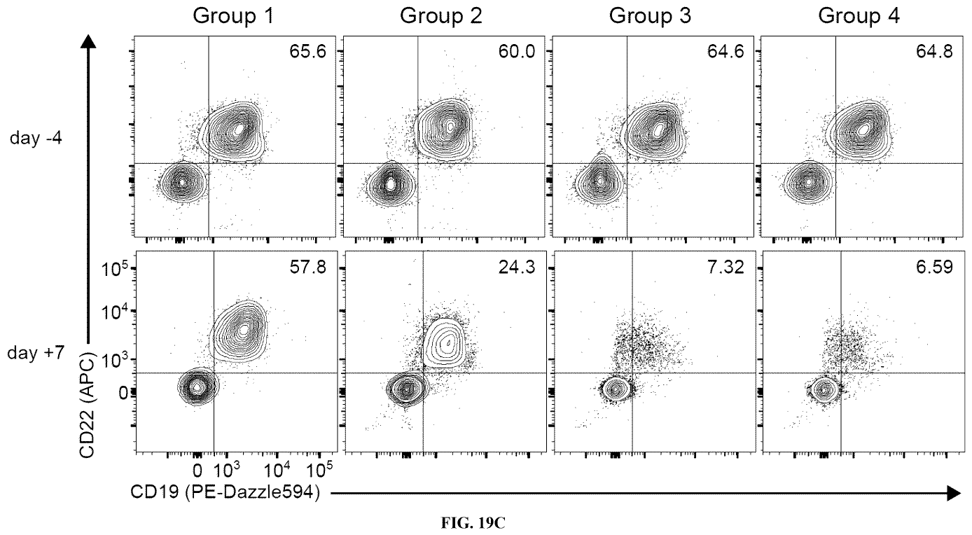

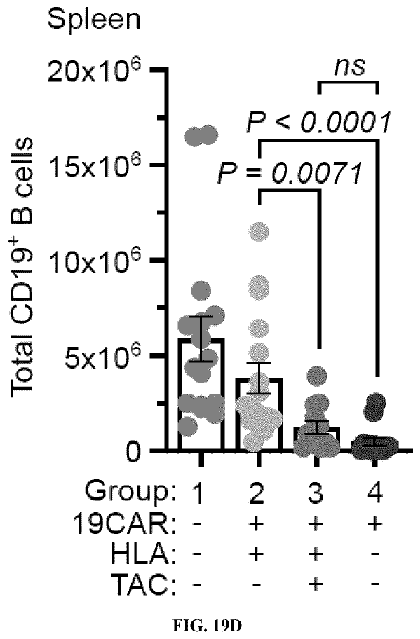

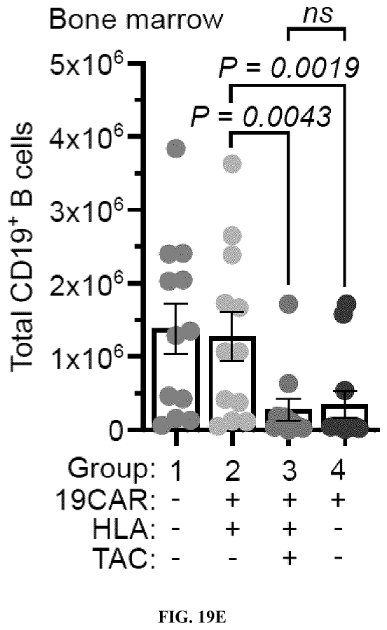

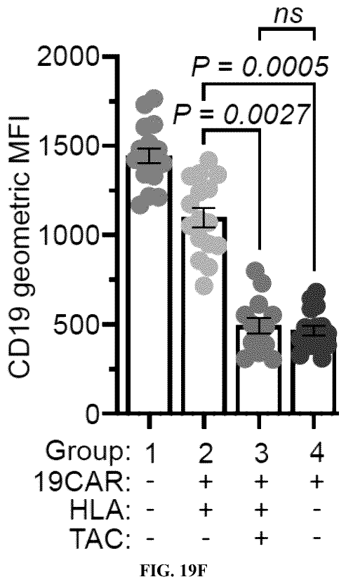

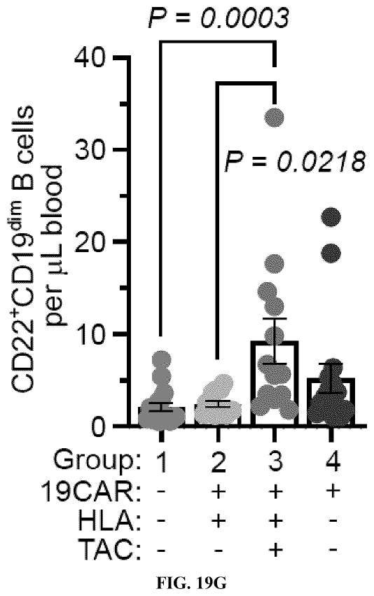

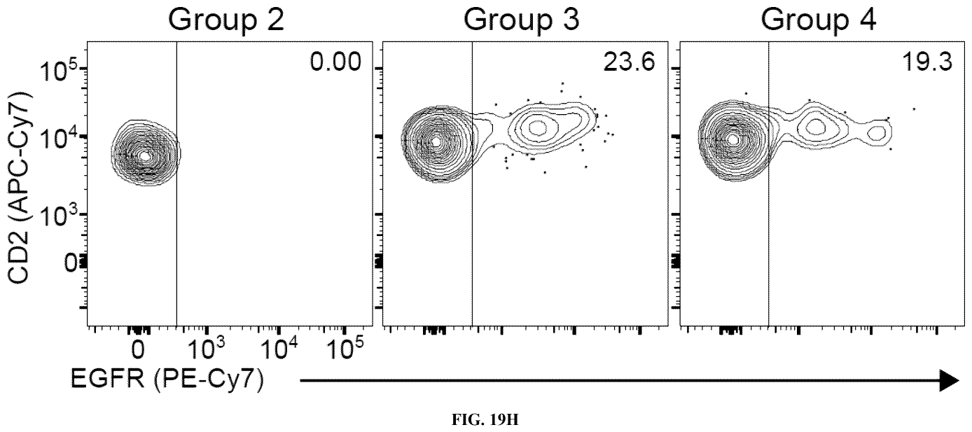

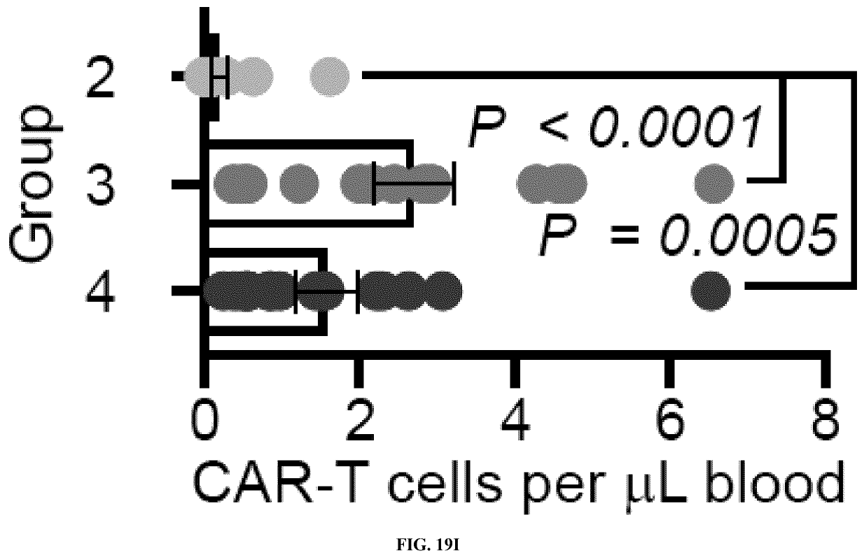

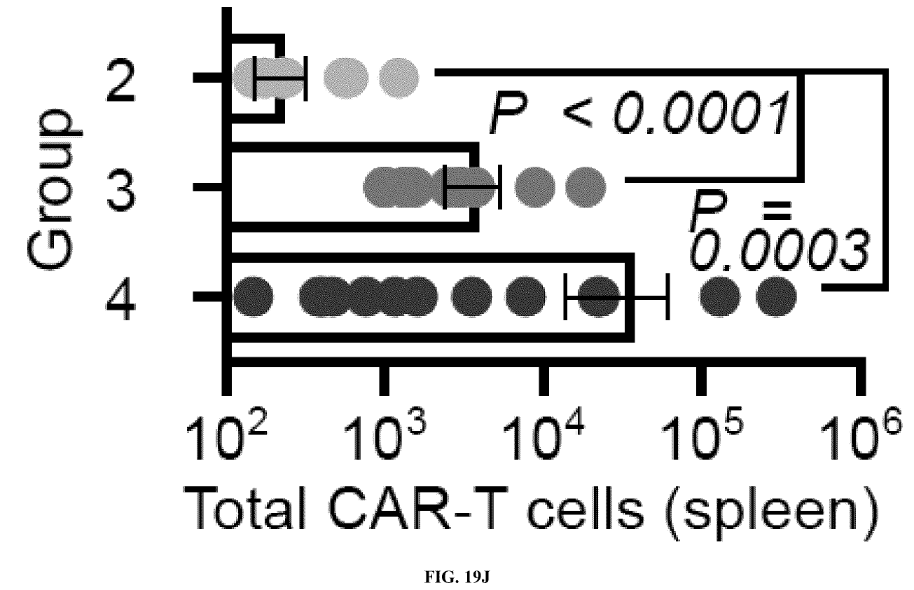

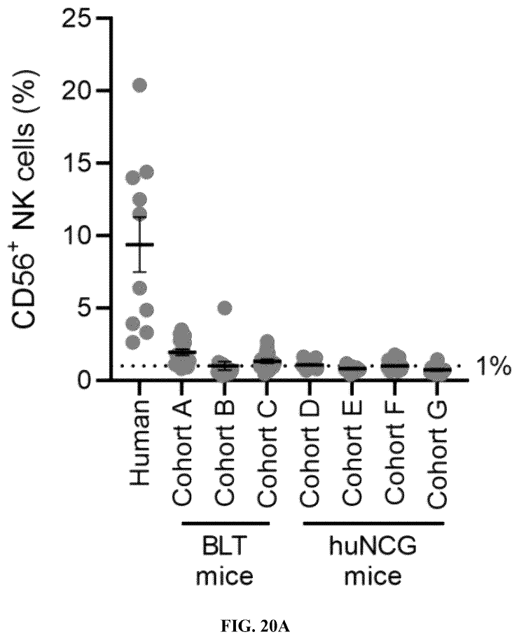

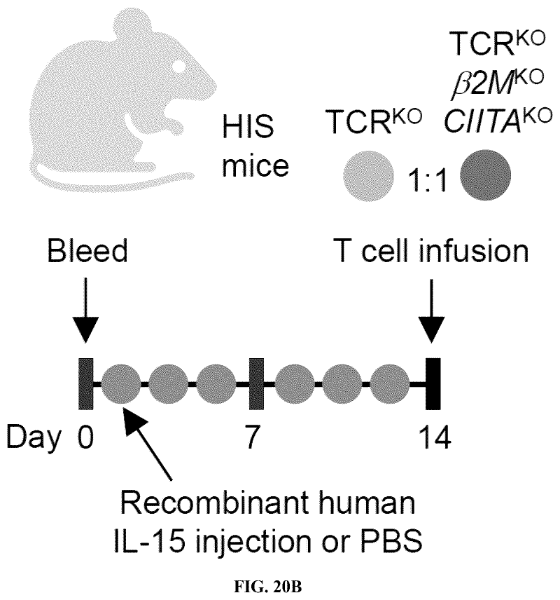

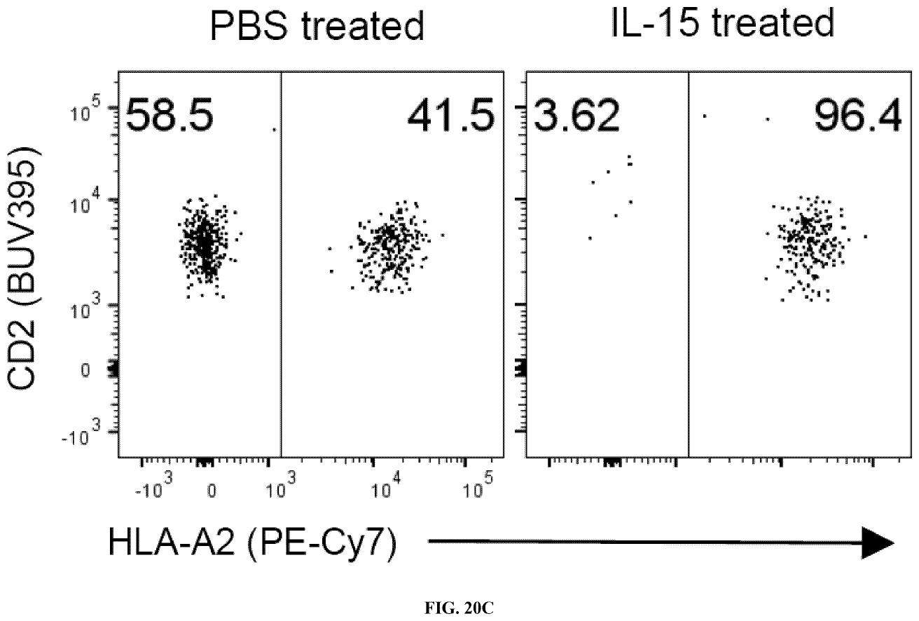

T cells were base edited to reduce or eliminate expression of NR3C1 by electroporating the cells with a guide polynucleotide selected from TSBT×1564, TSBT×1566, TSBT×1568, TSBT×1569, and TSBT×1575 (see Table 1 for guide sequences) and mRNA encoding the adenosine deaminase base editor ABE8.20m, or a guide polynucleotide selected from TSBT×1547, TSBT×1548, TSBT×1549, TSBT×1550, TSBT×1551, TSBT×1552, TSBT×1553, TSBT×1554, TSBT×1555, TSBT×1556, TSBT×1557, TSBT×1558, TSBT×1559, TSBT×1560, TSBT×1561, TSBT×1562, TSBT×1563, TSBT×1565, TSBT×1566, TSBT×1567, TSBT×1568, TSBT×1570, TSBT×1571, TSBT×1572, TSBT×1573, TSBT×1574, and TSBT×1576 (see Table 1 for guide sequences) and mRNA encoding the cytidine deaminase base editor rBE4. Base editing of the NR3C1 polypeptide in the T cells was measured using next generation sequencing (FIG. 5). A number of the base editor systems achieved maximum base editing rates in excess of 40% or even 80%.

### Example 3: Base Editing of T Cells to Reduce or Knock-Out Expression of PPIA

Given that immunosuppressants can hinder the proliferation and/or cytotoxicity of T cells, experiments were undertaken to identify base editor systems suitable for editing T cells to reduce or eliminate expression of PPIA so that the cells would become resistant to suppression by immunosuppressive agents, such as calcineurin inhibitors.

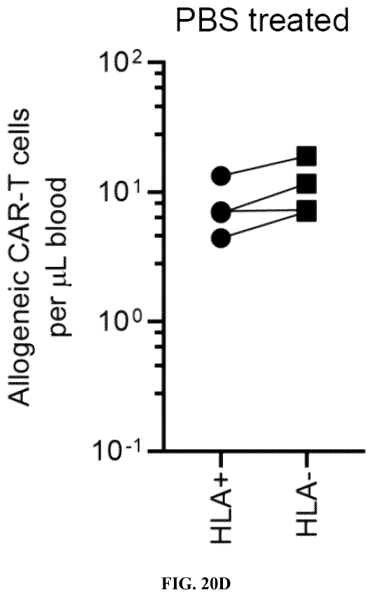

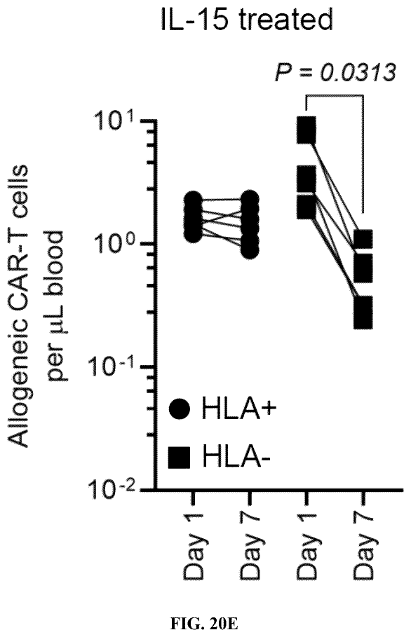

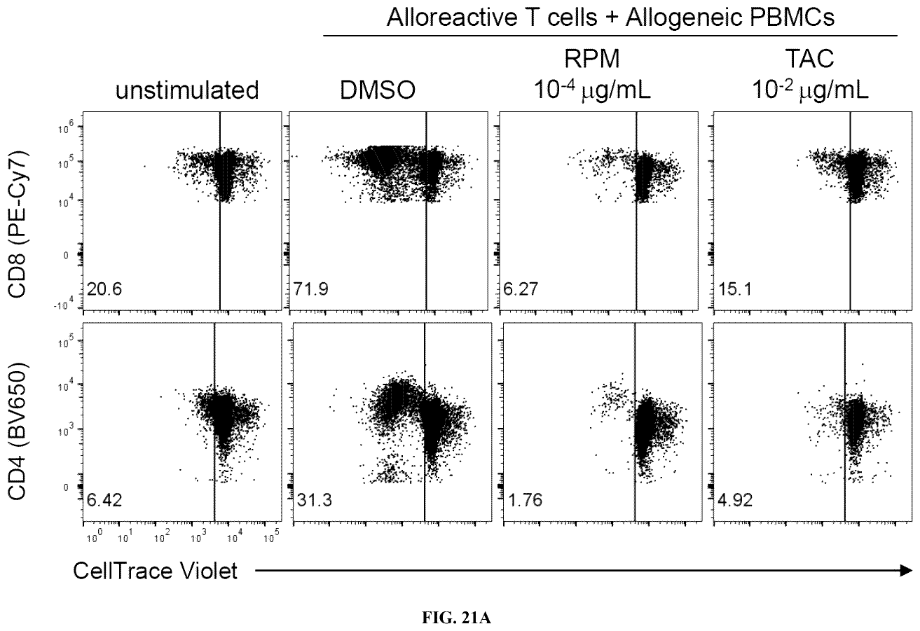

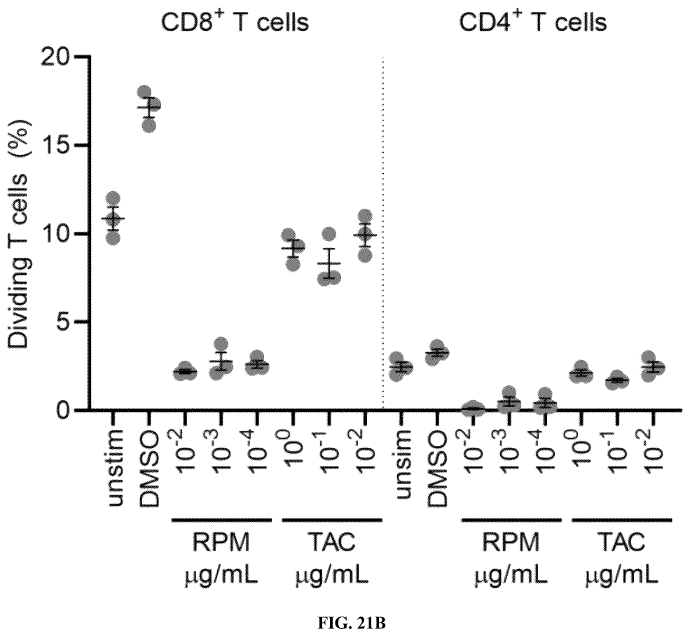

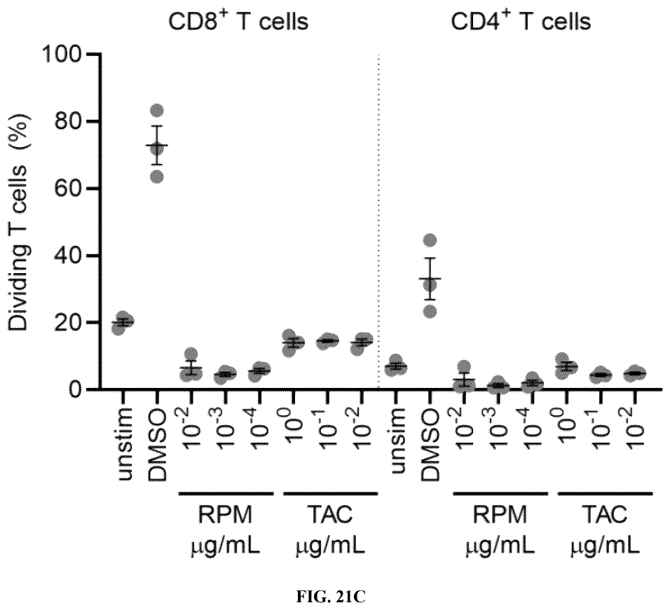

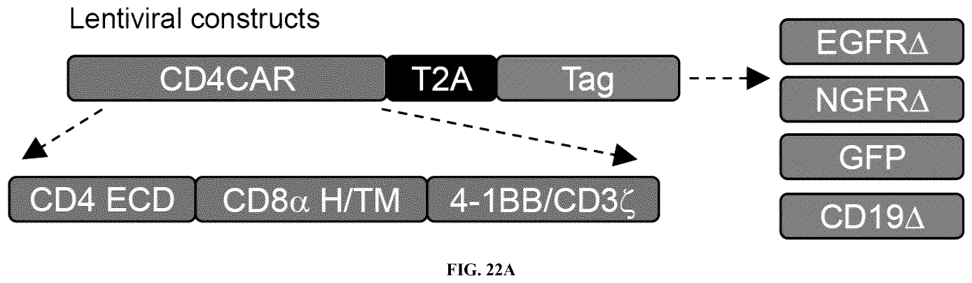

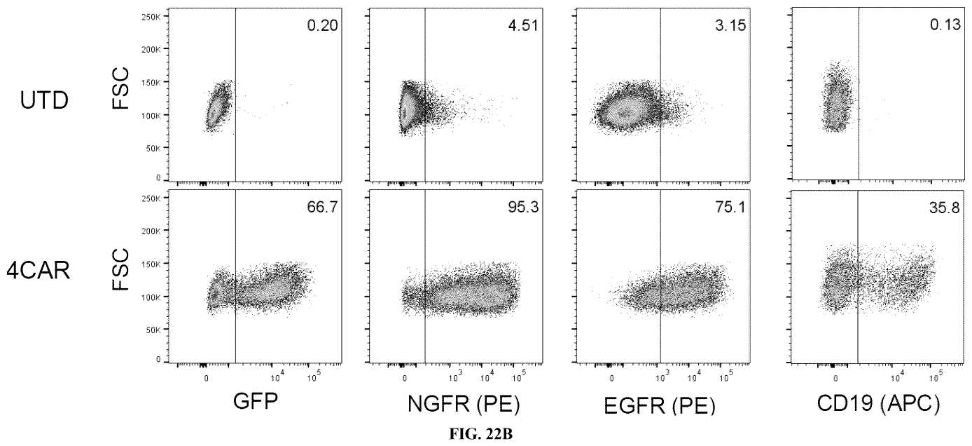

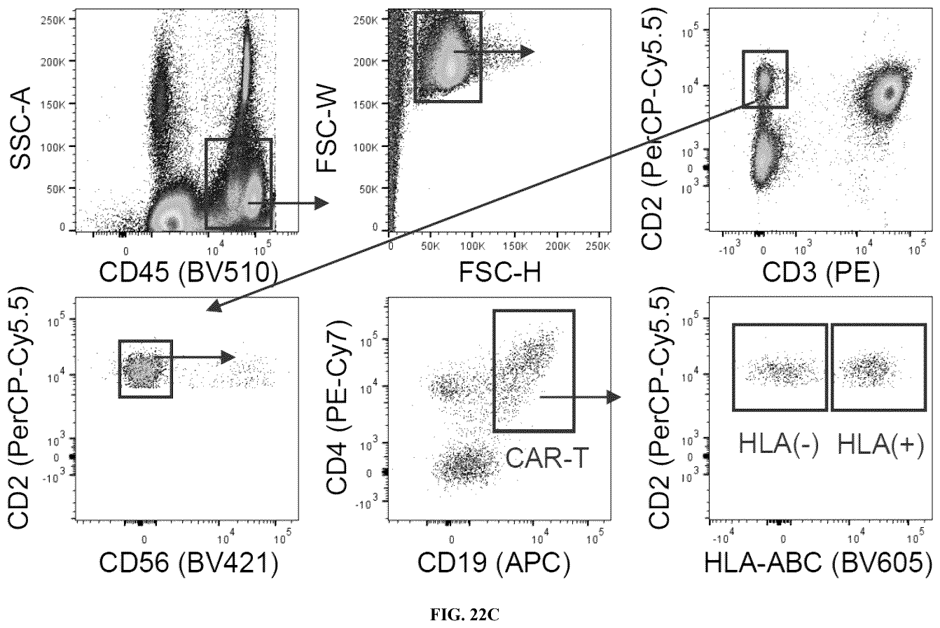

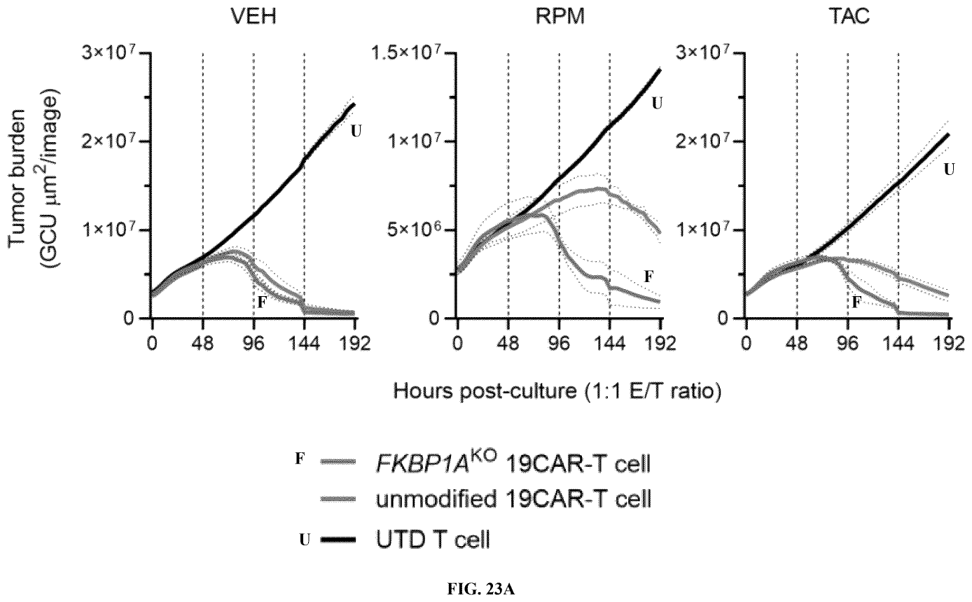

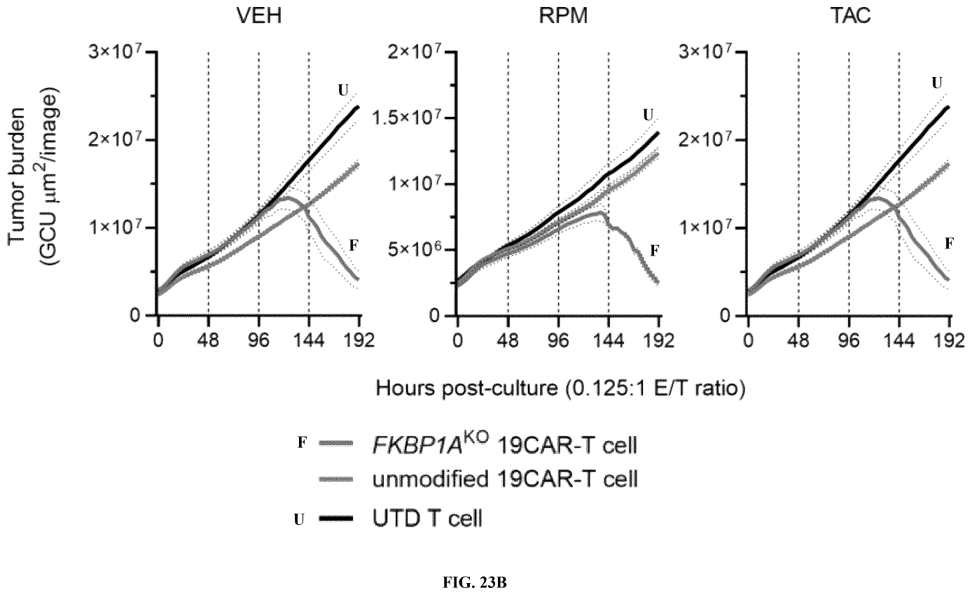

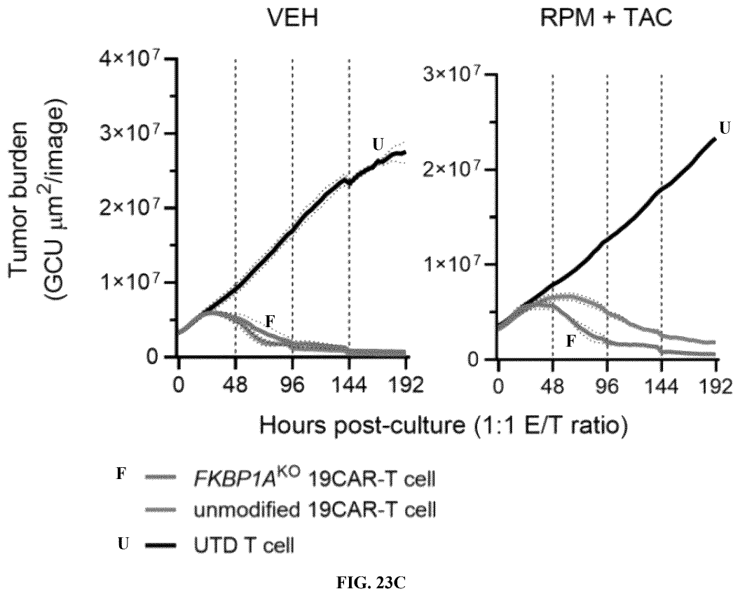

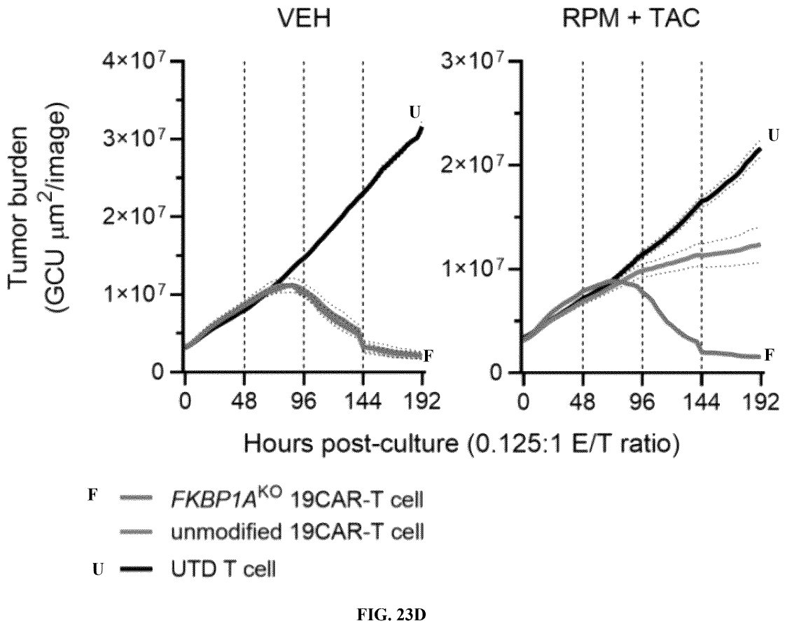

T cells were base edited to reduce or eliminate expression of PPIA by electroporating the cells with a guide polynucleotide selected from TSBT×6143, TSBT×6144, TSBT×6145, TSBT×6146, TSBT×6147, and TSBT×6148 (see Table 1 for guide sequences) and mRNA encoding the adenosine deaminase base editor ABE8.20m. Base editing of the NR3C1 polypeptide in the T cells was measured using next generation sequencing (FIG. 6). Two of the base editor systems achieved maximum base editing rates in excess of 40%, and one base editor system achieved maximum base editing rates in excess of 80%.

Knock-out of PPIA expression in T cells edited using a base editor system containing the guide TSBT×6143 or TSBT×6146 (see Table 1 for guide sequence) and the adenosine deaminase base editor ABE8.20m was confirmed using flow cytometry (FIG. 7).

### Example 4: Human Leukocyte Antigen Class I (HLA-I) Expression Dictated Susceptibility of Allogeneic Immune Cells to Rejection by T Cells or NK Cells

Allogeneic CAR-T cells expressing mismatched HLA haplotypes elicit responses by recipient T cells. Host T cell recognition was mitigated by ablating surface human leukocyte antigen class I (HLA-I) and class II (HLA-II) expression using base editing to knock-out (^{KO}) beta-2-microglobulin (b2M^{KO}) and class-II transcriptional activator (CIITA^{KO}), respectively (FIGS. 14A to 14C). HLA-deficient T cells were invisible to alloreactive T cells and resisted in vitro elimination in mixed leukocyte assays (FIGS. 14D and 14E).

Although the absence of surface HLA mitigated T cell-driven rejection, it was found that ablation of HLA-I, but not HLA-II, sensitized allogeneic T cells to in vitro NK cell-mediated lysis (FIGS. 14F and 14G) and elicited robust degranulation by NK cells (FIG. 14H). The data indicated that the NK cell compartment in human immune system (HIS) mice was underdeveloped and required treatment with recombinant human IL-15 to prime endogenous NK cells against b2M^{KO} T cells in vivo (FIGS. 20A to 20E). To avoid IL-15-induced T cell-driven GVHD in HIS mice, the impact of HLA-I deficiency was assessed instead in a humanized NK cell (huNK) only mouse model. To do so, primary human CD56⁺ NK cells were transplanted into NSG/IL-15 transgenic (tg) mice. After 2 weeks engraftment, allogeneic TCR^{KO} HLA-I⁺ and HLA-I⁻ (b2M^{KO}) T cells were co-infused into huNK mice and unreconstituted NSG/IL-15tg control mice (FIG. 14I). In contrast to HIS mice, huNK mice rapidly rejected b2M^{KO} T cells, while HLA-I⁺ T cells resisted elimination (FIG. 14J). Not intending to be bound by theory, together, these data support that the status of HLA-I expression on allogeneic cells largely dictated susceptibility to either T cell- or NK cell-mediated rejection.

### Example 5: Reconstitution of HLA-Like Molecules was Insufficient to Restrain NK Cell Activity Against HLA-I Deficient T Cells

Re-introduction of ligands for HLA-specific inhibitory receptors such as an invariant HLA single chain (^{SC}) molecule (i.e., single-chain dimers and trimers of the disclosure) is an attractive approach to restore tolerance against HLA-I deficient immune cells (FIG. 15A). However, broad implementation of this strategy may be challenged by the heterogeneous pattern of HLA-specific inhibitory receptors governing NK cell activity (FIG. 15B). Indeed, b2M^{KO} T cells expressing an HLA-Bw4^{SC}, HLA-C1^{SC}, HLA-C2^{SC}, or HLA-E^{SC} attenuated degranulation by NK cells that only express their respective HLA-specific inhibitory receptor (FIGS. 15C and 15D), but failed to reduce the net frequency of CD107a⁺ NK cells to levels observed by stimulation with unmodified allogeneic T cells (FIG. 15E). Furthermore, HLA^{SC} molecules did not substantially protect b2M^{KO} T cells from lysis by NK cells, unlike unmodified T cells which were not killed (FIG. 15F). Not intending to be bound by theory, these data suggest that the overall benefit of an HLA^{SC} was nullified due to the insufficient frequency of NK cell subsets expressing the corresponding HLA-specific inhibitory receptor, whereas broad inhibition required the full complement of HLA-I alleles.

### Example 6: Immunosuppression Treatment Mitigated T Cell-Mediated Allorejection of Human Leukocyte Antigen Class I Positive (HLA-I⁺) Chimeric Antigen Receptor-Expressing T Cells (CAR-T Cells)

Given that complete HLA-I expression broadly inhibited NK cell-driven rejection, an alternative strategy was investigated where allogeneic CAR-T cells retained HLA-I expression, while concomitant immunosuppressant treatment mitigated recipient T cell responses. It was first determined if immunosuppressants mitigate the in vitro priming of alloreactive T cells. To do so, peripheral blood mononuclear cells and CellTrace Violet-labeled T cells from HLA disparate donors were cultured in the presence of rapamycin (RPM), tacrolimus (TAC) or vehicle (VEH) control. As expected, the addition of immunosuppressants severely attenuated the proliferation of alloreactive T cells relative to vehicle (VEH) treatment (FIGS. 21A to 21C). Next, it was tested whether immunosuppressants alleviated in vivo T cell-driven rejection of allogeneic HLA-I CAR-T cells. Five cohorts of human immune system (HIS) mice derived from unrelated human donors were injected daily with RPM, TAC, or VEH for 2 weeks, and at 1-day post-treatment initiation all mice were co-infused with an equal amount of allogeneic HLA⁺ and HLA-deficient CAR-T cells (FIG. 16A). Cohorts #3 and #4 also received syngeneic human immune system (HIS) mouse-derived CAR-T cells to control for autologous T cell persistence. All T cell populations were engineered to express a non-targeting CD4-based CAR separated by a 2A self-cleaving peptide to a molecular tag that facilitated ex vivo identification by flow cytometry (FIGS. 22A to 22C) and allogeneic CAR-T cells were base-edited to disrupt TCR expression.

Both allogeneic CAR-T cell populations were equally detected in the peripheral blood of all groups at 1-day post-infusion, but thereafter, rapid depletion of circulating HLA⁺ CAR-T cells was observed in VEH-treated mice, whereas HLA-deficient CAR-T cells persisted for the study duration (FIGS. 16B and 16C). In contrast, RPM and TAC treatment significantly mitigated rejection of peripheral allogeneic HLA⁺ CAR-T cells at 1- and 2-weeks post-treatment initiation (FIGS. 16B and 16C). It was also observed that allogeneic HLA⁺ CAR-T cells from immunosuppressed mice persisted to the same degree as syngeneic CAR-T cells from within individual mice during the treatment window (FIG. 16D). Notably, the extent of circulating allogeneic HLA⁺ CAR-T cell preservation positively correlated with the contemporaneous plasma concentration of RPM and TAC (FIGS. 16E and 16F). Following treatment cessation, allogeneic HLA⁺ CAR-T cells were nearly undetectable in tissue indicating immunologic rejection (FIGS. 16G and 16H). Overall, at the chosen dosages TAC improved cumulative allogeneic HLA⁺ CAR-T cell persistence during the treatment window (FIG. 16I) and post-treatment withdrawal (FIG. 16J). These data demonstrated that RPM and TAC treatment extended the in vivo lifespan of allogeneic HLA⁺ CAR-T cells upon transfer into HLA-disparate lymphoreplete recipient mice.

An experiment was undertaken to further demonstrate that RPM and TAC treatment significantly mitigated allorejection of peripheral allogeneic HLA-ABC⁺ CAR-T cells by recipient humanized mice and that humanized mice treated with vehicle were capable of eliminating the HLA⁺ CAR-T, as evidenced by a reduction in HLA⁺ CAR-T levels in peripheral blood at 7 and 14 days post-infusion (FIG. 29). Humanized mice (huCD34-NCG; n=3) were treated with rapamycin (1 mg/kg) and tacrolimus (10 mg/kg) or received vehicle control (n=8) daily for 2 weeks via intraperitoneal injection. One day after drug-treatment initiation, mice were infused with a combination of 5 million HLA-positive allogeneic T cells expressing an anti-CD4 chimeric antigen receptor (i.e., HLA-ABC⁺ 4CAR-T cells) and base edited to knock-out (KO) T cell receptor (TCR) expression and 5 million HLA-negative 4CAR-T cells (i.e., HLA-ABC-4CAR-T cells) that were base-edited to KO TCR expression, beta-2-microglobulin (B2M) expression, and class-II transcriptional activator (CIITA) expression. The persistence of the 4CAR-T cells in peripheral blood in the mice was assessed by flow cytometry at 1, 7, and 14-days post-infusion using blood samples collected through puncture of the submandibular vein (FIG. 29).

The allogeneic 4CAR-T cells were generated via lentiviral transduction with a polynucleotide encoding an anti-CD4 chimeric antigen receptor (CAR) (i.e., 4CAR) containing an extracellular anti-CD4 antigen binding domain fused to a CD8a hinge domain, a CD8a transmembrane domain, a 4-1BB activating domain, and a CD3zeta activating domain. The 4CAR-T cells were base-edited by contacting the cells with a base editor system containing an mRNA molecule encoding the base editor ABE8.20m and one or more of the following guide RNAs: sgRNA TSBT×4073 (see Table 2B) to base edit the cells to knock out expression of CD3E and, thereby, knock out TCR expression; sgRNA TSBT×760 (see Table 2B) to base edit the cells to knock out expression of B2M and, thereby, knock out expression of HLA class-I polypeptides (e.g., HLA-A, HLA-B, and HLA-C); and sgRNA TSBT×763 (see Table 2B) to base edit the cells to knock out expression of CIITA and, thereby, knock out expression of HLA class-II polypeptides.

### Example 7: Genetic Disruption of FK506-Binding Protein 1A (FKBP1A) Rendered Chimeric-Antigen Receptor Expressing T Cells (CAR-T Cells) Resistant to Immunosuppression

To enable concomitant treatment with rapamycin (RPM) or tacrolimus (TAC), immunosuppressant resistant CAR-T cells were engineered by disrupting the gene encoding FK506-Binding Protein 1A (FKBP1A), which is an intracellular binding partner of RPM and TAC, and their interaction inhibits mTOR and calcineurin activation, respectively. FKBP1A-specific synthetic guide RNAs (sgRNA) were paired with mRNA encoding either an adenosine (ABE; ABE8.20m) or cytosine (CBE; rBE4) base editor and electroporated into activated primary human T cells (FIG. 3, and Tables 1A, 2A and 2B). This screen identified an ABE-sgRNA (TSBT×1538) complex targeting a conserved intron-exon splice junction that achieved a mean on-target genomic editing efficiency of 93.7% (FIG. 17A) and reduced protein expression (FIG. 4). Following activation, FKBP1A^{KO} T cells treated with RPM retained phosphorylation of the S6 ribosomal protein, a downstream substrate of the PI3K/Akt/mTOR pathway (FIGS. 17B and 17C), and maintained calcineurin induced NFAT-driven GFP expression after TAC treatment (FIGS. 17D and 17E) which, without intending to be bound by theory, indicates that FKBP 1A^{KO} abrogated proximal signaling events mediated by these immunosuppressants.

To evaluate whether FKBP1A^{KO} conferred T cells in vitro functional resistance to immunosuppressants, CD19-specific CAR-T cells (19CAR) were stimulated with JeKo-1 mantle cell tumors in the presence of RPM, TAC or dimethyl sulfoxide (DMSO) vehicle (VEH). Both FKBP1A^{KO} CD4⁺ and CD8⁺ 19CAR-T cells proliferated greater than unedited 19CAR-T cells, and notably, expanded to a similar extent as their VEH-treated counterparts (FIGS. 8, 9, and 17F). Furthermore, immunosuppressant treatment drastically reduced the magnitude and frequency of unedited 19CAR-T cells producing IFNg and TNFa, while FKBP 1A^{KO} 19CAR-T cells maintained high levels of cytokine production relative to VEH treatment (FIGS. 10, 11, 17G, and 17H). FKBP1A^{KO} 19CAR-T cells also overcame RPM and TAC inhibition to eradicate GFP⁺ JeKo-1 tumors with nearly the same kinetics as VEH-treated counterparts. In contrast, immunosuppressant treatment diminished the ability of unedited 19CAR-T cells to control tumor growth (FIGS. 12A, 12B, 171, 23A to 23D, 24A, and 24B). Additionally, it was evaluated whether FKBP1A^{KO} rendered 19CAR-T cells resistant to corticosteroids, an important treatment option for patients experiencing adverse events following CAR-T cell therapy. Both dexamethasone and prednisone suppressed antigen-driven proliferation and cytokine production of FKBP1A^{KO} 19CAR-T cells, indicating that FKBP1A^{KO} does not interfere with steroid-mediated suppression (FIG. 25). Collectively, these findings indicate that genetic ablation of FKBP1A renders CAR-T cells resistant to RPM and TAC induced immunosuppression. Further, these findings show that if the CAR-T cells cause side effects (e.g., cytokine release syndrome (CRS)) in a subject, steroids may be administered to the subject to reduce the side effects, and/or the side effects may be reduced by halting treatment of the subject with rapamycin/tacrolimus to facilitate clearance of the CAR-T cells via allorejection.

### Example 8: FK506-Binding Protein 1A Knock-Out (FKBP1A^{KO}) Anti-Cluster of Differentiation 19 Chimeric Antigen-Receptor-Expressing T Cells (19CAR-T Cells) Overcame Immunosuppression to Control Tumor Progression In Vivo

To evaluate whether FKBP1A^{KO} 19CAR-T cells resisted immunosuppressant treatment in vivo, luciferase expressing JeKo-1 tumors were transplanted into NSG mice and 1 week later daily injections of RPM, TAC or VEH were initiated for 2 weeks. One day post-treatment initiation, mice were infused with either FKBP1A^{KO} or unedited 19CAR-T cells, or untransduced (UTD) control T cells (FIG. 18A). Both 19CAR-T cell populations eradicated tumors in DMSO vehicle (VEH)-treated mice relative to mice that received untransduced (UTD) T cells (i.e., T cells not expressing any CAR). TAC treatment attenuated the ability of unedited 19CAR-T cells to control tumor outgrowth, and in stark contrast, FKBP1A^{KO} CAR-T cells potently suppressed tumor progression (FIGS. 13A, 13B, and 18B to 18D). Despite TAC treatment FKBP1A^{KO} CAR-T cells drastically reduced cumulative tumor burden during the treatment interval equivalent to their VEH-treated counterparts (FIGS. 18B to 18D), indicating that FKBP1A^{KO} conferred in vivo functional resistance to TAC.

Unlike TAC treatment, RPM administration mitigated JeKo-1 growth in control mice, which obscured the ability to perceive additive anti-tumor benefit in mice receiving FKBP1A^{KO} over unedited 19CAR-T cells (FIG. 18E). This observation is consistent with mature B cell malignancies exhibiting constitutive PI3K pathway activation and sensitivity to mTOR inhibitors. Indeed, RPM treatment attenuated protein phosphorylation of mTOR and downstream substrates S6Rp and 4E-BP1, as well as inhibited in vitro proliferation of JeKo-1, Raji and Nalm6 B cell cancer lines (FIGS. 26A to 26E). To overcome this obstacle, FKBP1A^{KO} JeKo-1 tumors were generated that exhibited comparable in vivo expansion kinetics to VEH treatment when in the presence of RPM (FIG. 18F). Now, FKBP1A^{KO} 19CAR-T cells in RPM-treated mice decreased cumulative tumor burden within the treatment interval to the same extent as in VEH-treated mice (FIG. 18G). These data demonstrate FKBP1A^{KO} 19CAR-T cells resisted immunosuppression and exhibit robust in vivo anti-tumor activity that mitigated disease progression.

### Example 9: Allogeneic FK506-Binding Protein 1A Knock-Out (FKBP1A^{KO}) Anti-Cluster of Differentiation 19 Chimeric Antigen-Receptor-Expressing T Cells (19CAR-T Cells) and Tacrolimus (TAC) Treatment Overcame Allorejection to Induce B Cell Aplasia In Vivo

It was assessed whether immunosuppressant treatment mitigated in vivo rejection of allogeneic HLA-I⁺ FKBP1A^{KO} 19CAR-T cells to an extent that permitted functional immune responses. Here, the endogenous B cell compartment of human immune system (HIS) mice was leveraged as an on-target population for 19CAR-T cells, where both the depth of B cell aplasia and the persistence of allogeneic cells measured treatment efficacy. Human immune system (HIS) mice from four human leukocyte antigen (HLA) disparate cohorts (#6-#9) were allocated into 4 groups that received UTD T cells and DMSO vehicle (VEH) treatment (Group 1), HLA⁺ FKBP1A^{KO} 19CAR-T cells and VEH treatment (Group 2), HLA⁺ FKBP1A^{KO} 19CAR-T cells and TAC treatment (Group 3), and HLA-deficient 19CAR-T cells and VEH treatment (Group 4) (FIG. 19A). TAC and FKBP1A^{KO} 19CAR-T cell treated mice (Group 3) exhibited lower CD19⁺ B cell counts in peripheral blood (FIGS. 19B and 19C), spleen (FIG. 19D) and bone marrow (FIG. 19E) compared to VEH-treated mice (Group 2), and to a near-equivalent level in mice treated with HLA-deficient 19CAR-T cells (Group 4). A significant reduction in CD19 surface density on residual B cells (FIG. 19F) and emergence of CD19^{dim}CD22⁺ B cells (FIG. 19G) from mice in Group 3 compared to the Group 2 control was also measured, indicating effective selection pressure by HLA-I⁺ 19CAR-T cells in the presence of TAC. Furthermore, TAC treatment protected HLA-I⁺ 19CAR-T cells in peripheral blood (FIG. 19I) and spleen (FIG. 19J) from recipient allorejection to an extent equivalent to HLA-deficient 19CAR-T cells. FIGS. 19I to 19H show that persistence of HLA-I⁺ 19CAR-T cells in mice treated with TAC was equivalent to persistence of HLA-I deficient 19CAR-T cells in mice treated with VEH. Together, these data demonstrate that TAC treatment conferred FKBP1A^{KO} CAR-T cells sufficient protection from immunologic rejection to deplete endogenous B cells.

The above results establish that the edited 19CAR-T cells could effectively eliminate tumor cells from a subject being administered an immunosuppressant agent. Administration of the immunosuppressant agent to the subject may have the advantage of suppressing the subject's immune system to prevent or reduce rejection of CAR-T cells by the subject's immune system.

### Example 10: Disruption of FKBP1A Expression in Immune Cells Using Cas12b

CRISPR-Cas12b nuclease and paired guide RNA (gRNA) may induce frameshift insertion/deletion (indel) mutations resulting in a premature stop codon that disrupts endogenous expression of FK506-Binding Protein 1a (FKBP1A) in allogeneic human immune cells (e.g. T cells). CRISPR-Cas12b may also be used to interrupt expression of a gene, such as FKBP1A, by inserting a polynucleotide (e.g., a polynucleotide encoding a chimeric antigen receptor or a transgene) into the gene locus. Accordingly, experiments are undertaken to demonstrate the disruption of FKBP1A expression in immune cells using a CRISPR-Cas12b nuclease.

Primary human T cells are first cultured at 10⁶ cells/mL in complete medium containing ImmunoCult™ XF T Cell Expansion Medium (Stem-Cell Technologies), 1% Penicillin-Streptomycin, 2 mM GlutaMax™, 25 mM HEPES Buffer (Life Technologies), and 5% CTS™ ImmuneCell SR (ThermoFisher). The complete medium also contains 5 ng/ml human IL-15 (Biolegend) and 10 ng/mL IL-7 (Biolegend). T cells are stimulated with ImmunoCult Human CD3/CD28/CD2 T Cell Activator (Stem-Cell Technologies) following the manufacturer's instructions and then incubated at 37° C., 5% CO², and 95% humidity. Two-days post-activation, T cells are counted, washed with sterile PBS (Gibco) and resuspended in P3 buffer (Lonza) at 10⁷ cells/mL. T cells are electroporated with 1 mg of FKBP1A-specific gRNA containing a Cas12b scaffold and a spacer listed in Table 2A, and 2 mg of mRNA encoding a Cas12b nuclease per 10⁶ cells using a Lonza 4D-Nucleofector® system (program DH-102). T cells are allowed to recover in complete medium at 10⁶ cells/mL, and the complete medium is exchanged every other day to adjust T cell concentration to 5×10⁵ cells mL-1 until day 10 when cells are cryopreserved in CS10 for later analysis.

### Example 11: Disruption of FKBP1A Expression in Immune Cells Using Prime Editing

A prime editor construct (see sequences provided in Table 12) and paired prime editing guide RNA (pegRNA), with or without a secondary nicking guide RNA (nRNA), can induce targeted, programmable changes to genomic DNA. These targeted changes may include frameshift insertions/deletions (indel mutations) resulting in a premature stop codon that disrupts endogenous expression of FK506-Binding Protein 1a (FKBP1A) in allogeneic human immune cells (e.g., T cells). Alternatively, or additionally, these targeted changes may include transversion and/or transition mutations that may disrupt expression of FKBP1A in allogeneic human immune cells (e.g. T cells) through various mechanisms, such as splice site disruption, start site disruption, active site disruption, promoter or enhancer disruption, protein structure disruption, or stop codon insertion. Additional mutations may be encoded into the prime editing guide RNA (pegRNA) to increase the frequency or product purity of a mutation introduced to a polynucleotide using prime editing. Methods for editing polynucleotide sequences using prime editing are well known in the art (see, e.g., Petrova I O, Smirnikhina S A. The Development, Optimization and Future of Prime Editing. Int J Mol Sci. 2023 Dec. 1; 24(23):17045. doi: 10.3390/ijms242317045, the disclosure of which is incorporated herein in its entirety by reference for all purposes).

Experiments are undertaken to disrupt expression of FKBP1A in T cells. Primary human T cells are first cultured at 10⁶ cells/mL in complete medium comprising ImmunoCult™ XF T Cell Expansion Medium (Stem-Cell Technologies), 1% Penicillin-Streptomycin, 2 mM GlutaMax™ and 25 mM HEPES Buffer (Life Technologies), and 5% CTS™ ImmuneCell SR (ThermoFisher). The complete medium also contains 5 ng/ml human IL-15 (Biolegend) and 10 ng/mL IL-7 (Biolegend). T cells are stimulated with ImmunoCult Human CD3/CD28/CD2 T Cell Activator (Stem-Cell Technologies) following the manufacturer's instructions and then incubated at 37° C., 5% CO², and 95% humidity. Two-days post-activation, T cells are counted, washed with sterile PBS (Gibco) and resuspended in P3 buffer (Lonza) at 10⁷ cells/mL. The T cells are electroporated with 1 mg of FKBP1A-specific prime editor guide RNA (pegRNA) (see sequences provided in Table 10), 0.5 mg of FKBP1A-specific nRNA (see sequences provided in Table 11), and 2 mg of mRNA encoding the prime editor construct (PE2 or PE3) per 10⁶ cells using the Lonza 4D-Nucleofector® system (program DH-102). T cells are allowed to recover in complete medium at 10⁶ cells/mL and medium is exchanged every other day to adjust T cell concentration to 5×10⁵ cells mL⁻¹ until day 10 when cells are cryopreserved in CS10 for later analysis.

The following materials and methods were employed in the above examples.

### Human Immune System (HIS) Mice

Female (aged 6-8 weeks) NOD.Cg-Prkdc^{scid}IL2rg^{tm1Wjl}/SzJ (NSG) mice (Jackson Laboratory) were maintained in a pathogen-free facility. Briefly, to generate BLT humanized mice, NSG mice were anesthetized and whole-body irradiated (2 Gy), and then implanted with 1 mm³ fragments of human fetal liver and thymus tissue beneath the murine kidney capsule. Following, 1×10⁵ autologous fetal liver-derived CD34⁺ hematopoietic stem cells (HSCs) were intravenously injected within 6 hours of transplantation. Beam Therapeutics commercially obtained female NOD-Prkdc^{em26Ca52} IL2rg^{em26CD22}/NjuCrl (NCG) CD34⁺ humanized (huNCG) mice from Charles River and female (aged 6-8 weeks) NSG-Tg(IL15)1Sz/SzJ (NSG-IL15tg) mice from Jackson Laboratory. Mice were maintained in a pathogen-free facility at CRADL. Briefly, huCD34-NCG mice were generated from female (aged 4-6 weeks) NCG mice that were myeloablated and then intravenously infused with human umbilical cord blood-derived CD34⁺ stem cells from a qualified source. Humanized NK (huNK) mice were generated by supplementing the water of NSG-IL-15tg with Baytril (Bayer) for 1 week followed by whole-body irradiation (2 Gy). 5×10⁶ primary human CD56⁺ NK cells were then intravenously injected 24 hours later and permitted to engraft. At both facilities, mice were housed in microisolator cages and fed autoclaved food and water. Animal rooms were maintained at 72±2° F., 30-70% relative humidity and were on a 12:12 h light/dark cycle. Human reconstitution was assessed from 12-17 weeks post-transplant in BLT and huNCG mice, and 2-3 weeks post-transplant in huNK mice. Mice were included in studies when ≥25% of cells in the lymphocyte gate were human CD45⁺ in BLT and huNCG mice, and when human CD56⁺ cells achieved 10 cells mL⁻¹ blood in huNK mice.

### HIS Mouse-Driven Allorejection Model

For the studies described in FIGS. 16A to 16J, BLT mice were used Cohort #2 (n=12) and Cohort #5 (n=13), and huNCG mice were used in Cohort #1 (n=10), Cohort #3 (n=12) and Cohort #4 (n=10). HIS mice from each cohort were allocated into groups via matched distribution based on degree of human T cell engraftment using StudyLog software (Studylog Systems) and received daily intraperitoneal injections of rapamycin (RPM; 1 mg kg-1), tacrolimus (TAC; 10 mg kg-1), or vehicle (VEH) control for 2 weeks. Each HIS mouse cohort represented an independent study where mice in Cohort #1 received VEH (n=5) or RPM (n=5); Cohort #2 received VEH (n=5) or RPM (7); Cohort #3 received VEH (n=4), RPM (n=4), or TAC (n=4); Cohort #4 received VEH (n=4), RPM (n=3), or TAC (n=3); and Cohort #5 received VEH (n=7) or TAC (n=6). One day post-drug treatment initiation, HIS mice were intravenously infused with a unique allogeneic human donor-derived T cell product comprising 5×10⁶ TCR^{KO} 4CAR-T cells and 5×10⁶ TCR^{KO}b2M^{KO}CIITA^{KO} 4CAR-T cells. Mice in Cohort #3 and Cohort #4 were also infused with 5×10⁶ syngeneic mouse-derived 4CAR-T cells. Persistence of 4CAR-T cells was monitored at 1-day post-T cell infusion and weekly thereafter via blood draws from the retro-orbital sinus or submandibular vein. HIS mice in Cohort #5 were necropsied 24 hours after final drug treatment and tissues were collected to analyze 4CAR-T cells.

For the studies described in FIGS. 19A to 19J, huCD34-NCG mice were used in Cohort #6 (n=16), Cohort #7 (n=12), Cohort #8 (n=24), and Cohort #9 (n=12) and were evenly distributed into 4 groups via matched distribution based on degree of human T cell engraftment using StudyLog. HIS mice in Group #1 received VEH and 5×10⁶ untransduced (UTD) T cells; Group #2 received VEH and 5×10⁶ TCR^{KO}b2M^{KO}CIITA^{KO} 19CAR-T cells; Group #3 received TAC and 5×10⁶ TCR^{KO}FKBP1A^{KO} 19CAR-T cells; and Group #4 received VEH and 5×10⁶ TCR^{KO}b2M^{KO}CIITA^{KO} 19CAR-T cells. HIS mice were treated daily with TAC (10 mg kg⁻¹) or VEH for 11 days via intraperitoneal injections and at 1-day post-drug treatment initiation mice were intravenously infused with T cells derived from an allogeneic human donor. Endogenous B cell aplasia and persistence of 19CAR-T cells was monitored at 7- and 11-days post-drug treatment via blood draw from the submandibular vein and tissue collection at necropsy.

### NK Cell Rejection Model

For the study described in FIGS. 20A to 20E, BLT mice received intraperitoneal injections every 2-3 days of 2.5 mg recombinant human IL-15 (BioLegend; n=6) or sterile PBS (n=3) for 6 total injections. Following, all mice were intravenously infused with an allogeneic human donor-derived T cell product comprising 5×10⁶ TCR^{KO} 4CAR-T cells and 5×10⁶ TCR^{KO}b2M^{KO}CIITA^{KO} 4CAR-T cells. Persistence of 4CAR-T cells was monitored at 1-, 4- and 7-days post-T cell infusion via blood draw from the retro-orbital sinus. For the study described in FIGS. 1H to 1J, huNK cell mice (n=5) and unreconstituted NSG-IL15tg control mice (n=5) were intravenously infused with an allogeneic human donor-derived T cell product comprising 5×10⁶ TCR^{KO} 4CAR-T cells and 5×10⁶ TCR^{KO}b2M^{KO}CIITA^{KO} 4CAR-T cells. Persistence of 4CAR-T cells was monitored at 1-day post-T cell infusion and weekly thereafter via blood draw from submandibular vein.

### Anti-Tumor Efficacy Model

For the study described in FIGS. 18A to 18G, NSG mice were intravenously injected with 5×10⁵ JeKo-1.Luc cells at day 0. On day 7, mice initiated daily intraperitoneal injections of VEH, RPM (1 mg kg⁻¹), or TAC (10 mg kg⁻¹) for 2 weeks. Mice from each treatment cohort were then injected on day 8 with 1×10⁶ UTD T cells, unmodified 19CAR-T cells, or FKBP1A^{KO} 19CAR-T cells (n=8 per group). For the study described in FIGS. 18F and 18G, NSG mice were intravenously injected with 5×10⁵ JeKo-1.FKBP1A^{KO}.Luc cells at day 0. On day 7, mice initiated daily intraperitoneal injections of VEH or RPM (1 mg kg⁻¹) for 2 weeks. Mice from each treatment cohort were then injected on day 8 with 1×10⁶ UTD T cells, unmodified 19CAR-T cells, or FKBP1A^{KO} 19CAR-T cells (n=10 per group). For all studies, tumor burden was measured every 3-4 days post-implant by bioluminescence imaging (IVIS Spectrum, Spectral Instruments Imaging) 30 minutes after intraperitoneally injecting mice with 150 mg kg⁻¹ XenoLight D-Luciferin (PerkinElmer). The acquisition time was automatically determined by the instrument for each exposure, and quantification of flux from imaging datasets was performed with the Living Image Studio software (Perkin Elmer). Briefly, a constant region-of-interest (ROI) was drawn over the mouse and flux was reported as total photon per second (ph/s).

### In Vivo Drug Formulation

Rapamycin (Thermo Fisher Scientific) and Tacrolimus (Cayman Chemical) were reconstituted in DMSO (Sigma-Aldrich; vehicle) at 10 mg mL⁻¹ and 25 mg mL⁻¹, respectively and 0.22 mm sterile-filtered. Rapamycin was diluted to 0.15 mg mL⁻¹ and Tacrolimus was diluted to 1.5 mg mL⁻¹ using sterile-filtered vehicle solution comprising a 1:1 ratio of 5% (v/v) Polyethylene Glycol (Sigma-Aldrich) and 5% (v/v) TWEEN-80 (Sigma Aldrich). PEG-TWEEN solution served as vehicle control and percent volume DMSO was normalized across all drug treatments.

### Mass Spectrometry

For the study described in FIGS. 3E and 3F, plasma concentrations of rapamycin and tacrolimus were determined using liquid chromatography-mass spectrometry (LC-MS). Mouse plasma from whole blood 1-week post-T cell infusion was isolated by centrifugation (2200×g, 5 minutes, 4° C.) and then cryopreserved. Thawed samples were injected into an Agilent Infinity 1290 II liquid chromatography coupled with a ScieX 6500+triple quad mass spectrometer. Briefly, the plasma, calibration curve (CC), and quality control (QC) samples were prepared by aliquoting 5 μL per sample into a 96-well plate. CC standards were prepared in mouse plasma (BioIVT, Part #) at 0.5, 1, 5, 25, 100, 500, 900, 1000 ng mL⁻¹. QC samples were prepared in mouse plasma at 1.5 ng mL⁻¹, 75 ng mL⁻¹, and 750 ng mL⁻¹. Tolbutamide was used as the internal standard and 100 μL of a 10 ng mL⁻¹ stock solution prepared in acetonitrile was added to each well except the wells containing 5 μL blank mouse plasma where 100 μL of acetonitrile was instead added. The plate was sealed and vortexed (1650 rpm, 3 minutes), and subsequently centrifuged (3500 rpm, 10 minutes) at room temperature. 50 μL of supernatant was transferred to a separate 96-well collection plate containing 50 μL water per well. The plate was vortexed (1600 rpm, 1 minute) and samples were injected into the LC-MS/MS system for analysis.

The separation column was a Zorbax SB-Phenyl, 1.7 μm, 40×2.1 mm column (Agilent). The mobile phase consisted of water containing 0.1% formic acid (Mobile Phase A) and methanol containing 0.1% formic acid (Mobile Phase B). The flow rate was 0.8 mL min-1 with an operating column temperature of 50° C. The gradient was from 25-80% B in 1 minute, then 80-98% B in 0.7 minute followed by a 1 minute hold, then 98-80% B in 0.7 minutes followed by a 0.5 minute hold, and finally brought back to 25% B in 0.5 minutes followed by 1.35 minutes of re-equilibration.

The MS Instrument was operated in multiple reaction monitoring (MRM) mode and positive electrospray ion mode with the following ion source conditions: curtain gas, 35 psi; gas 1, 70 psi; gas 2, 80 psi; ion spray voltage, 5500 V; and temperature, 500° C. The MRM transition and collision energy were m/z 936.6 >409.3 and 74 V for rapamycin, m/z 826.6 >616.4 and 48 V for tacrolimus, and m/z 271.0 >155.1 and 25 V for the internal standard Tolbutamide.

### Plasmid Construction

The transgene cassette comprising a CD4-based CAR (4CAR) construct containing the intracellular 4-1BB/CD3ζ intracellular domain and molecular tag comprising NGFRD, EGFRD, CD19D or GFP separated by an intervening T2A linker is described in Leibman, et al., PloS Pathog. 13:e1006613, (2017) PMID: 29023549, the disclosure of which is incorporated herein by reference in its entirety for all purposes. The CD19-specific CAR (19CAR) comprised the FMC63 single chain variable fragment (see Nicholson, et al., Mol Immunol., 34:1157-65 (1997), PMID: 9566763, the disclosure of which is incorporated herein by reference in its entirety for all purposes), CD8a hinge and transmembrane domains and 4-1BB/CD3ζ intracellular domain and was separated by an intervening T2A linker to truncated EGFR (see Wang, et al., Blood, 118:1255-63 (2011), PMID: 21653320, the disclosure of which is incorporated herein by reference in its entirety for all purposes). To generate the 6×NFAT reporter plasmid, six NFAT response elements (GGAGGAAAAACTGTTTCATACAGAAGGCGT (SEQ ID NO: 773)) were placed upstream of the murine IFN-b promoter driving expression of GFP. HLA single chain molecules were cloned into an expression cassette upstream of a T2A link and truncated EGFR selection tag. Single chain molecules are fusion proteins consisting of the b2M signal peptide, b2M, (G₄S)₃ (SEQ ID NO: 486) linker, and the HLA extracellular, transmembrane, and cytoplasmic domains. Identity of the HLA alleles include HLA-Bw4⁺: HLA-B*57:01; HLA-C1 group: HLA-C*01:02 and *07:02; HLA-C2 group: HLA-C*04:01, *05:01, *06:02 and *18:01; and HLA-E*01:03. HLA-E single chain comprised an HLA-G*01 leader peptide (VMAPRTLFL (SEQ ID NO: 774)) situated between the signal peptide and b2M chain. HLA allele amino acid sequences are from the IPD-IMGT/HLA database. All gene fragments were custom synthesized and cloned by GenScript into a third-generation self-inactivating (SIN) lentiviral vector.

### Lentivirus Production

Lentiviral particles were generated using packaging expression vectors from Aldevron: VSV glycoprotein (pALD-VSV-G), HIV Rev (pALD-Rev) and HIV Gag/Pol (pALD-GagPol). The packaging plasmids along with the appropriate SIN transfer vector were transfected into HEK293T cells using Lipofectamine 2000 (Life Technologies). At 24 hours post-transfection, the HEK293T cell supernatant was collected, filtered through a 0.45-mm syringe-driven filter, mixed with PEG-it Virus Precipitation Solution (System Biosciences) and stored at 4° C. overnight following the manufacturer's instructions. After incubation, the virus solution was concentrated by centrifugation for 30 minutes at 1,500×g, 4° C. The supernatant was aspirated and the virus pellet was resuspended in 600 μl of complete Immunocult-XF T Cell Expansion Medium and stored at −80° C.

### T Cell Generation, Base Editing and Lentiviral Transduction

Healthy donor adult human leukopaks were commercially obtained from HemaCare (Charles River). T cells were isolated using StraightFrom® Leukopak® CD4/CD8 MicroBead Kit (Milenyi Biotec) following the manufacturer's protocol and cryopreserved in CS10 (Stem-Cell Technologies). T cells were thawed and placed in culture at 10⁶ cells mL⁻¹ in complete medium comprising ImmunoCult XF T Cell Expansion Medium (Stem-Cell Technologies), 1% Penicillin-Streptomycin, 2 mM GlutaMax and 25 mM HEPES Buffer (Life Technologies), and 5% CTS ImmuneCell SR (ThermoFischer). Complete medium was complemented with 5 ng mL⁻¹ human IL-15 (Biolegend) and 10 ng mL⁻¹ IL-7 (Biolegend). T cells were stimulated with ImmunoCult Human CD3/CD28/CD2 T Cell Activator (Stem-Cell Technologies) following manufacturer's instructions and incubated at 37° C., 5% CO² and 95% humidity. Two-days post-activation, T cells were counted, washed with sterile PBS (Gibco) and resuspended in P3 buffer (Lonza) at 10⁷ cells mL⁻¹. T cells were electroporated with 1 mg synthetic guide RNA (sgRNA) from Agilent and 2 mg mRNA encoding ABE8.20m or rBE4 per 10⁶ cells using the Lonza 4D Nucleofector system (program DH-102). Base editing with greater than 1 sgRNA was achieved by adding 1 mg of the additional sgRNA(s) per 10⁶ cells to the electroporation reaction. T cells then recovered in complete medium at 10⁶ cells mL-1 and were transduced with 300 mL of concentrated lentiviral vector per 10⁶ cells. Medium was exchanged every other day to adjust T cell concentration to 5×10⁵ cells mL⁻¹ until day 10 when cells were cryopreserved in CS10. Sequences for the 20 nucleotide spacers specific for CD3E, TRAC, b2M, CIITA, and FKBP1A can be found in Tables 2A and 2B. T cell products for in vivo studies were generated using CD3E, b2M (TSBT×760), CIITA sgRNAs and ABE8.20m mRNA (FIGS. 14H to 14J and FIGS. 16A to 16J); FKBP1A sgRNA and ABE8.20m mRNA (FIG. 5); and CD3E, b2M, CIITA, FKBP1A sgRNAs and ABE8.20m mRNA (FIGS. 19A to 19J). All T cell products for in vitro assays were generating using b2M, CIITA, FKBP1A sgRNAs and ABE8.20m mRNA.

### Next-Generation Sequencing (NGS) or Genomic DNA Samples

Genomic DNA (gDNA) samples were prepared to determine base editing efficiency as described in Diorio, et al., Blood, 140:619-629 (2022), PMID: 35560156, the disclosure of which is herein incorporated by reference in its entirety for all purposes. Briefly, 0.5-1×10⁶ cells were lysed using QuickExtract DNA Extraction Solution (Lucigen) according to the manufacturer's protocol. Two microliters of gDNA were added to a 25 mL polymerase chain reaction (PCR) containing Q5 High-Fidelity DNA Polymerase (New England Biolabs) and 0.5 mM forward and reverse primers. Primer sequences for gDNA amplification are listed in Table 9. PCR amplicons were then amplified using unique Illumina barcoding primer pairs, and then the resulting product was purified using Solid Phase Reversible Immobilization beads (Beckman Coulter) and quantified using a NanoDrop 1000 Spectrophotometer (Thermo Fischer Scientific). Barcoded amplicons were sequenced on an Illumina MiSeq instrument according to manufacturer's instructions. PCR amplification conditions are described in Gaudelli, et al. Nature, 551:464-471 (2017), PMID: 29160308, the disclosure of which is incorporated herein by reference in its entirety for all purposes.

Base Editor mRNA Production

mRNA production for adenosine (ABE8.20m) and cytosine (rBE4) base editors was performed as described in Gaudelli, et al. Nat Biotechnol. 38:892-900 (2020) PMID: 32284586, the disclosure of which is incorporated herein by reference in its entirety for all purposes. Briefly, editors were cloned into a plasmid encoding a T7 promoter, 5′ UTR, Kozak sequence, open reading frame encoding the editor, and 3′ UTR. Plasmids were linearized using BbsI-HF (New England Biolabs) and purified using DNA Clean and Concentrate Columns (Zymo Research). Linearized plasmid served as template for in vitro transcription with HiScribe T7 High-Yield RNA Synthesis Kit (New England BioLabs) following the manufacturer's instructions except cotranscriptional capping was performed with CleanCap AG (TriLink Biotechnologies). mRNA was purified using lithium chloride precipitation.

### Tumor Cell Line Generation

JeKo-1 (CRL-3006™), Raji (CCL-86™) and Nalm6, clone G5 (CRL-3273™) were obtained from the ATCC. All three cell lines were transduced with a SIN lentiviral vector encoding both GFP and Click Beetle Green luciferase (Luc). Nalm6 cells were also transduced with a lentiviral encoding iRFP670. To generate JeKo-1.FKBP1A^{KO}.Luc cells, parental JeKo-1. Luc cells were electroporated with 1 mg TSBT×1538 sgRNA complexed and 2 mg ABE8.20m mRNA using the Lonza 4D Nucleofector system (SF buffer, program DJ-105). To generate Nalm6.CD19^{KO}.iRFP670 cells, parental Nalm6.iRFP670 cells were electroporated with 1 mg TSBT×3773 sgRNA complexed and 2 mg ABE8.20m mRNA using the Lonza 4D Nucleofector system (SF buffer, program CV-104). All tumor cells were sorted on GFP or iRFP670 positivity, or CD19-negative surface expression using the Aria Phusion (BD Bioscience) to obtain a clonal population. Single clones were analyzed by next-generation sequencing to confirm FKBP1A disruption.

### Mixed Leukocyte Reaction

Alloreactive T cells were generated in FIGS. 14D and 14E by culturing CD3⁺ T cells (effectors) with mismatched HLA-A*02⁺ CD3-depleted PBMCs (targets) from a separate donor in duplicate. CD3 selections were performed using CD3 MicroBeads (Milenyi Biotec) following manufacturer's protocol. Effector and target cells were mixed at a 1:1 ratio in complete medium supplemented with 300 U mL⁻¹ IL-2 (Sartorius) for 7-10 days. Activated HLA-A*02⁻ effector T cells were then cultured at different ratios with PBMC donor-matched HLA-A*02⁺ T cells that were unmodified HLA-A*02⁺ (on-target) or HLA-deficient b2M^{KO} CIITA^{KO} (off-target) and labelled with CellTrace Far Red (Thermo Fisher Scientific). On- and off-target T cells were combined at 1:1 ratio before adding effector T cells to measure the relative frequency of target T cells within the same well 48 hours post-culture using flow cytometry.

For the study described in FIGS. 21A to 21C, CellTrace Violet (Thermo Fisher Scientific) CD3⁺ T cells (effectors) and CD3-depleted PBMCs (targets) from HLA disparate donors were cultured in triplicate at a 1:1 ratio in complete medium supplemented with 300 U mL⁻¹ IL-2. Cells were cultured in the presence of RPM at 10⁻², 10⁻³ and 10⁻⁴ mg mL⁻¹, TAC at 10⁰, 10⁻¹, and 10⁻² mg mL⁻¹, and DMSO control. Effector T cells cultured in the absence of target cells served as an unstimulated control. Frequency of dividing effector CD8⁺ and CD4⁺ T cells was measured at day 5 and 7 post-stimulation by flow cytometry.

### NK Cell Cytotoxicity and Degranulation Assays

Human NK cells were isolated using StraightFrom® Leukopak® REAlease CD56 MicroBead Kit (Milenyi Biotec) or CD56 MicroBeads (Miltenyi Biotec). NK cells were primed for 3 days in complete medium with 5 ng mL⁻¹ IL-15 and 300 U mL⁻¹ IL-2. Primed NK cells were cultured at different ratios with allogeneic T cells that were unmodified HLA⁺ (off-target) or HLA-deficient b2M^{KO} CIITA^{KO} (on-target). On- and off-target T cells were combined at 1:1 ratio before adding primed NK cells to measure the relative change in frequency of target T cells compared to control wells in the absence of NK cells. Specific lysis of on-target T cells was measured 48 hours post-culture by flow cytometry using the formula: Specific Lysis (%)=100−(100×(% survival in the presence of NK cells/% survival in the absence of NK cells)). NK cell degranulation was measured by culturing primed NK cells with on-target or off-target allogeneic T cells, or alone. Anti-CD107a antibody was added at the start of stimulation followed by the addition of 1× Monensin Solution and 1× Brefeldin A (BioLegend) 1 hour later. Cells were incubated for 6 hours total before analysis by flow cytometry.

### CAR-T Cell Cytotoxicity Assays

Cryopreserved untransduced (UTD) T cells, unmodified 19CAR-T cells, and FKBP1A^{KO} 19CAR-T cells were thawed and rested overnight at 37° C., 5% CO₂. On the day of assay set-up, a 96-well flat bottom plate (Corning) was coated with 0.01% Poly-L-Ornithine solution (Sigma Aldrich) for 30 minutes, and then decanted and left to dry for 30 minutes. Afterwards, 2.5×10⁴ JeKo-1.FKBP1A^{KO}.GFP⁺ tumor cells were seeded into the 96-well plate and cultured in triplicate with T cells at 0:1, 1:1, 0.25:1 and 0.125:1 effector-to-target cell (E/T) ratios. Rapamycin (100 nM), tacrolimus (100 ng mL-1) and vehicle (DMSO) control were added at the start of culture and then again at 48, 96 and 144 hours post-culture. Samples were transferred to Incucyte SX5 Live-Cell Analysis System (Sartorius) and imaged every 4 hours to assess GFP fluorescence intensity.

To perform the VITAL cytotoxicity assay, 2.5×10⁴ Nalm6.CD19^{WT}.GFP⁺ cells and 2.5×10⁴ Nalm6.CD19^{KO}.iRFP670⁺ cells were seeded per well of a 96-well flat bottom plate. UTD T cells, unmodified 19CAR-T cells, and FKBP1A^{KO} 19CAR-T cells were added at 0:125:1, 0.06:1, 0.03:1, 0.015:1, and 0:1 E/T ratios. Samples were incubated at 37° C., 5% CO₂ for 48 hours before flow cytometric analysis to measure the frequency of viable GFP⁺ and iRFP670⁺ Nalm6 tumor cells. Specific lysis of Nalm6.GFP⁺ cells was measured using the formula: Specific Lysis (%)=100−(100×(% survival in the presence of T cells/% survival in the absence of T cells)).

### CAR-T Cell Cytokine Production Assay

Cryopreserved unmodified and FKBP1A^{KO} 19CAR-T cells were thawed into complete medium and rested overnight at 10⁶ cells mL⁻¹ in the incubator at 37° C., 5% CO₂. T cells were pre-treated overnight with rapamycin (100 nM), tacrolimus (100 ng mL⁻¹), or vehicle (DMSO) control prior to assay set-up. The following day, T cells were washed, counted and 1×10⁵ cells were seeded in duplicate into a 96-well flat bottom plate alone or with 2×10⁵ JeKo-1.GFP⁺ tumor cells. Anti-CD107a antibody was added at the start of stimulation followed by the addition of 1× Monensin Solution and 1X Brefeldin A (BioLegend) 1 hour later. Cells were incubated for 6 hours total before analysis by flow cytometry.

### CAR-T Cell Proliferation Assay

Cryopreserved unmodified and FKBP1A^{KO} 19CAR-T cells were thawed into complete medium and rested overnight at 10⁶ cells mL⁻¹ in the incubator at 37° C., 5% CO₂. The following day, T cells were washed, counted and 1×10⁴ cells were seeded in triplicate into a 96-well flat bottom plate alone or with 1×10⁴ JeKo-1.GFP⁺ tumor cells. Complete medium was supplemented with rapamycin (100 nM), tacrolimus (100 ng mL⁻¹), or vehicle (DMSO) control and refreshed on day 2, 4 and 6 post-culture. On day 7, samples were analyzed by flow cytometry and 19CAR-T cells were enumerated using CountBright Counting Beads (Thermo Fisher Scientific) following the manufacturer's protocol.

### Flow Cytometry

Cultured cells were washed and stained in 50 mL of 1×PBS containing 2 mM EDTA and 2% fetal calf serum, and whole blood was stained directly with anti-human antibodies from BioLegend: CD45 (HI30), CD3 (OKT3), CD2 (RPA-2.10), CD4 (OKT4), CD8 (SK1), CD56 (5.1H11), CD107a (H4A3), CD19 (HIB19), CD22 (HIB22), HLA-A2 (BB7.2), HLA-DR (L243), NGFR (ME20.4), EGFR (AY13), NKG2A (S19004C), LIR-1 (GHI/75), HLA-ABC (W6/32), KIR2DL1 (HP-DM1), KIR2DL2/L3 (DX27), KIR3DL1 (DX9), KIR2DL4 (mAB33), KIR2DL5 (UP—R1). Live cells were discriminated by staining negative for Fixable Viability Dye eFluor780 (eBioscience). CountBright Counting Beads were used according to the manufacturer's instructions to determine concentration of immune cells from whole blood. Intracellular cytokines were detected using Cell Fixation & Cell Permeability Kit (Invitrogen) following manufacturer's protocol with anti-human antibodies from Biolegend: TNF-a (MAb11), IFN-g (4S.B3), IL-2 (MQH-17H12) and GM-CSF (BVD2-21C11). Phosphorylated intracellular proteins using BD Cytofix Fixation Buffer and BD Phosflow Perm Buffer III (BD Biosciences) according to the manufacturer's instructions with anti-human antibodies from BD Biosciences: mTOR-pS2448 (021-404), S6-pS235/pS236 (N7-548), 4EBP1-pT36/pT45 (M31-16). Flow cytometry data was acquired on the MACSQuant Analyzer 16 (Miltenyi Biotec) and FACSymphony A3 Cell Analyzer (BD Biosciences). Data was analyzed using FlowJo software v.10 (Tree Star).

### Phosphorylated

Prior to staining T cells, the culture medium was supplemented with DMSO or rapamycin (10 mM) for 2 hours and then of Immunocult T Cell Activator (1:40 dilution) for an additional hour.

### Statistical Analysis

Comparison of matched samples were performed using two-sided non-parametric Wilcoxon matched-pairs signed rank test. Comparison of unmatched samples were performed using two-sided non-parametric Wilcoxon rank sum test or Kruskal-Wallace test followed by Dunn's test for multiple comparisons. Bivariate correlations were performed using two-sided Spearman's rank correlation. Area under the curve calculations were performed using either cell concentration per 1 mL blood or frequency of cells. All statistical analyses were performed using GraphPad Prism version 9.3.0 (GraphPad).

Design of pegRNA and nRNA Molecules

The pegRNA molecules of Example 11 were designed to have reverse transcriptase template (RTT) lengths of between 10 and 30 nucleotides and primer binding sequences (PBS) lengths of between 10 and 15 nucleotides. Software, such as pegFinder is commercially available to assist in the design of pegRNA molecules. Two spacer sequences were identified that had a nick-to-edit distance less than 15, and the pegRNA molecules were designed to contain one of these spacer sequences. The spacer sequences were GAAACCAUCUCCCCAGGAGA (Spacer 1; SEQ ID NO: 803) and CAGGUGGAAACCAUCUCCCC (Spacer 2; SEQ ID NO: 804). In some embodiments, Spacer 2 further comprises a 5′ G. The 228 pegRNA sequences of Table 10 (see, also FIGS. 27A, 27B, and 28) were designed to each containing Spacer 1 or Spacer 2 and different RTT/PBS lengths. In some cases, synonymous edits were introduced into the RTT to evade cellular mixed-match (MM) repair (MMR). The RTTs spanned 3 codons and 17 bases of intronic sequence of the FKPB1 gene (FIG. 28). The target sequence for editing was CTCaCCGTCTCCTGGGGAGA TGG (TSBT×1530; human chr20 positive strand positions 1392956-1392979; SEQ ID NO: 805), where a protospacer-adjacent motif is shown in bold, and where position 4, which is shown as a lowercase “a,” was the target nucleobase for editing. The pegRNA molecules contained the following scaffold sequence having the prefix (5′ terminal sequence) gtttt and having the suffix (3′ terminal sequence) gtgc: guuuuagaGCUAGAAAUAGCAAGUUAAAAUAAGGCUAGUCCGUUAUCAACUUGAAAAAGUGGCA CCGAGUCGgugc (SEQ ID NO: 806). In some embodiments, the pegRNA molecule contains a scaffold containing one of the following sequences:

(SEQ ID NO: 807)
GUUUUAGAGCUAGAAAUAGCAAGUUAAAAUAAGGCUAGUCCGUUAUCAA
CUUGAAAAAGUGGGACCGAGUCGGUCC,
(SEQ ID NO: 108)
agaGCUAGAAAUAGCAAGUUAAAAUAAGGCUAGUCCGUUAUCAACUUGA
AAAAGUGGCACCGAGUCG.

Since silent mutations at various positions in the RTT of pegRNA molecules are known to increase edit rates (X Li, et al., Nature Communications 2022), a number of the pegRNA sequences of Table 10 contain such silent mutations. For introducing silent mutations, any mismatch at any interval may be introduced within non-coding regions of a gene; however, mutations within a coding region (see FIG. 28) must correspond to synonymous mutations, as listed, e.g., in Table 8 below:

TABLE 8
Synonymous codon sequences.

| Codon | 13 | 12 | 11 | 10 | 9 | 8 |
|---|---|---|---|---|---|---|
| 1 | ACC | GTC | TCC | TGG | GGA | GAT |
| 2 | tCC | aTC | gCC | gGG | tGA | tAT |
| 3 | gCC | | cCC | cGG | cGA | aAT |
| 4 | cCC | | aCC | aGG | aGA | |

| Codon | 7 | 6 | 5 | 4 | 3 | 2 | 1 |
|---|---|---|---|---|---|---|---|
| 1 | GGT | TTC | CAC | CTG | CAC | TCC | CAT |
| 2 | tGT | cTC | tAC | tTG | tAC | gCC | |
| 3 | cGT | | gAC | | gAC | cCC | |
| 4 | aGT | | aAC | | aAC | aCC | |

The pegRNAs can also be used to introduce PAM immunizing edits. For example, a PAM immunizing edit for Spacer 1 may be introduced by mutating Codon 12 of the FKBP1 gene from GTC to aTC and, a PAM immunizing edit for Spacer 2 may be introduced by mutating Codon 10 of the FKBP1 gene from TGG to gGG, cGG, or aGG. By “immunizing edit” is meant an edit that, once introduced, alters the PAM corresponding to the spacer so as to prevent the prime editor construct complexed with a pegRNA containing a spacer corresponding to the PAM from binding to the sequence corresponding to the spacer.

The nRNA sequences of Table 11 were selected such that they could mediate a nick within 80 nt of the nick induced using Spacer 1 or Spacer 2.

In various embodiments, a pegRNA of Table 10 or an nRNA of Table 11 may include one or more modified nucleobases (e.g., a 2′-O-methyl (‘M’) and/or 3′-phosphorothioate modifications). In some embodiments, the three 5′ and/or 3′ terminal nucleobases of a pegRNA or an nRNA are modified nucleobases (e.g., having 2′-O-methyl (‘M’) and/or 3′-phosphorothioate modifications).

### Sequences

Table 9 below provides sequences for primers used in the above examples for sequencing of nucleobase edited sites. Tables 10 and 11 below provide sequences for prime editing guide RNAs (pegRNAs) and nicking RNAs (nRNAs) used in the above examples. Table 12 provides amino acid and nucleotide sequences for prime editor constructs used in the above examples.

TABLE 9
Primers used to sequence target sites altered using base editor systems containing
guide RNA's corresponding to the indicated sgRNA IDs.

| | PCR amplification primer | SEQ | PCR amplification primer | SEQ |
|---|---|---|---|---|
| sgRNA ID | (forward) | ID NO | (reverse) | ID NO |
| TSBTx760 | TGTCTTTCAGCAAGGACTGG | 752 | GACTCATTCAGGGTAGTATGG | 762 |
| | TCTTTCTA | | CCATAGA | |
| TSBTx845 | CGCGCTGGCGGGCATTCCTG | 753 | GCGGGCCACCAAGGAGAACTT | 754 |
| | AAGCTGA | | GGAGAA | |
| TSBTx763 | GCGGGCCACCAAGGAGAACT | 754 | GCGGGCCACCAAGGAGAACTT | 754 |
| | TGGAGAA | | GGAGAA | |
| TSBTx4073 | GGATCACCTGTCACTGAAGG | 755 | CGCAAAGACGCCTGGGCACTG | 763 |
| | AATTTTCA | | TGA | |
| TSBTx754 | GAGCTGCAGGCCTCCCCCAC | 756 | GCAGATTAAACCCGGCCACTT | 764 |
| | CCA | | TCAGG | |
| TSBTx1538 | CCGAGGTACTAGGCAGAGC | 757 | CCCTGAGGAGACAGAGACGG | 765 |
| TSBTx1542 | CCCAGGAGACGGTGAGTAGT | 758 | CCTCGACGGCCAGCC | 766 |
| TSBTx1545 | TATGCCTATGGTGCCACTGG | 759 | CTCTGCTACCCATCAAACGC | 767 |
| TSBTx1546 | GTCGCCACTGCACACAAAG | 760 | TCGGAAGCAAAGCTGAG | 768 |
| TSBTx1581 | TGATGAGTGCTCTGCTGCTG | 761 | GAGAGAGCATACCTGGGCAA | 769 |
| TSBTx1582 | CCGAGGTACTAGGCAGAGC | 757 | CCCTGAGGAGACAGAGACGG | 765 |
| TSBTx1537 | CCGAGGTACTAGGCAGAGC | 757 | CCCTGAGGAGACAGAGACGG | 765 |
| TSBTx1539 | CCCAGGAGACGGTGAGTAGT | 758 | CCTCGACGGCCAGCC | 766 |
| TSBTx1540 | CCCAGGAGACGGTGAGTAGT | 758 | CCTCGACGGCCAGCC | 766 |
| TSBTx1541 | CCCAGGAGACGGTGAGTAGT | 758 | CCTCGACGGCCAGCC | 766 |
| TSBTx1543 | GGCCAAGGAACACCTAGGTA | 770 | GAAGCTCCACATCGAAGACG | 772 |
| TSBTx1544 | TATGCCTATGGTGCCACTGG | 759 | CTCTGCTACCCATCAAACGC | 767 |
| TSBTx1577 | CACAGGGATGCTTGAAGATG | 771 | CACAGGGATGCTTGAAGATGG | 771 |
| | G | | | |
| TSBTx1578 | CACAGGGATGCTTGAAGATG | 771 | CACAGGGATGCTTGAAGATGG | 771 |
| | G | | | |
| TSBTx1579 | CACAGGGATGCTTGAAGATG | 771 | CACAGGGATGCTTGAAGATGG | 771 |
| | G | | | |
| TSBTx1580 | CACAGGGATGCTTGAAGATG | 771 | CACAGGGATGCTTGAAGATGG | 771 |
| | G | | | |

TABLE 10
pegRNA Sequences. In some embodiments, the pegRNA sequences further
comprise the nucleotide sequence “cacc” or “caccg” at the 5′-end.

| | SEQ |
|---|---|
| pegRNA Sequence | ID NO |
| GAAACCAUCUCCCCAGGAGAguuuuagaGCUAGAAAUAGCAAGUUAAAAUAAGGCUAGUCCG | 809 |
| UUAUCAACUUGAAAAAGUGGCACCGAGUCGgugcCUCgCCGUCUCCUGGGGAGA | |
| GAAACCAUCUCCCCAGGAGAguuuuagaGCUAGAAAUAGCAAGUUAAAAUAAGGCUAGUCCG | 810 |
| UUAUCAACUUGAAAAAGUGGCACCGAGUCGgugcCUCgCCGUCUCCUGGGGAGAU | |
| GAAACCAUCUCCCCAGGAGAguuuuagaGCUAGAAAUAGCAAGUUAAAAUAAGGCUAGUCCG | 811 |
| UUAUCAACUUGAAAAAGUGGCACCGAGUCGgugcCUCgCCGUCUCCUGGGGAGAUG | |
| GAAACCAUCUCCCCAGGAGAguuuuagaGCUAGAAAUAGCAAGUUAAAAUAAGGCUAGUCCG | 812 |
| UUAUCAACUUGAAAAAGUGGCACCGAGUCGgugcCUCgCCGUCUCCUGGGGAGAUGG | |
| GAAACCAUCUCCCCAGGAGAguuuuagaGCUAGAAAUAGCAAGUUAAAAUAAGGCUAGUCCG | 813 |
| UUAUCAACUUGAAAAAGUGGCACCGAGUCGgugcCUCgCCGUCUCCUGGGGAGAUGGU | |
| GAAACCAUCUCCCCAGGAGAguuuuagaGCUAGAAAUAGCAAGUUAAAAUAAGGCUAGUCCG | 814 |
| UUAUCAACUUGAAAAAGUGGCACCGAGUCGgugcCUCgCCGUCUCCUGGGGAGAUGGUU | |
| GAAACCAUCUCCCCAGGAGAguuuuagaGCUAGAAAUAGCAAGUUAAAAUAAGGCUAGUCCG | 815 |
| UUAUCAACUUGAAAAAGUGGCACCGAGUCGgugcACUCgCCGUCUCCUGGGGAGA | |
| GAAACCAUCUCCCCAGGAGAguuuuagaGCUAGAAAUAGCAAGUUAAAAUAAGGCUAGUCCG | 816 |
| UUAUCAACUUGAAAAAGUGGCACCGAGUCGgugcACUCgCCGUCUCCUGGGGAGAU | |
| GAAACCAUCUCCCCAGGAGAguuuuagaGCUAGAAAUAGCAAGUUAAAAUAAGGCUAGUCCG | 817 |
| UUAUCAACUUGAAAAAGUGGCACCGAGUCGgugcACUCgCCGUCUCCUGGGGAGAUG | |
| GAAACCAUCUCCCCAGGAGAguuuuagaGCUAGAAAUAGCAAGUUAAAAUAAGGCUAGUCCG | 818 |
| UUAUCAACUUGAAAAAGUGGCACCGAGUCGgugcACUCgCCGUCUCCUGGGGAGAUGG | |
| GAAACCAUCUCCCCAGGAGAguuuuagaGCUAGAAAUAGCAAGUUAAAAUAAGGCUAGUCCG | 819 |
| UUAUCAACUUGAAAAAGUGGCACCGAGUCGgugcACUCgCCGUCUCCUGGGGAGAUGGU | |
| GAAACCAUCUCCCCAGGAGAguuuuagaGCUAGAAAUAGCAAGUUAAAAUAAGGCUAGUCCG | 820 |
| UUAUCAACUUGAAAAAGUGGCACCGAGUCGgugcACUCgCCGUCUCCUGGGGAGAUGGUU | |
| GAAACCAUCUCCCCAGGAGAguuuuagaGCUAGAAAUAGCAAGUUAAAAUAAGGCUAGUCCG | 821 |
| UUAUCAACUUGAAAAAGUGGCACCGAGUCGgugcUACUCgCCGUCUCCUGGGGAGA | |
| GAAACCAUCUCCCCAGGAGAguuuuagaGCUAGAAAUAGCAAGUUAAAAUAAGGCUAGUCCG | 822 |
| UUAUCAACUUGAAAAAGUGGCACCGAGUCGgugcUACUCgCCGUCUCCUGGGGAGAU | |
| GAAACCAUCUCCCCAGGAGAguuuuagaGCUAGAAAUAGCAAGUUAAAAUAAGGCUAGUCCG | 823 |
| UUAUCAACUUGAAAAAGUGGCACCGAGUCGgugcUACUCgCCGUCUCCUGGGGAGAUG | |
| GAAACCAUCUCCCCAGGAGAguuuuagaGCUAGAAAUAGCAAGUUAAAAUAAGGCUAGUCCG | 824 |
| UUAUCAACUUGAAAAAGUGGCACCGAGUCGgugcUACUCgCCGUCUCCUGGGGAGAUGG | |
| GAAACCAUCUCCCCAGGAGAguuuuagaGCUAGAAAUAGCAAGUUAAAAUAAGGCUAGUCCG | 825 |
| UUAUCAACUUGAAAAAGUGGCACCGAGUCGgugcUACUCgCCGUCUCCUGGGGAGAUGGU | |
| GAAACCAUCUCCCCAGGAGAguuuuagaGCUAGAAAUAGCAAGUUAAAAUAAGGCUAGUCCG | 826 |
| UUAUCAACUUGAAAAAGUGGCACCGAGUCGgugcUACUCgCCGUCUCCUGGGGAGAUGGUU | |
| GAAACCAUCUCCCCAGGAGAguuuuagaGCUAGAAAUAGCAAGUUAAAAUAAGGCUAGUCCG | 827 |
| UUAUCAACUUGAAAAAGUGGCACCGAGUCGgugcCUACUCgCCGUCUCCUGGGGAGA | |
| GAAACCAUCUCCCCAGGAGAguuuuagaGCUAGAAAUAGCAAGUUAAAAUAAGGCUAGUCCG | 828 |
| UUAUCAACUUGAAAAAGUGGCACCGAGUCGgugcCUACUCgCCGUCUCCUGGGGAGAU | |
| GAAACCAUCUCCCCAGGAGAguuuuagaGCUAGAAAUAGCAAGUUAAAAUAAGGCUAGUCCG | 829 |
| UUAUCAACUUGAAAAAGUGGCACCGAGUCGgugcCUACUCgCCGUCUCCUGGGGAGAUG | |
| GAAACCAUCUCCCCAGGAGAguuuuagaGCUAGAAAUAGCAAGUUAAAAUAAGGCUAGUCCG | 830 |
| UUAUCAACUUGAAAAAGUGGCACCGAGUCGgugcCUACUCgCCGUCUCCUGGGGAGAUGG | |
| GAAACCAUCUCCCCAGGAGAguuuuagaGCUAGAAAUAGCAAGUUAAAAUAAGGCUAGUCCG | 831 |
| UUAUCAACUUGAAAAAGUGGCACCGAGUCGgugcCUACUCgCCGUCUCCUGGGGAGAUGGU | |
| GAAACCAUCUCCCCAGGAGAguuuuagaGCUAGAAAUAGCAAGUUAAAAUAAGGCUAGUCCG | 832 |
| UUAUCAACUUGAAAAAGUGGCACCGAGUCGgugcCUACUCgCCGUCUCCUGGGGAGAUGGUU | |
| GAAACCAUCUCCCCAGGAGAguuuuagaGCUAGAAAUAGCAAGUUAAAAUAAGGCUAGUCCG | 833 |
| UUAUCAACUUGAAAAAGUGGCACCGAGUCGgugcACUACUCgCCGUCUCCUGGGGAGA | |
| GAAACCAUCUCCCCAGGAGAguuuuagaGCUAGAAAUAGCAAGUUAAAAUAAGGCUAGUCCG | 834 |
| UUAUCAACUUGAAAAAGUGGCACCGAGUCGgugcACUACUCgCCGUCUCCUGGGGAGAU | |
| GAAACCAUCUCCCCAGGAGAguuuuagaGCUAGAAAUAGCAAGUUAAAAUAAGGCUAGUCCG | 835 |
| UUAUCAACUUGAAAAAGUGGCACCGAGUCGgugcACUACUCgCCGUCUCCUGGGGAGAUG | |
| GAAACCAUCUCCCCAGGAGAguuuuagaGCUAGAAAUAGCAAGUUAAAAUAAGGCUAGUCCG | 836 |
| UUAUCAACUUGAAAAAGUGGCACCGAGUCGgugcACUACUCgCCGUCUCCUGGGGAGAUGG | |
| GAAACCAUCUCCCCAGGAGAguuuuagaGCUAGAAAUAGCAAGUUAAAAUAAGGCUAGUCCG | 837 |
| UUAUCAACUUGAAAAAGUGGCACCGAGUCGgugcACUACUCgCCGUCUCCUGGGGAGAUGGU | |
| GAAACCAUCUCCCCAGGAGAguuuuagaGCUAGAAAUAGCAAGUUAAAAUAAGGCUAGUCCG | 838 |
| UUAUCAACUUGAAAAAGUGGCACCGAGUCGgugcACUACUCgCCGUCUCCUGGGGAGAUGGU | |
| U | |
| GAAACCAUCUCCCCAGGAGAguuuuagaGCUAGAAAUAGCAAGUUAAAAUAAGGCUAGUCCG | 839 |
| UUAUCAACUUGAAAAAGUGGCACCGAGUCGgugcCACUACUCgCCGUCUCCUGGGGAGA | |
| GAAACCAUCUCCCCAGGAGAguuuuagaGCUAGAAAUAGCAAGUUAAAAUAAGGCUAGUCCG | 840 |
| UUAUCAACUUGAAAAAGUGGCACCGAGUCGgugcCACUACUCgCCGUCUCCUGGGGAGAU | |
| GAAACCAUCUCCCCAGGAGAguuuuagaGCUAGAAAUAGCAAGUUAAAAUAAGGCUAGUCCG | 841 |
| UUAUCAACUUGAAAAAGUGGCACCGAGUCGgugcCACUACUCgCCGUCUCCUGGGGAGAUG | |
| GAAACCAUCUCCCCAGGAGAguuuuagaGCUAGAAAUAGCAAGUUAAAAUAAGGCUAGUCCG | 842 |
| UUAUCAACUUGAAAAAGUGGCACCGAGUCGgugcCACUACUCgCCGUCUCCUGGGGAGAUGG | |
| GAAACCAUCUCCCCAGGAGAguuuuagaGCUAGAAAUAGCAAGUUAAAAUAAGGCUAGUCCG | 843 |
| UUAUCAACUUGAAAAAGUGGCACCGAGUCGgugcCACUACUCgCCGUCUCCUGGGGAGAUGG | |
| U | |
| GAAACCAUCUCCCCAGGAGAguuuuagaGCUAGAAAUAGCAAGUUAAAAUAAGGCUAGUCCG | 844 |
| UUAUCAACUUGAAAAAGUGGCACCGAGUCGgugcCACUACUCgCCGUCUCCUGGGGAGAUGG | |
| UU | |
| GAAACCAUCUCCCCAGGAGAguuuuagaGCUAGAAAUAGCAAGUUAAAAUAAGGCUAGUCCG | 845 |
| UUAUCAACUUGAAAAAGUGGCACCGAGUCGgugcCCACUACUCgCCGUCUCCUGGGGAGA | |
| GAAACCAUCUCCCCAGGAGAguuuuagaGCUAGAAAUAGCAAGUUAAAAUAAGGCUAGUCCG | 846 |
| UUAUCAACUUGAAAAAGUGGCACCGAGUCGgugcCCACUACUCgCCGUCUCCUGGGGAGAU | |
| GAAACCAUCUCCCCAGGAGAguuuuagaGCUAGAAAUAGCAAGUUAAAAUAAGGCUAGUCCG | 847 |
| UUAUCAACUUGAAAAAGUGGCACCGAGUCGgugcCCACUACUCgCCGUCUCCUGGGGAGAUG | |
| GAAACCAUCUCCCCAGGAGAguuuuagaGCUAGAAAUAGCAAGUUAAAAUAAGGCUAGUCCG | 848 |
| UUAUCAACUUGAAAAAGUGGCACCGAGUCGgugcCCACUACUCgCCGUCUCCUGGGGAGAUG | |
| G | |
| GAAACCAUCUCCCCAGGAGAguuuuagaGCUAGAAAUAGCAAGUUAAAAUAAGGCUAGUCCG | 849 |
| UUAUCAACUUGAAAAAGUGGCACCGAGUCGgugcCCACUACUCgCCGUCUCCUGGGGAGAUG | |
| GU | |
| GAAACCAUCUCCCCAGGAGAguuuuagaGCUAGAAAUAGCAAGUUAAAAUAAGGCUAGUCCG | 850 |
| UUAUCAACUUGAAAAAGUGGCACCGAGUCGgugcCCACUACUCgCCGUCUCCUGGGGAGAUG | |
| GUU | |
| GAAACCAUCUCCCCAGGAGAguuuuagaGCUAGAAAUAGCAAGUUAAAAUAAGGCUAGUCCG | 851 |
| UUAUCAACUUGAAAAAGUGGCACCGAGUCGgugcGCCACUACUCgCCGUCUCCUGGGGAGA | |
| GAAACCAUCUCCCCAGGAGAguuuuagaGCUAGAAAUAGCAAGUUAAAAUAAGGCUAGUCCG | 852 |
| UUAUCAACUUGAAAAAGUGGCACCGAGUCGgugcGCCACUACUCgCCGUCUCCUGGGGAGAU | |
| GAAACCAUCUCCCCAGGAGAguuuuagaGCUAGAAAUAGCAAGUUAAAAUAAGGCUAGUCCG | 853 |
| UUAUCAACUUGAAAAAGUGGCACCGAGUCGgugcGCCACUACUCgCCGUCUCCUGGGGAGAU | |
| G | |
| GAAACCAUCUCCCCAGGAGAguuuuagaGCUAGAAAUAGCAAGUUAAAAUAAGGCUAGUCCG | 854 |
| UUAUCAACUUGAAAAAGUGGCACCGAGUCGgugcGCCACUACUCgCCGUCUCCUGGGGAGAU | |
| GG | |
| GAAACCAUCUCCCCAGGAGAguuuuagaGCUAGAAAUAGCAAGUUAAAAUAAGGCUAGUCCG | 855 |
| UUAUCAACUUGAAAAAGUGGCACCGAGUCGgugcGCCACUACUCgCCGUCUCCUGGGGAGAU | |
| GGU | |
| GAAACCAUCUCCCCAGGAGAguuuuagaGCUAGAAAUAGCAAGUUAAAAUAAGGCUAGUCCG | 856 |
| UUAUCAACUUGAAAAAGUGGCACCGAGUCGgugcGCCACUACUCgCCGUCUCCUGGGGAGAU | |
| GGUU | |
| GAAACCAUCUCCCCAGGAGAguuuuagaGCUAGAAAUAGCAAGUUAAAAUAAGGCUAGUCCG | 857 |
| UUAUCAACUUGAAAAAGUGGCACCGAGUCGgugcCGCCACUACUCgCCGUCUCCUGGGGAGA | |
| GAAACCAUCUCCCCAGGAGAguuuuagaGCUAGAAAUAGCAAGUUAAAAUAAGGCUAGUCCG | 858 |
| UUAUCAACUUGAAAAAGUGGCACCGAGUCGgugcCGCCACUACUCgCCGUCUCCUGGGGAGA | |
| U | |
| GAAACCAUCUCCCCAGGAGAguuuuagaGCUAGAAAUAGCAAGUUAAAAUAAGGCUAGUCCG | 859 |
| UUAUCAACUUGAAAAAGUGGCACCGAGUCGgugcCGCCACUACUCgCCGUCUCCUGGGGAGA | |
| UG | |
| GAAACCAUCUCCCCAGGAGAguuuuagaGCUAGAAAUAGCAAGUUAAAAUAAGGCUAGUCCG | 860 |
| UUAUCAACUUGAAAAAGUGGCACCGAGUCGgugcCGCCACUACUCgCCGUCUCCUGGGGAGA | |
| UGG | |
| GAAACCAUCUCCCCAGGAGAguuuuagaGCUAGAAAUAGCAAGUUAAAAUAAGGCUAGUCCG | 861 |
| UUAUCAACUUGAAAAAGUGGCACCGAGUCGgugcCGCCACUACUCgCCGUCUCCUGGGGAGA | |
| UGGU | |
| GAAACCAUCUCCCCAGGAGAguuuuagaGCUAGAAAUAGCAAGUUAAAAUAAGGCUAGUCCG | 862 |
| UUAUCAACUUGAAAAAGUGGCACCGAGUCGgugcCGCCACUACUCgCCGUCUCCUGGGGAGA | |
| UGGUU | |
| GAAACCAUCUCCCCAGGAGAguuuuagaGCUAGAAAUAGCAAGUUAAAAUAAGGCUAGUCCG | 863 |
| UUAUCAACUUGAAAAAGUGGCACCGAGUCGgugcGCGCCACUACUCgCCGUCUCCUGGGGAG | |
| A | |
| GAAACCAUCUCCCCAGGAGAguuuuagaGCUAGAAAUAGCAAGUUAAAAUAAGGCUAGUCCG | 864 |
| UUAUCAACUUGAAAAAGUGGCACCGAGUCGgugcGCGCCACUACUCgCCGUCUCCUGGGGAG | |
| AU | |
| GAAACCAUCUCCCCAGGAGAguuuuagaGCUAGAAAUAGCAAGUUAAAAUAAGGCUAGUCCG | 865 |
| UUAUCAACUUGAAAAAGUGGCACCGAGUCGgugcGCGCCACUACUCgCCGUCUCCUGGGGAG | |
| AUG | |
| GAAACCAUCUCCCCAGGAGAguuuuagaGCUAGAAAUAGCAAGUUAAAAUAAGGCUAGUCCG | 866 |
| UUAUCAACUUGAAAAAGUGGCACCGAGUCGgugcGCGCCACUACUCgCCGUCUCCUGGGGAG | |
| AUGG | |
| GAAACCAUCUCCCCAGGAGAguuuuagaGCUAGAAAUAGCAAGUUAAAAUAAGGCUAGUCCG | 867 |
| UUAUCAACUUGAAAAAGUGGCACCGAGUCGgugcGCGCCACUACUCgCCGUCUCCUGGGGAG | |
| AUGGU | |
| GAAACCAUCUCCCCAGGAGAguuuuagaGCUAGAAAUAGCAAGUUAAAAUAAGGCUAGUCCG | 868 |
| UUAUCAACUUGAAAAAGUGGCACCGAGUCGgugcGCGCCACUACUCgCCGUCUCCUGGGGAG | |
| AUGGUU | |
| GAAACCAUCUCCCCAGGAGAguuuuagaGCUAGAAAUAGCAAGUUAAAAUAAGGCUAGUCCG | 869 |
| UUAUCAACUUGAAAAAGUGGCACCGAGUCGgugcCGCGCCACUACUCgCCGUCUCCUGGGGA | |
| GA | |
| GAAACCAUCUCCCCAGGAGAguuuuagaGCUAGAAAUAGCAAGUUAAAAUAAGGCUAGUCCG | 870 |
| UUAUCAACUUGAAAAAGUGGCACCGAGUCGgugcCGCGCCACUACUCgCCGUCUCCUGGGGA | |
| GAU | |
| GAAACCAUCUCCCCAGGAGAguuuuagaGCUAGAAAUAGCAAGUUAAAAUAAGGCUAGUCCG | 871 |
| UUAUCAACUUGAAAAAGUGGCACCGAGUCGgugcCGCGCCACUACUCgCCGUCUCCUGGGGA | |
| GAUG | |
| GAAACCAUCUCCCCAGGAGAguuuuagaGCUAGAAAUAGCAAGUUAAAAUAAGGCUAGUCCG | 872 |
| UUAUCAACUUGAAAAAGUGGCACCGAGUCGgugcCGCGCCACUACUCgCCGUCUCCUGGGGA | |
| GAUGG | |
| GAAACCAUCUCCCCAGGAGAguuuuagaGCUAGAAAUAGCAAGUUAAAAUAAGGCUAGUCCG | 873 |
| UUAUCAACUUGAAAAAGUGGCACCGAGUCGgugcCGCGCCACUACUCgCCGUCUCCUGGGGA | |
| GAUGGU | |
| GAAACCAUCUCCCCAGGAGAguuuuagaGCUAGAAAUAGCAAGUUAAAAUAAGGCUAGUCCG | 874 |
| UUAUCAACUUGAAAAAGUGGCACCGAGUCGgugcCGCGCCACUACUCgCCGUCUCCUGGGGA | |
| GAUGGUU | |
| GAAACCAUCUCCCCAGGAGAguuuuagaGCUAGAAAUAGCAAGUUAAAAUAAGGCUAGUCCG | 875 |
| UUAUCAACUUGAAAAAGUGGCACCGAGUCGgugcGCGCGCCACUACUCgCCGUCUCCUGGGG | |
| AGA | |
| GAAACCAUCUCCCCAGGAGAguuuuagaGCUAGAAAUAGCAAGUUAAAAUAAGGCUAGUCCG | 876 |
| UUAUCAACUUGAAAAAGUGGCACCGAGUCGgugcGCGCGCCACUACUCgCCGUCUCCUGGGG | |
| AGAU | |
| GAAACCAUCUCCCCAGGAGAguuuuagaGCUAGAAAUAGCAAGUUAAAAUAAGGCUAGUCCG | 877 |
| UUAUCAACUUGAAAAAGUGGCACCGAGUCGgugcGCGCGCCACUACUCgCCGUCUCCUGGGG | |
| AGAUG | |
| GAAACCAUCUCCCCAGGAGAguuuuagaGCUAGAAAUAGCAAGUUAAAAUAAGGCUAGUCCG | 878 |
| UUAUCAACUUGAAAAAGUGGCACCGAGUCGgugcGCGCGCCACUACUCgCCGUCUCCUGGGG | |
| AGAUGG | |
| GAAACCAUCUCCCCAGGAGAguuuuagaGCUAGAAAUAGCAAGUUAAAAUAAGGCUAGUCCG | 879 |
| UUAUCAACUUGAAAAAGUGGCACCGAGUCGgugcGCGCGCCACUACUCgCCGUCUCCUGGGG | |
| AGAUGGU | |
| GAAACCAUCUCCCCAGGAGAguuuuagaGCUAGAAAUAGCAAGUUAAAAUAAGGCUAGUCCG | 880 |
| UUAUCAACUUGAAAAAGUGGCACCGAGUCGgugcGCGCGCCACUACUCgCCGUCUCCUGGGG | |
| AGAUGGUU | |
| GAAACCAUCUCCCCAGGAGAguuuuagaGCUAGAAAUAGCAAGUUAAAAUAAGGCUAGUCCG | 881 |
| UUAUCAACUUGAAAAAGUGGCACCGAGUCGgugcCGCGCGCCACUACUCgCCGUCUCCUGGG | |
| GAGA | |
| GAAACCAUCUCCCCAGGAGAguuuuagaGCUAGAAAUAGCAAGUUAAAAUAAGGCUAGUCCG | 882 |
| UUAUCAACUUGAAAAAGUGGCACCGAGUCGgugcCGCGCGCCACUACUCgCCGUCUCCUGGG | |
| GAGAU | |
| GAAACCAUCUCCCCAGGAGAguuuuagaGCUAGAAAUAGCAAGUUAAAAUAAGGCUAGUCCG | 883 |
| UUAUCAACUUGAAAAAGUGGCACCGAGUCGgugcCGCGCGCCACUACUCgCCGUCUCCUGGG | |
| GAGAUG | |
| GAAACCAUCUCCCCAGGAGAguuuuagaGCUAGAAAUAGCAAGUUAAAAUAAGGCUAGUCCG | 884 |
| UUAUCAACUUGAAAAAGUGGCACCGAGUCGgugcCGCGCGCCACUACUCgCCGUCUCCUGGG | |
| GAGAUGG | |
| GAAACCAUCUCCCCAGGAGAguuuuagaGCUAGAAAUAGCAAGUUAAAAUAAGGCUAGUCCG | 885 |
| UUAUCAACUUGAAAAAGUGGCACCGAGUCGgugcCGCGCGCCACUACUCgCCGUCUCCUGGG | |
| GAGAUGGU | |
| GAAACCAUCUCCCCAGGAGAguuuuagaGCUAGAAAUAGCAAGUUAAAAUAAGGCUAGUCCG | 886 |
| UUAUCAACUUGAAAAAGUGGCACCGAGUCGgugcCGCGCGCCACUACUCgCCGUCUCCUGGG | |
| GAGAUGGUU | |
| GAAACCAUCUCCCCAGGAGAguuuuagaGCUAGAAAUAGCAAGUUAAAAUAAGGCUAGUCCG | 887 |
| UUAUCAACUUGAAAAAGUGGCACCGAGUCGgugcCCGCGCGCCACUACUCgCCGUCUCCUGG | |
| GGAGA | |
| GAAACCAUCUCCCCAGGAGAguuuuagaGCUAGAAAUAGCAAGUUAAAAUAAGGCUAGUCCG | 888 |
| UUAUCAACUUGAAAAAGUGGCACCGAGUCGgugcCCGCGCGCCACUACUCgCCGUCUCCUGG | |
| GGAGAU | |
| GAAACCAUCUCCCCAGGAGAguuuuagaGCUAGAAAUAGCAAGUUAAAAUAAGGCUAGUCCG | 889 |
| UUAUCAACUUGAAAAAGUGGCACCGAGUCGgugcCCGCGCGCCACUACUCgCCGUCUCCUGG | |
| GGAGAUG | |
| GAAACCAUCUCCCCAGGAGAguuuuagaGCUAGAAAUAGCAAGUUAAAAUAAGGCUAGUCCG | 890 |
| UUAUCAACUUGAAAAAGUGGCACCGAGUCGgugcCCGCGCGCCACUACUCgCCGUCUCCUGG | |
| GGAGAUGG | |
| GAAACCAUCUCCCCAGGAGAguuuuagaGCUAGAAAUAGCAAGUUAAAAUAAGGCUAGUCCG | 891 |
| UUAUCAACUUGAAAAAGUGGCACCGAGUCGgugcCCGCGCGCCACUACUCgCCGUCUCCUGG | |
| GGAGAUGGU | |
| GAAACCAUCUCCCCAGGAGAguuuuagaGCUAGAAAUAGCAAGUUAAAAUAAGGCUAGUCCG | 892 |
| UUAUCAACUUGAAAAAGUGGCACCGAGUCGgugcCCGCGCGCCACUACUCgCCGUCUCCUGG | |
| GGAGAUGGUU | |
| GAAACCAUCUCCCCAGGAGAguuuuagaGCUAGAAAUAGCAAGUUAAAAUAAGGCUAGUCCG | 893 |
| UUAUCAACUUGAAAAAGUGGCACCGAGUCGgugcGCCGCGCGCCACUACUCgCCGUCUCCUG | |
| GGGAGA | |
| GAAACCAUCUCCCCAGGAGAguuuuagaGCUAGAAAUAGCAAGUUAAAAUAAGGCUAGUCCG | 894 |
| UUAUCAACUUGAAAAAGUGGCACCGAGUCGgugcGCCGCGCGCCACUACUCgCCGUCUCCUG | |
| GGGAGAU | |
| GAAACCAUCUCCCCAGGAGAguuuuagaGCUAGAAAUAGCAAGUUAAAAUAAGGCUAGUCCG | 895 |
| UUAUCAACUUGAAAAAGUGGCACCGAGUCGgugcGCCGCGCGCCACUACUCgCCGUCUCCUG | |
| GGGAGAUG | |
| GAAACCAUCUCCCCAGGAGAguuuuagaGCUAGAAAUAGCAAGUUAAAAUAAGGCUAGUCCG | 896 |
| UUAUCAACUUGAAAAAGUGGCACCGAGUCGgugcGCCGCGCGCCACUACUCgCCGUCUCCUG | |
| GGGAGAUGG | |
| GAAACCAUCUCCCCAGGAGAguuuuagaGCUAGAAAUAGCAAGUUAAAAUAAGGCUAGUCCG | 897 |
| UUAUCAACUUGAAAAAGUGGCACCGAGUCGgugcGCCGCGCGCCACUACUCgCCGUCUCCUG | |
| GGGAGAUGGU | |
| GAAACCAUCUCCCCAGGAGAguuuuagaGCUAGAAAUAGCAAGUUAAAAUAAGGCUAGUCCG | 898 |
| UUAUCAACUUGAAAAAGUGGCACCGAGUCGgugcGCCGCGCGCCACUACUCgCCGUCUCCUG | |
| GGGAGAUGGUU | |
| GAAACCAUCUCCCCAGGAGAguuuuagaGCUAGAAAUAGCAAGUUAAAAUAAGGCUAGUCCG | 899 |
| UUAUCAACUUGAAAAAGUGGCACCGAGUCGgugcCGCCGCGCGCCACUACUCgCCGUCUCCU | |
| GGGGAGA | |
| GAAACCAUCUCCCCAGGAGAguuuuagaGCUAGAAAUAGCAAGUUAAAAUAAGGCUAGUCCG | 900 |
| UUAUCAACUUGAAAAAGUGGCACCGAGUCGgugcCGCCGCGCGCCACUACUCgCCGUCUCCU | |
| GGGGAGAU | |
| GAAACCAUCUCCCCAGGAGAguuuuagaGCUAGAAAUAGCAAGUUAAAAUAAGGCUAGUCCG | 901 |
| UUAUCAACUUGAAAAAGUGGCACCGAGUCGgugcCGCCGCGCGCCACUACUCgCCGUCUCCU | |
| GGGGAGAUG | |
| GAAACCAUCUCCCCAGGAGAguuuuagaGCUAGAAAUAGCAAGUUAAAAUAAGGCUAGUCCG | 902 |
| UUAUCAACUUGAAAAAGUGGCACCGAGUCGgugcCGCCGCGCGCCACUACUCgCCGUCUCCU | |
| GGGGAGAUGG | |
| GAAACCAUCUCCCCAGGAGAguuuuagaGCUAGAAAUAGCAAGUUAAAAUAAGGCUAGUCCG | 903 |
| UUAUCAACUUGAAAAAGUGGCACCGAGUCGgugcCGCCGCGCGCCACUACUCgCCGUCUCCU | |
| GGGGAGAUGGU | |
| GAAACCAUCUCCCCAGGAGAguuuuagaGCUAGAAAUAGCAAGUUAAAAUAAGGCUAGUCCG | 904 |
| UUAUCAACUUGAAAAAGUGGCACCGAGUCGgugcCGCCGCGCGCCACUACUCgCCGUCUCCU | |
| GGGGAGAUGGUU | |
| GAAACCAUCUCCCCAGGAGAguuuuagaGCUAGAAAUAGCAAGUUAAAAUAAGGCUAGUCCG | 905 |
| UUAUCAACUUGAAAAAGUGGCACCGAGUCGgugcCCGCCGCGCGCCACUACUCgCCGUCUCC | |
| UGGGGAGA | |
| GAAACCAUCUCCCCAGGAGAguuuuagaGCUAGAAAUAGCAAGUUAAAAUAAGGCUAGUCCG | 906 |
| UUAUCAACUUGAAAAAGUGGCACCGAGUCGgugcCCGCCGCGCGCCACUACUCgCCGUCUCC | |
| UGGGGAGAU | |
| GAAACCAUCUCCCCAGGAGAguuuuagaGCUAGAAAUAGCAAGUUAAAAUAAGGCUAGUCCG | 907 |
| UUAUCAACUUGAAAAAGUGGCACCGAGUCGgugcCCGCCGCGCGCCACUACUCgCCGUCUCC | |
| UGGGGAGAUG | |
| GAAACCAUCUCCCCAGGAGAguuuuagaGCUAGAAAUAGCAAGUUAAAAUAAGGCUAGUCCG | 908 |
| UUAUCAACUUGAAAAAGUGGCACCGAGUCGgugcCCGCCGCGCGCCACUACUCgCCGUCUCC | |
| UGGGGAGAUGG | |
| GAAACCAUCUCCCCAGGAGAguuuuagaGCUAGAAAUAGCAAGUUAAAAUAAGGCUAGUCCG | 909 |
| UUAUCAACUUGAAAAAGUGGCACCGAGUCGgugcCCGCCGCGCGCCACUACUCgCCGUCUCC | |
| UGGGGAGAUGGU | |
| GAAACCAUCUCCCCAGGAGAguuuuagaGCUAGAAAUAGCAAGUUAAAAUAAGGCUAGUCCG | 910 |
| UUAUCAACUUGAAAAAGUGGCACCGAGUCGgugcCCGCCGCGCGCCACUACUCgCCGUCUCC | |
| UGGGGAGAUGGUU | |
| GAAACCAUCUCCCCAGGAGAguuuuagaGCUAGAAAUAGCAAGUUAAAAUAAGGCUAGUCCG | 911 |
| UUAUCAACUUGAAAAAGUGGCACCGAGUCGgugcGCCGCCGCGCGCCACUACUCgCCGUCUC | |
| CUGGGGAGA | |
| GAAACCAUCUCCCCAGGAGAguuuuagaGCUAGAAAUAGCAAGUUAAAAUAAGGCUAGUCCG | 912 |
| UUAUCAACUUGAAAAAGUGGCACCGAGUCGgugcGCCGCCGCGCGCCACUACUCgCCGUCUC | |
| CUGGGGAGAU | |
| GAAACCAUCUCCCCAGGAGAguuuuagaGCUAGAAAUAGCAAGUUAAAAUAAGGCUAGUCCG | 913 |
| UUAUCAACUUGAAAAAGUGGCACCGAGUCGgugcGCCGCCGCGCGCCACUACUCgCCGUCUC | |
| CUGGGGAGAUG | |
| GAAACCAUCUCCCCAGGAGAguuuuagaGCUAGAAAUAGCAAGUUAAAAUAAGGCUAGUCCG | 914 |
| UUAUCAACUUGAAAAAGUGGCACCGAGUCGgugcGCCGCCGCGCGCCACUACUCgCCGUCUC | |
| CUGGGGAGAUGG | |
| GAAACCAUCUCCCCAGGAGAguuuuagaGCUAGAAAUAGCAAGUUAAAAUAAGGCUAGUCCG | 915 |
| UUAUCAACUUGAAAAAGUGGCACCGAGUCGgugcGCCGCCGCGCGCCACUACUCgCCGUCUC | |
| CUGGGGAGAUGGU | |
| GAAACCAUCUCCCCAGGAGAguuuuagaGCUAGAAAUAGCAAGUUAAAAUAAGGCUAGUCCG | 916 |
| UUAUCAACUUGAAAAAGUGGCACCGAGUCGgugcGCCGCCGCGCGCCACUACUCgCCGUCUC | |
| CUGGGGAGAUGGUU | |
| GAAACCAUCUCCCCAGGAGAguuuuagaGCUAGAAAUAGCAAGUUAAAAUAAGGCUAGUCCG | 917 |
| UUAUCAACUUGAAAAAGUGGCACCGAGUCGgugcAGCCGCCGCGCGCCACUACUCgCCGUCU | |
| CCUGGGGAGA | |
| GAAACCAUCUCCCCAGGAGAguuuuagaGCUAGAAAUAGCAAGUUAAAAUAAGGCUAGUCCG | 918 |
| UUAUCAACUUGAAAAAGUGGCACCGAGUCGgugcAGCCGCCGCGCGCCACUACUCgCCGUCU | |
| CCUGGGGAGAU | |
| GAAACCAUCUCCCCAGGAGAguuuuagaGCUAGAAAUAGCAAGUUAAAAUAAGGCUAGUCCG | 919 |
| UUAUCAACUUGAAAAAGUGGCACCGAGUCGgugcAGCCGCCGCGCGCCACUACUCgCCGUCU | |
| CCUGGGGAGAUG | |
| GAAACCAUCUCCCCAGGAGAguuuuagaGCUAGAAAUAGCAAGUUAAAAUAAGGCUAGUCCG | 920 |
| UUAUCAACUUGAAAAAGUGGCACCGAGUCGgugcAGCCGCCGCGCGCCACUACUCgCCGUCU | |
| CCUGGGGAGAUGG | |
| GAAACCAUCUCCCCAGGAGAguuuuagaGCUAGAAAUAGCAAGUUAAAAUAAGGCUAGUCCG | 921 |
| UUAUCAACUUGAAAAAGUGGCACCGAGUCGgugcAGCCGCCGCGCGCCACUACUCgCCGUCU | |
| CCUGGGGAGAUGGU | |
| GAAACCAUCUCCCCAGGAGAguuuuagaGCUAGAAAUAGCAAGUUAAAAUAAGGCUAGUCCG | 922 |
| UUAUCAACUUGAAAAAGUGGCACCGAGUCGgugcAGCCGCCGCGCGCCACUACUCgCCGUCU | |
| CCUGGGGAGAUGGUU | |
| GAAACCAUCUCCCCAGGAGAguuuuagaGCUAGAAAUAGCAAGUUAAAAUAAGGCUAGUCCG | 923 |
| UUAUCAACUUGAAAAAGUGGCACCGAGUCGgugcGAGCCGCCGCGCGCCACUACUCgCCGUC | |
| UCCUGGGGAGA | |
| GAAACCAUCUCCCCAGGAGAguuuuagaGCUAGAAAUAGCAAGUUAAAAUAAGGCUAGUCCG | 924 |
| UUAUCAACUUGAAAAAGUGGCACCGAGUCGgugcGAGCCGCCGCGCGCCACUACUCgCCGUC | |
| UCCUGGGGAGAU | |
| GAAACCAUCUCCCCAGGAGAguuuuagaGCUAGAAAUAGCAAGUUAAAAUAAGGCUAGUCCG | 925 |
| UUAUCAACUUGAAAAAGUGGCACCGAGUCGgugcGAGCCGCCGCGCGCCACUACUCgCCGUC | |
| UCCUGGGGAGAUG | |
| GAAACCAUCUCCCCAGGAGAguuuuagaGCUAGAAAUAGCAAGUUAAAAUAAGGCUAGUCCG | 926 |
| UUAUCAACUUGAAAAAGUGGCACCGAGUCGgugcGAGCCGCCGCGCGCCACUACUCgCCGUC | |
| UCCUGGGGAGAUGG | |
| GAAACCAUCUCCCCAGGAGAguuuuagaGCUAGAAAUAGCAAGUUAAAAUAAGGCUAGUCCG | 927 |
| UUAUCAACUUGAAAAAGUGGCACCGAGUCGgugcGAGCCGCCGCGCGCCACUACUCgCCGUC | |
| UCCUGGGGAGAUGGU | |
| GAAACCAUCUCCCCAGGAGAguuuuagaGCUAGAAAUAGCAAGUUAAAAUAAGGCUAGUCCG | 928 |
| UUAUCAACUUGAAAAAGUGGCACCGAGUCGgugcGAGCCGCCGCGCGCCACUACUCgCCGUC | |
| UCCUGGGGAGAUGGUU | |
| GAAACCAUCUCCCCAGGAGAguuuuagaGCUAGAAAUAGCAAGUUAAAAUAAGGCUAGUCCG | 929 |
| UUAUCAACUUGAAAAAGUGGCACCGAGUCGgugcCGAGCCGCCGCGCGCCACUACUCgCCGU | |
| CUCCUGGGGAGA | |
| GAAACCAUCUCCCCAGGAGAguuuuagaGCUAGAAAUAGCAAGUUAAAAUAAGGCUAGUCCG | 930 |
| UUAUCAACUUGAAAAAGUGGCACCGAGUCGgugcCGAGCCGCCGCGCGCCACUACUCgCCGU | |
| CUCCUGGGGAGAU | |
| GAAACCAUCUCCCCAGGAGAguuuuagaGCUAGAAAUAGCAAGUUAAAAUAAGGCUAGUCCG | 931 |
| UUAUCAACUUGAAAAAGUGGCACCGAGUCGgugcCGAGCCGCCGCGCGCCACUACUCgCCGU | |
| CUCCUGGGGAGAUG | |
| GAAACCAUCUCCCCAGGAGAguuuuagaGCUAGAAAUAGCAAGUUAAAAUAAGGCUAGUCCG | 932 |
| UUAUCAACUUGAAAAAGUGGCACCGAGUCGgugcCGAGCCGCCGCGCGCCACUACUCgCCGU | |
| CUCCUGGGGAGAUGG | |
| GAAACCAUCUCCCCAGGAGAguuuuagaGCUAGAAAUAGCAAGUUAAAAUAAGGCUAGUCCG | 933 |
| UUAUCAACUUGAAAAAGUGGCACCGAGUCGgugcCGAGCCGCCGCGCGCCACUACUCgCCGU | |
| CUCCUGGGGAGAUGGU | |
| GAAACCAUCUCCCCAGGAGAguuuuagaGCUAGAAAUAGCAAGUUAAAAUAAGGCUAGUCCG | 934 |
| UUAUCAACUUGAAAAAGUGGCACCGAGUCGgugcCGAGCCGCCGCGCGCCACUACUCgCCGU | |
| CUCCUGGGGAGAUGGUU | |
| CAGGUGGAAACCAUCUCCCCguuuuagaGCUAGAAAUAGCAAGUUAAAAUAAGGCUAGUCCG | 935 |
| UUAUCAACUUGAAAAAGUGGCACCGAGUCGgugcCgCCGUCUCCUGGGGAGAUGGUUU | |
| CAGGUGGAAACCAUCUCCCCguuuuagaGCUAGAAAUAGCAAGUUAAAAUAAGGCUAGUCCG | 936 |
| UUAUCAACUUGAAAAAGUGGCACCGAGUCGgugcCgCCGUCUCCUGGGGAGAUGGUUUC | |
| CAGGUGGAAACCAUCUCCCCguuuuagaGCUAGAAAUAGCAAGUUAAAAUAAGGCUAGUCCG | 937 |
| UUAUCAACUUGAAAAAGUGGCACCGAGUCGgugcCgCCGUCUCCUGGGGAGAUGGUUUCC | |
| CAGGUGGAAACCAUCUCCCCguuuuagaGCUAGAAAUAGCAAGUUAAAAUAAGGCUAGUCCG | 938 |
| UUAUCAACUUGAAAAAGUGGCACCGAGUCGgugcCgCCGUCUCCUGGGGAGAUGGUUUCCA | |
| CAGGUGGAAACCAUCUCCCCguuuuagaGCUAGAAAUAGCAAGUUAAAAUAAGGCUAGUCCG | 939 |
| UUAUCAACUUGAAAAAGUGGCACCGAGUCGgugcCgCCGUCUCCUGGGGAGAUGGUUUCCAC | |
| CAGGUGGAAACCAUCUCCCCguuuuagaGCUAGAAAUAGCAAGUUAAAAUAAGGCUAGUCCG | 940 |
| UUAUCAACUUGAAAAAGUGGCACCGAGUCGgugcCgCCGUCUCCUGGGGAGAUGGUUUCCAC | |
| C | |
| CAGGUGGAAACCAUCUCCCCguuuuagaGCUAGAAAUAGCAAGUUAAAAUAAGGCUAGUCCG | 941 |
| UUAUCAACUUGAAAAAGUGGCACCGAGUCGgugcUCgCCGUCUCCUGGGGAGAUGGUUU | |
| CAGGUGGAAACCAUCUCCCCguuuuagaGCUAGAAAUAGCAAGUUAAAAUAAGGCUAGUCCG | 942 |
| UUAUCAACUUGAAAAAGUGGCACCGAGUCGgugcUCgCCGUCUCCUGGGGAGAUGGUUUC | |
| CAGGUGGAAACCAUCUCCCCguuuuagaGCUAGAAAUAGCAAGUUAAAAUAAGGCUAGUCCG | 943 |
| UUAUCAACUUGAAAAAGUGGCACCGAGUCGgugcUCgCCGUCUCCUGGGGAGAUGGUUUCC | |
| CAGGUGGAAACCAUCUCCCCguuuuagaGCUAGAAAUAGCAAGUUAAAAUAAGGCUAGUCCG | 944 |
| UUAUCAACUUGAAAAAGUGGCACCGAGUCGgugcUCgCCGUCUCCUGGGGAGAUGGUUUCCA | |
| CAGGUGGAAACCAUCUCCCCguuuuagaGCUAGAAAUAGCAAGUUAAAAUAAGGCUAGUCCG | 945 |
| UUAUCAACUUGAAAAAGUGGCACCGAGUCGgugcUCgCCGUCUCCUGGGGAGAUGGUUUCCA | |
| C | |
| CAGGUGGAAACCAUCUCCCCguuuuagaGCUAGAAAUAGCAAGUUAAAAUAAGGCUAGUCCG | 946 |
| UUAUCAACUUGAAAAAGUGGCACCGAGUCGgugcUCgCCGUCUCCUGGGGAGAUGGUUUCCA | |
| CC | |
| CAGGUGGAAACCAUCUCCCCguuuuagaGCUAGAAAUAGCAAGUUAAAAUAAGGCUAGUCCG | 947 |
| UUAUCAACUUGAAAAAGUGGCACCGAGUCGgugcCUCgCCGUCUCCUGGGGAGAUGGUUU | |
| CAGGUGGAAACCAUCUCCCCguuuuagaGCUAGAAAUAGCAAGUUAAAAUAAGGCUAGUCCG | 948 |
| UUAUCAACUUGAAAAAGUGGCACCGAGUCGgugcCUCgCCGUCUCCUGGGGAGAUGGUUUC | |
| CAGGUGGAAACCAUCUCCCCguuuuagaGCUAGAAAUAGCAAGUUAAAAUAAGGCUAGUCCG | 949 |
| UUAUCAACUUGAAAAAGUGGCACCGAGUCGgugcCUCgCCGUCUCCUGGGGAGAUGGUUUCC | |
| CAGGUGGAAACCAUCUCCCCguuuuagaGCUAGAAAUAGCAAGUUAAAAUAAGGCUAGUCCG | 950 |
| UUAUCAACUUGAAAAAGUGGCACCGAGUCGgugcCUCgCCGUCUCCUGGGGAGAUGGUUUCC | |
| A | |
| CAGGUGGAAACCAUCUCCCCguuuuagaGCUAGAAAUAGCAAGUUAAAAUAAGGCUAGUCCG | 951 |
| UUAUCAACUUGAAAAAGUGGCACCGAGUCGgugcCUCgCCGUCUCCUGGGGAGAUGGUUUCC | |
| AC | |
| CAGGUGGAAACCAUCUCCCCguuuuagaGCUAGAAAUAGCAAGUUAAAAUAAGGCUAGUCCG | 952 |
| UUAUCAACUUGAAAAAGUGGCACCGAGUCGgugcCUCgCCGUCUCCUGGGGAGAUGGUUUCC | |
| ACC | |
| CAGGUGGAAACCAUCUCCCCguuuuagaGCUAGAAAUAGCAAGUUAAAAUAAGGCUAGUCCG | 953 |
| UUAUCAACUUGAAAAAGUGGCACCGAGUCGgugcACUCgCCGUCUCCUGGGGAGAUGGUUU | |
| CAGGUGGAAACCAUCUCCCCguuuuagaGCUAGAAAUAGCAAGUUAAAAUAAGGCUAGUCCG | 954 |
| UUAUCAACUUGAAAAAGUGGCACCGAGUCGgugcACUCgCCGUCUCCUGGGGAGAUGGUUUC | |
| CAGGUGGAAACCAUCUCCCCguuuuagaGCUAGAAAUAGCAAGUUAAAAUAAGGCUAGUCCG | 955 |
| UUAUCAACUUGAAAAAGUGGCACCGAGUCGgugcACUCgCCGUCUCCUGGGGAGAUGGUUUC | |
| C | |
| CAGGUGGAAACCAUCUCCCCguuuuagaGCUAGAAAUAGCAAGUUAAAAUAAGGCUAGUCCG | 956 |
| UUAUCAACUUGAAAAAGUGGCACCGAGUCGgugcACUCgCCGUCUCCUGGGGAGAUGGUUUC | |
| CA | |
| CAGGUGGAAACCAUCUCCCCguuuuagaGCUAGAAAUAGCAAGUUAAAAUAAGGCUAGUCCG | 957 |
| UUAUCAACUUGAAAAAGUGGCACCGAGUCGgugcACUCgCCGUCUCCUGGGGAGAUGGUUUC | |
| CAC | |
| CAGGUGGAAACCAUCUCCCCguuuuagaGCUAGAAAUAGCAAGUUAAAAUAAGGCUAGUCCG | 958 |
| UUAUCAACUUGAAAAAGUGGCACCGAGUCGgugcACUCgCCGUCUCCUGGGGAGAUGGUUUC | |
| CACC | |
| CAGGUGGAAACCAUCUCCCCguuuuagaGCUAGAAAUAGCAAGUUAAAAUAAGGCUAGUCCG | 959 |
| UUAUCAACUUGAAAAAGUGGCACCGAGUCGgugcUACUCgCCGUCUCCUGGGGAGAUGGUUU | |
| CAGGUGGAAACCAUCUCCCCguuuuagaGCUAGAAAUAGCAAGUUAAAAUAAGGCUAGUCCG | 960 |
| UUAUCAACUUGAAAAAGUGGCACCGAGUCGgugcUACUCgCCGUCUCCUGGGGAGAUGGUUU | |
| C | |
| CAGGUGGAAACCAUCUCCCCguuuuagaGCUAGAAAUAGCAAGUUAAAAUAAGGCUAGUCCG | 961 |
| UUAUCAACUUGAAAAAGUGGCACCGAGUCGgugcUACUCgCCGUCUCCUGGGGAGAUGGUUU | |
| CC | |
| CAGGUGGAAACCAUCUCCCCguuuuagaGCUAGAAAUAGCAAGUUAAAAUAAGGCUAGUCCG | 962 |
| UUAUCAACUUGAAAAAGUGGCACCGAGUCGgugcUACUCgCCGUCUCCUGGGGAGAUGGUUU | |
| CCA | |
| CAGGUGGAAACCAUCUCCCCguuuuagaGCUAGAAAUAGCAAGUUAAAAUAAGGCUAGUCCG | 963 |
| UUAUCAACUUGAAAAAGUGGCACCGAGUCGgugcUACUCgCCGUCUCCUGGGGAGAUGGUUU | |
| CCAC | |
| CAGGUGGAAACCAUCUCCCCguuuuagaGCUAGAAAUAGCAAGUUAAAAUAAGGCUAGUCCG | 964 |
| UUAUCAACUUGAAAAAGUGGCACCGAGUCGgugcUACUCgCCGUCUCCUGGGGAGAUGGUUU | |
| CCACC | |
| CAGGUGGAAACCAUCUCCCCguuuuagaGCUAGAAAUAGCAAGUUAAAAUAAGGCUAGUCCG | 965 |
| UUAUCAACUUGAAAAAGUGGCACCGAGUCGgugcCUACUCgCCGUCUCCUGGGGAGAUGGUU | |
| U | |
| CAGGUGGAAACCAUCUCCCCguuuuagaGCUAGAAAUAGCAAGUUAAAAUAAGGCUAGUCCG | 966 |
| UUAUCAACUUGAAAAAGUGGCACCGAGUCGgugcCUACUCgCCGUCUCCUGGGGAGAUGGUU | |
| UC | |
| CAGGUGGAAACCAUCUCCCCguuuuagaGCUAGAAAUAGCAAGUUAAAAUAAGGCUAGUCCG | 967 |
| UUAUCAACUUGAAAAAGUGGCACCGAGUCGgugcCUACUCgCCGUCUCCUGGGGAGAUGGUU | |
| UCC | |
| CAGGUGGAAACCAUCUCCCCguuuuagaGCUAGAAAUAGCAAGUUAAAAUAAGGCUAGUCCG | 968 |
| UUAUCAACUUGAAAAAGUGGCACCGAGUCGgugcCUACUCgCCGUCUCCUGGGGAGAUGGUU | |
| UCCA | |
| CAGGUGGAAACCAUCUCCCCguuuuagaGCUAGAAAUAGCAAGUUAAAAUAAGGCUAGUCCG | 969 |
| UUAUCAACUUGAAAAAGUGGCACCGAGUCGgugcCUACUCgCCGUCUCCUGGGGAGAUGGUU | |
| UCCAC | |
| CAGGUGGAAACCAUCUCCCCguuuuagaGCUAGAAAUAGCAAGUUAAAAUAAGGCUAGUCCG | 970 |
| UUAUCAACUUGAAAAAGUGGCACCGAGUCGgugcCUACUCgCCGUCUCCUGGGGAGAUGGUU | |
| UCCACC | |
| CAGGUGGAAACCAUCUCCCCguuuuagaGCUAGAAAUAGCAAGUUAAAAUAAGGCUAGUCCG | 971 |
| UUAUCAACUUGAAAAAGUGGCACCGAGUCGgugcACUACUCgCCGUCUCCUGGGGAGAUGGU | |
| UU | |
| CAGGUGGAAACCAUCUCCCCguuuuagaGCUAGAAAUAGCAAGUUAAAAUAAGGCUAGUCCG | 972 |
| UUAUCAACUUGAAAAAGUGGCACCGAGUCGgugcACUACUCgCCGUCUCCUGGGGAGAUGGU | |
| UUC | |
| CAGGUGGAAACCAUCUCCCCguuuuagaGCUAGAAAUAGCAAGUUAAAAUAAGGCUAGUCCG | 973 |
| UUAUCAACUUGAAAAAGUGGCACCGAGUCGgugcACUACUCgCCGUCUCCUGGGGAGAUGGU | |
| UUCC | |
| CAGGUGGAAACCAUCUCCCCguuuuagaGCUAGAAAUAGCAAGUUAAAAUAAGGCUAGUCCG | 974 |
| UUAUCAACUUGAAAAAGUGGCACCGAGUCGgugcACUACUCgCCGUCUCCUGGGGAGAUGGU | |
| UUCCA | |
| CAGGUGGAAACCAUCUCCCCguuuuagaGCUAGAAAUAGCAAGUUAAAAUAAGGCUAGUCCG | 975 |
| UUAUCAACUUGAAAAAGUGGCACCGAGUCGgugcACUACUCgCCGUCUCCUGGGGAGAUGGU | |
| UUCCAC | |
| CAGGUGGAAACCAUCUCCCCguuuuagaGCUAGAAAUAGCAAGUUAAAAUAAGGCUAGUCCG | 976 |
| UUAUCAACUUGAAAAAGUGGCACCGAGUCGgugcACUACUCgCCGUCUCCUGGGGAGAUGGU | |
| UUCCACC | |
| CAGGUGGAAACCAUCUCCCCguuuuagaGCUAGAAAUAGCAAGUUAAAAUAAGGCUAGUCCG | 977 |
| UUAUCAACUUGAAAAAGUGGCACCGAGUCGgugcCACUACUCgCCGUCUCCUGGGGAGAUGG | |
| UUU | |
| CAGGUGGAAACCAUCUCCCCguuuuagaGCUAGAAAUAGCAAGUUAAAAUAAGGCUAGUCCG | 978 |
| UUAUCAACUUGAAAAAGUGGCACCGAGUCGgugcCACUACUCgCCGUCUCCUGGGGAGAUGG | |
| UUUC | |
| CAGGUGGAAACCAUCUCCCCguuuuagaGCUAGAAAUAGCAAGUUAAAAUAAGGCUAGUCCG | 979 |
| UUAUCAACUUGAAAAAGUGGCACCGAGUCGgugcCACUACUCgCCGUCUCCUGGGGAGAUGG | |
| UUUCC | |
| CAGGUGGAAACCAUCUCCCCguuuuagaGCUAGAAAUAGCAAGUUAAAAUAAGGCUAGUCCG | 980 |
| UUAUCAACUUGAAAAAGUGGCACCGAGUCGgugcCACUACUCgCCGUCUCCUGGGGAGAUGG | |
| UUUCCA | |
| CAGGUGGAAACCAUCUCCCCguuuuagaGCUAGAAAUAGCAAGUUAAAAUAAGGCUAGUCCG | 981 |
| UUAUCAACUUGAAAAAGUGGCACCGAGUCGgugcCACUACUCgCCGUCUCCUGGGGAGAUGG | |
| UUUCCAC | |
| CAGGUGGAAACCAUCUCCCCguuuuagaGCUAGAAAUAGCAAGUUAAAAUAAGGCUAGUCCG | 982 |
| UUAUCAACUUGAAAAAGUGGCACCGAGUCGgugcCACUACUCgCCGUCUCCUGGGGAGAUGG | |
| UUUCCACC | |
| CAGGUGGAAACCAUCUCCCCguuuuagaGCUAGAAAUAGCAAGUUAAAAUAAGGCUAGUCCG | 983 |
| UUAUCAACUUGAAAAAGUGGCACCGAGUCGgugcCCACUACUCgCCGUCUCCUGGGGAGAUG | |
| GUUU | |
| CAGGUGGAAACCAUCUCCCCguuuuagaGCUAGAAAUAGCAAGUUAAAAUAAGGCUAGUCCG | 984 |
| UUAUCAACUUGAAAAAGUGGCACCGAGUCGgugcCCACUACUCgCCGUCUCCUGGGGAGAUG | |
| GUUUC | |
| CAGGUGGAAACCAUCUCCCCguuuuagaGCUAGAAAUAGCAAGUUAAAAUAAGGCUAGUCCG | 985 |
| UUAUCAACUUGAAAAAGUGGCACCGAGUCGgugcCCACUACUCgCCGUCUCCUGGGGAGAUG | |
| GUUUCC | |
| CAGGUGGAAACCAUCUCCCCguuuuagaGCUAGAAAUAGCAAGUUAAAAUAAGGCUAGUCCG | 986 |
| UUAUCAACUUGAAAAAGUGGCACCGAGUCGgugcCCACUACUCgCCGUCUCCUGGGGAGAUG | |
| GUUUCCA | |
| CAGGUGGAAACCAUCUCCCCguuuuagaGCUAGAAAUAGCAAGUUAAAAUAAGGCUAGUCCG | 987 |
| UUAUCAACUUGAAAAAGUGGCACCGAGUCGgugcCCACUACUCgCCGUCUCCUGGGGAGAUG | |
| GUUUCCAC | |
| CAGGUGGAAACCAUCUCCCCguuuuagaGCUAGAAAUAGCAAGUUAAAAUAAGGCUAGUCCG | 988 |
| UUAUCAACUUGAAAAAGUGGCACCGAGUCGgugcCCACUACUCgCCGUCUCCUGGGGAGAUG | |
| GUUUCCACC | |
| CAGGUGGAAACCAUCUCCCCguuuuagaGCUAGAAAUAGCAAGUUAAAAUAAGGCUAGUCCG | 989 |
| UUAUCAACUUGAAAAAGUGGCACCGAGUCGgugcGCCACUACUCgCCGUCUCCUGGGGAGAU | |
| GGUUU | |
| CAGGUGGAAACCAUCUCCCCguuuuagaGCUAGAAAUAGCAAGUUAAAAUAAGGCUAGUCCG | 990 |
| UUAUCAACUUGAAAAAGUGGCACCGAGUCGgugcGCCACUACUCgCCGUCUCCUGGGGAGAU | |
| GGUUUC | |
| CAGGUGGAAACCAUCUCCCCguuuuagaGCUAGAAAUAGCAAGUUAAAAUAAGGCUAGUCCG | 991 |
| UUAUCAACUUGAAAAAGUGGCACCGAGUCGgugcGCCACUACUCgCCGUCUCCUGGGGAGAU | |
| GGUUUCC | |
| CAGGUGGAAACCAUCUCCCCguuuuagaGCUAGAAAUAGCAAGUUAAAAUAAGGCUAGUCCG | 992 |
| UUAUCAACUUGAAAAAGUGGCACCGAGUCGgugcGCCACUACUCgCCGUCUCCUGGGGAGAU | |
| GGUUUCCA | |
| CAGGUGGAAACCAUCUCCCCguuuuagaGCUAGAAAUAGCAAGUUAAAAUAAGGCUAGUCCG | 993 |
| UUAUCAACUUGAAAAAGUGGCACCGAGUCGgugcGCCACUACUCgCCGUCUCCUGGGGAGAU | |
| GGUUUCCAC | |
| CAGGUGGAAACCAUCUCCCCguuuuagaGCUAGAAAUAGCAAGUUAAAAUAAGGCUAGUCCG | 994 |
| UUAUCAACUUGAAAAAGUGGCACCGAGUCGgugcGCCACUACUCgCCGUCUCCUGGGGAGAU | |
| GGUUUCCACC | |
| CAGGUGGAAACCAUCUCCCCguuuuagaGCUAGAAAUAGCAAGUUAAAAUAAGGCUAGUCCG | 995 |
| UUAUCAACUUGAAAAAGUGGCACCGAGUCGgugcCGCCACUACUCgCCGUCUCCUGGGGAGA | |
| UGGUUU | |
| CAGGUGGAAACCAUCUCCCCguuuuagaGCUAGAAAUAGCAAGUUAAAAUAAGGCUAGUCCG | 996 |
| UUAUCAACUUGAAAAAGUGGCACCGAGUCGgugcCGCCACUACUCgCCGUCUCCUGGGGAGA | |
| UGGUUUC | |
| CAGGUGGAAACCAUCUCCCCguuuuagaGCUAGAAAUAGCAAGUUAAAAUAAGGCUAGUCCG | 997 |
| UUAUCAACUUGAAAAAGUGGCACCGAGUCGgugcCGCCACUACUCgCCGUCUCCUGGGGAGA | |
| UGGUUUCC | |
| CAGGUGGAAACCAUCUCCCCguuuuagaGCUAGAAAUAGCAAGUUAAAAUAAGGCUAGUCCG | 998 |
| UUAUCAACUUGAAAAAGUGGCACCGAGUCGgugcCGCCACUACUCgCCGUCUCCUGGGGAGA | |
| UGGUUUCCA | |
| CAGGUGGAAACCAUCUCCCCguuuuagaGCUAGAAAUAGCAAGUUAAAAUAAGGCUAGUCCG | 999 |
| UUAUCAACUUGAAAAAGUGGCACCGAGUCGgugcCGCCACUACUCgCCGUCUCCUGGGGAGA | |
| UGGUUUCCAC | |
| CAGGUGGAAACCAUCUCCCCguuuuagaGCUAGAAAUAGCAAGUUAAAAUAAGGCUAGUCCG | 1000 |
| UUAUCAACUUGAAAAAGUGGCACCGAGUCGgugcCGCCACUACUCgCCGUCUCCUGGGGAGA | |
| UGGUUUCCACC | |
| CAGGUGGAAACCAUCUCCCCguuuuagaGCUAGAAAUAGCAAGUUAAAAUAAGGCUAGUCCG | 1001 |
| UUAUCAACUUGAAAAAGUGGCACCGAGUCGgugcGCGCCACUACUCgCCGUCUCCUGGGGAG | |
| AUGGUUU | |
| CAGGUGGAAACCAUCUCCCCguuuuagaGCUAGAAAUAGCAAGUUAAAAUAAGGCUAGUCCG | 1002 |
| UUAUCAACUUGAAAAAGUGGCACCGAGUCGgugcGCGCCACUACUCgCCGUCUCCUGGGGAG | |
| AUGGUUUC | |
| CAGGUGGAAACCAUCUCCCCguuuuagaGCUAGAAAUAGCAAGUUAAAAUAAGGCUAGUCCG | 1003 |
| UUAUCAACUUGAAAAAGUGGCACCGAGUCGgugcGCGCCACUACUCgCCGUCUCCUGGGGAG | |
| AUGGUUUCC | |
| CAGGUGGAAACCAUCUCCCCguuuuagaGCUAGAAAUAGCAAGUUAAAAUAAGGCUAGUCCG | 1004 |
| UUAUCAACUUGAAAAAGUGGCACCGAGUCGgugcGCGCCACUACUCgCCGUCUCCUGGGGAG | |
| AUGGUUUCCA | |
| CAGGUGGAAACCAUCUCCCCguuuuagaGCUAGAAAUAGCAAGUUAAAAUAAGGCUAGUCCG | 1005 |
| UUAUCAACUUGAAAAAGUGGCACCGAGUCGgugcGCGCCACUACUCgCCGUCUCCUGGGGAG | |
| AUGGUUUCCAC | |
| CAGGUGGAAACCAUCUCCCCguuuuagaGCUAGAAAUAGCAAGUUAAAAUAAGGCUAGUCCG | 1006 |
| UUAUCAACUUGAAAAAGUGGCACCGAGUCGgugcGCGCCACUACUCgCCGUCUCCUGGGGAG | |
| AUGGUUUCCACC | |
| CAGGUGGAAACCAUCUCCCCguuuuagaGCUAGAAAUAGCAAGUUAAAAUAAGGCUAGUCCG | 1007 |
| UUAUCAACUUGAAAAAGUGGCACCGAGUCGgugcCGCGCCACUACUCgCCGUCUCCUGGGGA | |
| GAUGGUUU | |
| CAGGUGGAAACCAUCUCCCCguuuuagaGCUAGAAAUAGCAAGUUAAAAUAAGGCUAGUCCG | 1008 |
| UUAUCAACUUGAAAAAGUGGCACCGAGUCGgugcCGCGCCACUACUCgCCGUCUCCUGGGGA | |
| GAUGGUUUC | |
| CAGGUGGAAACCAUCUCCCCguuuuagaGCUAGAAAUAGCAAGUUAAAAUAAGGCUAGUCCG | 1009 |
| UUAUCAACUUGAAAAAGUGGCACCGAGUCGgugcCGCGCCACUACUCgCCGUCUCCUGGGGA | |
| GAUGGUUUCC | |
| CAGGUGGAAACCAUCUCCCCguuuuagaGCUAGAAAUAGCAAGUUAAAAUAAGGCUAGUCCG | 1010 |
| UUAUCAACUUGAAAAAGUGGCACCGAGUCGgugcCGCGCCACUACUCgCCGUCUCCUGGGGA | |
| GAUGGUUUCCA | |
| CAGGUGGAAACCAUCUCCCCguuuuagaGCUAGAAAUAGCAAGUUAAAAUAAGGCUAGUCCG | 1011 |
| UUAUCAACUUGAAAAAGUGGCACCGAGUCGgugcCGCGCCACUACUCgCCGUCUCCUGGGGA | |
| GAUGGUUUCCAC | |
| CAGGUGGAAACCAUCUCCCCguuuuagaGCUAGAAAUAGCAAGUUAAAAUAAGGCUAGUCCG | 1012 |
| UUAUCAACUUGAAAAAGUGGCACCGAGUCGgugcCGCGCCACUACUCgCCGUCUCCUGGGGA | |
| GAUGGUUUCCACC | |
| CAGGUGGAAACCAUCUCCCCguuuuagaGCUAGAAAUAGCAAGUUAAAAUAAGGCUAGUCCG | 1013 |
| UUAUCAACUUGAAAAAGUGGCACCGAGUCGgugcGCGCGCCACUACUCgCCGUCUCCUGGGG | |
| AGAUGGUUU | |
| CAGGUGGAAACCAUCUCCCCguuuuagaGCUAGAAAUAGCAAGUUAAAAUAAGGCUAGUCCG | 1014 |
| UUAUCAACUUGAAAAAGUGGCACCGAGUCGgugcGCGCGCCACUACUCgCCGUCUCCUGGGG | |
| AGAUGGUUUC | |
| CAGGUGGAAACCAUCUCCCCguuuuagaGCUAGAAAUAGCAAGUUAAAAUAAGGCUAGUCCG | 1015 |
| UUAUCAACUUGAAAAAGUGGCACCGAGUCGgugcGCGCGCCACUACUCgCCGUCUCCUGGGG | |
| AGAUGGUUUCC | |
| CAGGUGGAAACCAUCUCCCCguuuuagaGCUAGAAAUAGCAAGUUAAAAUAAGGCUAGUCCG | 1016 |
| UUAUCAACUUGAAAAAGUGGCACCGAGUCGgugcGCGCGCCACUACUCgCCGUCUCCUGGGG | |
| AGAUGGUUUCCA | |
| CAGGUGGAAACCAUCUCCCCguuuuagaGCUAGAAAUAGCAAGUUAAAAUAAGGCUAGUCCG | 1017 |
| UUAUCAACUUGAAAAAGUGGCACCGAGUCGgugcGCGCGCCACUACUCgCCGUCUCCUGGGG | |
| AGAUGGUUUCCAC | |
| CAGGUGGAAACCAUCUCCCCguuuuagaGCUAGAAAUAGCAAGUUAAAAUAAGGCUAGUCCG | 1018 |
| UUAUCAACUUGAAAAAGUGGCACCGAGUCGgugcGCGCGCCACUACUCgCCGUCUCCUGGGG | |
| AGAUGGUUUCCACC | |
| CAGGUGGAAACCAUCUCCCCguuuuagaGCUAGAAAUAGCAAGUUAAAAUAAGGCUAGUCCG | 1019 |
| UUAUCAACUUGAAAAAGUGGCACCGAGUCGgugcCGCGCGCCACUACUCgCCGUCUCCUGGG | |
| GAGAUGGUUU | |
| CAGGUGGAAACCAUCUCCCCguuuuagaGCUAGAAAUAGCAAGUUAAAAUAAGGCUAGUCCG | 1020 |
| UUAUCAACUUGAAAAAGUGGCACCGAGUCGgugcCGCGCGCCACUACUCgCCGUCUCCUGGG | |
| GAGAUGGUUUC | |
| CAGGUGGAAACCAUCUCCCCguuuuagaGCUAGAAAUAGCAAGUUAAAAUAAGGCUAGUCCG | 1021 |
| UUAUCAACUUGAAAAAGUGGCACCGAGUCGgugcCGCGCGCCACUACUCgCCGUCUCCUGGG | |
| GAGAUGGUUUCC | |
| CAGGUGGAAACCAUCUCCCCguuuuagaGCUAGAAAUAGCAAGUUAAAAUAAGGCUAGUCCG | 1022 |
| UUAUCAACUUGAAAAAGUGGCACCGAGUCGgugcCGCGCGCCACUACUCgCCGUCUCCUGGG | |
| GAGAUGGUUUCCA | |
| CAGGUGGAAACCAUCUCCCCguuuuagaGCUAGAAAUAGCAAGUUAAAAUAAGGCUAGUCCG | 1023 |
| UUAUCAACUUGAAAAAGUGGCACCGAGUCGgugcCGCGCGCCACUACUCgCCGUCUCCUGGG | |
| GAGAUGGUUUCCAC | |
| CAGGUGGAAACCAUCUCCCCguuuuagaGCUAGAAAUAGCAAGUUAAAAUAAGGCUAGUCCG | 1024 |
| UUAUCAACUUGAAAAAGUGGCACCGAGUCGgugcCGCGCGCCACUACUCgCCGUCUCCUGGG | |
| GAGAUGGUUUCCACC | |
| CAGGUGGAAACCAUCUCCCCguuuuagaGCUAGAAAUAGCAAGUUAAAAUAAGGCUAGUCCG | 1025 |
| UUAUCAACUUGAAAAAGUGGCACCGAGUCGgugcCCGCGCGCCACUACUCgCCGUCUCCUGG | |
| GGAGAUGGUUU | |
| CAGGUGGAAACCAUCUCCCCguuuuagaGCUAGAAAUAGCAAGUUAAAAUAAGGCUAGUCCG | 1026 |
| UUAUCAACUUGAAAAAGUGGCACCGAGUCGgugcCCGCGCGCCACUACUCgCCGUCUCCUGG | |
| GGAGAUGGUUUC | |
| CAGGUGGAAACCAUCUCCCCguuuuagaGCUAGAAAUAGCAAGUUAAAAUAAGGCUAGUCCG | 1027 |
| UUAUCAACUUGAAAAAGUGGCACCGAGUCGgugcCCGCGCGCCACUACUCgCCGUCUCCUGG | |
| GGAGAUGGUUUCC | |
| CAGGUGGAAACCAUCUCCCCguuuuagaGCUAGAAAUAGCAAGUUAAAAUAAGGCUAGUCCG | 1028 |
| UUAUCAACUUGAAAAAGUGGCACCGAGUCGgugcCCGCGCGCCACUACUCgCCGUCUCCUGG | |
| GGAGAUGGUUUCCA | |
| CAGGUGGAAACCAUCUCCCCguuuuagaGCUAGAAAUAGCAAGUUAAAAUAAGGCUAGUCCG | 1029 |
| UUAUCAACUUGAAAAAGUGGCACCGAGUCGgugcCCGCGCGCCACUACUCgCCGUCUCCUGG | |
| GGAGAUGGUUUCCAC | |
| CAGGUGGAAACCAUCUCCCCguuuuagaGCUAGAAAUAGCAAGUUAAAAUAAGGCUAGUCCG | 1030 |
| UUAUCAACUUGAAAAAGUGGCACCGAGUCGgugcCCGCGCGCCACUACUCgCCGUCUCCUGG | |
| GGAGAUGGUUUCCACC | |
| CAGGUGGAAACCAUCUCCCCguuuuagaGCUAGAAAUAGCAAGUUAAAAUAAGGCUAGUCCG | 1031 |
| UUAUCAACUUGAAAAAGUGGCACCGAGUCGgugcGCCGCGCGCCACUACUCgCCGUCUCCUG | |
| GGGAGAUGGUUU | |
| CAGGUGGAAACCAUCUCCCCguuuuagaGCUAGAAAUAGCAAGUUAAAAUAAGGCUAGUCCG | 1032 |
| UUAUCAACUUGAAAAAGUGGCACCGAGUCGgugcGCCGCGCGCCACUACUCgCCGUCUCCUG | |
| GGGAGAUGGUUUC | |
| CAGGUGGAAACCAUCUCCCCguuuuagaGCUAGAAAUAGCAAGUUAAAAUAAGGCUAGUCCG | 1033 |
| UUAUCAACUUGAAAAAGUGGCACCGAGUCGgugcGCCGCGCGCCACUACUCgCCGUCUCCUG | |
| GGGAGAUGGUUUCC | |
| CAGGUGGAAACCAUCUCCCCguuuuagaGCUAGAAAUAGCAAGUUAAAAUAAGGCUAGUCCG | 1034 |
| UUAUCAACUUGAAAAAGUGGCACCGAGUCGgugcGCCGCGCGCCACUACUCgCCGUCUCCUG | |
| GGGAGAUGGUUUCCA | |
| CAGGUGGAAACCAUCUCCCCguuuuagaGCUAGAAAUAGCAAGUUAAAAUAAGGCUAGUCCG | 1035 |
| UUAUCAACUUGAAAAAGUGGCACCGAGUCGgugcGCCGCGCGCCACUACUCgCCGUCUCCUG | |
| GGGAGAUGGUUUCCAC | |
| CAGGUGGAAACCAUCUCCCCguuuuagaGCUAGAAAUAGCAAGUUAAAAUAAGGCUAGUCCG | 1036 |
| UUAUCAACUUGAAAAAGUGGCACCGAGUCGgugcGCCGCGCGCCACUACUCgCCGUCUCCUG | |
| GGGAGAUGGUUUCCACC | |

TABLE 11
nickRNA (nRNA) Sequences.

| | DNA target | | | | | SEQ |
|---|---|---|---|---|---|---|
| | Sequence | SEQ | Spacer | SEQ | | ID |
| Descrip- | (including | ID | sequence | ID | nickRNA Sequence (nRNA) (no | NO |
| tion | PAM) | NO | (RNA) | NO | chemical modifications) | |
| nRNA | GCGCCCTGAG | 1037 | GCGCCCU | 1063 | GCGCCCUGAGGAGACAGAGAGUUUUAGA | 1089 |
| | GAGACAGAGA | | GAGGAGA | | GCUAGAAAUAGCAAGUUAAAAUAAGGCU | |
| | CGG | | CAGAGA | | AGUCCGUUAUCAACUUGAAAAAGUGGCA | |
| | | | | | CCGAGUCGGUGCUUUU | |
| nRNA | CGCCCTGAGG | 1038 | CGCCCUG | 1064 | CGCCCUGAGGAGACAGAGACGUUUUAGA | 1090 |
| | AGACAGAGAC | | AGGAGAC | | GCUAGAAAUAGCAAGUUAAAAUAAGGCU | |
| | GGG | | AGAGAC | | AGUCCGUUAUCAACUUGAAAAAGUGGCA | |
| | | | | | CCGAGUCGGUGCUUUU | |
| nRNA | GCATGCTGAG | 1039 | GCAUGCU | 1065 | GCAUGCUGAGCCGAUGCGCGGUUUUAGA | 1091 |
| | CCGATGCGCG | | GAGCCGA | | GCUAGAAAUAGCAAGUUAAAAUAAGGCU | |
| | CGG | | UGCGCG | | AGUCCGUUAUCAACUUGAAAAAGUGGCA | |
| | | | | | CCGAGUCGGUGCUUUU | |
| nRNA | TGCTGAGCCG | 1040 | UGCUGAG | 1066 | UGCUGAGCCGAUGCGCGCGGGUUUUAGA | 1092 |
| | ATGCGCGCGG | | CCGAUGC | | GCUAGAAAUAGCAAGUUAAAAUAAGGCU | |
| | CGG | | GCGCGG | | AGUCCGUUAUCAACUUGAAAAAGUGGCA | |
| | | | | | CCGAGUCGGUGCUUUU | |
| nRNA | GCCGATGCGC | 1041 | GCCGAUG | 1067 | GCCGAUGCGCGCGGCGGCAGGUUUUAGA | 1093 |
| | GCGGCGGCAG | | CGCGCGG | | GCUAGAAAUAGCAAGUUAAAAUAAGGCU | |
| | AGG | | CGGCAG | | AGUCCGUUAUCAACUUGAAAAAGUGGCA | |
| | | | | | CCGAGUCGGUGCUUUU | |
| nRNA | CGCCACTACT | 1042 | CGCCACU | 1068 | CGCCACUACUCACCGUCUCCGUUUUAGA | 1094 |
| | CACCGTCTCC | | ACUCACC | | GCUAGAAAUAGCAAGUUAAAAUAAGGCU | |
| | TGG | | GUCUCC | | AGUCCGUUAUCAACUUGAAAAAGUGGCA | |
| | | | | | CCGAGUCGGUGCUUUU | |
| nRNA | GCCACTACTC | 1043 | GCCACUA | 1069 | GCCACUACUCACCGUCUCCUGUUUUAGA | 1095 |
| | ACCGTCTCCT | | CUCACCG | | GCUAGAAAUAGCAAGUUAAAAUAAGGCU | |
| | GGG | | UCUCCU | | AGUCCGUUAUCAACUUGAAAAAGUGGCA | |
| | | | | | CCGAGUCGGUGCUUUU | |
| nRNA | CCACTACTCA | 1044 | CCACUAC | 1070 | CCACUACUCACCGUCUCCUGGUUUUAGA | 1096 |
| | CCGTCTCCTG | | UCACCGU | | GCUAGAAAUAGCAAGUUAAAAUAAGGCU | |
| | GGG | | CUCCUG | | AGUCCGUUAUCAACUUGAAAAAGUGGCA | |
| | | | | | CCGAGUCGGUGCUUUU | |
| nRNA | CTCACCGTCT | 1045 | CUCACCG | 1071 | CUCACCGUCUCCUGGGGAGAGUUUUAGA | 1097 |
| | CCTGGGGAGA | | UCUCCUG | | GCUAGAAAUAGCAAGUUAAAAUAAGGCU | |
| | TGG | | GGGAGA | | AGUCCGUUAUCAACUUGAAAAAGUGGCA | |
| | | | | | CCGAGUCGGUGCUUUU | |
| nRNA | GGTTTCCACC | 1046 | GGUUUCC | 1072 | GGUUUCCACCUGCACUCCCAGUUUUAGA | 1098 |
| | TGCACTCCCA | | ACCUGCA | | GCUAGAAAUAGCAAGUUAAAAUAAGGCU | |
| | TGG | | CUCCCA | | AGUCCGUUAUCAACUUGAAAAAGUGGCA | |
| | | | | | CCGAGUCGGUGCUUUU | |
| nRNA | TTCCACCTGC | 1047 | UUCCACC | 1073 | UUCCACCUGCACUCCCAUGGGUUUUAGA | 1099 |
| | ACTCCCATGG | | UGCACUC | | GCUAGAAAUAGCAAGUUAAAAUAAGGCU | |
| | CGG | | CCAUGG | | AGUCCGUUAUCAACUUGAAAAAGUGGCA | |
| | | | | | CCGAGUCGGUGCUUUU | |
| nRNA | CACCTGCACT | 1048 | CACCUGC | 1074 | CACCUGCACUCCCAUGGCGGGUUUUAGA | 1100 |
| | CCCATGGCGG | | ACUCCCA | | GCUAGAAAUAGCAAGUUAAAAUAAGGCU | |
| | CGG | | UGGCGG | | AGUCCGUUAUCAACUUGAAAAAGUGGCA | |
| | | | | | CCGAGUCGGUGCUUUU | |
| nRNA | CTGCACTCCC | 1049 | CUGCACU | 1075 | CUGCACUCCCAUGGCGGCGGGUUUUAGA | 1101 |
| | ATGGCGGCGG | | CCCAUGG | | GCUAGAAAUAGCAAGUUAAAAUAAGGCU | |
| | CGG | | CGGCGG | | AGUCCGUUAUCAACUUGAAAAAGUGGCA | |
| | | | | | CCGAGUCGGUGCUUUU | |
| nRNA | TGGCGGCGGC | 1050 | UGGCGGC | 1076 | UGGCGGCGGCGGACGCUGAGGUUUUAGA | 1102 |
| | GGACGCTGAG | | GGCGGAC | | GCUAGAAAUAGCAAGUUAAAAUAAGGCU | |
| | CGG | | GCUGAG | | AGUCCGUUAUCAACUUGAAAAAGUGGCA | |
| | | | | | CCGAGUCGGUGCUUUU | |
| nRNA | GGCGGCGGCG | 1051 | GGCGGCG | 1077 | GGCGGCGGCGGACGCUGAGCGUUUUAGA | 1103 |
| | GACGCTGAGC | | GCGGACG | | GCUAGAAAUAGCAAGUUAAAAUAAGGCU | |
| | GGG | | CUGAGC | | AGUCCGUUAUCAACUUGAAAAAGUGGCA | |
| | | | | | CCGAGUCGGUGCUUUU | |
| nRNA | GGCGGCGGAC | 1052 | GGCGGCG | 1078 | GGCGGCGGACGCUGAGCGGGGUUUUAGA | 1104 |
| | GCTGAGCGGG | | GACGCUG | | GCUAGAAAUAGCAAGUUAAAAUAAGGCU | |
| | CGG | | AGCGGG | | AGUCCGUUAUCAACUUGAAAAAGUGGCA | |
| | | | | | CCGAGUCGGUGCUUUU | |
| nRNA | GCGGCGGACG | 1053 | GCGGCGG | 1079 | GCGGCGGACGCUGAGCGGGCGUUUUAGA | 1105 |
| | CTGAGCGGGC | | ACGCUGA | | GCUAGAAAUAGCAAGUUAAAAUAAGGCU | |
| | GGG | | GCGGGC | | AGUCCGUUAUCAACUUGAAAAAGUGGCA | |
| | | | | | CCGAGUCGGUGCUUUU | |
| nRNA | GCGGACGCTG | 1054 | GCGGACG | 1080 | GCGGACGCUGAGCGGGGGGGGUUUUAGA | 1106 |
| | AGCGGGCGGG | | CUGAGCG | | GCUAGAAAUAGCAAGUUAAAAUAAGGCU | |
| | CGG | | GGCGGG | | AGUCCGUUAUCAACUUGAAAAAGUGGCA | |
| | | | | | CCGAGUCGGUGCUUUU | |
| nRNA | TGAGCGGGCG | 1055 | UGAGCGG | 1081 | UGAGCGGGCGGGCGGCGCGAGUUUUAGA | 1107 |
| | GGCGGCGCGA | | GCGGGCG | | GCUAGAAAUAGCAAGUUAAAAUAAGGCU | |
| | CGG | | GCGCGA | | AGUCCGUUAUCAACUUGAAAAAGUGGCA | |
| | | | | | CCGAGUCGGUGCUUUU | |
| nRNA | GAGCGGGCGG | 1056 | GAGCGGG | 1082 | GAGCGGGGGGCGGCGCGACGUUUUAGA | 1108 |
| | GCGGCGCGAC | | CGGGCGG | | GCUAGAAAUAGCAAGUUAAAAUAAGGCU | |
| | GGG | | CGCGAC | | AGUCCGUUAUCAACUUGAAAAAGUGGCA | |
| | | | | | CCGAGUCGGUGCUUUU | |
| nRNA | CGGGCGGGCG | 1057 | CGGGCGG | 1083 | CGGGCGGGCGGCGCGACGGGGUUUUAGA | 1109 |
| | GCGCGACGGG | | GCGGCGC | | GCUAGAAAUAGCAAGUUAAAAUAAGGCU | |
| | CGG | | GACGGG | | AGUCCGUUAUCAACUUGAAAAAGUGGCA | |
| | | | | | CCGAGUCGGUGCUUUU | |
| nRNA | GGGCGGCGCG | 1058 | GGGCGGC | 1084 | GGGCGGCGCGACGGGCGGCGGUUUUAGA | 1110 |
| | ACGGGCGGCG | | GCGACGG | | GCUAGAAAUAGCAAGUUAAAAUAAGGCU | |
| | TGG | | GCGGCG | | AGUCCGUUAUCAACUUGAAAAAGUGGCA | |
| | | | | | CCGAGUCGGUGCUUUU | |
| nRNA | CGCCACTACT | 1059 | CGCCACU | 1085 | CGCCACUACUCGCCGUCUCCGUUUUAGA | 1111 |
| (PE3b) | CGCCGTCTCC | | ACUCGCC | | GCUAGAAAUAGCAAGUUAAAAUAAGGCU | |
| | TGG | | GUCUCC | | AGUCCGUUAUCAACUUGAAAAAGUGGCA | |
| | | | | | CCGAGUCGGUGCUUUU | |
| nRNA | GCCACTACTC | 1060 | GCCACUA | 1086 | GCCACUACUCGCCGUCUCCUGUUUUAGA | 1112 |
| (PE3b) | GCCGTCTCCT | | CUCGCCG | | GCUAGAAAUAGCAAGUUAAAAUAAGGCU | |
| | GGG | | UCUCCU | | AGUCCGUUAUCAACUUGAAAAAGUGGCA | |
| | | | | | CCGAGUCGGUGCUUUU | |
| nRNA | CCACTACTCG | 1061 | CCACUAC | 1087 | CCACUACUCGCCGUCUCCUGGUUUUAGA | 1113 |
| (PE3b) | CCGTCTCCTG | | UCGCCGU | | GCUAGAAAUAGCAAGUUAAAAUAAGGCU | |
| | GGG | | CUCCUG | | AGUCCGUUAUCAACUUGAAAAAGUGGCA | |
| | | | | | CCGAGUCGGUGCUUUU | |
| nRNA | CTCGCCGTCT | 1062 | CUCGCCG | 1088 | CUCGCCGUCUCCUGGGGAGAGUUUUAGA | 1114 |
| (PE3b) | CCTGGGGAGA | | UCUCCUG | | GCUAGAAAUAGCAAGUUAAAAUAAGGCU | |
| | TGG | | GGGAGA | | AGUCCGUUAUCAACUUGAAAAAGUGGCA | |
| | | | | | CCGAGUCGGUGCUUUU | |

TABLE 12
Amino acid and nucleotide sequences for primer editor constructs.

| | | SEQ |
|---|---|---|
| | | ID |
| Description | Sequence | NO |
| Prime Editor | MKRTADGSEFESPKKKRKVDKKYSIGLDIGTNSVGWAVITDEYKVPSKKFKV | 1115 |
| 2 (PE2) | LGNTDRHSIKKNLIGALLFDSGETAEATRLKRTARRRYTRRKNRICYLQEIF | |
| amino acid | SNEMAKVDDSFFHRLEESFLVEEDKKHERHPIFGNIVDEVAYHEKYPTIYHL | |
| sequence | RKKLVDSTDKADLRLIYLALAHMIKFRGHFLIEGDLNPDNSDVDKLFIQLVQ | |
| | TYNQLFEENPINASGVDAKAILSARLSKSRRLENLIAQLPGEKKNGLFGNLI | |
| | ALSLGLTPNFKSNFDLAEDAKLQLSKDTYDDDLDNLLAQIGDQYADLFLAAK | |
| | NLSDAILLSDILRVNTEITKAPLSASMIKRYDEHHQDLTLLKALVRQQLPEK | |
| | YKEIFFDQSKNGYAGYIDGGASQEEFYKFIKPILEKMDGTEELLVKLNREDL | |
| | LRKQRTFDNGSIPHQIHLGELHAILRRQEDFYPFLKDNREKIEKILTFRIPY | |
| | YVGPLARGNSRFAWMTRKSEETITPWNFEEVVDKGASAQSFIERMTNFDKNL | |
| | PNEKVLPKHSLLYEYFTVYNELTKVKYVTEGMRKPAFLSGEQKKAIVDLLFK | |
| | TNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTG | |
| | WGRLSRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLY | |
| | YLQNGRDMYVDQELDINRLSDYDVDAIVPQSFLKDDSIDNKVLTRSDKNRGK | |
| | SDNVPSEEVVKKMKNYWRQLLNAKLITQRKFDNLTKAERGGLSELDKAGFIK | |
| | RQLVETRQITKHVAQILDSRMNTKYDENDKLIREVKVITLKSKLVSDFRKDF | |
| | QFYKVREINNYHHAHDAYLNAVVGTALIKKYPKLESEFVYGDYKVYDVRKMI | |
| | AKSEQEIGKATAKYFFYSNIMNFFKTEITLANGEIRKRPLIETNGETGEIVW | |
| | DKGRDFATVRKVLSMPQVNIVKKTEVQTGGFSKESILPKRNSDKLIARKKDW | |
| | DPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPS | |
| | KYVNFLYLASHYEKLKGSPEDNEQKQLFVEQHKHYLDEIIEQISEFSKRVIL | |
| | ADANLDKVLSAYNKHRDKPIREQAENIIHLFTLTNLGAPAAFKYFDTTIDRK | |
| | RYTSTKEVLDATLIHQSITGLYETRIDLSQLGGDSGGSSGGSSGSETPGTSE | |
| | SATPESSGGSSGGSSTLNIEDEYRLHETSKEPDVSLGSTWLSDFPQAWAETG | |
| | GMGLAVRQAPLIIPLKATSTPVSIKQYPMSQEARLGIKPHIQRLLDQGILVP | |
| | CQSPWNTPLLPVKKPGTNDYRPVQDLREVNKRVEDIHPTVPNPYNLLSGLPP | |
| | SHQWYTVLDLKDAFFCLRLHPTSQPLFAFEWRDPEMGISGQLTWTRLPQGFK | |
| | NSPTLFNEALHRDLADFRIQHPDLILLQYVDDLLLAATSELDCQQGTRALLQ | |
| | TLGNLGYRASAKKAQICQKQVKYLGYLLKEGQRWLTEARKETVMGQPTPKTP | |
| | RQLREFLGKAGFCRLFIPGFAEMAAPLYPLTKPGTLFNWGPDQQKAYQEIKQ | |
| | ALLTAPALGLPDLTKPFELFVDEKQGYAKGVLTQKLGPWRRPVAYLSKKLDP | |
| | VAAGWPPCLRMVAAIAVLTKDAGKLTMGQPLVILAPHAVEALVKQPPDRWLS | |
| | NARMTHYQALLLDTDRVQFGPVVALNPATLLPLPEEGLQHNCLDILAEAHGT | |
| | RPDLTDQPLPDADHTWYTDGSSLLQEGQRKAGAAVTTETEVIWAKALPAGTS | |
| | AQRAELIALTQALKMAEGKKLNVYTDSRYAFATAHIHGEIYRRRGWLTSEGK | |
| | EIKNKDEILALLKALFLPKRLSIIHCPGHQKGHSAEARGNRMADQAARKAAI | |
| | TETPDTSTLLIENSSPSGGSKRTADGSEFEPKKKRKV | |
| Prime Editor | ATGAAACGGACAGCCGACGGAAGCGAGTTCGAGTCACCAAAGAAGAAGCGGA | 1116 |
| 2 (PE2) | AAGTCGACAAGAAGTACAGCATCGGCCTGGACATCGGCACCAACTCTGTGGG | |
| nucleotide | CTGGGCCGTGATCACCGACGAGTACAAGGTGCCCAGCAAGAAATTCAAGGTG | |
| sequence | CTGGGCAACACCGACCGGCACAGCATCAAGAAGAACCTGATCGGAGCCCTGC | |
| | TGTTCGACAGCGGCGAAACAGCCGAGGCCACCCGGCTGAAGAGAACCGCCAG | |
| | AAGAAGATACACCAGACGGAAGAACCGGATCTGCTATCTGCAAGAGATCTTC | |
| | AGCAACGAGATGGCCAAGGTGGACGACAGCTTCTTCCACAGACTGGAAGAGT | |
| | CCTTCCTGGTGGAAGAGGATAAGAAGCACGAGCGGCACCCCATCTTCGGCAA | |
| | CATCGTGGACGAGGTGGCCTACCACGAGAAGTACCCCACCATCTACCACCTG | |
| | AGAAAGAAACTGGTGGACAGCACCGACAAGGCCGACCTGCGGCTGATCTATC | |
| | TGGCCCTGGCCCACATGATCAAGTTCCGGGGCCACTTCCTGATCGAGGGCGA | |
| | CCTGAACCCCGACAACAGCGACGTGGACAAGCTGTTCATCCAGCTGGTGCAG | |
| | ACCTACAACCAGCTGTTCGAGGAAAACCCCATCAACGCCAGCGGCGTGGACG | |
| | CCAAGGCCATCCTGTCTGCCAGACTGAGCAAGAGCAGACGGCTGGAAAATCT | |
| | GATCGCCCAGCTGCCCGGCGAGAAGAAGAATGGCCTGTTCGGAAACCTGATT | |
| | GCCCTGAGCCTGGGCCTGACCCCCAACTTCAAGAGCAACTTCGACCTGGCCG | |
| | AGGATGCCAAACTGCAGCTGAGCAAGGACACCTACGACGACGACCTGGACAA | |
| | CCTGCTGGCCCAGATCGGCGACCAGTACGCCGACCTGTTTCTGGCCGCCAAG | |
| | AACCTGTCCGACGCCATCCTGCTGAGCGACATCCTGAGAGTGAACACCGAGA | |
| | TCACCAAGGCCCCCCTGAGCGCCTCTATGATCAAGAGATACGACGAGCACCA | |
| | CCAGGACCTGACCCTGCTGAAAGCTCTCGTGCGGCAGCAGCTGCCTGAGAAG | |
| | TACAAAGAGATTTTCTTCGACCAGAGCAAGAACGGCTACGCCGGCTACATTG | |
| | ACGGCGGAGCCAGCCAGGAAGAGTTCTACAAGTTCATCAAGCCCATCCTGGA | |
| | AAAGATGGACGGCACCGAGGAACTGCTCGTGAAGCTGAACAGAGAGGACCTG | |
| | CTGCGGAAGCAGCGGACCTTCGACAACGGCAGCATCCCCCACCAGATCCACC | |
| | TGGGAGAGCTGCACGCCATTCTGCGGCGGCAGGAAGATTTTTACCCATTCCT | |
| | GAAGGACAACCGGGAAAAGATCGAGAAGATCCTGACCTTCCGCATCCCCTAC | |
| | TACGTGGGCCCTCTGGCCAGGGGAAACAGCAGATTCGCCTGGATGACCAGAA | |
| | AGAGCGAGGAAACCATCACCCCCTGGAACTTCGAGGAAGTGGTGGACAAGGG | |
| | CGCTTCCGCCCAGAGCTTCATCGAGCGGATGACCAACTTCGATAAGAACCTG | |
| | CCCAACGAGAAGGTGCTGCCCAAGCACAGCCTGCTGTACGAGTACTTCACCG | |
| | TGTATAACGAGCTGACCAAAGTGAAATACGTGACCGAGGGAATGAGAAAGCC | |
| | CGCCTTCCTGAGCGGCGAGCAGAAAAAGGCCATCGTGGACCTGCTGTTCAAG | |
| | ACCAACCGGAAAGTGACCGTGAAGCAGCTGAAAGAGGACTACTTCAAGAAAA | |
| | TCGAGTGCTTCGACTCCGTGGAAATCTCCGGCGTGGAAGATCGGTTCAACGC | |
| | CTCCCTGGGCACATACCACGATCTGCTGAAAATTATCAAGGACAAGGACTTC | |
| | CTGGACAATGAGGAAAACGAGGACATTCTGGAAGATATCGTGCTGACCCTGA | |
| | CACTGTTTGAGGACAGAGAGATGATCGAGGAACGGCTGAAAACCTATGCCCA | |
| | CCTGTTCGACGACAAAGTGATGAAGCAGCTGAAGCGGCGGAGATACACCGGC | |
| | TGGGGCAGGCTGAGCCGGAAGCTGATCAACGGCATCCGGGACAAGCAGTCCG | |
| | GCAAGACAATCCTGGATTTCCTGAAGTCCGACGGCTTCGCCAACAGAAACTT | |
| | CATGCAGCTGATCCACGACGACAGCCTGACCTTTAAAGAGGACATCCAGAAA | |
| | GCCCAGGTGTCCGGCCAGGGCGATAGCCTGCACGAGCACATTGCCAATCTGG | |
| | CCGGCAGCCCCGCCATTAAGAAGGGCATCCTGCAGACAGTGAAGGTGGTGGA | |
| | CGAGCTCGTGAAAGTGATGGGCCGGCACAAGCCCGAGAACATCGTGATCGAA | |
| | ATGGCCAGAGAGAACCAGACCACCCAGAAGGGACAGAAGAACAGCCGCGAGA | |
| | GAATGAAGCGGATCGAAGAGGGCATCAAAGAGCTGGGCAGCCAGATCCTGAA | |
| | AGAACACCCCGTGGAAAACACCCAGCTGCAGAACGAGAAGCTGTACCTGTAC | |
| | TACCTGCAGAATGGGGGGGATATGTACGTGGACCAGGAACTGGACATCAACC | |
| | GGCTGTCCGACTACGATGTGGACGCTATCGTGCCTCAGAGCTTTCTGAAGGA | |
| | CGACTCCATCGACAACAAGGTGCTGACCAGAAGCGACAAGAACCGGGGCAAG | |
| | AGCGACAACGTGCCCTCCGAAGAGGTCGTGAAGAAGATGAAGAACTACTGGC | |
| | GGCAGCTGCTGAACGCCAAGCTGATTACCCAGAGAAAGTTCGACAATCTGAC | |
| | CAAGGCCGAGAGAGGCGGCCTGAGCGAACTGGATAAGGCCGGCTTCATCAAG | |
| | AGACAGCTGGTGGAAACCCGGCAGATCACAAAGCACGTGGCACAGATCCTGG | |
| | ACTCCCGGATGAACACTAAGTACGACGAGAATGACAAGCTGATCCGGGAAGT | |
| | GAAAGTGATCACCCTGAAGTCCAAGCTGGTGTCCGATTTCCGGAAGGATTTC | |
| | CAGTTTTACAAAGTGCGCGAGATCAACAACTACCACCACGCCCACGACGCCT | |
| | ACCTGAACGCCGTCGTGGGAACCGCCCTGATCAAAAAGTACCCTAAGCTGGA | |
| | AAGCGAGTTCGTGTACGGCGACTACAAGGTGTACGACGTGCGGAAGATGATC | |
| | GCCAAGAGCGAGCAGGAAATCGGCAAGGCTACCGCCAAGTACTTCTTCTACA | |
| | GCAACATCATGAACTTTTTCAAGACCGAGATTACCCTGGCCAACGGCGAGAT | |
| | CCGGAAGCGGCCTCTGATCGAGACAAACGGCGAAACCGGGGAGATCGTGTGG | |
| | GATAAGGGCCGGGATTTTGCCACCGTGCGGAAAGTGCTGAGCATGCCCCAAG | |
| | TGAATATCGTGAAAAAGACCGAGGTGCAGACAGGCGGCTTCAGCAAAGAGTC | |
| | TATCCTGCCCAAGAGGAACAGCGATAAGCTGATCGCCAGAAAGAAGGACTGG | |
| | GACCCTAAGAAGTACGGCGGCTTCGACAGCCCCACCGTGGCCTATTCTGTGC | |
| | TGGTGGTGGCCAAAGTGGAAAAGGGCAAGTCCAAGAAACTGAAGAGTGTGAA | |
| | AGAGCTGCTGGGGATCACCATCATGGAAAGAAGCAGCTTCGAGAAGAATCCC | |
| | ATCGACTTTCTGGAAGCCAAGGGCTACAAAGAAGTGAAAAAGGACCTGATCA | |
| | TCAAGCTGCCTAAGTACTCCCTGTTCGAGCTGGAAAACGGCCGGAAGAGAAT | |
| | GCTGGCCTCTGCCGGCGAACTGCAGAAGGGAAACGAACTGGCCCTGCCCTCC | |
| | AAATATGTGAACTTCCTGTACCTGGCCAGCCACTATGAGAAGCTGAAGGGCT | |
| | CCCCCGAGGATAATGAGCAGAAACAGCTGTTTGTGGAACAGCACAAGCACTA | |
| | CCTGGACGAGATCATCGAGCAGATCAGCGAGTTCTCCAAGAGAGTGATCCTG | |
| | GCCGACGCTAATCTGGACAAAGTGCTGTCCGCCTACAACAAGCACCGGGATA | |
| | AGCCCATCAGAGAGCAGGCCGAGAATATCATCCACCTGTTTACCCTGACCAA | |
| | TCTGGGAGCCCCTGCCGCCTTCAAGTACTTTGACACCACCATCGACCGGAAG | |
| | AGGTACACCAGCACCAAAGAGGTGCTGGACGCCACCCTGATCCACCAGAGCA | |
| | TCACCGGCCTGTACGAGACACGGATCGACCTGTCTCAGCTGGGAGGTGACTC | |
| | TGGAGGATCTAGCGGAGGATCCTCTGGCAGCGAGACACCAGGAACAAGCGAG | |
| | TCAGCAACACCAGAGAGCAGTGGCGGCAGCAGCGGCGGCAGCAGCACCCTAA | |
| | ATATAGAAGATGAGTATCGGCTACATGAGACCTCAAAAGAGCCAGATGTTTC | |
| | TCTAGGGTCCACATGGCTGTCTGATTTTCCTCAGGCCTGGGCGGAAACCGGG | |
| | GGCATGGGACTGGCAGTTCGCCAAGCTCCTCTGATCATACCTCTGAAAGCAA | |
| | CCTCTACCCCCGTGTCCATAAAACAATACCCCATGTCACAAGAAGCCAGACT | |
| | GGGGATCAAGCCCCACATACAGAGACTGTTGGACCAGGGAATACTGGTACCC | |
| | TGCCAGTCCCCCTGGAACACGCCCCTGCTACCCGTTAAGAAACCAGGGACTA | |
| | ATGATTATAGGCCTGTCCAGGATCTGAGAGAAGTCAACAAGCGGGTGGAAGA | |
| | TATCCACCCCACCGTGCCCAACCCTTACAACCTCTTGAGCGGGCTCCCACCG | |
| | TCCCACCAGTGGTACACTGTGCTTGATTTAAAGGATGCCTTTTTCTGCCTGA | |
| | GACTCCACCCCACCAGTCAGCCTCTCTTCGCCTTTGAGTGGAGAGATCCAGA | |
| | GATGGGAATCTCAGGACAATTGACCTGGACCAGACTCCCACAGGGTTTCAAA | |
| | AACAGTCCCACCCTGTTTAATGAGGCACTGCACAGAGACCTAGCAGACTTCC | |
| | GGATCCAGCACCCAGACTTGATCCTGCTACAGTACGTGGATGACTTACTGCT | |
| | GGCCGCCACTTCTGAGCTAGACTGCCAACAAGGTACTCGGGCCCTGTTACAA | |
| | ACCCTAGGGAACCTCGGGTATCGGGCCTCGGCCAAGAAAGCCCAAATTTGCC | |
| | AGAAACAGGTCAAGTATCTGGGGTATCTTCTAAAAGAGGGTCAGAGATGGCT | |
| | GACTGAGGCCAGAAAAGAGACTGTGATGGGGCAGCCTACTCCTAAGACCCCT | |
| | CGACAACTAAGGGAGTTCCTAGGGAAGGCAGGCTTCTGTCGCCTCTTCATCC | |
| | CTGGGTTTGCAGAAATGGCAGCCCCCCTGTACCCTCTCACCAAACCGGGGAC | |
| | TCTGTTTAATTGGGGCCCAGACCAACAAAAGGCCTATCAAGAAATCAAGCAA | |
| | GCTCTTCTAACTGCCCCAGCCCTGGGGTTGCCAGATTTGACTAAGCCCTTTG | |
| | AACTCTTTGTCGACGAGAAGCAGGGCTACGCCAAAGGTGTCCTAACGCAAAA | |
| | ACTGGGACCTTGGCGTCGGCCGGTGGCCTACCTGTCCAAAAAGCTAGACCCA | |
| | GTAGCAGCTGGGTGGCCCCCTTGCCTACGGATGGTAGCAGCCATTGCCGTAC | |
| | TGACAAAGGATGCAGGCAAGCTAACCATGGGACAGCCACTAGTCATTCTGGC | |
| | CCCCCATGCAGTAGAGGCACTAGTCAAACAACCCCCCGACCGCTGGCTTTCC | |
| | AACGCCCGGATGACTCACTATCAGGCCTTGCTTTTGGACACGGACCGGGTCC | |
| | AGTTCGGACCGGTGGTAGCCCTGAACCCGGCTACGCTGCTCCCACTGCCTGA | |
| | GGAAGGGCTGCAACACAACTGCCTTGATATCCTGGCCGAAGCCCACGGAACC | |
| | CGACCCGACCTAACGGACCAGCCGCTCCCAGACGCCGACCACACCTGGTACA | |
| | CGGATGGAAGCAGTCTCTTACAAGAGGGACAGCGTAAGGCGGGAGCTGCGGT | |
| | GACCACCGAGACCGAGGTAATCTGGGCTAAAGCCCTGCCAGCCGGGACATCC | |
| | GCTCAGCGGGCTGAACTGATAGCACTCACCCAGGCCCTAAAGATGGCAGAAG | |
| | GTAAGAAGCTAAATGTTTATACTGATAGCCGTTATGCTTTTGCTACTGCCCA | |
| | TATCCATGGAGAAATATACAGAAGGCGTGGGTGGCTCACATCAGAAGGCAAA | |
| | GAGATCAAAAATAAAGACGAGATCTTGGCCCTACTAAAAGCCCTCTTTCTGC | |
| | CCAAAAGACTTAGCATAATCCATTGTCCAGGACATCAAAAGGGACACAGCGC | |
| | CGAGGCTAGAGGCAACCGGATGGCTGACCAAGCGGCCCGAAAGGCAGCCATC | |
| | ACAGAGACTCCAGACACCTCTACCCTCCTCATAGAAAATTCATCACCCTCTG | |
| | GCGGCTCAAAAAGAACCGCCGACGGCAGCGAATTCGAGCCCAAGAAGAAGAG | |
| | GAAAGTCTAA | |
| Prime Editor | MKRTADGSEFESPKKKRKVDKKYSIGLDIGTNSVGWAVITDEYKVPSKKFKV | 1117 |
| 3 (PE3) | LGNTDRHSIKKNLIGALLFDSGETAEATRLKRTARRRYTRRKNRICYLQEIF | |
| amino acid | SNEMAKVDDSFFHRLEESFLVEEDKKHERHPIFGNIVDEVAYHEKYPTIYHL | |
| sequence | RKKLVDSTDKADLRLIYLALAHMIKFRGHFLIEGDLNPDNSDVDKLFIQLVQ | |
| | TYNQLFEENPINASGVDAKAILSARLSKSRRLENLIAQLPGEKKNGLFGNLI | |
| | ALSLGLTPNFKSNFDLAEDAKLQLSKDTYDDDLDNLLAQIGDQYADLFLAAK | |
| | NLSDAILLSDILRVNTEITKAPLSASMIKRYDEHHQDLTLLKALVRQQLPEK | |
| | YKEIFFDQSKNGYAGYIDGGASQEEFYKFIKPILEKMDGTEELLVKLNREDL | |
| | LRKQRTFDNGSIPHQIHLGELHAILRRQEDFYPFLKDNREKIEKILTFRIPY | |
| | YVGPLARGNSRFAWMTRKSEETITPWNFEEVVDKGASAQSFIERMTNFDKNL | |
| | PNEKVLPKHSLLYEYFTVYNELTKVKYVTEGMRKPAFLSGEQKKAIVDLLFK | |
| | TNRKVTVKQLKEDYFKKIECFDSVEISGVEDRFNASLGTYHDLLKIIKDKDF | |
| | LDNEENEDILEDIVLTLTLFEDREMIEERLKTYAHLFDDKVMKQLKRRRYTG | |
| | WGRLSRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKPENIVIE | |
| | MARENQTTQKGQKNSRERMKRIEEGIKELGSQILKEHPVENTQLQNEKLYLY | |
| | YLQNGRDMYVDQELDINRLSDYDVDAIVPQSFLKDDSIDNKVLTRSDKNRGK | |
| | SDNVPSEEVVKKMKNYWRQLLNAKLITQRKFDNLTKAERGGLSELDKAGFIK | |
| | RQLVETRQITKHVAQILDSRMNTKYDENDKLIREVKVITLKSKLVSDFRKDF | |
| | QFYKVREINNYHHAHDAYLNAVVGTALIKKYPKLESEFVYGDYKVYDVRKMI | |
| | AKSEQEIGKATAKYFFYSNIMNFFKTEITLANGEIRKRPLIETNGETGEIVW | |
| | DKGRDFATVRKVLSMPQVNIVKKTEVQTGGFSKESILPKRNSDKLIARKKDW | |
| | DPKKYGGFDSPTVAYSVLVVAKVEKGKSKKLKSVKELLGITIMERSSFEKNP | |
| | IDFLEAKGYKEVKKDLIIKLPKYSLFELENGRKRMLASAGELQKGNELALPS | |
| | KYVNFLYLASHYEKLKGSPEDNEQKQLFVEQHKHYLDEIIEQISEFSKRVIL | |
| | ADANLDKVLSAYNKHRDKPIREQAENIIHLFTLINLGAPAAFKYFDTTIDRK | |
| | RYTSTKEVLDATLIHQSITGLYETRIDLSQLGGDSGGSEAAAKEAAAKEAAA | |
| | KEAAAKSGGSTLNIEDEYRLHETSKEPDVSLGSTWLSDFPQAWAETGGMGLA | |
| | VRQAPLIIPLKATSTPVSIKQYPMSQEARLGIKPHIQRLLDQGILVPCQSPW | |
| | NTPLLPVKKPGTNDYRPVQDLREVNKRVEDIHPTVPNPYNLLSGLPPSHQWY | |
| | TVLDLKDAFFCLRLHPTSQPLFAFEWRDPEMGISGQLTWTRLPQGFKNSPTL | |
| | FNEALHRDLADFRIQHPDLILLQYVDDLLLAATSELDCQQGTRALLQTLGNL | |
| | GYRASAKKAQICQKQVKYLGYLLKEGQRWLTEARKETVMGQPTPKTPRQLRE | |
| | FLGKAGFCRLFIPGFAEMAAPLYPLTKPGTLFNWGPDQQKAYQEIKQALLTA | |
| | PALGLPDLTKPFELFVDEKQGYAKGVLTQKLGPWRRPVAYLSKKLDPVAAGW | |
| | PPCLRMVAAIAVLTKDAGKLTMGQPLVILAPHAVEALVKQPPDRWLSNARMT | |
| | HYQALLLDTDRVQFGPVVALNPATLLPLPEEGLQHNCLDILAEAHGGGSKRT | |
| | ADGSEFEPKKKRKV | |
| Prime Editor | ATGAAACGGACAGCCGACGGAAGCGAGTTCGAGTCACCAAAGAAGAAGCGGA | 1118 |
| 3 (PE3) | AAGTCGACAAGAAGTACAGCATCGGCCTGGACATCGGCACCAACTCTGTGGG | |
| nucleotide | CTGGGCCGTGATCACCGACGAGTACAAGGTGCCCAGCAAGAAATTCAAGGTG | |
| sequence | CTGGGCAACACCGACCGGCACAGCATCAAGAAGAACCTGATCGGAGCCCTGC | |
| | TGTTCGACAGCGGCGAAACAGCCGAGGCCACCCGGCTGAAGAGAACCGCCAG | |
| | AAGAAGATACACCAGACGGAAGAACCGGATCTGCTATCTGCAAGAGATCTTC | |
| | AGCAACGAGATGGCCAAGGTGGACGACAGCTTCTTCCACAGACTGGAAGAGT | |
| | CCTTCCTGGTGGAAGAGGATAAGAAGCACGAGCGGCACCCCATCTTCGGCAA | |
| | CATCGTGGACGAGGTGGCCTACCACGAGAAGTACCCCACCATCTACCACCTG | |
| | AGAAAGAAACTGGTGGACAGCACCGACAAGGCCGACCTGCGGCTGATCTATC | |
| | TGGCCCTGGCCCACATGATCAAGTTCCGGGGCCACTTCCTGATCGAGGGCGA | |
| | CCTGAACCCCGACAACAGCGACGTGGACAAGCTGTTCATCCAGCTGGTGCAG | |
| | ACCTACAACCAGCTGTTCGAGGAAAACCCCATCAACGCCAGCGGCGTGGACG | |
| | CCAAGGCCATCCTGTCTGCCAGACTGAGCAAGAGCAGACGGCTGGAAAATCT | |
| | GATCGCCCAGCTGCCCGGCGAGAAGAAGAATGGCCTGTTCGGAAACCTGATT | |
| | GCCCTGAGCCTGGGCCTGACCCCCAACTTCAAGAGCAACTTCGACCTGGCCG | |
| | AGGATGCCAAACTGCAGCTGAGCAAGGACACCTACGACGACGACCTGGACAA | |
| | CCTGCTGGCCCAGATCGGCGACCAGTACGCCGACCTGTTTCTGGCCGCCAAG | |
| | AACCTGTCCGACGCCATCCTGCTGAGCGACATCCTGAGAGTGAACACCGAGA | |
| | TCACCAAGGCCCCCCTGAGCGCCTCTATGATCAAGAGATACGACGAGCACCA | |
| | CCAGGACCTGACCCTGCTGAAAGCTCTCGTGCGGCAGCAGCTGCCTGAGAAG | |
| | TACAAAGAGATTTTCTTCGACCAGAGCAAGAACGGCTACGCCGGCTACATTG | |
| | ACGGCGGAGCCAGCCAGGAAGAGTTCTACAAGTTCATCAAGCCCATCCTGGA | |
| | AAAGATGGACGGCACCGAGGAACTGCTCGTGAAGCTGAACAGAGAGGACCTG | |
| | CTGCGGAAGCAGCGGACCTTCGACAACGGCAGCATCCCCCACCAGATCCACC | |
| | TGGGAGAGCTGCACGCCATTCTGCGGCGGCAGGAAGATTTTTACCCATTCCT | |
| | GAAGGACAACCGGGAAAAGATCGAGAAGATCCTGACCTTCCGCATCCCCTAC | |
| | TACGTGGGCCCTCTGGCCAGGGGAAACAGCAGATTCGCCTGGATGACCAGAA | |
| | AGAGCGAGGAAACCATCACCCCCTGGAACTTCGAGGAAGTGGTGGACAAGGG | |
| | CGCTTCCGCCCAGAGCTTCATCGAGCGGATGACCAACTTCGATAAGAACCTG | |
| | CCCAACGAGAAGGTGCTGCCCAAGCACAGCCTGCTGTACGAGTACTTCACCG | |
| | TGTATAACGAGCTGACCAAAGTGAAATACGTGACCGAGGGAATGAGAAAGCC | |
| | CGCCTTCCTGAGCGGCGAGCAGAAAAAGGCCATCGTGGACCTGCTGTTCAAG | |
| | ACCAACCGGAAAGTGACCGTGAAGCAGCTGAAAGAGGACTACTTCAAGAAAA | |
| | TCGAGTGCTTCGACTCCGTGGAAATCTCCGGCGTGGAAGATCGGTTCAACGC | |
| | CTCCCTGGGCACATACCACGATCTGCTGAAAATTATCAAGGACAAGGACTTC | |
| | CTGGACAATGAGGAAAACGAGGACATTCTGGAAGATATCGTGCTGACCCTGA | |
| | CACTGTTTGAGGACAGAGAGATGATCGAGGAACGGCTGAAAACCTATGCCCA | |
| | CCTGTTCGACGACAAAGTGATGAAGCAGCTGAAGCGGCGGAGATACACCGGC | |
| | TGGGGCAGGCTGAGCCGGAAGCTGATCAACGGCATCCGGGACAAGCAGTCCG | |
| | GCAAGACAATCCTGGATTTCCTGAAGTCCGACGGCTTCGCCAACAGAAACTT | |
| | CATGCAGCTGATCCACGACGACAGCCTGACCTTTAAAGAGGACATCCAGAAA | |
| | GCCCAGGTGTCCGGCCAGGGCGATAGCCTGCACGAGCACATTGCCAATCTGG | |
| | CCGGCAGCCCCGCCATTAAGAAGGGCATCCTGCAGACAGTGAAGGTGGTGGA | |
| | CGAGCTCGTGAAAGTGATGGGCCGGCACAAGCCCGAGAACATCGTGATCGAA | |
| | ATGGCCAGAGAGAACCAGACCACCCAGAAGGGACAGAAGAACAGCCGCGAGA | |
| | GAATGAAGCGGATCGAAGAGGGCATCAAAGAGCTGGGCAGCCAGATCCTGAA | |
| | AGAACACCCCGTGGAAAACACCCAGCTGCAGAACGAGAAGCTGTACCTGTAC | |
| | TACCTGCAGAATGGGGGGGATATGTACGTGGACCAGGAACTGGACATCAACC | |
| | GGCTGTCCGACTACGATGTGGACGCTATCGTGCCTCAGAGCTTTCTGAAGGA | |
| | CGACTCCATCGACAACAAGGTGCTGACCAGAAGCGACAAGAACCGGGGCAAG | |
| | AGCGACAACGTGCCCTCCGAAGAGGTCGTGAAGAAGATGAAGAACTACTGGC | |
| | GGCAGCTGCTGAACGCCAAGCTGATTACCCAGAGAAAGTTCGACAATCTGAC | |
| | CAAGGCCGAGAGAGGCGGCCTGAGCGAACTGGATAAGGCCGGCTTCATCAAG | |
| | AGACAGCTGGTGGAAACCCGGCAGATCACAAAGCACGTGGCACAGATCCTGG | |
| | ACTCCCGGATGAACACTAAGTACGACGAGAATGACAAGCTGATCCGGGAAGT | |
| | GAAAGTGATCACCCTGAAGTCCAAGCTGGTGTCCGATTTCCGGAAGGATTTC | |
| | CAGTTTTACAAAGTGCGCGAGATCAACAACTACCACCACGCCCACGACGCCT | |
| | ACCTGAACGCCGTCGTGGGAACCGCCCTGATCAAAAAGTACCCTAAGCTGGA | |
| | AAGCGAGTTCGTGTACGGCGACTACAAGGTGTACGACGTGCGGAAGATGATC | |
| | GCCAAGAGCGAGCAGGAAATCGGCAAGGCTACCGCCAAGTACTTCTTCTACA | |
| | GCAACATCATGAACTTTTTCAAGACCGAGATTACCCTGGCCAACGGCGAGAT | |
| | CCGGAAGCGGCCTCTGATCGAGACAAACGGCGAAACCGGGGAGATCGTGTGG | |
| | GATAAGGGCCGGGATTTTGCCACCGTGCGGAAAGTGCTGAGCATGCCCCAAG | |
| | TGAATATCGTGAAAAAGACCGAGGTGCAGACAGGCGGCTTCAGCAAAGAGTC | |
| | TATCCTGCCCAAGAGGAACAGCGATAAGCTGATCGCCAGAAAGAAGGACTGG | |
| | GACCCTAAGAAGTACGGCGGCTTCGACAGCCCCACCGTGGCCTATTCTGTGC | |
| | TGGTGGTGGCCAAAGTGGAAAAGGGCAAGTCCAAGAAACTGAAGAGTGTGAA | |
| | AGAGCTGCTGGGGATCACCATCATGGAAAGAAGCAGCTTCGAGAAGAATCCC | |
| | ATCGACTTTCTGGAAGCCAAGGGCTACAAAGAAGTGAAAAAGGACCTGATCA | |
| | TCAAGCTGCCTAAGTACTCCCTGTTCGAGCTGGAAAACGGCCGGAAGAGAAT | |
| | GCTGGCCTCTGCCGGCGAACTGCAGAAGGGAAACGAACTGGCCCTGCCCTCC | |
| | AAATATGTGAACTTCCTGTACCTGGCCAGCCACTATGAGAAGCTGAAGGGCT | |
| | CCCCCGAGGATAATGAGCAGAAACAGCTGTTTGTGGAACAGCACAAGCACTA | |
| | CCTGGACGAGATCATCGAGCAGATCAGCGAGTTCTCCAAGAGAGTGATCCTG | |
| | GCCGACGCTAATCTGGACAAAGTGCTGTCCGCCTACAACAAGCACCGGGATA | |
| | AGCCCATCAGAGAGCAGGCCGAGAATATCATCCACCTGTTTACCCTGACCAA | |
| | TCTGGGAGCCCCTGCCGCCTTCAAGTACTTTGACACCACCATCGACCGGAAG | |
| | AGGTACACCAGCACCAAAGAGGTGCTGGACGCCACCCTGATCCACCAGAGCA | |
| | TCACCGGCCTGTACGAGACACGGATCGACCTGTCTCAGCTGGGAGGTGACTC | |
| | CGGCGGCAGCGAGGCCGCCGCCAAGGAAGCCGCCGCCAAGGAAGCCGCTGCC | |
| | AAGGAGGCCGCTGCTAAAAGCGGCGGATCTACCCTGAACATCGAGGACGAGT | |
| | ACAGGCTGCACGAGACCAGCAAGGAGCCCGACGTGAGCCTGGGCAGCACCTG | |
| | GCTGAGCGATTTCCCTCAGGCTTGGGCCGAGACCGGCGGCATGGGCCTGGCC | |
| | GTGCGGCAGGCCCCCCTGATTATCCCCCTGAAGGCCACCAGCACCCCCGTGA | |
| | GCATCAAGCAGTACCCAATGTCCCAGGAGGCCAGGCTGGGCATCAAGCCTCA | |
| | CATCCAGAGGCTGCTGGACCAGGGCATCCTGGTGCCATGCCAGTCCCCCTGG | |
| | AACACCCCTCTGCTGCCCGTGAAGAAGCCTGGCACCAACGACTACCGGCCCG | |
| | TGCAGGACCTGAGAGAAGTGAACAAGCGGGTGGAGGACATCCACCCAACCGT | |
| | GCCCAACCCTTACAACCTGCTGTCCGGCCTGCCCCCCAGCCACCAGTGGTAC | |
| | ACCGTGCTGGACCTGAAGGACGCCTTCTTCTGCCTGAGACTGCACCCCACCT | |
| | CTCAGCCCCTGTTCGCCTTCGAGTGGCGCGACCCCGAGATGGGCATCAGCGG | |
| | CCAGCTGACCTGGACCAGACTGCCACAGGGCTTTAAGAATAGCCCAACCCTG | |
| | TTTAACGAGGCCCTGCACAGGGACCTGGCCGACTTCAGGATCCAGCACCCCG | |
| | ACCTGATTCTGCTGCAGTACGTGGACGACCTGCTGCTGGCCGCTACCAGCGA | |
| | GCTGGACTGCCAGCAGGGCACCAGAGCCCTGCTGCAGACCCTGGGCAACCTG | |
| | GGCTACAGAGCCAGCGCCAAGAAGGCCCAGATCTGTCAGAAGCAGGTGAAGT | |
| | ATCTGGGCTACCTGCTGAAGGAAGGCCAGAGATGGCTGACCGAGGCCAGAAA | |
| | GGAGACTGTGATGGGCCAGCCCACCCCCAAGACCCCCAGGCAGCTGCGGGAG | |
| | TTCCTGGGCAAGGCCGGCTTTTGCAGACTGTTTATCCCTGGCTTCGCCGAGA | |
| | TGGCCGCCCCACTGTACCCTCTGACCAAGCCTGGCACCCTGTTTAACTGGGG | |
| | CCCCGACCAGCAGAAGGCCTACCAGGAGATCAAGCAGGCCCTGCTGACCGCC | |
| | CCCGCCCTGGGCCTGCCCGACCTGACCAAGCCTTTCGAGCTGTTCGTGGACG | |
| | AGAAGCAGGGATACGCCAAAGGCGTGCTGACCCAGAAGCTGGGCCCCTGGCG | |
| | GAGGCCCGTGGCCTACCTGAGCAAAAAACTGGACCCTGTGGCCGCCGGCTGG | |
| | CCCCCATGCCTGCGGATGGTGGCCGCCATCGCTGTGCTGACCAAGGACGCCG | |
| | GCAAGCTGACCATGGGCCAGCCCCTGGTGATCCTGGCCCCTCACGCCGTGGA | |
| | GGCTCTGGTGAAGCAGCCTCCAGACAGGTGGCTGTCCAACGCCAGGATGACC | |
| | CACTACCAGGCCCTGCTGCTGGACACCGACCGGGTGCAGTTCGGCCCTGTGG | |
| | TGGCCCTGAACCCCGCCACCCTGCTGCCTCTGCCAGAGGAGGGCCTGCAGCA | |
| | CAACTGCCTGGACATCCTGGCCGAGGCCCACGGGGGGGGCTCCAAACGCACC | |
| | GCCGACGGGAGCGAGTTCGAGCCCAAGAAGAAGAGGAAAGTCTAA | |

### OTHER EMBODIMENTS

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosure may be related to one or more of International Patent Applications No. PCT/US22/75021, PCT/US20/13964, PCT/US20/52822, PCT/US20/18178, PCT/US21/52035, PCT/US22/81241, PCT/US23/67780, PCT/US23/68543, PCT/US23/72911, and/or PCT/US24/18668 and/or U.S. Provisional Patent Applications Nos. 63/592,339, 63/612,146, 63/618,520, and/or 63/621,953, the disclosures of which are incorporated herein by reference in their entirety for all purposes. All patents and publications mentioned in this specification are herein incorporated by reference to the same extent as if each independent patent and publication was specifically and individually indicated to be incorporated by reference.

What is claimed:

1. A method for treating a neoplasia in a subject in need thereof, the method comprising: administering to the subject
(i) an immunosuppressant agent; and
(ii) a modified allogeneic immune effector cell comprising a chimeric antigen receptor capable of specifically binding a marker expressed by a neoplastic cell present in the subject, wherein the allogeneic immune effector cell further comprises a base edit in its genome that confers resistance to the immunosuppressant agent relative to an unmodified allogeneic immune effector cell.

2. The method of claim 1, wherein the base edit reduces expression or activity of an FK506-binding protein 1A (FKBP1A), nuclear receptor subfamily 3, group C, member 1 (NR3C1), and/or peptidyl-prolyl isomerase A (PPIA) polypeptide relative to an unmodified allogeneic immune effector cell.

3. The method of claim 1, wherein the immunosuppressant agent is selected from the group consisting of mTOR inhibitors, calcineurin inhibitors, and glucocorticoids.

4. The method of claim 1, further comprising introducing the base edit to the modified allogeneic immune effector cell using a base editor system comprising:
(i) a base editor, or a polynucleotide encoding the base editor, wherein the base editor comprises a programmable DNA binding domain and a deaminase domain; and
(ii) a guide polynucleotide, or a polynucleotide encoding the guide polynucleotide, wherein the guide polynucleotide directs the base editor to effect a nucleobase alteration in a polynucleotide encoding a polypeptide selected from the group consisting of FK506-binding protein 1A (FKBP1A), nuclear receptor subfamily 3, group C, member 1 (NR3C1), and peptidyl-prolyl isomerase A (PPIA).

5. The method of claim 4, wherein the deaminase domain is an adenosine deaminase and/or a cytidine deaminase.

6. The method of claim 5, wherein the deaminase domain is TadA*8.20 or rAPOBEC1, and/or the base editor is ABE8.20m or rBE4 and the guide polynucleotide comprises at least 10 contiguous nucleotides of a spacer sequence listed in Table 2A.

7. A method for producing a modified allogeneic immune effector cell having increased resistance to an immunosuppressant agent, the method comprising contacting the cell with:
(i) a base editor, or a polynucleotide encoding the base editor, wherein the base editor comprises a programmable DNA binding domain and a deaminase domain; and
(ii) a guide polynucleotide, or a polynucleotide encoding the guide polynucleotide, wherein the guide polynucleotide directs the base editor to effect a nucleobase alteration in a polynucleotide encoding a polypeptide selected from the group consisting of FK506-binding protein 1A (FKBP1A), nuclear receptor subfamily 3, group C, member 1 (NR3C1), and peptidyl-prolyl isomerase A (PPIA);
wherein each nucleobase alteration effects a reduction in expression or activity of the encoded polypeptide, thereby increasing resistance of the modified immune effector cell to immunosuppression by an immunosuppressant agent selected from the group consisting of an mTOR inhibitor, calcineurin inhibitor, and glucocorticoid relative to an unmodified allogeneic immune effector cell.

8. The method of claim 7, wherein the deaminase domain is TadA*8.20 or rAPOBEC1, and/or the base editor is ABE8.20m or rBE4.

9. The method of claim 1, wherein the modified allogeneic immune effector cells further comprise a base edit that reduces expression of one or more polypeptides selected from the group consisting of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC) relative to an unmodified allogeneic immune effector cell.

10. A cell prepared according to the method of claim 7.

11. A pharmaceutical composition comprising the cell of claim 10 and a pharmaceutically acceptable excipient.

12. A base editor system comprising:
(i) a base editor, or a polynucleotide encoding the base editor, wherein the base editor comprises a programmable DNA binding domain and a deaminase domain; and
(ii) a guide polynucleotide, or a polynucleotide encoding the guide polynucleotide, wherein the guide polynucleotide directs the base editor to effect a nucleobase alteration in a polynucleotide encoding a polypeptide selected from the group consisting of FK506-binding protein 1A (FKBP1A), nuclear receptor subfamily 3, group C, member 1 (NR3C1), and peptidyl-prolyl isomerase A (PPIA).

13. The base editor system of claim 12, wherein the deaminase domain is TadA*8.20, and/or the base editor is ABE8.20m or rBE4.

14. A guide polynucleotide comprising a spacer with a sequence comprising at least 10 contiguous nucleotides selected from those sequences listed in Table 2A.

15. The guide polynucleotide of claim 14, wherein the guide polynucleotide comprises a scaffold comprising the following nucleotide sequence: GUUUUAGAGCUAGAAAUAGCAAGUUAAAAUAAGGCUAGUCCGUUAUCAACUUGAAAAAGUGGCA CCGAGUCGGUGCUUUU (SEQ ID NO: 317; SpCas9 scaffold sequence), or a fragment thereof capable of binding a Cas9 polypeptide.

16. The guide polynucleotide of claim 15, wherein the guide polynucleotide comprises a sequence comprising at least 10 contiguous nucleotides from the following sequence:
(SEQ ID NO: 553; TSBTx1538)
CUCACCGUCUCCUGGGGAGA.

17. A polynucleotide encoding the base editor system of claim 13.

18. A vector comprising the polynucleotide of claim 18.

19. A cell containing the base editor system of claim 7.

20. A pharmaceutical composition comprising the base editor system of claim 7.

21. A kit suitable for use in the method of claim 1.

22. A method for treating a leukemia or a lymphoma in a subject in need thereof, the method comprising administering to the subject
i) an immunosuppressant agent selected from the group consisting of mTOR inhibitors, calcineurin inhibitors, and glucocorticoids; and
ii) a modified allogeneic chimeric antigen receptor (CAR)-expressing T cell, wherein the CAR is capable of specifically binding a marker expressed by a leukemia or a lymphoma cell in the subject, wherein the modified allogeneic CAR T cell has increased resistance to the immunosuppressant agent relative to an unmodified allogeneic immune effector cell, and wherein the modified CAR T cell has been modified using a base editor system comprising:
(a) a base editor, or a polynucleotide encoding the base editor, wherein the base editor comprises an SpCas9 domain and a TadA*8.20 adenosine deaminase domain or an rAPOBEC1 domain; and
(b) a guide polynucleotide, or a polynucleotide encoding the guide polynucleotide, wherein the guide polynucleotide comprises a nucleotide sequence selected from those listed in Table 2A and directs the base editor to effect a nucleobase alteration in a polynucleotide encoding a polypeptide selected from the group consisting of FK506-binding protein 1A (FKBP1A), nuclear receptor subfamily 3, group C, member 1 (NR3C1), and peptidyl-prolyl isomerase A (PPIA), wherein the nucleobase alteration reduces or eliminates activity or expression of the polypeptide in the cell.
